SEQUENCE LISTING (10 sequences)

SEQ ID NO: 1 - protein, 5 aa, Homo sapiens
NYWIG

SEQ ID NO: 2 - protein, 17 aa, Homo sapiens
IIDPSNSYTRYSPSFQG

SEQ ID NO: 3 - protein, 8 aa, Homo sapiens
WYYKPFDV

SEQ ID NO: 4 - protein, 14 aa, Homo sapiens
TGSSSNIGSGYDVH

SEQ ID NO: 5 - protein, 7 aa, Homo sapiens
GNSKRPS

SEQ ID NO: 6 - protein, 11 aa, Homo sapiens
ASWTDGLSLVV

SEQ ID NO: 7 - protein, 117 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 8 - protein, 111 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 9 - protein, 447 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2020/0385453 A1
(43) Pub. Date: Dec. 10, 2020

(54) Safe and Effective Method of Treating Psoriatic Arthritis with Anti-IL23 Specific Antibody

(71) Applicant: Janssen Biotech, Inc., Horsham, PA (US)

(72) Inventors: Elizabeth Hsia, Kennett Square, PA (US); Alexa Kollmeier, San Diego, CA (US); Xie Xu, San Marcos, CA (US)

(21) Appl. No.: 16/892,533

(22) Filed: Jun. 4, 2020

Related U.S. Application Data
(60) Provisional application No. 62/856,997, filed on Jun. 4, 2019.
(60) Provisional application No. 62/993,259, filed on Mar. 23, 2020.

(51) Int. Cl. C07K 16/24; A61P 19/02

(52) U.S. Cl. CPC C07K 16/244; C07K 2317/21; C07K 2317/56; A61P 19/02

(57) ABSTRACT

A method of treating psoriatic arthritis in a patient by administering an IL-23 specific antibody, e.g., guselkumab, in a clinically proven safe and clinically proven effective amount and the patient achieves significant improvement in clinical endpoints, such as ACR20/50/70, IGA, HAQ-DI, CRP, SF-36 PCS/MCS, MDA, VLDA, enthesitis, dactylitis, and LEI/dactylitis, as measured 16 and 24 weeks after initial treatment.

### CROSS-REFERENCE TO RELATED APPLICATIONS

This application claims the benefit of U.S. Provisional Application Ser. No. 62/856,997, filed 4 Jun. 2019, and U.S. Provisional Application Ser. No. 62/993,259, filed 23 Mar. 2020. The entire contents of the aforementioned applications are incorporated herein by reference in their entireties

### SEQUENCE LISTING

The instant application contains a Sequence Listing which has been submitted electronically in ASCII format and is hereby incorporated by reference in its entirety. Said ASCII copy, created on 27 May 2020, is named JBI6102USNP1SEQLIST.txt and is 12,288 bytes in size.

### FIELD OF THE INVENTION

The present invention concerns methods for treating psoriatic arthritis with an antibody that binds the human IL-23 protein. In particular, it relates to a method of administering an anti-IL-23 specific antibody, e.g., guselkumab, which is safe and effective for patients suffering from psoriatic arthritis.

### BACKGROUND OF THE INVENTION

Interleukin (IL)-12 is a secreted heterodimeric cytokine comprised of 2 disulfide-linked glycosylated protein subunits, designated p35 and p40 for their approximate molecular weights. IL-12 is produced primarily by antigen-presenting cells and drives cell-mediated immunity by binding to a two-chain receptor complex that is expressed on the surface of T cells or natural killer (NK) cells. The IL-12 receptor beta-1 (IL-12Rβ1) chain binds to the p40 subunit of IL-12, providing the primary interaction between IL-12 and its receptor. However, it is IL-12p35 ligation of the second receptor chain, IL-12102, that confers intracellular signaling (e.g. STAT4 phosphorylation) and activation of the receptor-bearing cell. IL-12 signaling concurrent with antigen presentation is thought to invoke T cell differentiation towards the T helper 1 (Th1) phenotype, characterized by interferon gamma (IFNγ) production. Th1 cells are believed to promote immunity to some intracellular pathogens, generate complement-fixing antibody isotypes, and contribute to tumor immunosurveillance. Thus, IL-12 is thought to be a significant component to host defense immune mechanisms.

It was discovered that the p40 protein subunit of IL-12 can also associate with a separate protein subunit, designated p19, to form a novel cytokine, IL-23. IL-23 also signals through a two-chain receptor complex. Since the p40 subunit is shared between IL-12 and IL-23, it follows that the IL-12Rβ1 chain is also shared between IL-12 and IL-23. However, it is the IL-23p19 ligation of the second component of the IL-23 receptor complex, IL-23R, that confers IL-23 specific intracellular signaling (e.g., STAT3 phosphorylation) and subsequent IL-17 production by T cells. Recent studies have demonstrated that the biological functions of IL-23 are distinct from those of IL-12, despite the structural similarity between the two cytokines.

Abnormal regulation of IL-12 and Th1 cell populations has been associated with many immune-mediated diseases since neutralization of IL-12 by antibodies is effective in treating animal models of psoriasis, multiple sclerosis (MS), rheumatoid arthritis, inflammatory bowel disease, insulin-dependent (type 1) diabetes mellitus, and uveitis. However, since these studies targeted the shared p40 subunit, both IL-12 and IL-23 were neutralized in vivo. Therefore, it was unclear whether IL-12 or IL-23 was mediating disease, or if both cytokines needed to be inhibited to achieve disease suppression. Studies have confirmed through IL-23p19 deficient mice or specific antibody neutralization of IL-23 that IL-23 inhibition can provide equivalent benefit as anti-IL-12p40 strategies. Therefore, there is increasing evidence for the specific role of IL-23 in immune-mediated disease. Neutralization of IL-23 without inhibition of IL-12 pathways could then provide effective therapy of immune-mediated disease with limited impact on important host defense immune mechanism. This would represent a significant improvement over current therapeutic options.

Psoriasis is a common, chronic immune-mediated skin disorder with significant co-morbidities, such as psoriatic arthritis (PsA), depression, cardiovascular disease, hypertension, obesity, diabetes, metabolic syndrome, and Crohn's disease. Plaque psoriasis is the most common form of the disease and manifests in well demarcated erythematous lesions topped with white silver scales. Plaques are pruritic, painful, often disfiguring and disabling, and a significant proportion of psoriatic patients have plaques on hands/nails face, feet and genitalia. As such, psoriasis negatively impacts health-related quality of life (HRQoL) to a significant extent, including imposing physical and psychosocial burdens that extend beyond the physical dermatological symptoms and interfere with everyday activities. For example, psoriasis negatively impacts familial, spousal, social, and work relationships, and is associated with a higher incidence of depression and increased suicidal tendencies.

Psoriatic arthritis (PsA) is a multi-system disease characterized by joint inflammation and psoriasis, with diverse clinical and radiographic manifestations including dactylitis, enthesitis, sacroiliitis, and/or joint deformity. Functional impairment, decreased quality of life, and increased health-care resource utilization associated with poorly-controlled PsA present significant economic burden. Despite availability of biologics (e.g., tumor-necrosis-factor [TNF]α inhibitors, ustekinumab, secukinumab), and other agents (e.g., apremilast), significant unmet needs exist for new PsA therapies that can provide high levels of efficacy and safety in treating heterogeneous disease components

Histologic characterization of psoriasis lesions reveals a thickened epidermis resulting from aberrant keratinocyte proliferation and differentiation as well as dermal infiltration and co-localization of CD3+ T lymphocytes and dendritic cells. While the etiology of psoriasis is not well defined, gene and protein analysis have shown that IL-12, IL-23 and their downstream molecules are over-expressed in psoriatic lesions, and some may correlate with psoriasis disease severity. Some therapies used in the treatment of psoriasis modulate IL-12 and IL-23 levels, which is speculated to contribute to their efficacy. Th1 and Th17 cells can produce effector cytokines that induce the production of vasodilators, chemoattractants and expression of adhesion molecules on endothelial cells which in turn, promote monocyte and neutrophil recruitment, T cell infiltration, neovascularization and keratinocyte activation and hyperplasia. Activated keratinocytes can produce chemoattractant factors that promote neutrophil, monocyte, T cell, and dendritic cell trafficking, thus establishing a cycle of inflammation and keratinocyte hyperproliferation.

Elucidation of the pathogenesis of psoriasis has led to effective biologic treatments targeting tumor necrosis factor-alpha (TNF-α), both interleukin (IL)-12 and IL-23 and, most recently, IL-17 as well as IL-23 alone (including in Phase 1 and 2 clinical trials using guselkumab). Guselkumab (also known as CNTO 1959, marketed as TRENIFYA®) is a fully human IgG1 lambda monoclonal antibody that binds to the p19 subunit of IL-23 and inhibits the intracellular and downstream signaling of IL-23, required for terminal differentiation of T helper (Th)17 cells. Guselkumab is currently approved in the United States, European Union, and other countries worldwide for the treatment of moderate to severe plaque psoriasis. In addition, guselkumab is being evaluated in several other immune-mediated disorders, including generalized pustular psoriasis, erythrodermic psoriasis, palmoplantar pustulosis, hidradenitis suppurativa, psoriatic arthritis (PsA), and Crohn's disease.

### SUMMARY OF THE INVENTION

The invention relates to treatment of psoriastic arthritis (PsA). In particular, the invention relates to a clinically proven safe and effective method of treating PsA by administering an anti-IL-23 specific antibody to the subject.

In one general aspect, the invention relates to a method of treating psoriastic arthritis (PsA) in a subject in need thereof, comprising subcutaneously administering an effective amount of an anti-IL-23 antibody (also referred to as IL-23p19 antibody), such as guselkumab, to the subject, wherein the anti-IL-23 antibody is administered once every 4 weeks (q4w). Preferably, the subject achieves at least a 20% improvement in the American College of Rheumatology core set disease index (ACR20) after the treatment, without having a clinically apparent adverse event.

In certain embodiments, the anti-IL-23 antibody comprises a heavy chain variable region and a light chain variable region, the heavy chain variable region comprising a complementarity determining region heavy chain 1 (CDRH1) amino acid sequence of SEQ ID NO: 1, a CDRH2 of SEQ ID NO: 2, and a CDRH3 of SEQ ID NO: 3; and the light chain variable region comprising a complementarity determining region light chain 1 (CDRL1) amino acid sequence of SEQ ID NO: 4, a CDRL2 of SEQ ID NO: 5, and a CDRL3 of SEQ ID NO: 6.

In certain embodiments, the anti-IL-23 antibody comprises the heavy chain variable region of the amino acid sequence of SEQ ID NO: 7, and the light chain variable region of the amino acid sequence of SEQ ID NO: 8.

In certain embodiments, the anti-IL-23 antibody comprises the heavy chain amino acid sequence of SEQ ID NO: 9, and the light chain amino acid sequence of SEQ ID NO: 10.

In certain embodiments, the anti-IL-23 antibody is administered at a total dosage of 25 mg to 200 mg, preferably about 50 mg to about 150 mg, more preferably about 100 mg, per administration.

In certain embodiments, the subject is a responder to the treatment with the anti-IL-23 antibody and is identified as having a statistically significant improvement in disease activity, wherein the disease activity is determined by one or more criteria selected from the group consisting of a 20% improvement in the American College of Rheumatology core set disease index (ACR20), a 50% improvement in the American College of Rheumatology core set disease index (ACR50), a 70% improvement in the American College of Rheumatology core set disease index (ACR70), Health Assessment Questionnaire Disability Index (HAQ-DI), Investigator's Global Assessment (IGA), Disease Activity Score 28 (DAS28)C-reactive protein (CRP), resolution of enthesitis, resolution of dactylitis, Leeds enthesitis index (LEI), dactylitis assessment score, Short Form Health survey (SF-36) in the mental and physical component summary (MCS and PCS), achievement of minimal disease activity (MDA), LS mean change from baseline in total modified vdH-S score and achievement of very low disease activity (VLDA).

In a particular embodiment, a subject achieves a significant improvement in ACR20 response for guselkumab vs. placebo by week 24 (62.9% v. 32.9%) of the treatment.

In another general aspect, the invention relates to a method of treating psoriastic arthritis in a subject in need thereof comprising subcutaneously administering an anti-IL-23 antibody to the subject, wherein the anti-IL-23 antibody is administered at an initial dose, a dose 4 weeks thereafter, and at a dosing interval of once every 8 weeks (q8w) thereafter, and wherein the subject has at least one psoriatic plaque of ≥2 cm diameter or nail changes consistent with psoriasis or documented history of plaque psoriasis. Preferably, the subject achieves at least a 20% improvement in the American College of Rheumatology core set disease index (ACR20) after the treatment, without having a clinically apparent adverse event.

In certain embodiments, the anti-IL-23 antibody comprises a heavy chain variable region and a light chain variable region, the heavy chain variable region comprising a complementarity determining region heavy chain 1 (CDRH1) amino acid sequence of SEQ ID NO: 1, a CDRH2 of SEQ ID NO: 2, and a CDRH3 of SEQ ID NO: 3; and the light chain variable region comprising a complementarity determining region light chain 1 (CDRL1) amino acid sequence of SEQ ID NO: 4, a CDRL2 of SEQ ID NO: 5, and a CDRL3 of SEQ ID NO: 6.

In certain embodiments, the anti-IL-23 antibody comprises the heavy chain variable region of the amino acid sequence of SEQ ID NO: 7, and the light chain variable region of the amino acid sequence of SEQ ID NO: 8, or the anti-IL-23 antibody comprises the heavy chain amino acid sequence of SEQ ID NO: 9, and the light chain amino acid sequence of SEQ ID NO: 10.

In certain embodiments, the anti-IL-23 antibody is administered at a total dosage of 25 mg to 200 mg, preferably about 50 mg to about 150 mg, more preferably about 100 mg, per administration.

In certain embodiments, the subject has had inadequate response to a standard therapy for the PsA. Optionally, the subject is also administered with the standard therapy during a treatment according to embodiments of the invention.

The details of one or more embodiments of the invention are set forth in the description below. Other features and advantages will be apparent from the following detailed description, figures, and the appended claims.

### BRIEF DESCRIPTION OF THE DRAWINGS

In the Figures:

FIG. 1. Shows a shematic overview of a clinical study according to an embodiment of the application.

FIG. 2. Shows the median and IQ Range of serum Guselkumab concentration (μg/mL) through week 24 for Study CNTO1959PSA3002.

FIG. 3. Shows the median and IQ Range of serum Guselkumab concentrations (μg/mL) through Week 24 by antibody status for study CNTO1959PSA3002.

FIG. 4. Shows the line plot of the number of subjects achieving ACR 20 response by visit through week 24 based on the composite estimand for Study CNTO1959PSA3002.

FIG. 5. Shows line plot of the number of subjects achieving ACR 50 Response by visit through week 24 based on the composite estimand for study CNTO1959PSA3002.

FIG. 6. Shows the line plot of the number of subjects achieving ACR 70 Response by visit through Week 24 based on the composite estimand for study CNTO1959PSA3002.

FIG. 7. Shows the Proportion of Subjects Who Achieved ACR 20 Response (Composite Estimand) at Week 24 by trough serum Guselkumab (Combined) concentrations (Quartiles) at Week 20 for Study CNTO1959PSA3002.

FIG. 8. Shows the proportion of subjects who achieved ACR 50 Response (composite Estimand) at Week 24 by trough serum Guselkumab (Combined) concentrations (Quartiles) at Week 20 for study CNTO1959PSA3002.

FIG. 9. Shows the proportion of subjects who achieved IGA Response (Composite Estimand) at Week 24 by trough serum Guselkumab (Combined) concentrations (Quartiles) at Week 20; PK Analysis Set Among the Subjects with ≥3% Body Surface Area (BSA) Psoriatic Involvement and an IGA score of ≥2 (mild) at Baseline (Study CNTO1959PSA3002).

FIG. 10. Shows a schematic overview of another clinical study according to an embodiment of the invention.

FIG. 11. Shows the median and IQ Range of serum Guselkumab concentration (μg/mL) through week 24 for Study CNTO1959PSA3001.

FIG. 12. Shows the median and IQ Range of serum Guselkumab concentrations (μg/mL) through Week 24 by antibody status for study CNTO1959PSA3001.

FIG. 13. Shows the line plot of the number of subjects achieving ACR 20 response by visit through week 24 based on the composite estimand for Study CNTO1959PSA3001.

FIG. 14. Shows the line plot of the number of subjects achieving ACR 50 Response by visit through week 24 based on the composite estimand for study CNTO1959PSA3001.

FIG. 15. Shows the line plot of the number of subjects achieving ACR 70 Response by visit through week 24 based on the composite estimand for study CNTO1959PSA3001.

FIG. 16. Shows the Proportion of Subjects Who Achieved ACR 20 Response (Composite Estimand) at Week 24 by trough serum Guselkumab (Combined) concentrations (Quartiles) at Week 20 for Study CNTO1959PSA3001.

FIG. 17. Shows the proportion of subjects who achieved ACR 50 Response (composite Estimand) at Week 24 by through serum Guselkumab (Combined) concentrations (Quartiles) at Week 20 for study CNTO1959PSA3001.

FIG. 18. Shows the proportion of subjects who achieved IGA Response (Composite Estimand) at Week 24 by Trough Serum Guselkumab (Combined) concentrations (Quartiles) at Week 20; PK Analysis Set Among the Subjects with ≥3% Body Surface Area (BSA) Psoriatic Involvement and an IGA score of ≥2 (mild) at Baseline (Study CNTO1959PSA3001).

FIG. 19. Shows mean PROMIS-29 T-scores at baseline (dashed lines) and Week 24 (solid lines).

FIG. 20. Shows clinically meanigfull improvement (5 points) in PROMIS-29 T-scores at week 24.

FIGS. 21A-B. Shows Week 24 changes from baseline in FACIT-Fatigue in the in patients with psoriatic arthritis in Discover 1 (A) and Discover 2 (B) trials.

FIGS. 22A-B. Shows (A) NRI and (B) observed ACR20 responses through Week 52. Patients randomized to PBO crossed over to GUS q4w at Week 25.

FIGS. 23A-B. Shows (A) NRI and (B) observed ACR50 responses through Week 52. Patients randomized to PBO crossed over to GUS q4w at Week 25.

FIGS. 24A-B. Shows (A) NRI and (B) observed ACR70 responses through Week 52. Patients randomized to PBO crossed over to GUS q4w at Week 25.

FIGS. 25A-B. Shows observed ACR20 response rates from Week 24 through Week 52 by (A) prior TNFi use and (B) TNFi-naïve patients.

FIGS. 26A-B. Shows observed ACR50 response rates from Week 24 through Week 52 by (A) prior TNFi use and (B) TNFi-naïve patients.

FIGS. 27A-B. Shows observed ACR70 response rates from Week 24 through Week 52 by (A) prior TNFi use and (B) TNFi-naïve patients.

FIG. 28. Shows the number of subjects achieving an Investigator Global Assessment (IGA) Response by visit from Week 24 through week 52, based on observed data.

FIG. 29. Shows the number of subjects achieving an PASI90 Response by visit from Week 24 through week 52, based on observed data.

FIG. 30. Shows the summary of the change from baseline in HAQ-DI Score by visit from Week 24 through week 52, based on observed data.

FIG. 31. Shows the number of subjects achieving resolution of dactylitis by visit from Week 24 through week 52, based on observed data.

FIG. 32. Shows the number of subjects achieving resolution of enthesitis by visit from Week 24 through week 52, based on observed data.

FIG. 33. Shows the summary of the change from baseline in SF-36 PCS Score by visit from Week 24 through week 52, based on observed data.

FIG. 34. Shows the summary of the change from baseline in SF-36 MCS Score by visit from Week 24 through week 52, based on observed data.

### DETAILED DESCRIPTION OF THE INVENTION

As used herein the method of treatment of psoriasis arthritis comprises administering isolated, recombinant and/or synthetic anti-IL-23 specific human antibodies and diagnostic and therapeutic compositions, methods and devices.

As used herein, an “anti-IL-23 specific antibody,” “anti-IL-23 antibody,” “antibody portion,” or “antibody fragment” and/or “antibody variant” and the like include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an IL-23 receptor or binding protein, which can be incorporated into an antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one IL-23 activity or binding, or with IL-23 receptor activity or binding, in vitro, in situ and/or in vivo. As a non-limiting example, a suitable anti-IL-23 antibody, specified portion or variant of the present invention can bind at least one IL-23 molecule, or specified portions, variants or domains thereof. A suitable anti-IL-23 antibody, specified portion, or variant can also optionally affect at least one of IL-23 activity or function, such as but not limited to, RNA, DNA or protein synthesis, IL-23 release, IL-23 receptor signaling, membrane IL-23 cleavage, IL-23 activity, IL-23 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-23. For example, antibody fragments capable of binding to IL-23 or portions thereof, including, but not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the C_{H}1 domain and/or hinge region of the heavy chain. The various portions of antibodies can be joined together chemically by conventional techniques or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A “human antibody” may also be an antibody that is derived from or closely matches human germline immunoglobulin sequences. Human antibodies may include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably, human or humanized, antibodies that have binding specificities for at least two different antigens. In the present case, one of the binding specificities is for at least one IL-23 protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

Anti-IL-23 specific (also termed IL-23 specific antibodies) (or antibodies to IL-23) useful in the methods and compositions of the present invention can optionally be characterized by high affinity binding to IL-23 and, optionally and preferably, having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-23 antibody in patients treated with anti-IL-23 antibody as occurring in less than 25% of patients treated, preferably, in less than 10% of patients treated with the recommended dose for the recommended course of therapy during the treatment period.

The terms “clinically proven efficacy” and “clinically proven effective” as used herein in the context of a dose, dosage regimen, treatment or method refer to the clinically proven effectiveness of a particular dose, dosage or treatment regimen. Efficacy can be measured based on change in the course of the disease in response to an agent of the present invention based on the clinical trials conducted, e.g., Phase 3 clinical trials and earlier. For example, an anti-IL-23 antibody of the present invention (e.g., the anti-IL-23 antibody guselkumab) is administered to a patient in an amount and for a time sufficient to induce an improvement, preferably a sustained improvement, in at least one indicator that reflects the severity of the disorder that is being treated. Various indicators that reflect the extent of the subject's illness, disease or condition may be assessed for determining whether the amount and time of the treatment is sufficient. Such indicators include, for example, clinically recognized indicators of disease severity, symptoms, or manifestations of the disorder in question. The degree of improvement generally is determined by a physician, who may make this determination based on signs, symptoms, biopsies, or other test results, and who may also employ questionnaires that are administered to the subject, such as quality-of-life questionnaires developed for a given disease. For example, an anti-IL-23 antibody of the present invention can be administered to achieve an improvement in a patient's condition related to psoriatic arthritis. Improvement can be indicated by an improvement in an index of disease activity, by amelioration of clinical symptoms or by any other measure of disease activity.

In one embodiment, the efficacy of a treatment of psoriatic arthritis in a subject can be determined using the American College of Rheumatology (ACR) preliminary criteria for improvement in rheumatoid arthritis. ACR criteria measures improvement in tender or swollen joint counts and improvement in three of the following five parameters: acute phase reactant (such as sedimentation rate); patient assessment; physician assessment; pain scale; and disability/functional questionnaire. ACR criteria is indicated as ACR 20 (a 20 percent improvement in tender or swollen joint counts as well as 20 percent improvement in three of the other five criteria), ACR 50 (a 50 percent improvement in tender or swollen joint counts as well as 50 percent improvement in three of the other five criteria), and ACR 70 (a 70 percent improvement in tender or swollen joint counts as well as 70 percent improvement in three of the other five criteria) (see Felson D T, et al. Arthritis Rheum 1995; 38:727-35).

In another embodiment, the efficacy of a treatment of psoriatic arthritis in a subject is determined by the Psoriasis Area and Severity Index (PAST), which is an index of disease used to assess skin disease severity/extent, e.g., PASI75=75% improvement, PASI90=90% improvement and PASI100=substantially cleared of plaques. The measure of efficacy can also comprise one or more of the Health Assessment Questionnaire Disability Index (HAQ-DI), enthesitis/dactylitis improvements in patients with baseline enthesitis/dactylitis, changes in SF-36 mental and physical component summary (MCS and PCS) scores, and achievement of minimal disease activity (MDA) criteria score.

In another embodiment, the efficiency of a treatment of psoriatic arthritis in a subject is determined by the vdH-S score.

The term “clinically proven safe,” as it relates to a dose, dosage regimen, treatment or method with an anti-IL-23 antibody of the present invention (e.g., the anti-IL-23 antibody guselkumab), refers to a relatively low or reduced frequency and/or low or reduced severity of treatment-emergent adverse events (referred to as AEs or TEAEs) from the clinical trials conducted, e.g., Phase 2 clinical trials and earlier, compared to the standard of care or to another comparator. An adverse event is an untoward medical occurrence in a patient administered a medicinal product. In particular, clinically proven safe as it relates to a dose, dosage regimen or treatment with an anti-IL-23 antibody of the present invention refers to a relatively low or reduced frequency and/or low or reduced severity of adverse events associated with administration of the antibody if attribution is considered to be possible, probable, or very likely due to the use of the anti-IL-23 antibody.

As used herein, unless otherwise noted, the term “clinically proven” (used independently or to modify the terms “safe” and/or “effective”) shall mean that it has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the drug.

### Utility

The isolated nucleic acids of the present invention can be used for production of at least one anti-IL-23 antibody or specified variant thereof, which can be used to measure or effect in a cell, tissue, organ or animal (including mammals and humans), to diagnose, monitor, modulate, treat, alleviate, help prevent the incidence of, or reduce the symptoms of psoriasis.

Such a method can comprise administering an effective amount of a composition or a pharmaceutical composition comprising at least one anti-IL-23 antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

### Citations

All publications or patents cited herein, whether or not specifically designated, are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

### Antibodies Useful for the Present Invention—Production and Generation

At least one anti-IL-23 antibody used in the method of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001), each entirely incorporated herein by reference.

Human antibodies that are specific for human IL-23 proteins or fragments thereof can be raised against an appropriate immunogenic antigen, such as an isolated IL-23 protein and/or a portion thereof (including synthetic molecules, such as synthetic peptides). Other specific or general mammalian antibodies can be similarly raised. Preparation of immunogenic antigens, and monoclonal antibody production can be performed using any suitable technique.

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, L243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like, or heteromylomas, fusion products thereof, or any cell or fusion cell derived therefrom, or any other suitable cell line as known in the art) (see, e.g., www.atcc.org, www.lifetech.com, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; Biolnvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260(5/12/94); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (Biolnvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, predecessor of Applied Molecular Evolution (AME), each entirely incorporated herein by reference)) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammals. These non-human amino acid residues are replaced by residues often referred to as “import” residues, which are typically taken from an “import” variable, constant or other domain of a known human sequence.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions may be replaced with human or other amino acids.

Antibodies can also optionally be humanized or human antibodies engineered with retention of high affinity for the antigen and other favorable biological properties. To achieve this goal, humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In addition, the human IL-23 specific antibody used in the method of the present invention may comprise a human germline light chain framework. In particular embodiments, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain embodiments, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6.

In other embodiments, the human IL-23 specific antibody used in the method of the present invention may comprise a human germline heavy chain framework. In particular embodiments, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81.

In particular embodiments, the light chain variable region and/or heavy chain variable region comprises a framework region or at least a portion of a framework region (e.g., containing 2 or 3 subregions, such as FR2 and FR3). In certain embodiments, at least FRL1, FRL2, FRL3, or FRL4 is fully human. In other embodiments, at least FRH1, FRH2, FRH3, or FRH4 is fully human. In some embodiments, at least FRL1, FRL2, FRL3, or FRL4 is a germline sequence (e.g., human germline) or comprises human consensus sequences for the particular framework (readily available at the sources of known human Ig sequences described above). In other embodiments, at least FRH1, FRH2, FRH3, or FRH4 is a germline sequence (e.g., human germline) or comprises human consensus sequences for the particular framework. In preferred embodiments, the framework region is a fully human framework region.

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766886, 5714352, 6204023, 6180370, 5693762, 5530101, 5585089, 5225539; 4816567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype. Alternatively, or additionally, it may be useful to combine amino acid modifications with one or more further amino acid modifications that alter C1q binding and/or the complement dependent cytotoxicity function of the Fc region of an IL-23 binding molecule. The starting polypeptide of particular interest may be one that binds to C1q and displays complement dependent cytotoxicity (CDC). Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human IL-23 specific antibody of the present invention with altered effector function, e.g., by modifying C1q binding and/or FcγR binding and thereby changing complement dependent cytotoxicity (CDC) activity and/or antibody-dependent cell-mediated cytotoxicity (ADCC) activity. “Effector functions” are responsible for activating or diminishing a biological activity (e.g., in a subject). Examples of effector functions include, but are not limited to: C1q binding; CDC; Fc receptor binding; ADCC; phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of the human IL-23 (or anti-IL-23) antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human IL-23 specific antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation sites that are not present in the polypeptide. Addition of glycosylation sites to the Fc region of a human IL-23 specific antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human IL-23 specific antibody of the present invention is expressed in cells that express beta (1,4)-N-acetylglucosaminyltransferase III (GnT III), such that GnT III adds GlcNAc to the human IL-23 antibody. Methods for producing antibodies in such a fashion are provided in WO/9954342, WO/03011878, patent publication 20030003097A1, and Umana et al., Nature Biotechnology, 17:176-180, February 1999; all of which are herein specifically incorporated by reference in their entireties.

The anti-IL-23 antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-IL-23 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, 5427908, 5580717, assigned to Affymax; 5885793, assigned to Cambridge antibody Technologies; 5750373, assigned to Genentech, 5618920, 5595898, 5576195, 5698435, 5693493, 5698417, assigned to Xoma, Colligan, supra; Ausubel, supra; or Sambrook, supra, each of the above patents and publications entirely incorporated herein by reference.

Antibodies used in the method of the present invention can also be prepared using at least one anti-IL23 antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies used in the method of the present invention can additionally be prepared using at least one anti-IL23 antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize has been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and references cited therein. Thus, antibodies of the present invention can also be produced using transgenic plants, according to known methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies used in the method of the invention can bind human IL-23 with a wide range of affinities (K_{D}). In a preferred embodiment, a human mAb can optionally bind human IL-23 with high affinity. For example, a human mAb can bind human IL-23 with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

### Nucleic Acid Molecules

Using the information provided herein, for example, the nucleotide sequences encoding at least 70-100% of the contiguous amino acids of at least one of the light or heavy chain variable or CDR regions described herein, among other sequences disclosed herein, specified fragments, variants or consensus sequences thereof, or a deposited vector comprising at least one of these sequences, a nucleic acid molecule of the present invention encoding at least one anti-IL-23 antibody can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules used in the method of the present invention can include nucleic acid molecules comprising an open reading frame (ORF), optionally, with one or more introns, e.g., but not limited to, at least one specified portion of at least one CDR, such as CDR1, CDR2 and/or CDR3 of at least one heavy chain or light chain; nucleic acid molecules comprising the coding sequence for an anti-IL-23 antibody or variable region; and nucleic acid molecules which comprise a nucleotide sequence substantially different from those described above but which, due to the degeneracy of the genetic code, still encode at least one anti-IL-23 antibody as described herein and/or as known in the art. Of course, the genetic code is well known in the art. Thus, it would be routine for one skilled in the art to generate such degenerate nucleic acid variants that code for specific anti-IL-23 antibodies used in the method of the present invention. See, e.g., Ausubel, et al., supra, and such nucleic acid variants are included in the present invention. Non-limiting examples of isolated nucleic acid molecules include nucleic acids encoding HC CDR1, HC CDR2, HC CDR3, LC CDR1, LC CDR2, and LC CDR3, respectively.

As indicated herein, nucleic acid molecules which comprise a nucleic acid encoding an anti-IL-23 antibody can include, but are not limited to, those encoding the amino acid sequence of an antibody fragment, by itself; the coding sequence for the entire antibody or a portion thereof; the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

### Polynucleotides Selectively Hybridizing to a Polynucleotide as Described Herein

The method of the present invention uses isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of an antibody. The polynucleotides embrace nucleic acid sequences that can be employed for selective hybridization to a polynucleotide encoding an antibody of the present invention. See, e.g., Ausubel, supra; Colligan, supra, each entirely incorporated herein by reference.

### Construction of Nucleic Acids

The isolated nucleic acids can be made using (a) recombinant methods, (b) synthetic techniques, (c) purification techniques, and/or (d) combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention, excluding the coding sequence, is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers are well known in the art. (See, e.g., Ausubel, supra; or Sambrook, supra)

### Recombinant Methods for Constructing Nucleic Acids

The isolated nucleic acid compositions, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, are well known to those of ordinary skill in the art. (See, e.g., Ausubel, supra; or Sambrook, supra)

### Nucleic Acid Screening and Isolation Methods

A cDNA or genomic library can be screened using a probe based upon the sequence of a polynucleotide used in the method of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides used in the method of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids used in the method of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The present invention uses recombinant expression cassettes comprising a nucleic acid. A nucleic acid sequence, for example, a cDNA or a genomic sequence encoding an antibody used in the method of the present invention, can be used to construct a recombinant expression cassette that can be introduced into at least one desired host cell. A recombinant expression cassette will typically comprise a polynucleotide operably linked to transcriptional initiation regulatory sequences that will direct the transcription of the polynucleotide in the intended host cell. Both heterologous and non-heterologous (i.e., endogenous) promoters can be employed to direct expression of the nucleic acids.

In some embodiments, isolated nucleic acids that serve as promoter, enhancer, or other elements can be introduced in the appropriate position (upstream, downstream or in the intron) of a non-heterologous form of a polynucleotide of the present invention so as to up or down regulate expression of a polynucleotide. For example, endogenous promoters can be altered in vivo or in vitro by mutation, deletion and/or substitution.

### Vectors and Host Cells

The present invention also relates to vectors that include isolated nucleic acid molecules, host cells that are genetically engineered with the recombinant vectors, and the production of at least one anti-IL-23 antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody used in the method of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein used in the method of the present invention. Alternatively, nucleic acids can be expressed in a host cell by turning on (by manipulation) in a host cell that contains endogenous DNA encoding an antibody. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Purification of an Antibody

An anti-IL-23 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies used in the method of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Anti-IL-23 Antibodies.

An anti-IL-23 antibody, also referred to herein as “anti-IL-23 specific antibody,” useful for a method according to embodiments of the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, further optionally comprising at least one substitution, insertion or deletion), a heavy chain or light chain constant region, (e.g., comprising at least one C_{H}1, hinge1, hinge2, hinge3, hinge4, C_{H}2, or C_{H}3 or fragment thereof, further optionally comprising at least one substitution, insertion or deletion), or any portion thereof, that can be incorporated into an antibody. An antibody can include or be derived from any mammal, such as but not limited to, a human, a mouse, a rabbit, a rat, a rodent, a primate, or any combination thereof, and the like.

The isolated antibodies used in a method of the present invention comprise the antibody amino acid sequences disclosed herein encoded by any suitable polynucleotide, or any isolated or prepared antibody. Preferably, the human antibody or antigen-binding fragment binds human IL-23 and, thereby, partially or substantially neutralizes at least one biological activity of the protein. An antibody, or specified portion or variant thereof, that partially or preferably substantially neutralizes at least one biological activity of at least one IL-23 protein or fragment can bind the protein or fragment and thereby inhibit activities mediated through the binding of IL-23 to the IL-23 receptor or through other IL-23-dependent or mediated mechanisms. As used herein, the term “neutralizing antibody” refers to an antibody that can inhibit an IL-23-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-23 antibody to inhibit an IL-23-dependent activity is preferably assessed by at least one suitable IL-23 protein or receptor assay, as described herein and/or as known in the art. A human antibody can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, γ4). Antibodies of this type can be prepared by employing a transgenic mouse or other trangenic non-human mammal comprising at least one human light chain (e.g., IgG, IgA, and IgM) transgenes as described herein and/or as known in the art. In another embodiment, the anti-IL-23 human antibody comprises an IgG1 heavy chain and an IgG1 light chain.

An antibody binds at least one specified epitope specific to at least one IL-23 protein, subunit, fragment, portion or any combination thereof. The at least one epitope can comprise at least one antibody binding region that comprises at least one portion of the protein, which epitope is preferably comprised of at least one extracellular, soluble, hydrophillic, external or cytoplasmic portion of the protein.

Generally, the human antibody or antigen-binding fragment will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences may be derived from human germline sequences or closely match the germline sequences. For example, the CDRs from a synthetic library derived from the original non-human CDRs can be used. These CDRs may be formed by incorporation of conservative substitutions from the original non-human sequence. In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3.

Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

In one embodiment, an anti-IL-23 antibody useful for the present invention comprises a heavy chain variable region and a light chain variable region, the heavy chain variable region comprising a complementarity determining region heavy chain 1 (CDRH1) amino acid sequence of SEQ ID NO: 1, a CDRH2 of SEQ ID NO: 2, and a CDRH3 of SEQ ID NO: 3; and the light chain variable region comprising a complementarity determining region light chain 1 (CDRL1) amino acid sequence of SEQ ID NO: 4, a CDRL2 of SEQ ID NO: 5, and a CDRL3 of SEQ ID NO: 6.

A preferred anti-IL-23 antibody useful for the present invention comprises a heavy chain variable region having the amino acid sequence of SEQ ID NO: 7 and a light chain variable region having the amino acid sequence of SEQ ID NO: 8.

A more preferred anti-IL-23 antibody useful for the present invention is guselkumab (also referred to as CNTO1959, marketed as TREMFYA).

Other anti-IL-23 antibodies useful for the present invention include, but are not limited to, those having sequences described in U.S. Pat. No. 7,935,344, the entire contents of which are incorporated herein by reference.

### Antibody Compositions Comprising Further Therapeutically Active Ingredients

The antibody compositions used in the method of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21^{st} edition, Springhouse Corp., Springhouse, P A, 2001; Health Professional's Drug Guide 2001, ed., Shannon, Wilson, Stang, Prentice-Hall, Inc, Upper Saddle River, N.J.; Pharmcotherapy Handbook, Wells et al., ed., Appleton & Lange, Stamford, Conn., each entirely incorporated herein by reference).

By way of example of the drugs that can be combined with the antibodies for the method of the present invention, the anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

The at least one coricosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate. The at least one androgen or anabolic steroid can be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, and tacrolimus.

The at least one local anti-infective can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. The at least one scabicide or pediculicide can be at least one selected from crotamiton, lindane, permethrin, and pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. (See, e.g., pp. 1098-1136 of Nursing 2001 Drug Handbook.)

Anti-IL-23 antibody compositions can further comprise at least one of any suitable and effective amount of a composition or pharmaceutical composition comprising at least one anti-IL-23 antibody contacted or administered to a cell, tissue, organ, animal or patient in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), an immunization, an immunoglobulin, an immunosuppressive (e.g., basiliximab, cyclosporine, daclizumab), a cytokine or a cytokine antagonist. Non-limiting examples of such cytokines include, but are not limited to, any of IL-1 to IL-23 et al. (e.g., IL-1, IL-2, etc.). Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Anti-IL-23 antibody compounds, compositions or combinations used in the method of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the anti-IL-23 antibody, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-23 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-23 antibody compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-23 antibody, portion or variant compositions according to the invention are known in the art, e.g., as listed in “Remington: The Science & Practice of Pharmacy,” 19^{th} ed., Williams & Williams, (1995), and in the “Physician's Desk Reference,” 52^{nd} ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

### Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-23 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the method of the invention uses an article of manufacture, comprising packaging material and at least one vial comprising a solution of at least one anti-IL-23 specific antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further uses an article of manufacture, comprising packaging material, a first vial comprising lyophilized anti-IL-23 specific antibody, and a second vial comprising an aqueous diluent of prescribed buffer or preservative, wherein said packaging material comprises a label that instructs a patient to reconstitute the anti-IL-23 specific antibody in the aqueous diluent to form a solution that can be held over a period of twenty-four hours or greater.

The anti-IL-23 specific antibody used in accordance with the present invention can be produced by recombinant means, including from mammalian cell or transgenic preparations, or can be purified from other biological sources, as described herein or as known in the art.

The range of the anti-IL-23 specific antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations can be prepared by a process which comprises mixing at least one anti-IL-23 specific antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-23 specific antibody and preservative in an aqueous diluent is carried out using conventional dissolution and mixing procedures. To prepare a suitable formulation, for example, a measured amount of at least one anti-IL-23 specific antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations can be provided to patients as clear solutions or as dual vials comprising a vial of lyophilized anti-IL-23 specific antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biologically activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of anti-IL-23 specific antibody can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-IL-23 specific antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one anti-IL-23 specific antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, Smartject® e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com), and similary suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors, and needle free IV infusion sets.

The products may include packaging material. The packaging material provides, in addition to the information required by the regulatory agencies, the conditions under which the product can be used. The packaging material of the present invention provides instructions to the patient, as applicable, to reconstitute the at least one anti-IL-23 antibody in the aqueous diluent to form a solution and to use the solution over a period of 2-24 hours or greater for the two vial, wet/dry, product. For the single vial, solution product, pre-filled syringe or auto-injector, the label indicates that such solution can be used over a period of 2-24 hours or greater. The products are useful for human pharmaceutical product use.

The formulations used in the method of the present invention can be prepared by a process that comprises mixing an anti-IL-23 antibody and a selected buffer, preferably, a phosphate buffer containing saline or a chosen salt. Mixing the anti-IL-23 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The method of the invention provides pharmaceutical compositions comprising various formulations useful and acceptable for administration to a human or animal patient. Such pharmaceutical compositions are prepared using water at “standard state” as the diluent and routine methods well known to those of ordinary skill in the art. For example, buffering components such as histidine and histidine monohydrochloride hydrate, may be provided first followed by the addition of an appropriate, non-final volume of water diluent, sucrose and polysorbate 80 at “standard state.” Isolated antibody may then be added. Last, the volume of the pharmaceutical composition is adjusted to the desired final volume under “standard state” conditions using water as the diluent. Those skilled in the art will recognize a number of other methods suitable for the preparation of the pharmaceutical compositions.

The pharmaceutical compositions may be aqueous solutions or suspensions comprising the indicated mass of each constituent per unit of water volume or having an indicated pH at “standard state.” As used herein, the term “standard state” means a temperature of 25° C.+/−2° C. and a pressure of 1 atmosphere. The term “standard state” is not used in the art to refer to a single art recognized set of temperatures or pressure, but is instead a reference state that specifies temperatures and pressure to be used to describe a solution or suspension with a particular composition under the reference “standard state” conditions. This is because the volume of a solution is, in part, a function of temperature and pressure. Those skilled in the art will recognize that pharmaceutical compositions equivalent to those disclosed here can be produced at other temperatures and pressures. Whether such pharmaceutical compositions are equivalent to those disclosed here should be determined under the “standard state” conditions defined above (e.g. 25° C.+/−2° C. and a pressure of 1 atmosphere).

Importantly, such pharmaceutical compositions may contain component masses “about” a certain value (e.g. “about 0.53 mg L-histidine”) per unit volume of the pharmaceutical composition or have pH values about a certain value. A component mass present in a pharmaceutical composition or pH value is “about” a given numerical value if the isolated antibody present in the pharmaceutical composition is able to bind a peptide chain while the isolated antibody is present in the pharmaceutical composition or after the isolated antibody has been removed from the pharmaceutical composition (e.g., by dilution). Stated differently, a value, such as a component mass value or pH value, is “about” a given numerical value when the binding activity of the isolated antibody is maintained and detectable after placing the isolated antibody in the pharmaceutical composition.

Competition binding analysis is performed to determine if the IL-23 specific mAbs bind to similar or different epitopes and/or compete with each other. Abs are individually coated on ELISA plates. Competing mAbs are added, followed by the addition of biotinylated hrIL-23. For positive control, the same mAb for coating may be used as the competing mAb (“self-competition”). IL-23 binding is detected using streptavidin. These results demonstrate whether the mAbs recognize similar or partially overlapping epitopes on IL-23.

One aspect of the method of the invention administers to a patient a pharmaceutical composition comprising

In one embodiment of the pharmaceutical compositions, the isolated antibody concentration is from about 77 to about 104 mg per ml of the pharmaceutical composition. In another embodiment of the pharmaceutical compositions the pH is from about 5.5 to about 6.5.

The stable or preserved formulations can be provided to patients as clear solutions or as dual vials comprising a vial of lyophilized at least one anti-IL-23 antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the anti-IL-23 antibody may result in other than a clear solution of lyophilized powder comprising the antibody. Among non-clear solutions are formulations comprising particulate suspensions, said particulates being a composition containing the anti-IL-23 antibody in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gleatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(B-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried antibody preparation is taught in U.S. Pat. No. 6,019,968. The antibody-based dry powder compositions may be produced by spray drying solutions or slurries of the antibody and, optionally, excipients, in a solvent under conditions to provide a respirable dry powder. Solvents may include polar compounds, such as water and ethanol, which may be readily dried. Antibody stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

An anti-IL-23 antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Therapeutic Applications

In one general aspect, the present application provides a method for modulating or treating psoriatic arthritis, in a cell, tissue, organ, animal, or patient, as known in the art or as described herein, using at least one IL-23 antibody of the present invention, e.g., administering or contacting the cell, tissue, organ, animal, or patient with a therapeutic effective amount of IL-23 specific antibody.

Any method of the present invention can comprise administering an effective amount of a composition or pharmaceutical composition comprising an anti-IL-23 antibody to a cell, tissue, organ, animal or patient in need of such modulation, treatment or therapy. Such a method can optionally further comprise co-administration or combination therapy for treating such diseases or disorders, wherein the administering of said at least one anti-IL-23 antibody, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimulab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, C A (2000); Nursing 2001 Handbook of Drugs, 21^{st} edition, Springhouse Corp., Springhouse, P A, 2001; Health Professional's Drug Guide 2001, ed., Shannon, Wilson, Stang, Prentice-Hall, Inc, Upper Saddle River, N.J., each of which references are entirely incorporated herein by reference.

### Therapeutic Treatments

Typically, treatment of psoriatic arthritis is achieved by administering an effective amount or dosage of an anti-IL-23 antibody composition that total, on average, a range from at least about 0.01 to 500 milligrams of an anti-IL-23 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 μg/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and, preferably, 0.1 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or, alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or, alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Alternative Administration

Many known and developed modes can be used according to the present invention for administering pharmaceutically effective amounts of an anti-IL-23 antibody. While pulmonary administration is used in the following description, other modes of administration can be used according to the present invention with suitable results. IL-23 specific antibodies of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of an anti-IL-23 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL-23 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

Having generally described the invention, the same will be more readily understood by reference to the following Examples, which are provided by way of illustration and are not intended as limiting. Further details of the invention are illustrated by the following non-limiting Examples. The disclosures of all citations in the specification are expressly incorporated herein by reference.

### Embodiments

Embodiment 1 is a method of treating psoriatic arthritis (PsA) in a subject in need thereof, the method comprising subtaneously administering to the subject a pharmaceutical composition comprising a safe and effective amount of an anti-IL-23 antibody and a pharmaceutically acceptable carrier.

Embodiment 1a is the method of embodiment 1, wherein the anti-IL-23 antibody comprises a heavy chain variable region and a light chain variable region, the heavy chain variable region comprising a complementarity determining region heavy chain 1 (CDRH1) amino acid sequence of SEQ ID NO: 1, a CDRH2 of SEQ ID NO: 2, and a CDRH3 of SEQ ID NO: 3; and the light chain variable region comprising a complementarity determining region light chain 1 (CDRL1) amino acid sequence of SEQ ID NO: 4, a CDRL2 of SEQ ID NO: 5, and a CDRL3 of SEQ ID NO: 6.

Embodiment 1b is the method of embodiment 1, wherein the antibody comprises the heavy chain variable region of the amino acid sequence of SEQ ID NO: 7, and the light chain variable region of the amino acid sequence of SEQ ID NO: 8.

Embodiment 1c is the method of embodiment 1, wherein the antibody comprises the heavy chain of the amino acid sequence of SEQ ID NO: 9, and the light chain of the amino acid sequence of SEQ ID NO: 10.

Embodiment 1d is the method of embodiment 1 to 1c, wherein the pharmaceutical composition is administered once every 4 four weeks (4w).

Embodiment 2 is the method of any one of embodiments 1 to 1b, wherein the antibody is administered at a total dosage of 25 mg to 200 mg per administration, such as 25 mg, 50 mg, 75 mg, 100 mg, 125 mg, 150 mg, 175 mg, and 200 mg per administration, or any dosage in between.

Embodiment 2a is the method of embodiment 2, wherein the total dosage is about 50 to about 150 mg per administration.

Embodiment 2b is the method of embodiment 2, wherein the total dosage is about 100 mg per administration.

Embodiment 3 is the method of any one of embodiments 1 to 2b, wherein the subject has inadequate response to a standard therapy for PsA.

Embodiment 3a is the method of embodiment 3, wherein the standard therapy is at least one selected form the group consisting of non-biological disease-modifying antirheumatic drugs (DMARDs), oral corticosteroid, apremilast, nonsteroidal anti-inflammatory drugs (NSAIDs).

Embodiment 3b is the method of embodiment 3, wherein the the standard therapy is a DMARD selected from the group consisting of methotrexate (MTX) administered to the subject at ≤25 mg/week, sulfasalazine (SSZ) administered to the subject at ≤3 g/day, hydroxychloroquine (HCQ) administered to the subject at ≤400 mg/day or leflunomide (LEF) administered to the subject at ≤20 mg/day.

Embodiment 3c is the method of embodiment 3, wherein the the standard therapy is an oral corticosteroid administered to the subject at an amount equivalent to ≤10 mg/day of prednisone.

Embodiment 3d is the method of embodiment 3, wherein the the standard therapy is a NSAID or other analgesic administered to the subject at the marketed dose approved by a regulatory authority.

Embodiment 3e is the method of embodiment 3, wherein the the standard therapy is apremilast administered to the subject at the marketed dose approved by a regulatory authority.

Embodiment 3f is the method of any one of embodiments 3 to 3e, wherein the subject is biologic treatment naïve.

Embodiment 3g is the method of any one of embodiments 3 to 3e, wherein the subject has previously received at least one biologic treatment for PsA.

Embodiment 3h is the method of embodiment 3g, wherein the subject has inadequate response to the at least one biologic treatment.

Embodiment 3i is the method of embodiment 3g or 3h, wherein the biologic treatment is selected from the group consisting of guselkumab, ustekinumab, secukinumab (AIN457), anti-tumor necrosis factor alpha (TNFα) agents (such as adalimumab, etanercept, infliximab, golimumab subcutaneous [SC] or intravenous [IV], certolizumab pegol, or their respective biosimilars), tildrakizumab (MK3222), ixekizumab (LY2439821), brodalumab (AMG827), risankizumab (BI-655066), or other investigative biologic treatment for PsA or psoriasis.

Embodiment 3j is the method of embodiment 3i, wherein the subject is a non-responder to an anti-tumor necrosis factor alpha (TNFα) treatment.

Embodiment 3k is the method of any one of embodiments 1 to 3j, wherein the subject has at least 3% body surface area (BSA) of plaque psoriasis prior to the treatment.

Embodiment 3l is the method of any one of embodiments 1 to 3j, wherein the subject has at least one psoriatic plaque of ≥2 cm diameter or nail changes consistent with psoriasis or documented history of plaque psoriasis prior to the treatment.

Embodiment 3m is the method of any one of embodiments 1 to 31, optionally further comprising administering to the subject a standard therapy for PsA.

Embodiment 3n is the method of any one of embodiments 1 to 31, optionally further comprising administering to the subject a biologic treatment for PsA.

Embodiment 4 is the method of any one of embodiments 1 to 3n, wherein the subject is a responder to the treatment with the antibody and is identified as having a statistically significant improvement in disease activity, wherein disease activity is determined by one or more criteria selected from the group consisting of a 20% improvement in the American College of Rheumatology core set disease index (ACR20), a 50% improvement in the American College of Rheumatology core set disease index (ACR50), a 70% improvement in the American College of Rheumatology core set disease index (ACR70), Health Assessment Questionnaire Disability Index (HAQ-DI), Investigator's Global Assessment (IGA), Disease Activity Score 28 (DAS28) C-reactive protein (CRP), resolution of enthesitis, resolution of dactylitis, Leeds enthesitis index (LEI), dactylitis assessment score, Short Form Health survey (SF-36) in the mental and physical component summary (MCS and PCS), achievement of minimal disease activity (MDA), LS mean change from baseline in total modified vdH-S score and achievement of very low disease activity (VLDA).

Embodiment 4a is the method of embodiment 4, wherein the improvement is measured 16, 20, 24 or 28 weeks after initial treatment.

Embodiment 4b is the method of any one of embodiments 4-4a, wherein the improvement is measured 16 weeks after initial treatment.

Embodiment 4c is the method of any one of embodiments 4-4a, wherein the improvement is measured 24 weeks after initial treatment.

Embodiment 5 is the method of any one of embodiments 4-4c, wherein the subject is a responder to the treatment with the antibody and is identified as having a statistically significant improvement in disease activity as determined by a 20% improvement in the American College of Rheumatology core set disease index (ACR20) by week 24 of treatment with the antibody.

Embodiment 5a is the method of any one of embodiments 4-4c, wherein the subject is a responder to the treatment with the antibody and is identified as having a statistically significant improvement in disease activity as determined by a 20% improvement in the American College of Rheumatology core set disease index (ACR20) by week 16 of treatment with the antibody.

Embodiment 5b is the method of any one of embodiments 4-4c, wherein the subject is a responder to the treatment with the antibody and is identified as having a statistically significant improvement in disease activity as determined by a 50% improvement in the American College of Rheumatology core set disease index (ACR50) by week 24 of treatment with the antibody.

Embodiment 5c is the method of any one of embodiments 4-4c, wherein the subject is a responder to the treatment with the antibody and is identified as having a statistically significant improvement in disease activity as determined by a 50% improvement in the American College of Rheumatology core set disease index (ACR50) by week 16 of treatment with the antibody.

Embodiment 5d is the method of any one of embodiments 4-4c, wherein the subject is a responder to the treatment with the antibody and is identified as having a statistically significant improvement in disease activity as determined by a 70% improvement in the American College of Rheumatology core set disease index (ACR70) by week 24 of treatment with the antibody.

Embodiment 5e is the method of any one of embodiments 4-4c, wherein the subject is a responder to the treatment with the antibody and is identified as having a statistically significant improvement in disease activity as determined by the Health Assessment Questionnaire Disability Index (HAQ-DI) by week 24 of treatment with the antibody.

Embodiment 5f is the method of any one of embodiments 4-4c, wherein the subject is a responder to the treatment with the antibody and is identified as having a statistically significant improvement in disease activity as determined by Disease Activity Score 28 (DAS28)C-reactive protein (CRP) by week 24 of treatment with the antibody.

Embodiment 5g is the method of any one of embodiments 4-4c, wherein the subject is a responder to the treatment with the antibody and is identified as achieving Investigator's Global Assessment (IGA) of 0 (clear) or 1 (minimal) and/or ≥2 grade reduction of the IGA from baseline by week 24 of treatment with the antibody, wherein the subject has >=3% BSA psoriatic involvement and an IGA score of >=2 at the baseline.

Embodiment 5h is the method of any one of embodiments 4-4c, wherein the subject is a responder to the treatment with the antibody and is identified as having a statistically significant improvement in disease activity as determined by resolution of enthesitis by week 24 of treatment with the antibody.

Embodiment 5i is the method of any one of embodiments 4-4c, wherein the subject is a responder to the treatment with the antibody and is identified as having a statistically significant improvement in disease activity as determined by resolution of dactylitis by week 24 of treatment with the antibody.

Embodiment 5j is the method of any one of embodiments 4-4c, wherein the subject is a responder to the treatment with the antibody and is identified as having a statistically significant improvement in disease activity as determined by Leeds enthesitis index (LEI) by week 24 of treatment with the antibody.

Embodiment 5k is the method of any one of embodiments 4-4c, wherein the subject is a responder to the treatment with the antibody and is identified as having statistically significant improvement in disease activity as determined by the dactylitis assessment score of 0-3 ((0=absent, 1=mild, 2=moderate, 3=severe) by week 24 of treatment with the antibody.

Embodiment 5l is the method of any one of embodiments 4-4c, wherein the subject is a responder to the treatment with the antibody and is identified as having a statistically significant improvement in disease activity as determined by the Short-Form 36 (SF-36) health survey by week 24 of treatment with the antibody.

Embodiment 5m is the method of any one of embodiments 4-4c, wherein the subject is a responder to the treatment with the antibody and is identified as having a statistically significant improvement in disease activity as determined by the mental and physical component summary (MCS and PCS) scores by week 24 of treatment with the antibody.

Embodiment 5n is the method of any one of embodiments 4-4c, wherein the subject is a responder to the treatment with the antibody and is identified as having a statistically significant improvement in disease activity as determined by the minimal disease activity (MDA) criteria by week 24 of treatment with the antibody.

Embodiment 5o is the method of any one of embodiments 4-4c, wherein the subject is a responder to the treatment with the antibody and is identified as having a statistically significant improvement in disease activity as determined by achievement of very low disease activity (VLDA).

Embodiment 5p is the method of any one of embodiments 4-4c, wherein the subject is a responder to the treatment with the antibody and is identified as having a statistically significant improvement in disease activity as determined by LS mean change from baseline in total modified vdH-S score.

Embodiment 6 is the method of any one of embodiments 4-5o, wherein the improvement is maintained for at least 12 weeks, 24 weeks, 36 weeks, 48 weeks, 60 weeks, 72 weeks, or 84 weeks, or any time in between.

Embodiment 7 is the method of any one of embodiments 1-6, wherein the anti-IL-23 antibody is guselkumab.

Embodiment 8 is the method of any one of embodiments 1-7, further comprising administering to the subject one or more additional drugs used to treat psoriasis arthritis.

Embodiment 8a is the method of embodiment 8, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.

Embodiment 9 is a method of treating psoriatic arthritis (PsA) in a subject, the method comprising subtaneously administering to the subject a pharmaceutical composition comprising a safe and effective amount of an anti-IL-23 antibody and a pharmaceutically acceptable carrier, wherein the pharmaceutical composition is administered at an initial dose, a dose 4 weeks thereafter, and at a dosing interval of once every 8 weeks (q8w) thereafter, and wherein the subject has at least one psoriatic plaque of ≥2 cm diameter or nail changes consistent with psoriasis or documented history of plaque psoriasis before the treatment.

Embodiment 9a is the method of embodiment 9, wherein the anti-IL-23 antibody comprises a heavy chain variable region and a light chain variable region, the heavy chain variable region comprising a complementarity determining region heavy chain 1 (CDRH1) amino acid sequence of SEQ ID NO: 1, a CDRH2 of SEQ ID NO: 2, and a CDRH3 of SEQ ID NO: 3; and the light chain variable region comprising a complementarity determining region light chain 1 (CDRL1) amino acid sequence of SEQ ID NO: 4, a CDRL2 of SEQ ID NO: 5, and a CDRL3 of SEQ ID NO: 6.

Embodiment 9b is the method of embodiment 9, wherein the antibody comprises the heavy chain variable region of the amino acid sequence of SEQ ID NO: 7, and the light chain variable region of the amino acid sequence of SEQ ID NO: 8.

Embodiment 9c is the method of embodiment 9, wherein the anti-IL-23 antibody comprises the heavy chain amino acid sequence of SEQ ID NO: 9, and the light chain amino acid sequence of SEQ ID NO: 10.

Embodiment 10 is the method of any one of embodiments 9 to 9c, wherein the antibody is administered at a total dosage of 25 mg to 200 mg per administration, such as 25 mg, 50 mg, 75 mg, 100 mg, 125 mg, 150 mg, 175 mg, and 200 mg per administration, or any dosage in between.

Embodiment 10a is the method of embodiment 10, wherein the total dosage is about 50 to about 150 mg per administration.

Embodiment 10b is the method of embodiment 10, wherein the total dosage is about 100 mg per administration.

Embodiment 11 is the method of any one of embodiments 9 to 10b, wherein the subject has inadequate response to a standard therapy for PsA.

Embodiment 11a is the method of embodiment 11, wherein the standard therapy is at least one selected form the group consisting of non-biological disease-modifying antirheumatic drugs (DMARDs), oral corticosteroid, apremilast, nonsteroidal anti-inflammatory drugs (NSAIDs).

Embodiment 11b is the method of embodiment 11, wherein the the standard therapy is a DMARD selected from the group consisting of methotrexate (MTX) administered to the subject at ≤25 mg/week, sulfasalazine (SSZ) administered to the subject at ≤3 g/day, hydroxychloroquine (HCQ) administered to the subject at ≤400 mg/day or leflunomide (LEF) administered to the subject at ≤20 mg/day.

Embodiment 11c is the method of embodiment 11, wherein the the standard therapy is an oral corticosteroid administered to the subject at an amount equivalent to ≤10 mg/day of prednisone.

Embodiment 11d is the method of embodiment 11, wherein the the standard therapy is a NSAID or other analgesic administered to the subject at the marketed dose approved by a regulatory authority.

Embodiment 11e is the method of embodiment 11, wherein the the standard therapy is apremilast administered to the subject at the marketed dose approved by a regulatory authority.

Embodiment 11f is the method of any one of embodiments 11 to 11e, wherein the subject is biologic treatment naïve.

Embodiment 11g is the method of any one of embodiments 11 to 11e, wherein the subject has previously received at least one biologic treatment for PsA.

Embodiment 11h is the method of embodiment 11g, wherein the subject has inadequate response to the at least one biologic treatment.

Embodiment 11i is the method of embodiment 11g or 11h, wherein the biologic treatment is selected from the group consisting of guselkumab, ustekinumab, secukinumab (AIN457), anti-tumor necrosis factor alpha (TNFα) agents (such as adalimumab, etanercept, infliximab, golimumab subcutaneous [SC] or intravenous [IV], certolizumab pegol, or their respective biosimilars), tildrakizumab (MK3222), ixekizumab (LY2439821), brodalumab (AMG827), risankizumab (BI-655066), or other investigative biologic treatment for PsA or psoriasis.

Embodiment 11j is the method of embodiment 11i, wherein the subject is a non-responder to an anti-tumor necrosis factor alpha (TNFα) treatment.

Embodiment 11k is the method of any one of embodiments 9 to 11j, wherein the subject has at least 3% body surface area (BSA) of plaque psoriasis prior to the treatment.

Embodiment 11l is the method of any one of embodiments 9 to 11j, wherein the subject has at least one psoriatic plaque of ≥2 cm diameter or nail changes consistent with psoriasis or documented history of plaque psoriasis prior to the treatment.

Embodiment 11m is the method of any one of embodiments 9 to 111, optionally further comprising administering to the subject a standard therapy for PsA.

Embodiment 11n is the method of any one of embodiments 9 to 111, optionally further comprising administering to the subject a biologic treatment for PsA.

Embodiment 12 is the method of any one of embodiments 9 to 11n, wherein the subject is a responder to the treatment with the antibody and is identified as having a statistically significant improvement in disease activity, wherein disease activity is determined by one or more criteria selected from the group consisting of a 20% improvement in the American College of Rheumatology core set disease index (ACR20), a 50% improvement in the American College of Rheumatology core set disease index (ACR50), a 70% improvement in the American College of Rheumatology core set disease index (ACR70), Health Assessment Questionnaire Disability Index (HAQ-DI), Investigator's Global Assessment (IGA), Disease Activity Score 28 (DAS28) C-reactive protein (CRP), resolution of enthesitis, resolution of dactylitis, Leeds enthesitis index (LEI), dactylitis assessment score, Short Form Health survey (SF-36) in the mental and physical component summary (MCS and PCS), achievement of minimal disease activity (MDA), and achievement of very low disease activity (VLDA).

Embodiment 12a is the method of embodiment 12, wherein the improvement is measured 16, 20, 24 or 28 weeks after initial treatment.

Embodiment 12b is the method of any one of embodiments 12-12a, wherein the improvement is measured 16 weeks after initial treatment.

Embodiment 12c is the method of any one of embodiments 12-12a, wherein the improvement is measured 24 weeks after initial treatment.

Embodiment 13 is the method of any one of embodiments 12-12c, wherein the subject is a responder to the treatment with the antibody and is identified as having a statistically significant improvement in disease activity as determined by a 20% improvement in the American College of Rheumatology core set disease index (ACR20) by week 24 of treatment with the antibody.

Embodiment 13a is the method of any one of embodiments 12-12c, wherein the subject is a responder to the treatment with the antibody and is identified as having a statistically significant improvement in disease activity as determined by a 20% improvement in the American College of Rheumatology core set disease index (ACR20) by week 16 of treatment with the antibody.

Embodiment 13b is the method of any one of embodiments 12-12c, wherein the subject is a responder to the treatment with the antibody and is identified as having a statistically significant improvement in disease activity as determined by the American College of Rheumatology 130% improvement criteria (ACR130) by week 24 of treatment with the antibody.

Embodiment 13c is the method of any one of embodiments 12-12c, wherein the subject is a responder to the treatment with the antibody and is identified as having a statistically significant improvement in disease activity as determined by the American College of Rheumatology 130% improvement criteria (ACR130) by week 16 of treatment with the antibody.

Embodiment 13d is the method of any one of embodiments 12-12c, wherein the subject is a responder to the treatment with the antibody and is identified as having a statistically significant improvement in disease activity as determined by the A 70% improvement in the American College of Rheumatology core set disease index (ACR70) by week 24 of treatment with the antibody.

Embodiment 13e is the method of any one of embodiments 12-12c, wherein the subject is a responder to the treatment with the antibody and is identified as having a statistically significant improvement in disease activity as determined by the Health Assessment Questionnaire Disability Index (HAQ-DI) by week 24 of treatment with the antibody.

Embodiment 13f in the method of any one of embodiments 12-12c, wherein the subject is a responder to the treatment with the antibody and is identified as having a statistically significant improvement in disease activity as determined by Disease Activity Score 28 (DAS28) C-reactive protein (CRP) by week 24 of treatment with the antibody.

Embodiment 13g is the method of any one of embodiments 12-12c, wherein the subject is a responder to the treatment with the antibody and is identified as achieving Investigator's Global Assessment (IGA) of 0 (clear) or 1 (minimal) and/or ≥2 grade reduction from baseline by week 24 of treatment with the antibody, wherein the subject has >=3% BSA psoriatic involvement and an IGA score of >=2 at the baseline.

Embodiment 13h is the method of any one of embodiments 12-12c, wherein the subject is a responder to the treatment with the antibody and is identified as having a statistically significant improvement in disease activity as determined by resolution of enthesitis by week 24 of treatment with the antibody.

Embodiment 13i is the method of any one of embodiments 12-12c, wherein the subject is a responder to the treatment with the antibody and is identified as having a statistically significant improvement in disease activity as determined by resolution of dactylitis by week 24 of treatment with the antibody.

Embodiment 13j is the method of any one of embodiments 12-12c, wherein the subject is a responder to the treatment with the antibody and is identified as having a statistically significant improvement in disease activity as determined by Leeds enthesitis index (LEI) by week 24 of treatment with the antibody.

Embodiment 13k is the method of any one of embodiments 12-12c, wherein the subject is a responder to the treatment with the antibody and is identified as having statistically significant improvement in disease activity as determined by the dactylitis assessment score of 0-3 ((0=absent, 1=mild, 2=moderate, 3=severe) by week 24 of treatment with the antibody.

Embodiment 13l is the method of any one of embodiments 12-12c, wherein the subject is a responder to the treatment with the antibody and is identified as having a statistically significant improvement in disease activity as determined by the Short-Form 36 (SF-36) health survey by week 24 of treatment with the antibody.

Embodiment 13m is the method of any one of embodiments 12-12c, wherein the subject is a responder to the treatment with the antibody and is identified as having a statistically significant improvement in disease activity as determined by the mental and physical component summary (MCS and PCS) scores by week 24 of treatment with the antibody.

Embodiment 13n is the method of any one of embodiments 12-12c, wherein the subject is a responder to the treatment with the antibody and is identified as having a statistically significant improvement in disease activity as determined by the minimal disease activity (MDA) criteria by week 24 of treatment with the antibody.

Embodiment 13o is the method of any one of embodiments 12-12c, wherein the subject is a responder to the treatment with the antibody and is identified as having a statistically significant improvement in disease activity as determined by achievement of very low disease activity (VLDA).

Embodiment 14 is the method of any one of embodiments 12-13o, wherein the improvement is maintained for at least 12 weeks, 24 weeks, 36 weeks, 48 weeks, 60 weeks, 72 weeks, or 84 weeks, or any time in between.

Embodiment 15 is the method of any one of embodiments 9-14, wherein the anti-IL-23 antibody is guselkumab.

Embodiment 16 is the method of any one of embodiments 9-15, further comprising administering to the subject one or more additional drugs used to treat psoriasis arthritis.

Embodiment 16a is the method of embodiment 16, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.

### EXAMPLES

### Abbreviations and Acronyms

### ACR American College of Rheumatology

### AMDF Arithmetic Mean of the Desirability Function

AE adverse event
ALT alanine aminotransferase
ANOVA analysis of variance

### ARC Anticipated Event Review Committee

AST aspartate aminotransferase

### BASDAI Bath Ankylosing Spondylitis Disease Activity Index

BCG bacillus Calmette-Guérin
BQL below the lowest quantifiable sample concentration of the assay
BSA body surface area
CASPAR ClASsification criteria for Psoriatic Arthritis
CRF case report form(s) (paper or electronic as appropriate for this study)
CRP C-reactive protein

### DAS28 Disease Activity Score 28

DBL database lock

### DLQI Dermatology Life Quality Index

DMARDs disease-modifying antirheumatic drugs

### DMC Data Monitoring Committee

DNA deoxyribonucleic acid
ECG electrocardiogram
eC-SSRS electronic Columbia-Suicide Severity Rating Scale
eDC electronic data capture
EDTA ethylenediaminetetraacetic acid
EQ-5D EuroQol five dimensions questionnaire

### FACIT Functional Assessment of Chronic Illness Therapy

### FAS Full Analysis Set

FSH follicle stimulating hormone

### GCP Good Clinical Practice

GRACE GRAppa Composite score

### GRAppa Group for Research and Assessment of Psoriasis and Psoriatic Arthritis

### HAQ Health Assessment Questionnaire

### HAQ-DI Disability Index of the Health Assessment Questionnaire

HBV hepatitis B virus
HCP healthcare professional

### HCQ Hydroxychloroquine

HCV hepatitis C virus
HIV human immunodeficiency virus
ICF informed consent form

### ICH International Conference on Harmonisation

### IEC Independent Ethics Committee

### IGA Investigator's Global Assessment

IJA independent joint assessor
IL interleukin

### IRB Institutional Review Board

IV intravenous
IWRS interactive web response system
JAK Janus kinase
JSN joint space narrowing
LEF leflunomide

### LEI Leeds Enthesitis Index

mAb monoclonal antibody
MCP metacarpophalangeal
mCPDAI modified Composite Psoriatic Disease Activity Index

### MCS Mental Component Summary

MDA minimal disease activity
MI multiple imputation
Mill magnetic resonance imaging
MTX methotrexate
NAb neutralizing antibody
NSAID nonsteroidal anti-inflammatory drug

### PASDAS Psoriatic Arthritis Disease Activity Score

### PASI Psoriatic Area and Severity Index

### PCS Physical Component Summary

PD pharmacodynamic(s)
PFS prefilled syringe
PFS-U prefilled syringe with an UltraSafe PLUS™ Passive Needle Guard

### PGA Physician's Global Assessment

PIP proximal interphalangeal
PK pharmacokinetic(s)

### PQC Product Quality Complaint

PRO patient-reported outcome(s) (paper or electronic as appropriate for this
study)

### PROMIS-29 Patient-Reported Outcomes Measurement Information System-29

PsA psoriatic arthritis

### PsARC Psoriatic Arthritis Response Criteria

q4w every 4 weeks
q8w every 8 weeks
RA rheumatoid arthritis
RNA ribonucleic acid
SAE serious adverse event

### SAP Statistical Analysis Plan

SC subcutaneous
SD standard deviation
SDC smallest detectable change
SF-36 36-item Short Form Health Survey
SSZ sulfasalazine
SUSAR suspected unexpected serious adverse reaction
TB tuberculosis Th17 T helper 17
TNFα tumor necrosis factor alpha
UV ultraviolet

### VAS Visual Analogue Scale

vdH-S van der Heijde-Sharp (score)

### WPAI Work Productivity and Activity Impairment Questionnaire

### Example 1: A Phase 3, Multicenter, Randomized, Double-Blind, Placebo-Controlled Study Evaluating the Efficacy and Safety of Guselkumab Administered Subcutaneously in Subjects with Active Psoriatic Arthritis (CNTO1959PSA3002)

(CNTO1959PSA3002) is was a Phase 3 randomized, double-blind, placebo-controlled, multicenter, 3-arm study of guselkumab in subjects with active PsA who were biologic naïve and had an inadequate response to standard therapies (eg, non-biologic DMARDs, apremilast, NSAIDs). The study consists of a screening phase of up to 6 weeks, a blinded treatment phase of approximately 2 years (ie, 100 weeks) including a placebo-controlled period from Week 0 to Week 24 and an active treatment phase from Week 24 to Week 100, and a safety follow-up phase of 12 weeks after the last administration of study agent. The study was to enroll approximately 684 subjects. Stable doses of concomitant NSAIDs, oral corticosteroids, and selected non biologic DMARDs (limited to MTX, SSZ, hydroxychloroquine [HCQ], LEF) were allowed but not required.

The purpose of this Phase 3 study was to define the clinical efficacy of guselkumab in the reduction of signs and symptoms, improvement in physical function, inhibition of progression of structural damage, and to evaluate the safety profile of guselkumab in the treatment of PsA.

### Methods

### Study Design

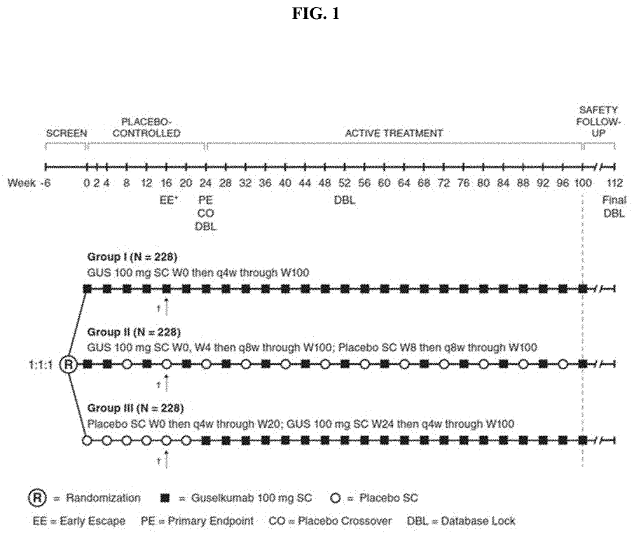

A diagrammatic representation of the study design is presented in FIG. 1. At Week 0, approximately 684 subjects who satisfied all inclusion and exclusion criteria were to be randomly assigned to 1 of the following 3 treatment groups in a 1:1:1 ratio using permuted block randomization stratified by baseline non-biologic DMARD use (yes, no) and the most recent available CRP value prior to randomization (<2.0 mg/dL versus ≥2.0 mg/dL):
Group I (n=228): Guselkumab 100 mg SC every 4 weeks (q4w) from Week 0 through Week 100.
Group II (n=228): Guselkumab 100 mg SC at Weeks 0 and 4 then q8w (Weeks 12, 20, 28, 36, 44, 52, 60, 68, 76, 84, 92, and 100) and placebo injections at other visits (Weeks 8, 16, 24, 32, 40, 48, 56, 64, 72, 80, 88, and 96) to maintain the blind.
Group III (n=228): Placebo SC q4w from Week 0 to Week 20 and cross over at Week 24 to receive guselkumab 100 mg SC q4w from Week 24 through Week 100.

At Week 16, all subjects in Groups I, II and III with <5% improvement from baseline in both tender and swollen joint counts were considered as meeting early escape (EE) criteria. These subjects remained on the dosing regimen they were randomized to at Week 0 but were allowed to initiate or increase the dose of one of the permitted concomitant medications up to the maximum allowed dose as specified in the protocol with titration to a stable dose of the medication to be completed by the Week 24 visit.

Efficacy evaluations included joint assessments (swollen and tender joint counts), patient's assessment of pain, patient's global assessment of disease activity (arthritis and psoriasis), patient's global assessment of disease activity (arthritis), physician's global assessment of disease activity, Health Assessment Questionnaire-Disability Index (HAQ-DI), CRP, patient's assessment of skin disease activity, body surface area (BSA) of psoriasis, Psoriasis Area and Severity Index (PAST), Investigator's Global Assessment of Psoriasis (IGA), Dermatology Life Quality Index (DLQI), dactylitis assessment, enthesitis assessment, Bath Ankylosing Spondylitis Disease Activity Index (BASDAI; in subjects with primary PsA subtype of spondylitis with peripheral arthritis), imaging evaluation (van der Heijde Sharp [vdH-S] score), American College of Rheumatology (ACR) response, Minimal Disease Activity (MDA) and Very Low Disease Activity (VLDA), Psoriatic Arthritis Disease Activity Score (PASDAS), Group Research and Assessment of Psoriasis and Psoriatic Arthritis (GRAPPA) Composite Score (GRACE) index, Disease Activity Index Score 28 (DAS28) using CRP, Modified Composite Psoriatic Disease Activity Index (mCPDAI), Disease Activity Index for Psoriatic Arthritis (DAPSA), Modified Psoriatic Arthritis Responder Criteria (PsARC), 36 Item Short-form Health Survey (SF-36), EuroQol five dimensions questionnaire (EQ 5D Questionnaire), and Functional Assessment of Chronic Illness Therapy (FACIT) Fatigue.

### Study Population

The target population consisted of adult men or women with active PsA who were biologic naïve and had an inadequate response to standard therapies (eg, non-biologic DMARDs, apremilast, and/or NSAIDs). Additionally, a biologic naïve population with a CRP≥0.6 mg/dL was required to enrich the population for radiographic progression and increase the power for detection of treatment effect on radiographic endpoints.

### Inclusion Criteria

To be eligible for this study, subjects had to be at least 18 years of age at the time of informed consent, diagnosed with PsA for at least 6 months prior to the first administration of study agent, and met ClASsification criteria for Psoriatic ARthritis (CASPAR)48 at screening. Subjects must have had active PsA as defined by ≥5 tender and ≥5 swollen joints at both screening and baseline, and CRP≥0.6 mg/dL at screening. Subjects must have had documented evidence of inadequate response or evidence of intolerance to standard PsA therapies including non-biologic DMARDs (≥3 months), apremilast (≥4 months), and/or NSAIDs (≥4 weeks) prior to the first administration of study agent.

Subjects had to have at least 1 of the PsA subsets: distal interphalangeal (DIP) joint involvement, polyarticular arthritis with absence of rheumatoid nodules, arthritis mutilans, asymmetric peripheral arthritis, or spondylitis with peripheral arthritis. In addition, subjects must have had active plaque psoriasis, with at least 1 psoriatic plaque of ≥2 cm diameter or nail changes consistent with psoriasis or documented history of plaque psoriasis.

Subjects were permitted to continue stable doses of non-biologic DMARDs (limited to MTX [≤25 mg/week], SSZ [≤3 g/day], HCQ [≤400 mg/day], or LEF [≤20 mg/day]), low-dose oral corticosteroids (≤10 mg of prednisone per day or equivalent), or NSAIDs and other analgesics treatment during the study. If subjects were not using these medications at baseline, these medications must have been stopped≥4 weeks (for MTX, SSZ, or HCQ), ≥12 week (LEF), or ≥2 weeks (for NSAIDs and other analgesics or oral corticosteroids) prior to the first administration of study agent. In addition, subjects had to meet criteria for screening laboratory test results and TB history and testing results, agree to use adequate birth control measures, avoid prolonged sun exposure, and avoid the use of tanning booths or other ultraviolet light sources during the study.

### Dosage and Administration

All study agents (guselkumab and placebo) were administered through SC injection. Based upon guselkumab clinical efficacy, safety, PK data, and exposure response modeling analysis using data from the Phase 2 study (CNTO1959PSA2001) in subjects with PsA, 2 dose regimens were chosen for evaluation in the guselkumab Phase 3 PsA program, and eligible subjects were randomly assigned to receive 1 of the following 3 treatments at Week 0:
Guselkumab 100 mg q4w: Guselkumab 100 mg SC q4w from Week 0 through Week 100.
Guselkumab 100 mg at Weeks 0 and 4 then q8w (hereafter referred to as the guselkumab 100 mg q8w group): Guselkumab 100 mg SC at Weeks 0 and 4, then q8w (at Weeks 12, 20, 28, 36, 44, 52, 60, 68, 76, 84, 92, and 100) and placebo injections at other visits (Weeks 8, 16, 24, 32, 40, 48, 56, 64, 72, 80, 88, and 96) to maintain the blind.
Placebo: Placebo SC q4w from Week 0 to Week 20, and cross over at Week 24 to receive guselkumab 100 mg SC q4w from Week 24 through Week 100.
   Rationale for Guselkumab 100 mg at Weeks 0 and 4 then Every 8 Weeks Dose Regimen
This dose regimen was evaluated in the Phase 2 PsA study (CNTO1959PSA2001) and in the 3 global Phase 3 studies in psoriasis. In the CNTO1959PSA2001 study, robust efficacy and clinically meaningful improvement was observed with this dose regimen in all important domains of PsA including joint signs and symptoms, physical function, psoriasis, enthesitis, dactylitis, and quality of life in patients with active PsA and ≥3% body surface area (BSA) of psoriasis. Additionally, significant benefit was also observed with this dose regimen on plaque psoriasis in patients with moderate-to-severe psoriasis in the Phase 3 psoriasis studies.
An additional dose was included at Week 4 to ensure that trough guselkumab levels do not fall below those obtained at steady state levels. This additional Week 4 dose results in a slightly higher Cmax and Ctrough in the first 12 weeks than those at steady state (˜21% and ˜18%, respectively) and may result in a more rapid onset of response. However, this dosing regimen is not expected to result in substantially higher levels of efficacy at Week 24 than would be achieved by q8w dosing during maintenance, ie, from Week 24 and onwards.
The safety of this dosing regimen has been established in a large psoriasis development program. Furthermore, the safety profile in the Phase 2 studies in patients with PsA and RA is consistent with that seen in the psoriasis program.

### Rationale for Guselkumab 100 mg Every 4 Weeks Dose Regimen

A dose regimen of 100 mg q4w was included to determine if more frequent dosing may achieve higher efficacy in PsA, including the inhibition of structural damage.
Modeling analyses based on data from CNTO1959PSA2001 suggested that a higher or more frequent dose regimen may achieve better efficacy in PsA.
Treatment with the 100 mg q4w dose regimen was expected to result in acceptable safety based on the exposure-safety analysis in the Phase 3 psoriasis program.
Guselkumab has been shown to have an acceptable safety profile in multiple patient populations, including with a higher dose regimen that was studied in a Phase 2 rheumatoid arthritis study (200 mg q8w).

Overall, the 2 dose regimens of guselkumab (100 mg q4w and 100 mg q8w) selected for this study were expected to provide an adequate assessment of the optimal benefit/risk profile of guselkumab in PsA.

Study agent was administered at the site by a health care professional (HCP) at Week 0 and Week 4. Beginning at Week 8, at the discretion of the investigator and subject, and after appropriate and documented training, subjects had the option to self administer study agent at the investigative site under the supervision of a HCP or continue to have study agent injections performed by a HCP.

Through Week 24, study agent administration at the site was to occur ±4 days from the scheduled day of study agent administration. Study agent administrations were to be at least 14 days apart.

### Efficacy Evaluations

### Primary Endpoint

The primary endpoint is proportion of subjects who achieve an ACR 20 response at Week 24.

### Major Secondary Endpoints

1. Change from baseline in HAQ-DI score at Week 24.
2. Proportion of subjects who achieve an ACR 50 response at Week 24.
3. Proportion of subjects with a psoriasis response of an IGA (ie, an IGA psoriasis score of 0 [cleared] or 1 [minimal] AND ≥2-grade reduction from baseline) at Week 24 among the subjects with ≥3% BSA psoriatic involvement and an IGA score of ≥2 (mild) at baseline.
4. Proportion of subjects who achieve an ACR 20 response at Week 16.
5. Change from baseline in modified vdH-S score at Week 24.
6. Proportion of subjects with resolution of enthesitis at Week 24 among the subjects with enthesitis at baseline.
7. Proportion of subjects with resolution of dactylitis at Week 24 among the subjects with dactylitis at baseline.
8. Change from baseline in enthesitis score (based on LEI) at Week 24 among the subjects with enthesitis at baseline.
9. Change from baseline in dactylitis score at Week 24 among the subjects with dactylitis at baseline.
10. Change from baseline in SF-36 PCS at Week 24.
11. Change from baseline in DAS28 (CRP) at Week 24.
12. Change from baseline in SF-36 MCS at Week 24.
13. Proportion of subjects who achieve an ACR 50 response at Week 16.
14. Proportion of subjects who achieve an ACR 70 response at Week 24.

### Other Secondary Endpoints

### Endpoints Related to Reduction of Signs and Symptoms and Physical Function

1. Proportions of subjects who achieve an ACR 20, ACR 50, and ACR 70 responses by visit over time through Week 24.
2. Percent change from baseline in ACR components by visit over time through Week 24.
3. Change from baseline in HAQ-DI score by visit over time through Week 24.
4. Proportion of subjects who achieve a clinically meaningful improvement (a ≥0.35 improvement from baseline) in HAQ-DI score by visit over time through Week 24 among those subjects with HAQ-DI score≥0.35 at baseline.
5. Proportion of subjects who achieve a DAS28 (CRP) response by visit over time through Week 24.
6. Proportion of subjects who achieve a DAS28 (CRP) remission by visit over time through Week 24.
7 Change from baseline in DAS28 (CRP) by visit over time through Week 24.
8. Proportion of subjects who achieve a response based on modified PsARC by visit over time through Week 24.
9. Proportion of subjects with resolution of enthesitis by visit by visit over time through Week 24 among the subjects with enthesitis at baseline.
10. Proportion of subjects with resolution of dactylitis by visit by visit over time through Week 24 among the subjects with dactylitis at baseline.
11. Change from baseline in enthesitis score (based on LEI) by visit over time through Week 24 among the subjects with enthesitis at baseline.
12. Change from baseline in dactylitis score by visit over time through Week 24 among the subjects with dactylitis at baseline.
13. Change from baseline in PASDAS by visit over time through Week 24.
14. Change from baseline in GRACE Index by visit over time through Week 24.
15. Change from baseline in WPAI scores by visit over time through Week 24.
16. Change from baseline in mCPDAI score by visit over time through Week 24.
17. Change from baseline in DAPSA score by visit over time through Week 24.
18. Proportion of subjects who achieve MDA by visit over time through Week 24.
19. Proportions of subjects who achieve a ≥20%, ≥50%, ≥70%, and ≥90% improvement from baseline in BASDAI score by visit over time through Week 24 among the subjects with spondylitis and peripheral joint involvement as their primary arthritic presentation of PsA.

### Endpoints Related to Skin Disease

1. Proportions of subjects who achieve ≥75%, ≥90%, and 100% improvement in PASI score from baseline by visit over time through Week 24 among the subjects with ≥3% BSA psoriatic involvement and an IGA score of ≥2 (mild) at baseline.
2. Proportion of subjects with an IGA score of 0 (cleared) by visit over time through Week 24 among the subjects with ≥3% BSA psoriatic involvement and an IGA score of ≥2 (mild) at baseline.
3. Change from baseline in PASI score by visit over time through Week 24 among the subjects with ≥3% BSA psoriatic involvement and an IGA score of ≥2 (mild) at baseline.
4. Proportion of subjects who achieve a DLQI score of 0 or 1 by visit over time through Week 24 among the subjects with baseline DLQI score>1 and with ≥3% BSA psoriatic involvement and an IGA score of ≥2 (mild) at baseline.
5. Proportion of subjects who achieve ≥5-point improvement from baseline in DLQI score by visit over time through Week 24 among the subjects with baseline DLQI score≥5 and with ≥3% BSA psoriatic involvement and an IGA score of ≥2 (mild) at baseline.
6. Change from baseline in DLQI score by visit over time through Week 24 among the subjects with ≥3% BSA psoriatic involvement and an IGA score of ≥2 (mild) at baseline.
7. Proportion of subjects who achieve both PASI 75 and ACR 20 responses by visit over time through Week 24 among the subjects with ≥3% BSA psoriatic involvement and an IGA score of ≥2 (mild) at baseline.
8. Proportion of subjects who achieve both PASI 75 and modified PsARC response by visit over time through Week 24 among the subjects with ≥3% BSA psoriatic involvement and an IGA score of ≥2 (mild) at baseline.

### Endpoints Related to Joint Structural Damage

Change from baseline in modified vdH-S score at Week 24.
2. Change from baseline in modified vdH-S erosion score at Week 24.
3. Change from baseline in modified vdH-S JSN score at Week 24.
4. Change from baseline in modified vdH-S score by region and type of damage (ie, hand erosion, hand JSN, foot erosion, foot JSN subscores) at Week 24.
5. Proportion of subjects with a change of ≤0 from baseline and proportion of subjects with a change of ≤0.5 from baseline in modified vdH-S score at Week 24.
6. Proportion of subjects with a change of ≤0 from baseline and proportion of subjects with a change of ≤0.5 from baseline in modified vdH-S erosion score at Week 24.
7. Proportion of subjects with a change of ≤0 from baseline and proportion of subjects with a change of ≤0.5 from baseline in modified vdH-S JSN score at Week 24.
8. Proportion of subjects with radiographic progression (based on the SDC) from baseline at Week 24.
9. Proportion of subjects with radiographic joint erosion progression (based on SDC) from baseline at Week 24.
10. Proportion of subjects with radiographic JSN progression (based on the SDC) from baseline at Week 24.
11. Proportion of subjects with pencil in cup or gross osteolysis deformities at Week 24.

### Endpoints Related to Health-Related Quality of Life

Change from baseline in PCS score of the SF-36 by visit over time through Week 24.
2. Change from baseline in MCS score of the SF-36 by visit over time through Week 24.
3. Change from baseline in domain scales scores of SF-36 by visit over time through Week 24.
4. Proportion of subjects who achieve ≥5-point improvement from baseline in SF-36 MCS score by visit over time through Week 24.
5. Proportion of subjects who achieve ≥5-point improvement from baseline in SF 36 PCS score by visit over time through Week 24.
6. Change from baseline in FACIT Fatigue by visit over time through Week 24.
7. Proportion of subjects who achieve ≥4-point improvement from baseline in FACIT Fatigue score improvement by visit over time through Week 24.
8. Change from baseline in EQ-5D VAS and in EQ-5D index scores by visit over time through Week 24.

### Baseline Disease Characteristics of PsA for ACR Core Set of Measurements

Baseline clinical characteristics of PsA from the ACR core set of outcome measurements were indicative of subjects with PsA of moderate to severe activity and were comparable across the treatment groups; however, median CRP was slightly higher in the guselkumab 100 mg q8w group (1.310 mg/dL) compared with the guselkumab 100 mg q4w group (1.160 mg/dL) and the placebo group (1.155 mg/dL; Table 1).

TABLE 1
Summary of PsA Disease Characteristics for ACR Components at Baseline; Full Analysis
Set 1 (Study CNTO1959P5A3002)

| | Guselkumab | | | | |
|---|---|---|---|---|---|
| | Placebo | 100 mg q8w | 100 mg q4w | Combined | Total |
| Analysis set: Full Analysis Set 1 | 246 | 248 | 245 | 493 | 739 |
| Number of swollen joints (0-66) | | | | | |
| N | 246 | 248 | 245 | 493 | 739 |
| Mean (SD) | 12.3 (6.86) | 11.7 (6.82) | 12.9 (7.83) | 12.3 (7.36) | 12.3 (7.19) |
| Median | 10.0 | 9.5 | 11.0 | 10.0 | 10.0 |
| Range | (5; 55) | (5; 46) | (5; 56) | (5; 56) | (5; 56) |
| IQ range | (8.0; 15.0) | (7.0; 14.0) | (7.0; 16.0) | (7.0; 15.0) | (7.0; 15.0) |
| Number of tender joints (0-68) | | | | | |
| N | 246 | 248 | 245 | 493 | 739 |
| Mean (SD) | 21.6 (13.06) | 19.8 (11.86) | 22.4 (13.54) | 21.1 (12.78) | 21.3 (12.87) |
| Median | 18.0 | 16.0 | 19.0 | 18.0 | 18.0 |
| Range | (5; 68) | (5; 64) | (5; 66) | (5; 66) | (5; 68) |
| IQ range | (12.0; 27.0) | (11.0; 25.0) | (12.0; 28.0) | (12.0; 27.0) | (12.0; 27.0) |
| Patient's assessment of pain | | | | | |
| (VAS; 0-10 cm) | | | | | |
| N | 246 | 248 | 245 | 493 | 739 |
| Mean (SD) | 6.28 (1.773) | 6.31 (1.958) | 6.15 (1.987) | 6.23 (1.972) | 6.25 (1.907) |
| Median | 6.50 | 6.45 | 6.50 | 6.50 | 6.50 |
| Range | (0.8; 10.0) | (1.0; 10.0) | (0.5; 10.0) | (0.5; 10.0) | (0.5; 10.0) |
| IQ range | (5.00; 7.50) | (4.90; 7.90) | (4.90; 7.50) | (4.90; 7.70) | (4.90; 7.60) |
| Patient's global assessment | | | | | |
| of disease activity (arthritis, | | | | | |
| VAS; 0-10 cm) | | | | | |
| N | 246 | 248 | 245 | 493 | 739 |
| Mean (SD) | 6.51 (1.790) | 6.53 (1.932) | 6.39 (1.943) | 6.46 (1.937) | 6.48 (1.888) |
| Median | 6.65 | 6.60 | 6.70 | 6.60 | 6.60 |
| Range | (1.3; 10.0) | (0.9; 10.0) | (0.3; 10.0) | (0.3; 10.0) | (0.3; 10.0) |
| IQ range | (5.30; 7.80) | (5.15; 8.10) | (5.20; 7.90) | (5.20; 7.90) | (5.20; 7.90) |
| Physician's global assessment | | | | | |
| of disease activity | | | | | |
| (VAS; 0-10 cm) | | | | | |
| N | 246 | 248 | 245 | 493 | 739 |
| Mean (SD) | 6.65 (1.490) | 6.56 (1.606) | 6.62 (1.538) | 6.59 (1.571) | 6.61 (1.544) |
| Median | 6.70 | 6.70 | 6.80 | 6.70 | 6.70 |
| Range | (2.8; 9.8) | (1.5; 10.0) | (1.8; 9.8) | (1.5; 10.0) | (1.5; 10.0) |
| IQ range | (5.70; 7.80) | (5.45; 7.80) | (5.70; 7.60) | (5.50; 7.70) | (5.50; 7.70) |
| HAQ disability index (0-3) | | | | | |
| N | 245 | 248 | 245 | 493 | 738 |
| Mean (SD) | 1.2949 | 1.2848 | 1.2490 | 1.2670 | 1.2763 |
| | (0.55755) | (0.62676) | (0.56732) | (0.59762) | (0.58439) |
| Median | 1.3750 | 1.2500 | 1.2500 | 1.2500 | 1.2500 |
| Range | (0.000; 2.750) | (0.000; 2.750) | (0.000; 2.750) | (0.000; 2.750) | (0.000; 2.750) |
| IQ range | (0.8750; | (0.8750; | (0.8750; | (0.8750; | (0.8750; |
| | 1.6250) | 1.7500) | 1.7500) | 1.7500) | 1.7500) |
| CRP (mg/dL) | | | | | |
| N | 246 | 248 | 245 | 493 | 739 |
| Mean (SD) | 2.116 | 2.036 | 1.807 | 1.922 | 1.986 |
| | (2.6652) | (2.3528) | (2.2247) | (2.2906) | (2.4217) |
| Median | 1.155 | 1.310 | 1.160 | 1.210 | 1.200 |
| Range | (0.01; 19.30) | (0.03; 18.80) | (0.01; 19.00) | (0.01; 19.00) | (0.01; 19.30) |
| IQ range | (0.514; 2.590) | (0.688; 2.530) | (0.591; 2.270) | (0.649; 2.410) | (0.600; 2.510) |

Key: IQ = interquartile

### Results

### Pharmacokinetic, Immunogenicity, Pharmacodynamic, and Pharmacogenomic Results

A total of 492 subjects who received at least 1 dose of guselkumab and had at least 1 valid sample collected after guselkumab administration were included in the PK evaluation. Subjects who received placebo only were excluded from the PK evaluation.

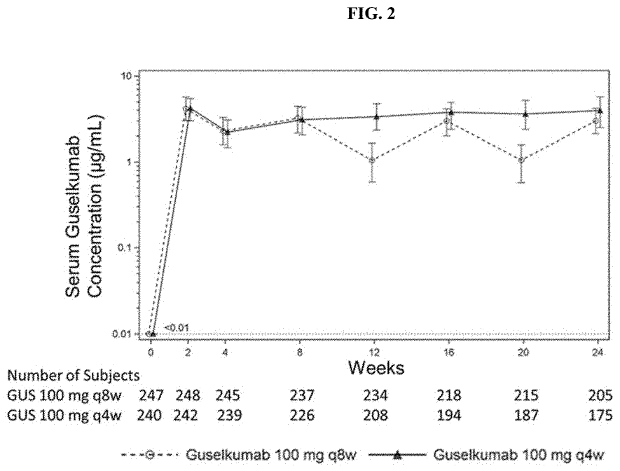

The median and IQ range of trough serum guselkumab concentrations by guselkumab treatment group and visit through Week 24 are graphically displayed in FIG. 2. Following SC administration of guselkumab, trough serum guselkumab concentrations generally reached steady state by Week 20 for the guselkumab 100 mg q8w group and by Week 12 for the guselkumab 100 mg q4w group (FIG. 2). In the guselkumab 100 mg q8w group, the median steady-state trough serum guselkumab concentration was 1.05 μg/mL at Week 20. In the guselkumab 100 mg q4w group, the median steady-state trough serum guselkumab concentration was 3.35 μg/mL at Week 12 and was maintained through Week 24 (3.98 μg/mL). The steady-state trough serum guselkumab concentrations in the guselkumab 100 mg q4w group were approximately 3- to 4-fold higher compared with those in the guselkumab 100 mg q8w group (FIG. 2).

In the guselkumab 100 mg q8w group, the median steady-state trough guselkumab concentrations at Week 20 in subjects who met or did not meet EE criteria were 0.58 and 1.06 μg/mL, respectively. In the guselkumab 100 mg q4w group, median steady-state trough guselkumab concentrations at Week 12 in subjects who met or did not meet EE criteria were 2.86 and 3.43 μg/mL. Median steady-state trough guselkumab concentrations appeared to be lower in subjects who met EE criteria. However, it should be noted that the number of subjects who met EE criteria was low for each treatment group (n≤13).

### Incidence of Antibodies to Guselkumab

A total of 490 subjects who received at least 1 dose of guselkumab and had appropriate samples for the detection of antibodies to guselkumab were included in the antibodies to guselkumab evaluation.

The overall incidence of antibodies to guselkumab through Week 24 was low (2.0%, 10/490) in subjects with PsA (Table 2). In the guselkumab 100 mg q8w group, the incidence of antibodies to guselkumab through Week 24 was 2.0% (5/247). In the guselkumab 100 mg q4w group, the incidence of antibodies to guselkumab through Week 24 was 2.1% (5/243). The highest titer of antibodies to guselkumab observed was 1:640 in the 100 mg q4w group.

The incidence of antibodies to guselkumab with or without MTX at baseline was 1.4% (4/284) and 2.9% (6/206), respectively. The incidence of antibodies to guselkumab with or without DMARD use at baseline was 1.8% (6/337) and 2.6% (4/153), respectively. Overall, the incidence of antibodies to guselkumab through Week 24 appeared to be lower in subjects with concomitant use of MTX or DMARDs compared with subjects without concomitant use of MTX or DMARDs. However, it should be noted that the number of subjects with positive antibodies to guselkumab was small and the incidence of antibodies to guselkumab was low regardless of concomitant MTX or DMARD use.

TABLE 2
Summary of Anti-Guselkumab Antibodies Status through Week 24; Immunogenicity
Analysis Set (Study CNTO1959PSA3002)

| | Guselkumab | | |
|---|---|---|---|
| | 100 mg q8w | 100 mg q4w | Combined |
| Analysis set: Immunogenicity Analysis Set | 247 | 243 | 490 |
| Subjects with appropriate samples^{a} | 247 | 243 | 490 |
| Subjects positive for anti-Guselkumab antibodies^{b,c} | 5 (2.0%) | 5 (2.1%) | 10 (2.0%) |
| Peak titers | | | |
| 1:10 | 3 | 1 | 4 |
| 1:40 | 1 | 0 | 1 |
| 1:160 | 1 | 1 | 2 |
| 1:640 | 0 | 3 | 3 |
| Subjects negative for anti-Guselkumab antibodies^{b,d} | 242 (98.0%) | 238 (97.9%) | 480 (98.0%) |

^{a}Subjects with appropriate samples had 1 or more evaluable samples obtained after their first Guselkumab administration
^{b}Denominator is subjects with appropriate samples.
^{c}Includes all subjects who had at least 1 positive sample at any time post-baseline through Week 24.
^{d}Includes all subjects with negative samples at all times through Week 24 and excludes subjects who were positive at any time through Week 24.

### Antibodies to Guselkumab and Pharmacokinetics

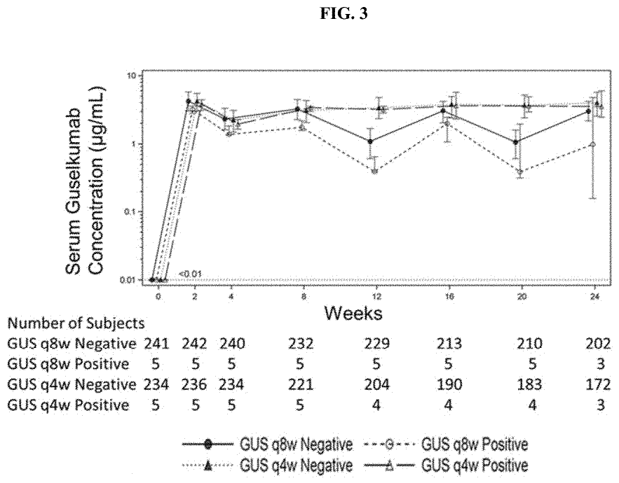

Serum guselkumab concentrations in subjects treated with guselkumab are summarized by treatment group and antibody to guselkumab status through Week 24. The median and IQ range of serum guselkumab concentrations through Week 24 by antibody to guselkumab status through Week 24 are presented graphically in FIG. 3. Individual serum guselkumab concentrations through Week 24 are also listed for subjects who were positive for antibodies to guselkumab.

Median serum guselkumab concentrations appeared to be lower in subjects with positive antibody to guselkumab status compared with subjects with negative antibody to guselkumab status in the guselkumab 100 mg q8w group (FIG. 3). However, it should be noted that the number of subjects who were positive for antibodies to guselkumab was very small (n=10), which limits a definitive conclusion of the effect of immunogenicity on guselkumab PK.

### Efficacy Results

### Primary Efficacy Endpoint Analysis

### ACR 20 Response at Week 24

A significantly greater proportion of subjects in both the guselkumab 100 mg q4w and guselkumab 100 mg q8w groups (63.7% and 64.1%, respectively) achieved an ACR 20 response at Week 24 compared with subjects in the placebo group (32.9%) based on both the global (ex-US) and US-specific multiplicity testing procedures (both global and US specific adjusted p<0.001), (Table 3).

TABLE 3
Number of Subjects Achieving ACR 20 Response at Week 24 (Primary Analysis) Based on
the Composite Estimand; Full Analysis Set 1 (Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Subjects evaluable for ACR 20 Response at | 245 | 246 | 245 |
| Week 24^{a} | | | |
| Subjects with ACR 20 Response^{b,h} | 81 (33.1%) | 159 (64.6%) | 156 (63.7%) |
| All subjects (including those with imputed | 246 | 248 | 245 |
| data) | | | |
| Subjects with ACR 20 Response^{b,c,h} | 81 (32.9%) | 159 (64.1%) | 156 (63.7%) |
| % Difference (95% CI)^{d} | | 31.2 (22.9, 39.5) | 30.8 (22.4, 39.1) |
| p-value^{e} | | <0.001 | <0.001 |

^{a}Subjects either have an observed ACR 20 response status or met a Treatment Failure (TF) criterion.
^{b}Defined as observed responders who had not met any TF criteria prior to Week 24.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and CRP prior to randomization (<2.0 mg/dL vs ≥2.0 mg/dL).
^{h}ACR 20 response is defined as ≥20% improvement from baseline in both tender joint count (68 joints) and swollen joint count (66 joints), and ≥20% improvement from baseline in at least 3 of the 5 assessments: patient's assessment of pain, patient's global assessment of disease activity, physician's global assessment of disease activity, HAQ-DI, and CRP.

### Major Secondary Endpoint Analyses

Change from Baseline in HAQ-DI Score at Week 24

At Week 24, a significantly greater reduction from baseline in HAQ-DI score was observed in both the guselkumab 100 mg q4w and the guselkumab 100 mg q8w groups compared with the placebo group (both global and US specific adjusted p<0.001; Table 4,) based on the composite estimand.

TABLE 4
Summary of the Change from Baseline in HAQ-DI Score at Week 24 Based on the
Composite Estimand Using MI and an ANCOVA Model; Full Analysis Set 1
(Study CNT01959P5A3002)

| | | Guselkumab | |
|---|---|---|---|
| | Placebo | 100 mg q8w | 100 mg q4w |
| Analysis set: Full Analysis Set 1 | 246 | 248 | 245 |
| Change from baseline in HAQ-DI^{a,h} | | | |
| Subjects evaluable^{b} | | | |
| N | 244 | 246 | 245 |
| Mean (SD) | −0.1527 (0.51258) | −0.3892 (0.53778) | −0.4097 (0.50084) |
| Median | −0.1250 | −0.2500 | −0.3750 |
| Range | (−2.250; 1.375) | (−2.250; 1.125) | (−2.000; 1.000) |
| IQ range | (−0.3750; 0.1250) | (−0.6250; 0.0000) | (−0.7500; 0.0000) |
| All subjects (including those with | | | |
| imputed data)^{a,c,h} | | | |
| N | 246 | 248 | 245 |
| Mean (SE)^{d} | −0.1557 (0.03280) | −0.3891 (0.03407) | −0.4097 (0.03200) |
| Model Based Estimates of the | | | |
| Mean Change^{a,c,h} | | | |
| LSMean (95% CI)^{e} | −0.1300 (−0.1912, −0.0687) | −0.3672 (−0.4282, −0.3062) | −0.4004 (−0.4617, −0.3390) |
| LSMean difference (95% CI) | | −0.2372 (−0.3210, −0.1534) | −0.2704 (−0.3544, −0.1864) |
| p-value^{f} | | <0.001 | <0.001 |

^{a}Defined as the change from baseline using observed data or 0 (no improvement) if a subject met Treatment Failure (TF) criteria prior to Week 24.
^{b}Subjects either have an observed change from baseline at this visit or met TF criteria prior to the visit.
^{c}Missing data is assumed to be Missing at Random (MAR) and is imputed using Multiple Imputation (MI).
^{d}The average of the mean, taken over all the MI data sets, is presented. The variance of the mean is the weighted sum of the average within-imputation variance and the between-imputation variance.
^{e}The LSmean for each MI data set is calculated based on an Analysis of Covariance (ANCOVA) model for the change from baseline at Week 24. The combined LSmean which is the average of the LSmean, taken over all the MI data sets, is presented.
^{f}The p-values (nominal) are based on the approximately normal distribution of the combined LSmean.
^{h}The HAQ score is the average of the computed categories scores (dressing, arising, eating, walking, hygiene, gripping and daily living). Lower scores are indicative of better functioning.

### Psoriasis IGA Response at Week 24

Among the 543 (73.5%) subjects with ≥3% BSA of psoriatic involvement and an IGA score≥2, a significantly greater proportion of subjects in both the guselkumab 100 mg q4w and the guselkumab 100 mg q8w groups achieved a psoriasis IGA response of 0 (cleared) or 1 (minimal) and >2-grade reduction from baseline in the IGA psoriasis score at Week 24 compared with the placebo group (both global and US-specific adjusted p<0.001; Table 5) based on the composite estimand.

TABLE 5
Number of Subjects Achieving an Investigator Global Assessment (IGA) Score of 0
(Cleared) or 1 (Minimal), and ≥2 Grade Reduction from Baseline at Week 24, Based on the
Composite Estimand; Full Analysis Set 1 Among the Subjects with ≥3% Body Surface Area
(BSA) of Psoriatic Involvement and an IGA Score ≥2 (mild) at Baseline
(Study CNTO1959PSA3002)

| | | Guselkumab | |
|---|---|---|---|
| | Placebo | 100 mg q8w | 100 mg q4w |
| Analysis set: Full Analysis Set 1 Among | 183 | 176 | 184 |
| the Subjects with ≥3% Body Surface Area | | | |
| (BSA) Psoriatic Involvement and an IGA | | | |
| score of ≥2 (mild) at Baseline | | | |
| Subjects evaluable for IGA response at | 182 | 175 | 183 |
| Week 24^{a} | | | |
| Subjects with IGA response^{b,h} | 35 (19.2%) | 124 (70.9%) | 126 (68.9%) |
| All subjects (including those with imputed | 183 | 176 | 184 |
| data) | | | |
| Subjects with IGA response^{b,c,h} | 35 (19.1%) | 124 (70.5%) | 126 (68.5%) |
| % Difference (95% CI)^{d} | | 50.9 (42.2, 59.7) | 49.8 (41.2, 58.4) |
| p-value^{e} | | <0.001 | <0.001 |

^{a}Subjects either have an observed IGA response status or met a Treatment Failure (TF) criterion.
^{b}Defined as observed responders who had not met any TF criteria prior to Week 24.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and CRP prior to randomization (<2.0 mg/dL vs ≥2.0 mg/dL).
^{h}The IGA documents the investigator's assessment of the patient's psoriasis and lesions are graded for induration, erythema and scaling, each using a 5 point scale: 0 (no evidence), 1 (minimal), 2 (mild), 3 (moderate), and 4 (severe). The IGA score of psoriasis is based upon the average of induration, erythema and scaling scores. An IGA response is defined as an IGA score of 0 (cleared) or 1 (minimal) and ≥2 grade reduction from baseline.

Change from Baseline in Modified vdH-S Score at Week 24
At Week 24, a numerically smaller (less progression) change from baseline in modified vdH-S score was observed in both the guselkumab 100 mg q4w and the guselkumab 100 mg q8w groups compared with the placebo group based on the treatment policy estimand (Table 6).

TABLE 6
Summary of the Change from Baseline in the Modified vdH-S score at Week 24 Based on
the Treatment Policy Estimand, Using MI and an ANCOVA Model (Read Campaign 1);
Full Analysis Set 1 for Structural Damage (Study CNTO1959PSA3002)

| | | Guselkumab | |
|---|---|---|---|
| | Placebo | 100 mg q8w | 100 mg q4w |
| Analysis set: Full Analysis Set 1 for | | | |
| Structural Damage | 246 | 248 | 245 |
| Change from baseline in modified vdH-S | | | |
| score^{a,h} | | | |
| Subjects evaluable^{b} | | | |
| N | 245 | 247 | 240 |
| Mean (SD) | 0.90 (3.142) | 0.45 (2.376) | 0.25 (2.521) |
| Median | 0.00 | 0.00 | 0.00 |
| Range | (−4.5; 28.5) | (−8.5; 17.5) | (−17.5; 13.5) |
| IQ range | (0.00; 1.00) | (−0.50; 1.00) | (−0.50; 0.50) |
| All subjects (including those with imputed | | | |
| data)^{a,c,h} | | | |
| N | 246 | 248 | 245 |
| Mean (SE)^{d} | 0.90 (0.201) | 0.46 (0.151) | 0.28 (0.163) |
| Model Based Estimates of the Mean | | | |
| Change^{a,b,h} | | | |
| LSMean (95% CI)^{e} | 0.95 (0.61, 1.29) | 0.52 (0.18, 0.86) | 0.29 (−0.05, 0.63) |
| LSMean difference (95% CI) | | −0.43 (−0.90, 0.03) | −0.66 (−1.13, −0.19) |
| p-value^{f} | | 0.068 | 0.006 |

^{a}Defined as the change from baseline using observed data regardless of meeting Treatment Failure (TF) criteria.
^{b}Subjects have an observed change from baseline.
^{c}Missing data is assumed to be Missing at Random (MAR) and is imputed using Multiple Imputation (MI).
^{d}The average of the mean, taken over all the MI data sets, is presented. The variance of the mean is the weighted sum of the average within-imputation variance and the between-imputation variance.
^{e}The LSmean for each MI data set is calculated based on an Analysis of Covariance (ANCOVA) model for the change from baseline at Week 24. The combined LSmean which is the average of the LSmean, taken over all the MI data sets, is presented.
^{f}The p-values (nominal) are based on the approximately normal distribution of the combined LSmean.
^{h}The modified vdH-S score is the sum of the erosion score (hand, feet) and joint space narrowing (JSN) score (hand, feet). The joint erosion score is the total erosion severity in 40 joints of the two hands and 12 joints of the 2 feet, for a maximum erosion score of 320. Each joint is scored from 0-5 with 0 indicating no erosion, and 5 indicating complete collapse of the bone. The JSN score is the total JSN score in the same 52 joints as above. Each joint is scored from 0-4 with 0 indicating no JSN, and 4 indicating an absence of joint space, for a maximum JSN score of 208. The maximum modified vdH-S score is 528.

Change from Baseline in SF-36 PCS at Week 24
At Week 24, a numerically greater improvement from baseline in SF-36 PCS score was observed in both the guselkumab 100 mg q4w and guselkumab 100 mg q8w groups compared with the placebo group based on the composite estimand (Table 7)

TABLE 7
Summary of the Change from Baseline in SF-36 PCS Score at Week 24 Based on the
Composite Estimand Using MI and an ANCOVA Model; Full Analysis Set 1
(Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Change from baseline in SF-36 PCS | | | |
| score^{a,h} | | | |
| Subjects evaluable^{b} | | | |
| N | 244 | 246 | 245 |
| Mean (SD) | 3.639 (6.8590) | 7.525 (8.0557) | 6.935 (6.9780) |
| Median | 3.590 | 7.085 | 6.210 |
| Range | (−17.33; 29.22) | (−11.63; 33.13) | (−9.23; 27.39) |
| IQ range | (−0.240; 7.765) | (1.310; 12.080) | (1.450; 11.350) |
| All subjects (including those with imputed | | | |
| data)^{a,c,h} | | | |
| N | 246 | 248 | 245 |
| Mean (SE)^{d} | 3.630 (0.4374) | 7.511 (0.5108) | 6.935 (0.4458) |
| Model Based Estimates of the Mean | | | |
| Change^{a,c,h} | | | |
| LSMean (95% CI)^{e} | 3.42 (2.53, 4.32) | 7.39 (6.50, 8.29) | 7.04 (6.14, 7.94) |
| LSMean difference (95% CI) | | 3.97 (2.74, 5.20) | 3.62 (2.39, 4.85) |
| p-value^{f} | | <0.001 | <0.001 |

^{a}Defined as the change from baseline using observed data or 0 (no improvement) if a subject met Treatment Failure (TF) criteria prior to Week 24.
^{b}Subjects either have an observed change from baseline at this visit or met TF criteria prior to the visit.
^{c}Missing data is assumed to be Missing at Random (MAR) and is imputed using Multiple Imputation (MI).
^{d}The average of the mean, taken over all the MI data sets, is presented. The variance of the mean is the weighted sum of the average within-imputation variance and the between-imputation variance.
^{e}The LSmean for each MI data set is calculated based on an Analysis of Covariance (ANCOVA) model for the change from baseline at Week 24. The combined LSmean which is the average of the LSmean, taken over all the MI data sets, is presented.
^{f}The p-values (nominal) are based on the approximately normal distribution of the combined LSmean.
^{h}The physical component summary (PCS) and mental component summary (MCS) scores are calculated based on the 8 scales of the SF-36 Health Related Quality of Life instrument with 36 questions. Higher scores indicate better health.

CHANGE from Baseline in SF-36 MCS at Week 24
At Week 24, a numerically greater improvement from baseline in SF-36 MCS score was observed in both the guselkumab 100 mg q4w and guselkumab 100 mg q8w groups compared with the placebo group based on the composite estimand (Table 8).

TABLE 8
Summary of the Change from Baseline in SF-36 MCS Score at Week 24 Based on the
Composite Estimand Using MI and an ANCOVA Model; Full Analysis Set 1
(Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Change from baseline in SF-36 MCS | | | |
| score^{a,h} | | | |
| Subjects evaluable^{b} | | | |
| N | 244 | 246 | 245 |
| Mean (SD) | 2.132 (9.5188) | 4.128 (9.7835) | 3.793 (8.9873) |
| Median | 0.210 | 2.630 | 2.100 |
| Range | (−36.92; 37.06) | (−30.75; 34.78) | (−23.21; 39.88) |
| IQ range | (−3.310; 7.925) | (−1.450; 9.920) | (−0.910; 8.070) |
| All subjects (including those with imputed | | | |
| data)^{a,c,h} | | | |
| N | 246 | 248 | 245 |
| Mean (SE)^{d} | 2.198 (0.6097) | 4.116 (0.6210) | 3.793 (0.5742) |
| Model Based Estimates of the Mean | | | |
| Change^{a,c,h} | | | |
| LSMean (95% CI)^{e} | 2.14 (1.07, 3.21) | 4.17 (3.10, 5.23) | 4.22 (3.14, 5.29) |
| LSMean difference (95% CI) | | 2.02 (0.56, 3.49) | 2.07 (0.60, 3.54) |
| p-value^{f} | | 0.007 | 0.006 |

^{a}Defined as the change from baseline using observed data or 0 (no improvement) if a subject met Treatment Failure (TF) criteria prior to Week 24.
^{b}Subjects either have an observed change from baseline at this visit or met TF criteria prior to the visit.
^{c}Missing data is assumed to be Missing at Random (MAR) and is imputed using Multiple Imputation (MI).
^{d}The average of the mean, taken over all the MI data sets, is presented. The variance of the mean is the weighted sum of the average within-imputation variance and the between-imputation variance.
^{e}The LSmean for each MI data set is calculated based on an Analysis of Covariance (ANCOVA) model for the change from baseline at Week 24. The combined LSmean which is the average of the LSmean, taken over all the MI data sets, is presented.
^{f}The p-values (nominal) are based on the approximately normal distribution of the combined LSmean.
^{h}The physical component summary (PCS) and mental component summary (MCS) scores are calculated based on the 8 scales of the SF-36 Health Related Quality of Life instrument with 36 questions. Higher scores indicate better health.

### Resolution of Enthesitis at Week 24

Among the 506 (68.5%) subjects with enthesitis at baseline, a numerically greater proportion of subjects in both the guselkumab 100 mg q4w and the guselkumab 100 mg q8w groups (43.5% and 53.8%, respectively) achieved enthesitis resolution at Week 24 compared with the placebo group (30.3%; nominal p=0.017 and p<0.001, respectively; Table 9). Based on CNTO1959PSA3001 data only, among the 222 (58.3%) subjects with enthesitis at baseline based on LEI, numerically greater proportions of subjects in the guselkumab 100 mg q4w group (47.9%) and the guselkumab 100 mg q8w group (40.3%) achieved enthesitis resolution at Week 24 compared to the placebo group (27.3%, nominal p=0.013 and p=0.094, respectively; Table 9). For both studies, the treatment effect was numerically greater in both guselkumab groups compared with the placebo group and allowed for the pooled analysis to be performed for both doses for this endpoint.

TABLE 9
Number of subjects with Resolution of Enthesitis (based on LEI) at Week 24 Based on the
Composite Estimand; Full Analysis Set 1 among the Subjects with Enthesitis (based on LEI) at
Baseline (Studies CNTO1959PSA3001 and CNTO1959P5A3002)

| | CNTO1959PSA3001 | | | CNTO1959PSA3002 | | |
|---|---|---|---|---|---|---|
| | | Guselkumab | | | Guselkumab | |
| | Placebo | 100 mg q8w | 100 mg q4w | Placebo | 100 mg q8w | 100 mg q4w |
| Analysis set: Full | 77 | 72 | 73 | 178 | 158 | 170 |
| Analysis Set 1 among | | | | | | |
| the Subjects with | | | | | | |
| Enthesitis (based on | | | | | | |
| LEI) at Baseline | | | | | | |
| Subjects evaluable for | 77 | 72 | 73 | 178 | 158 | 170 |
| enthesitis resolution at | | | | | | |
| Week 24^{a} | | | | | | |
| Subjects with | 21 | 29 | 35 | 54 | 85 | 74 |
| enthesitis resolution | (27.3%) | (40.3%) | (47.9%) | (30.3%) | (53.8%) | (43.5%) |
| 95% CI of | (16.7%, | (28.3%, | (35.8%, | (23.3%, | (45.7%, | (35.8%, |
| response rate^{b} | 37.9%) | 52.3%) | 60.1%) | 37.4%) | 61.9%) | 51.3%) |
| Difference | | 13.0 | 19.8 | | 23.3 | 12.3 |
| (95% CI) in | | (−1.6, | (4.9, | | (13.1, | (2.6, |
| response rates^{b} | | 27.5) | 34.6) | | 33.5) | 22.1) |
| p-value^{c} | | 0.094 | 0.013 | | <0.001 | 0.017 |
| | 2-Study Combined | | | | | |
| | | Guselkumab | | | | |
| | Placebo | 100 mg q8w | 100 mg q4w | | | |
| Analysis set: | 255 | 230 | 243 | | | |
| Full Analysis | | | | | | |
| Set 1 among | | | | | | |
| the Subjects | | | | | | |
| with Enthesitis | | | | | | |
| (based on LEI) | | | | | | |
| at Baseline | | | | | | |
| Subjects | 255 | 230 | 243 | | | |
| evaluable for | | | | | | |
| enthesitis | | | | | | |
| resolution at | | | | | | |
| Week 24^{a} | | | | | | |
| Subjects with | 75 | 114 | 109 | | | |
| enthesitis | (29.4%) | (49.6%) | (44.9%) | | | |
| resolution | (23.6%, | (42.9%, | (38.4%, | | | |
| 95% CI of | 35.2%) | 56.2%) | 51.3%) | | | |
| response rate^{b} | | | | | | |
| Difference | | 20.1 | 14.6 | | | |
| (95% CI) in | | (11.8, | (6.4, | | | |
| response rates^{b} | | 28.5) | 22.7) | | | |
| p-value^{c} | | <0.001 | <0.001 | | | |

### Resolution of Dactylitis at Week 24

Based on CNTO1959PSA3002 data only, among the 331 (44.8%) subjects with dactylitis at baseline, a numerically greater proportion of subjects in the guselkumab 100 mg q4w and the guselkumab 100 mg q8w groups (63.6% and 56.8%, respectively) achieved dactylitis resolution at Week 24 compared with the placebo group (38.4%; nominal p<0.001 and p=0.007, respectively; Table 10 and 11). Based on CNTO1959PSA3001 data only, among the 142 (37.3%) subjects with dactylitis at baseline, numerically greater proportions of subjects in the guselkumab 100 mg q4w group (63.2%) and the guselkumab 100 mg q8w group (65.3%) achieved dactylitis resolution at Week 24 compared to the placebo group (49.1%; nominal p=0.212 and p=0.088, respectively; Table 10 and 11). For both studies, the treatment effect was numerically greater in both guselkumab groups compared with the placebo group and allowed for the pooled analysis to be performed for both doses for this endpoint.

TABLE 10
Number of subjects with Resolution of Enthesitis (based on LEI) at Week 24 Based
on the Composite Estimand; Full Analysis Set 1 among the Subjects with Enthesitis (based on LEI)
at Baseline (Studies CNTO1959PSA3001 and CNTO1959P5A3002)

| | CNTO1959PSA3001 | | | CNTO1959PSA3002 | | |
|---|---|---|---|---|---|---|
| | | Guselkumab | | | Guselkumab | |
| | Placebo | 100 mg q8w | 100 mg q4w | Placebo | 100 mg q8w | 100 mg q4w |
| Analysis set: Full | 55 | 49 | 38 | 99 | 111 | 121 |
| Analysis Set 1 among | | | | | | |
| the Subjects with | | | | | | |
| Dactylitis at Baseline | | | | | | |
| Subjects evaluable for | 55 | 49 | 38 | 99 | 111 | 121 |
| dactylitis resolution at | | | | | | |
| Week 24^{a} | | | | | | |
| Subjects with | 27 | 32 | 24 | 38 | 63 | 77 |
| dactylitis resolution | (49.1%) | (65.3%) | (63.2%) | (38.4%) | (56.8%) | (63.6%) |
| 95% CI of | (35.0%, | (51.0%, | (46.5%, | (28.3%, | (47.1%, | (54.7%, |
| response rate^{b} | 63.2%) | 79.7%) | 79.8%) | 48.5%) | 66.4%) | 72.6%) |
| Difference | | 16.6 | 13.4 | | 18.7 | 24.5 |
| (95% CI) in | | (−1.5, | (−6.9, | | (5.7, | (11.8, |
| response rates^{b} | | 34.8) | 33.7) | | 31.7) | 37.1) |
| p-value^{c} | | 0.088 | 0.212 | | 0.007 | <0.001 |
| Difference (95% | | | −1.9 | | | 6.2 |
| CI) in response | | | (−22.0, | | | (−6.3, |
| rates^{d} | | | 18.3) | | | 18.8) |
| p-value^{c} | | | 0.859 | | | 0.338 |

TABLE 11
Number of subjects with Resolution of Enthesitis (based on LEI) at Week 24 Based on the
Composite Estimand; Full Analysis Set 1 among the Subjects with Enthesitis (based on LEI) at Baseline
(Study CNTO1959PSA3001 and CNTO1959PSA3002 combined)

| | 2-study Combined | | |
|---|---|---|---|
| | | Gesulkemab | |
| | Placebo | 100 mg q8w | 100 mg q4w |
| Analysis set: Full Analysis | 154 | 160 | 159 |
| Set 1 among the Subjects | | | |
| with Dactylitis at Baseline | | | |
| Subjects evaluable for | 154 | 160 | 159 |
| dactylitis resolution at Week | | | |
| 24a | | | |
| Subjects with dactylitis | 65 (42.2%) | 95 (59.4%) | 101 (63.5%) |
| resolution | | | |
| 95% CI of response rate^{b} | (34.1%, 50.3%) | (51.5%, 67.3%) | (55.7%, 71.3%) |
| Difference (95% CI) in | | 18.0 (7.4, 28.6) | 21.3 (10.5, 32.0) |
| response rates^{b} | | | |
| p-value^{c} | | 0.001 | <0.001 |
| Difference (95% CI) in | | | |
| response rates^{d} | | | 4.1 (−6.6, 14.7) |
| p-value^{e} | | | 0.461 |

### Major Secondary Endpoints Controlled for Multiplicity in the Global (Ex-US) Testing Procedure and Conditionally Controlled in the US Specific Testing Procedure

Change from Baseline in DAS28 (CRP) at Week 24
A significantly greater reduction from baseline in DAS28 (CRP) score at Week 24 was observed in both the guselkumab 100 mg q4w and guselkumab 100 mg q8w groups compared with the placebo group (both global adjusted p<0.001;) based on the composite estimand (Table 12).

TABLE 12
Summary of the Change from Baseline in DAS 28 (CRP) Score at Week 24 Based on the
Composite Estimand Using MI and an ANCOVA Model; Full Analysis Set 1
(Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Change from baseline in DAS28 (CRP)^{a,h} | | | |
| Subjects evaluable^{b} | | | |
| N | 243 | 246 | 245 |
| Mean (SD) | −0.99 (1.102) | −1.56 (1.085) | −1.61 (1.016) |
| Median | −0.82 | −1.41 | −1.54 |
| Range | (−4.5; 1.3) | (−4.2; 0.5) | (−5.0; 0.2) |
| IQ range | (−1.64; −0.09) | (−2.42; −0.71) | (−2.33; −0.92) |
| All subjects (including those with imputed | | | |
| data)^{a,c,h} | | | |
| N | 246 | 248 | 245 |
| Mean (SE)^{d} | −0.98 (0.070) | −1.56 (0.069) | −1.61 (0.065) |
| Model Based Estimates of the Mean | | | |
| Change^{a,c,h} | | | |
| LSMean (95% CI)^{e} | −0.97 (−1.11, −0.84) | −1.59 (−1.72, −1.45) | −1.62 (−1.76, −1.49) |
| LSMean difference (95% CI) | −0.61 (−0.80, −0.43) | −0.65 (−0.83, −0.47) | |
| p-value^{f} | | <0.001 | <0.001 |

^{a}Defined as the change from baseline using observed data or 0 (no improvement) if a subject met Treatment Failure (TF) criteria prior to Week 24.
^{b}Subjects either have an observed change from baseline at this visit or met TF criteria prior to the visit.
^{c}Missing data is assumed to be Missing at Random (MAR) and is imputed using Multiple Imputation (MI).
^{d}The average of the mean, taken over all the MI data sets, is presented. The variance of the mean is the weighted sum of the average within-imputation variance and the between-imputation variance.
^{e}The LSmean for each MI data set is calculated based on an Analysis of Covariance (ANCOVA) model for the change from baseline at Week 24. The combined LSmean which is the average of the LSmean, taken over all the MI data sets, is presented.
^{f}The p-values (nominal) are based on the approximately normal distribution of the combined LSmean.
^{h}The DAS score is calculated based on the tender joints (28), swollen joints (28), patient's global assessment of disease activity, and CRP.

### ACR 20 Response at Week 16

The proportion of subjects who achieved an ACR 20 response at Week 16 was numerically higher in both the guselkumab 100 mg q4w and guselkumab 100 mg q8w groups compared with the placebo group based on the composite estimand (Table 13).

TABLE 13
Number of Subjects Achieving ACR 20 Response at Week 16 Based on the Composite
Estimand; Full Analysis Set 1 (Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Subjects evaluable for ACR 20 Response at | | | |
| Week 16^{a} | 244 | 247 | 242 |
| Subjects with ACR 20 Response^{b,h} | 83 (34.0%) | 137 (55.5%) | 137 (56.6%) |
| All subjects (including those with imputed | | | |
| data) | 246 | 248 | 245 |
| Subjects with ACR 20 Response^{b,c,h} | 83 (33.7%) | 137(55.2%) | 137(55.9%) |
| % Difference (95% CI)^{d} | 21.5 (13.1, 30.0) | 22.2 (13.7, 30.7) | |
| p-value^{e} | | <0.001 | <0.001 |

^{a}Subjects either have an observed ACR 20 response status or met a Treatment Failure (TF) criterion.
^{b}Defined as observed responders who had not met any TF criteria prior to Week 16.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and CRP prior to randomization (<2.0 mg/dL vs ≥2.0 mg/dL). The p-values for the global multiplicity adjustment are provided in table [TEFMULT01].
^{h}ACR 20 response is defined as ≥20% improvement from baseline in both tender joint count (68 joints) and swollen joint count (66 joints), and ≥20% improvement from baseline in at least 3 of the 5 assessments: patient's assessment of pain,patient's global assessment of disease activity, physician's global assessment of disease activity, HAQ-DI, and CRP.

### ACR 50 Response at Week 24

The proportion of subjects who achieved an ACR 50 response at Week 24 was numerically higher in both the guselkumab 100 mg q4w and the guselkumab 100 mg q8w groups compared with the placebo group based on the composite estimand (Table 14).

TABLE 14
Number of Subjects Achieving ACR 50 Response at Week 24 Based on the Composite
Estimand; Full Analysis Set 1 (Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Subjects evaluable for ACR 50 Response at | 244 | 246 | 244 |
| Week 24^{a} | | | |
| Subjects with ACR 50 Response^{b,h} | 35 (14.3%) | 78 (31.7%) | 81 (33.2%) |
| All subjects (including those with imputed | | | |
| data) | 246 | 248 | 245 |
| Subjects with ACR 50 Response^{b,c,h} | 35 (14.2%) | 78 (31.5%) | 81 (33.1%) |
| % Difference (95% CI)^{d} | | 17.2 (10.0, 24.4) | 18.8 (11.5, 26.1) |
| p-value^{e} | | <0.001 | <0.001 |

^{a}Subjects either have an observed ACR 50 response status or met a Treatment Failure (TF) criterion.
^{b}Defined as observed responders who had not met any TF criteria prior to Week 24.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and CRP prior to randomization (<2.0 mg/dL vs ≥2.0 mg/dL).
^{h}ACR 50 response is defined as ≥50% improvement from baseline in both tender joint count (68 joints) and swollen joint count (66 joints), and ≥50% improvement from baseline in at least 3 of the 5 assessments: patient's assessment of pain, patient's global assessment of disease activity, physician's global assessment of disease activity, HAQ-DI, and CRP.

### ACR 50 Response at Week 16

The proportion of subjects who achieved an ACR 50 response at Week 16 was numerically higher in both the guselkumab 100 mg q4w and the guselkumab 100 mg q8w groups compared with the placebo group based on the composite estimand (Table 15).

TABLE 15
Number of Subjects Achieving ACR 50 Response at Week 16 Based on the Composite
Estimand; Full Analysis Set 1 (Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Subjects evaluable for ACR 50 Response at | | | |
| Week 16^{a} | 245 | 248 | 241 |
| Subjects with ACR 50 Response^{b,h} | 23 (9.4%) | 71 (28.6%) | 51 (21.2%) |
| All subjects (including those with imputed | | | |
| data) | 246 | 248 | 245 |
| Subjects with ACR 50 Response^{b,c,h} | 23 (9.3%) | 71 (28.6%) | 51 (20.8%) |
| % Difference (95% CI)^{d} | | 19.3 (12.6, 25.9) | 11.5 (5.2, 17.7) |
| p-value^{e} | | <0.001 | <0.001 |

^{a}Subjects either have an observed ACR 50 response status or met a Treatment Failure (TF) criterion.
^{b}Defined as observed responders who had not met any TF criteria prior to Week 16.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and CRP prior to randomization (<2.0 mg/dL vs ≥2.0 mg/dL).
^{h}ACR 50 response is defined as ≥50% improvement from baseline in both tender joint count (68 joints) and swollen joint count (66 joints), and ≥50% improvement from baseline in at least 3 of the 5 assessments: patient's assessment of pain, patient's global assessment of disease activity, physician's global assessment of disease activity. HAQ-DI, and CRP.

### ACR 70 Response at Week 24

The proportion of subjects who achieved an ACR 70 response at Week 24 was numerically higher in both the guselkumab 100 mg q4w and the guselkumab 100 mg q8w groups compared with the placebo group based on the composite estimand (Table 16).

TABLE 16
Number of Subjects Achieving ACR 70 Response at Week 24 Based on the Composite
Estimand; Full Analysis Set 1 (Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Subjects evaluable for ACR 70 Response at | 245 | 246 | 244 |
| Week 24^{a} | | | |
| Subjects with ACR 70 Response^{b,h} | 10 (4.1%) | 46 (18.7%) | 32 (13.1%) |
| All subjects (including those with imputed | 246 | 248 | 245 |
| data) | | | |
| Subjects with ACR 70 Response^{b,c,h} | 10 (4.1%) | 46 (18.5%) | 32 (13.1%) |
| % Difference (95% CI)^{d} | | 14.5 (9.1, 19.9) | 9.0 (4.1, 13.8) |
| p-value^{e} | | <0.001 | <0.001 |

^{a}Subjects either have an observed ACR 70 response status or met a Treatment Failure (TF) criterion.
^{b}Defined as observed responders who had not met any TF criteria prior to Week 24.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and CRP prior to randomization (<2.0 mg/dL vs ≥2.0 mg/dL).
^{h}ACR 70 response is defined as ≥70% improvement from baseline in both tender joint count (68 joints) and swollen joint count (66 joints), and ≥70% improvement from baseline in at least 3 of the 5 assessments: patient's assessment of pain, patient's global assessment of disease activity, physician's global assessment of disease activity, HAQ-DI, and CRP.

Major Secondary Endpoints Conditionally Controlled Only in the US specific Testing

### Procedure

Change from Baseline in Enthesitis Score at Week 24

Based on CNTO1959PSA3002 data only, among the 506 (68.5%) subjects with enthesitis at baseline, a numerically greater reduction from baseline in LEI score at Week 24 was observed in both the guselkumab 100 mg q4w and the guselkumab 100 mg q8w groups compared with the placebo group (nominal p=0.002 and p<0.001, respectively; Table 17). Based on CNTO1959PSA3001 data only, among the 222 (58.3%) subjects with enthesitis at baseline, a numerically greater reduction from baseline in LEI score at Week 24 was observed in both the guselkumab 100 mg q4w group and the guselkumab 100 mg q8w group compared with the placebo group (nominal p=0.004 and p=0.185, respectively; Table 17). For both studies, the treatment effect was numerically greater in both guselkumab groups compared with the placebo group and allowed for the pooled analysis to be performed for both doses for this endpoint.

TABLE 17
Change from Baseline in Enthesitis Score (based on LEI) at Week 24 Based on the Composite
Estimand Using MI and an ANCOVA Model; Full Analysis Set 1 among the Subjects with
Enthesitis (based on LEI) at Baseline (Studies CNTO1959PSA3001 and CNTO1959P5A3002)

| | CNTO1959PSA3001 | | | CNTO1959PSA3002 | | | 2-Study Combined | | |
|---|---|---|---|---|---|---|---|---|---|
| | | Guselkumab | | | Guselkumab | | | Guselkumab | |
| | | 100 mg | 100 mg | | 100 mg | 100 mg | | 100 mg | 100 mg |
| | Placebo | q8w | q4w | Placebo | q8w | q4w | Placebo | q8w | q4w |
| Analysis set: Full | 77 | 72 | 73 | 178 | 158 | 170 | 255 | 230 | 243 |
| Analysis Set 1 | | | | | | | | | |
| Among the Subjects | | | | | | | | | |
| with Enthesitis (LEI) | | | | | | | | | |
| at Baseline | | | | | | | | | |
| Week 24 | 77 | 72 | 73 | 178 | 158 | 170 | 255 | 230 | 243 |
| All subjects at | | | | | | | | | |
| Week 24 | | | | | | | | | |
| (including | | | | | | | | | |
| those whose | | | | | | | | | |
| missing change | | | | | | | | | |
| imputed by mI)^{a, b} | | | | | | | | | |
| N | | | | | | | | | |
| Mean (SE)^{c} | −0.883 | −1.194 | −1.726 | −1.033 | −1.519 | −1.620 | −0.987 | −1.418 | −1.652 |
| | (0.1783) | (0.2190) | (0.2252) | (0.1244) | (0.1390) | (0.1255) | (0.1020) | (0.1177) | (0.1106) |
| Model Based | −1.01 | −1.35 | −1.75 | −1.03 | −1.60 | −1.52 | −1.02 | −1.52 | −1.59 |
| Estimates | (−1.37, | (−1.72, | (−2.13, | (−1.25, | (−1.84, | (−1.75, | (−1.22, | (−1.73, | (−1.79, |
| LSMean (95% CI)^{d} | −0.66) | −0.98) | −1.38) | −0.81) | −1.37) | −1.29) | −0.82) | −1.31) | −1.38) |
| LSMean | | −0.33 | −0.74 | | −0.57 | −0.49 | | −0.50 | −0.57 |
| Difference | | (−0.83, | (−1.24, | | (−0.89, | (−0.80, | | (−0.77, | (−0.83, |
| (95% CI)^{d} | | 0.16) | −0.24) | | −0.26) | −0.19) | | −0.23) | −0.31) |
| p-value^{e} | | 0.185 | 0.004 | | <0.001 | 0.002 | | <0.001 | <0.001 |
| LSMean | | | −0.41 | | | 0.08 | | | −0.07 |
| Difference | | | (−0.91, | | | (−0.24, | | | (−0.34, |
| (95% CI)^{d} | | | 0.10) | | | 0.40) | | | 0.20) |
| p-value^{e} | | | 0.114 | | | 0.617 | | | 0.623 |

^{a}The estimand is defined as the change from baseline using observed data prior to meeting TF criteria and 0 (no improvement from baseline) after meeting TF criteria. The missing data were assumed to be missing at random (MAR).
^{b}Subjects with missing change value were imputed by multiple imputations (MI). Data at Week 2, which were only collected in Study CNTO1959P5A3002, were included in the MI procedure to impute missing change value for Study CNTO1959P5A3002, however, were excluded from the pooled data analyses for 2-study combined.
^{c}The average of the mean, taken over all the MI data sets, was presented. The variance of the mean was the weighted sum of the average within-imputation variance and the between-imputation variance.
^{d}The LSmean for each MI data set was calculated based on an Analysis of Covariance (ANCOVA) model for the change from baseline at the visit. The combined LSmean which was the average of the LSmean, taken over all the MI data sets, was presented.
^{e}The p-values were based on the approximately normal distribution of the combined LSmean. The enthesitis score (based on LEI) is a total score of 6 evaluated sites (left and right: lateral epicondyle humerus, medial femoral condyle, achilles tendon insertion) with a range from 0 to 6. A negative change from baseline indicates improvement.

Change from Baseline in Dactylitis Score at Week 24

Based on CNTO1959PSA3002 data only, among the 331 (44.8%) subjects with dactylitis at baseline, a numerically greater reduction from baseline in dactylitis score at Week 24 was observed in both the guselkumab 100 mg q4w group and the guselkumab 100 mg q8w group compared with the placebo group (both nominal p=0.002; Table 18). Based on CNTO1959PSA3001 data only, among the 142 (37.3%) subjects with dactylitis at baseline, a numerically greater reduction from baseline in dactylitis score at Week 24 was observed in both the guselkumab 100 mg q4w group and the guselkumab 100 mg q8w group compared with the placebo group (nominal p=0.225 and p=0.121, respectively; Table 18). For both studies, the treatment effect was numerically greater in both guselkumab groups compared with the placebo group and allowed for the pooled analysis to be performed for both doses for this endpoint.

TABLE 18
Change from Baseline in Dactylitis Score at Week 24 Based on the Composite Estimand Using
MI and an ANCOVA Model; Full Analysis Set 1 among the Subjects with Dactylitis at
Baseline (Studies CNTO1959PSA3001 and CNTO1959P5A3002)

| | CNTO1959PSA3001 | | | CNTO1959PSA3002 | | | 2-Study Combined | | |
|---|---|---|---|---|---|---|---|---|---|
| | | Guselkumab | | | Guselkumab | | | Guselkumab | |
| | | 100 mg | 100 mg | | 100 mg | 100 mg | | 100 mg | 100 mg |
| | Placebo | q8w | q4w | Placebo | q8w | q4w | Placebo | q8w | q4w |
| Analysis set: Full | 55 | 49 | 38 | 99 | 111 | 121 | 154 | 160 | 159 |
| Analysis Set 1 | | | | | | | | | |
| Among the | | | | | | | | | |
| Subjects with | | | | | | | | | |
| Dactylitis at | | | | | | | | | |
| Baseline | | | | | | | | | |
| Week 24 | 55 | 49 | 38 | 99 | 111 | 121 | 154 | 160 | 159 |
| All subjects at | | | | | | | | | |
| Week 24 | | | | | | | | | |
| (including | | | | | | | | | |
| those whose | | | | | | | | | |
| missing change | | | | | | | | | |
| imputed by Mi)^{a, b} | | | | | | | | | |
| N | | | | | | | | | |
| Mean (SE)^{c} | −3.018 | −6.102 | −6.474 | −4.151 | −5.809 | −6.215 | −3.746 | −5.899 | −6.277 |
| | (0.7365) | (1.4772) | (1.7809) | (0.7686) | (0.7410) | (0.7099) | (0.5599) | (0.6822) | (0.6848) |
| Model Based | −4.30 | −6.11 | −5.82 | −4.03 | −5.95 | −5.88 | −4.21 | −6.10 | −5.97 |
| Estimates | (−5.96, | (−7.81, | (−7.82, | (−4.96, | (−6.83, | (−6.74, | (−5.05, | (−6.92, | (−6.84, |
| LSMean | −2.63) | −4.41) | −3.83) | −3.10) | −5.08) | −5.01) | −3.36) | −5.27) | −5.11) |
| (95% CI)^{d} | | | | | | | | | |
| LSMean | | −1.82 | −1.53 | | −1.92 | −1.85 | | −1.89 | −1.77 |
| Difference | | (−4.12, | (−4.00, | | (−3.15, | (−3.04, | | (−2.99, | (−2.87, |
| (95% CI)^{d} | | 0.49) | 0.95) | | −0.70) | −0.65) | | −0.79) | −0.66) |
| p-value^{e} | | 0.121 | 0.225 | | 0.002 | 0.002 | | <0.001 | 0.002 |
| LSMean | | | 0.29 | | | 0.08 | | | 0.12 |
| Difference | | | (−2.25, | | | (−1.09, | | | (−0.97, |
| (95% CI)^{d} | | | 2.83) | | | 1.24) | | | 1.22) |
| p-value^{e} | | | 0.822 | | | 0.897 | | | 0.823 |

^{a}The estimand is defined as the change from baseline using observed data prior to meeting TF criteria and 0 (no improvement from baseline) after meeting TF criteria. The missing data were assumed to be missing at random (MAR).
^{b}Subjects with missing change value were imputed by multiple imputations (MI). Data at Week 2, which were only collected in Study CNTO1959PSA3002, were included in the MI procedure to impute missing change value for Study CNTO1959PSA3002, however, were excluded from the pooled data analyses for 2-study combined.
^{c}The average of the mean, taken over all the MI data sets, was presented. The variance of the mean was the weighted sum of the average within-imputation variance and the between-imputation variance.
^{d}The LSmean for each MI data set was calculated based on an Analysis of Covariance (ANCOVA) model for the change from baseline at the visit. The combined LSmean which was the average of the LSmean, taken over all the MI data sets, was presented.
^{e}The p-values were based on the approximately normal distribution of the combined LSmean. The dactylitis score is a total score of presence and severity of dactylitis in each digit using a scoring system from 0 (no dactylitis) to 3 (severe dactylitis). The final dactylitis score ranges from 0 to 60. A negative change from baseline indicates improvement.

### Other Efficacy Endpoints Related to Reduction of Joint Signs and Symptoms

### ACR 20, ACR 50, and ACR 70 Responses Through Week 24

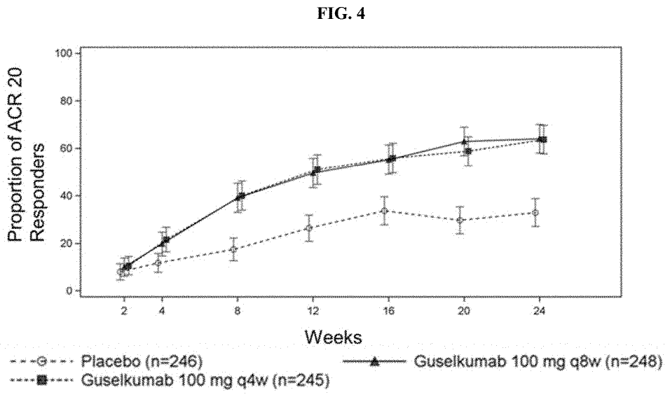

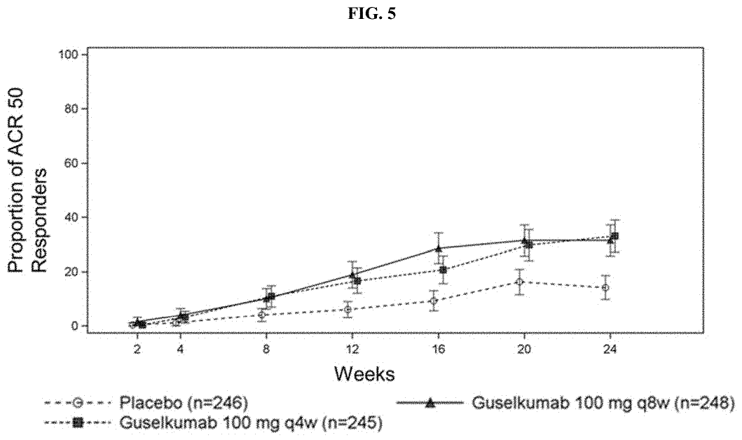

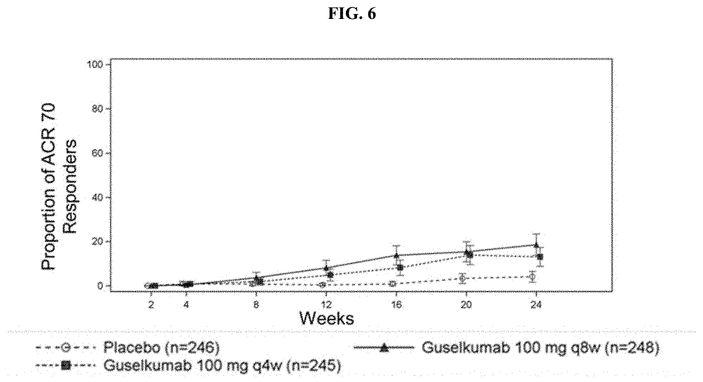

At Week 24, both guselkumab treatment groups had a numerically greater proportion of subjects with ACR 20, ACR 50, and ACR 70 responses compared with the placebo group (all nominal p<0.001) based on the composite estimand (FIG. 4, FIG. 5, FIG. 6).

### ACR Component Measurements Through Week 24

The 7 components of the ACR response are swollen and tender joint counts, patient's assessment of pain (by VAS), patient's and physician's global assessment of disease activity (by VAS), HAQ DI, and CRP. As early as Week 4, numerically greater improvements in all ACR components were seen in both guselkumab groups compared with the placebo group, with the exception of swollen join count, in which numerically greater improvements in the guselkumab groups compared with the placebo group were seen at Week 8. The improvement in each ACR component continued to increase over time through Week 24 in both guselkumab groups compared with the placebo group.

At Week 24, the median percent change from baseline in ACR components in the guselkumab 100 mg q4w and guselkumab 100 mg q8w groups compared with the placebo group were as follows:
Number of swollen joints: −81.5% and −85.7% compared with −65.5%, respectively
Number of tender joints: −66.7% and −60.0% compared with −33.3%, respectively
Patient's assessment of pain: −38.45% and −37.21% compared with −11.59%, respectively
Patient's global assessment of disease activity: −37.09% and −34.04% compared with −13.33%, respectively
Physician's global assessment of disease activity: −63.86% and −62.87% compared with −34.57%, respectively
HAQ-DI: −33.3333% and −27.2727% compared with −8.3333%, respectively
CRP: −48.218% and −53.175% compared with −17.494%, respectively

### PASI 50, PASI 75, PASI 90, and PASI 100 Responses Through Week 24

At Week 24, the proportions of subjects who achieved PASI 50, PASI 75, PASI 90, and PASI 100 responses in the guselkumab 100 mg q4w and guselkumab 100 mg q8w groups compared with the placebo group (all nominal p<0.001) were as follows:
PASI 50: 90.2% and 92.6% compared with 37.7%, respectively
PASI 75: 78.3% and 79.0% compared with 23.0%, respectively
PASI 90: 60.9% and 68.8% compared with 9.8%, respectively
PASI 100: 44.6% and 45.5% compared with 2.7%, respectively PASI 75 and ACR 20 Responses Through Week 24

Among the 543 (73.5%) subjects with ≥3% BSA psoriasis skin involvement and an IGA score of ≥2 at baseline, the proportion of subjects who achieved both a PASI 75 response and an

ACR 20 response was numerically greater in both guselkumab groups at Week 16 and Week 24 compared with the placebo group (all nominal p<0.001; Table 19). Consistent with PASI and ACR responses over time, the proportions of subjects achieving both PASI 75 and ACR 20 increased from Week 16 to Week 24 and were generally similar between the guselkumab 100 mg q4w group and the guselkumab 100 mg q8w group.

At Week 24, the proportions of subjects who achieved a PASI 75 and an ACR 20 response were numerically higher in both guselkumab groups compared with the placebo group (both nominal p<0.001) based on the composite estimand.

TABLE 19
Number of Subjects Achieving Both PASI 75 and ACR 20 Responses by Visit Through
Week 24, Based on the Composite Estimand; Full Analysis Set 1 Among the Subjects with
≥3% Body Surface Area (BSA) of Psoriatic Involvement and an IGA Score ≥2 (mild) at
Baseline (Study CNTO1959PSA3002)

| | | Guselkumab | |
|---|---|---|---|
| | Placebo | 100 mg q8w | 100 mg q4w |
| Analysis set: Full Analysis Set 1 Among | 183 | 176 | 184 |
| the Subjects Who had ≥3% Body Surface | | | |
| Area (BSA) of Psoriatic Involvement and | | | |
| an IGA Score ≥2 (mild) at Baseline | | | |
| Week 16 | | | |
| Subjects evaluable for PASI 75 and | 181 | 175 | 181 |
| ACR 20 responses^{a} | | | |
| Subjects with PASI 75 and ACR 20 | 19 (10.5%) | 86 (49.1%) | 89 (49.2%) |
| responses^{b,h} | | | |
| All subjects (including those with | 183 | 176 | 184 |
| imputed data) | | | |
| Subjects with PASI 75 and ACR 20 | 19 (10.4%) | 86 (48.9%) | 89 (48.4%) |
| response^{b,c,h} | | 38.4 (29.9, 46.9) | 37.7 (29.4, 46.1) |
| % Difference (95% CI)^{d} | | <0.001 | <0.001 |
| p-value^{e} | | | |
| Week 24 | | | |
| Subjects evaluable for PASI 75 and | 182 | 175 | 183 |
| ACR 20 responses^{a} | | | |
| Subjects with PASI 75 and ACR 20 | 21 (11.5%) | 100 (57.1%) | 105 (57.4%) |
| responses^{b,h} | | | |
| All subjects (including those with | 183 | 176 | 184 |
| imputed data) | | | |
| Subjects with PASI 75 and ACR 20 | 21 (11.5%) | 100 (56.8%) | 105 (57.1%) |
| response^{b,c,h} | | | |
| % Difference (95% CI)^{d} | | 45.1 (36.5, 53.6) | 45.8 (37.4, 54.2) |
| p-value^{e} | | <0.001 | <0.001 |

^{a}Subjects either have an observed PASI 75 and ACR 20 responses status or met a Treatment Failure (TF) criterion.
^{b}Defined as observed responders who had not met any TF criteria prior to the specific visit at which the endpoint was assessed.
^{c}Subjects with missing data at a visit are assumed to be non-responders at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and CRP prior to randomization (<2.0 mg/dL vs ≥2.0 mg/dL) is used to calculate the p-values. The symbol “†” if will be attached as a superscript to those p-values that are calculated using the Fisher's exact test.
^{h}The PASI score is a composite of the state of erythema, induration and scaling over the body along with the area of the involvement of psoriatic lesions. The PASI score ranges from 0 to 72, with a higher score indicating more severe disease. PASI 75 response is defined as ≥75% improvement from baseline in PASI score. ACR 20 response is defined as ≥20% improvement from baseline in both tender joint count (68 joints) and swollen joint count (66 joints), and ≥20% improvement from baseline in at least 3 of the 5 assessments: patient's assessment of pain, patient's global assessment of disease activity, physician's global assessment of disease activity, HAQ-DI, and CRP.

### PASI 75 and Modified PsARC Responses Through Week 24

Among the 543 (73.5%) subjects with ≥3% BSA psoriasis skin involvement and an IGA score of ≥2 at baseline, the proportion of subjects who achieved both a PASI 75 response and a modified PsARC response was numerically greater in both guselkumab treatment groups at Week 16 and Week 24 compared with the placebo group (all nominal p<0.001). The proportions increased from Week 16 to Week 24 and were generally similar between the guselkumab 100 mg q4w group and the guselkumab 100 mg q8w group.

At Week 24, the proportions of subjects who achieved a PASI 75 and a modified PsARC response were 60.9% and 65.3% in the guselkumab 100 mg q4w and guselkumab 100 mg q8w groups, respectively, compared with 15.3% in the placebo group (both nominal p<0.001).

### Psoriasis IGA Response Through Week 24

Among the 543 (73.5%) subjects with ≥3% BSA psoriasis skin involvement and an IGA score of ≥2 at baseline, numerically greater proportion of subjects achieved a psoriasis IGA response of 0 (clear) or 1 (minimal) and ≥2 grade reduction from baseline in both guselkumab groups at Week 16 and Week 24 compared with the placebo group.

At Week 16, a numerically greater proportion of subjects in both the guselkumab 100 mg q4w and the guselkumab 100 mg q8w groups (65.8% and 62.5%, respectively) achieved a psoriasis IGA response compared with the placebo group (15.3%; both nominal p<0.001). The proportions increased from Week 16 to Week 24 and were generally similar between the guselkumab 100 mg q4w group and the guselkumab 100 mg q8w group.

### Psoriasis IGA Score of 0 (Clear) Through Week 24

Among the 543 (73.5%) subjects with ≥3% BSA psoriasis skin involvement and an IGA score of ≥2 at baseline, numerically greater proportions of subjects achieved an IGA score of 0 (clear) in both guselkumab groups at Week 16 and Week 24 compared with the placebo group (Table 20). The proportions increased from Week 16 to Week 24 and were similar between the guselkumab 100 mg q4w group and the guselkumab 100 mg q8w group.

At Week 24, the proportions of subjects who achieved an IGA score of 0 (clear) were 50.5% and 50.0% in the guselkumab 100 mg q4w and guselkumab 100 mg q8w groups, respectively, compared with 7.7% in the placebo group (both nominal p<0.001).

TABLE 20
Number of Subjects with an IGA Score of 0 by Visit Through Week 24, Based on the
Composite Estimand; Full Analysis Set 1 Among the Subjects with ≥3% Body Surface Area (BSA) of
Psoriatic Involvement and an IGA Score ≥2 (mild) at Baseline (Study CNTO1959PSA3002)

| | | Guselkumab | |
|---|---|---|---|
| | Placebo | 100 mg q8w | 100 mg q4w |
| Analysis set: Full Analysis Set 1 | 183 | 176 | 184 |
| Among the Subjects Who had | | | |
| ≥3% Body Surface Area (BSA) of | | | |
| Psoriatic Involvement and an IGA | | | |
| Score ≥2 (mild) at Baseline | | | |
| Week 16 | | | |
| Subjects evaluable for an IGA | 182 | 176 | 182 |
| score of 0^{a} | | | |
| Subjects with an IGA score of | 11 (6.0%) | 68 (38.6%) | 75 (41.2%) |
| 0^{b,h} | | | |
| All subjects (including those | 183 | 176 | 184 |
| with imputed data) | | | |
| Subjects with an IGA score of | 11 (6.0%) | 68 (38.6%) | 75 (40.8%) |
| 0^{b,c,h} | | | |
| % Difference (95% CI)^{d} | | 32.4 (24.6, 40.2) | 34.8 (27.0, 42.6) |
| p-value^{e} | | <0.001 | <0.001 |
| Week 24 | | | |
| Subjects evaluable for an IGA | 182 | 175 | 183 |
| score of 0^{a} | | | |
| Subjects with an IGA score of | 14 (7.7%) | 88 (50.3%) | 93 (50.8%) |
| 0^{b,h} | | | |
| All subjects (including those | 183 | 176 | 184 |
| with imputed data) | | | |
| Subjects with an IGA score of | 14 (7.7%) | 88 (50.0%) | 93 (50.5%) |
| 0^{b,c,h} | | | |
| % Difference (95% CI)^{d} | | 42.2 (33.9, 50.4) | 43.1 (35.0, 51.1) |
| p-value^{e} | | <0.001 | <0.001 |

^{a}Subjects either have an observed IGA response status or met a Treatment Failure (TF) criterion.
^{b}Defined as observed responders who had not met any TF criteria prior to the specific visit at which the endpoint was assessed.
^{c}Subjects with missing data at a visit are assumed to be non-responders at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and CRP prior to randomization (<2.0 mg/dL vs ≥2.0 mg/dL) is used to calculate the p-values. The symbol “†” if will be attached as a superscript to those p-values that are calculated using the Fisher's exact test.
^{h}The IGA documents the investigator's assessment of the patient's psoriasis and lesions are graded for induration, erythema and scaling, each using a 5 point scale: 0 (no evidence), 1 (minimal), 2 (mild), 3 (moderate), and 4 (severe). The IGA score of psoriasis is based upon the average of induration, erythema and scaling scores.

### Other Efficacy Endpoints Related to Enthesitis

### Resolution of Enthesitis Over Time Through Week 24

At Week 16, subjects achieving enthesitis resolution were 40.6% and 47.5% in the guselkumab 100 mg q4w and guselkumab 100 mg q8w groups, respectively, compared with 30.9% in the placebo group (nominal p=0.070 and p=0.002, respectively) based on the composite estimand. The response rates increased from Week 16 to Week 24 for both guselkumab groups. The response rates were numerically higher in the guselkumab 100 mg q8w group compared with the guselkumab 100 mg q4w group from Week 8 through Week 24.

At Week 16 based on CNTO1959PSA3001 data only, among the 222 (58.3%) subjects with enthesitis at baseline, the proportion of subjects with resolution of enthesitis was numerically smaller in the guselkumab q8w group compared with the placebo group; therefore, pooling of the data at Week 16 from these studies was not justified for the guselkumab 100 mg q8w group. However, the treatment effect was numerically greater in the guselkumab 100 mg q4w group compared with the placebo group for both studies and allowed for the pooled analysis to be performed for the guselkumab 100 mg q4w group for this endpoint.

Among the 728 (65.0%) subjects with enthesitis at baseline based on pooled data from CNTO1959PSA3001 and CNTO1959PSA3002, a numerically greater proportion of subjects in the guselkumab 100 mg q4w group (42.0%) achieved enthesitis resolution at Week 16 compared with the placebo group based on the composite estimand.

Analysis based on the treatment policy estimand at Week 16 based on pooled data where all observed data collected for the endpoint were used and no treatment failure rules were applied confirmed the results of the main analysis.

Change from Baseline in the Enthesitis Score Over Time

Consistent with data on the proportion of subjects achieving enthesitis resolution over time, a numerically greater reduction from baseline in LEI score was observed in both guselkumab groups compared with the placebo group at each visit when enthesitis was assessed through Week 24 based on data from CNTO1959PSA3002 only.

At Week 16, a numerically greater reduction from baseline in LEI score was observed in both guselkumab groups compared with the placebo group based on the composite estimand. The reduction in LEI score continued to increase from Week 16 to Week 24 in both guselkumab groups. The effect was generally greater in the guselkumab 100 mg q4w group compared with the guselkumab 100 mg q8w group.

At Week 16 based on CNTO1959PSA3001 data only, among the 222 (58.3%) subjects with enthesitis at baseline, the reduction in change from baseline in LEI score was numerically greater in both the guselkumab groups compared with the placebo group based on the composite estimand. For both studies, the treatment effect was numerically greater in both guselkumab groups compared with the placebo group and allowed for the pooled analysis to be performed for both doses for this endpoint.

Among the 728 (65.0%) subjects with enthesitis at baseline based on pooled data from CNTO1959PSA3001 and CNTO1959PSA3002, a numerically greater reduction from baseline in LEI score at Week 16 was observed in both the guselkumab 100 mg q4w (−1.42) and guselkumab 100 mg q8w groups (−1.23) compared with the placebo group (−0.93; nominal p<0.001 and p=0.038, respectively) based on the composite estimand

### Other Efficacy Endpoints Related to Dactylitis

Resolution of Dactylitis Over Time Through Week 24

Based on CNTO1959PSA3002 data only, among the 331 (44.8%) subjects with dactylitis at baseline, the number of subjects achieving dactylitis resolution was numerically higher in both guselkumab groups compared with the placebo group at each visit from Week 2 through Week 24.

At Week 16, subjects achieving dactylitis resolution were 52.1% and 45.0% in the guselkumab 100 mg q4w and guselkumab 100 mg q8w groups, respectively, compared with 36.4% in the placebo group (nominal p=0.024 and p=0.192, respectively) based on the composite estimand. The response rates increased from Week 16 to Week 24 for both guselkumab groups. The response rates were numerically higher in the guselkumab 100 mg q4w group compared with the guselkumab 100 mg q8w group from Week 4 through Week 24.

At Week 16 based on CNTO1959PSA3001 data only, among the 142 (37.3%) subjects with dactylitis at baseline, a numerically greater proportion of subjects in both the guselkumab 100 mg q4w and the guselkumab 100 mg q8w groups (57.9% and 59.2%, respectively) achieved dactylitis resolution at Week 16 compared with the placebo group (43.6%; nominal p=0.169 and p=0.124, respectively) based on the composite estimand. For both studies, the treatment effect was numerically greater in both guselkumab groups compared with the placebo group and allowed for the pooled analysis to be performed for both doses for this endpoint.

Among the 473 (42.2%) subjects with dactylitis at baseline based on pooled data from CNTO1959PSA3001 and CNTO1959PSA3002, a numerically greater proportion of subjects in both the guselkumab 100 mg q4w and the guselkumab 100 mg q8w groups (53.5% and 49.4%, respectively) achieved dactylitis resolution at Week 16 compared with the placebo group (39.0%; nominal p=0.008 and p=0.053, respectively) based on the composite estimand.

Change from Baseline in the Dactylitis Score Through Week 24

Consistent with data on the proportion of subjects achieving dactylitis resolution over time, a numerically greater reduction from baseline in dactylitis score was observed in both guselkumab groups compared with the placebo group at each visit when dactylitis was assessed from Week 2 through Week 24 based on data from CNTO1959PSA3002 only. The effect was greater in the guselkumab 100 mg q4w group compared with the guselkumab 100 mg q8w group at Week 16 and Week 24.

### Other Efficacy Endpoints Related to BASDAI

Only subjects with spondylitis with peripheral arthritis as their primary arthritic presentation of PsA completed the BASDAI. Subjects with spondylitis and peripheral arthritis at baseline included 86, 73, and 99 subjects in the guselkumab 100 mg q4w, guselkumab 100 mg q8w, and placebo. Subjects with spondylitis and peripheral arthritis at baseline and BASDAI score>0 at baseline included 83, 67, and 92 subjects in the guselkumab 100 mg q4w, guselkumab 100 mg q8w, and placebo groups, respectively.

Among the 258 (34.9%) subjects with spondylitis and peripheral arthritis at baseline, a numerically greater reduction from baseline in BASDAI was observed in both guselkumab groups compared with the placebo group at each visit BASDAI was evaluated from Week 8 through Week 24 (Table 21). The reduction in BASDAI scores was generally similar between the guselkumab treatment groups.

At Week 24, a numerically greater reduction from baseline in BASDAI was observed in both the guselkumab 100 mg q4w group and the guselkumab 100 mg q8w group compared with the placebo group (both nominal p<0.001) based on the composite estimand.

TABLE 21
Summary of the Change from Baseline in the Bath Ankylosing Spondylitis Disease Activity
Index (BASDAI) by Visit Through Week 24, Based on the Composite Estimand Using an
MMRM Model; Full Analysis Set 1 Among the Subjects with Spondylitis and Peripheral
Arthritis at Baseline (Study CNTO1959PSA3002)

| | | Guselkumab | |
|---|---|---|---|
| | Placebo | 100 mg q8w | 100 mg q4w |
| Analysis set: Full Analysis Set 1 Among | 99 | 73 | 86 |
| the Subjects with Spondylitis and | | | |
| Peripheral Arthritis at Baseline | | | |
| Subjects with a baseline BASDAI = 0^{a,h} | 0 | 0 | 0 |
| Subjects with a baseline BASDAI > 0^{a,h} | 92 | 67 | 83 |
| Week 8 | | | |
| Subjects evaluable^{b} | | | |
| N | 92 | 66 | 82 |
| Mean (SD) | −0.790 (1.8049) | −1.602 (2.2637) | −1.582 (1.7255) |
| Median | −0.765 | −1.120 | −1.370 |
| Range | (−6.67; 3.24) | (−8.46; 4.54) | (−6.42; 1.56) |
| IQ range | (−1.900; 0.510) | (−2.550; 0.040) | (−2.510; −0.130) |
| Model Based Estimates of the Mean | | | |
| Change^{a,c} | | | |
| LSMean (95% CI)^{d} | −0.645 (−1.039, −0.251) | −1.429 (−1.914, −0.944) | −1.523 (−1.937, −1.109) |
| LSMean difference (95% CI) | | −0.784 (−1.347, −0.220) | −0.878 (−1.404, −0.352) |
| p-value^{d} | | 0.007 | 0.001 |
| Week 16 | | | |
| Subjects evaluable^{b} | | | |
| N | 92 | 66 | 81 |
| Mean (SD) | −1.168 (2.1668) | −2.312 (2.5152) | −2.265 (1.9895) |
| Median | −0.810 | −2.105 | −2.060 |
| Range | (−7.93; 2.91) | (−7.07; 2.65) | (−7.62; 2.50) |
| IQ range | (−2.610; 0.270) | (−4.240; −0.440) | (−3.510; −0.950) |
| Model Based Estimates of the Mean | | | |
| Change^{a,c} | | | |
| LSMean (95% CI)^{d} | −1.023 (−1.466, −0.580) | −2.139 (−2.680, −1.597) | −2.207 (−2.675, −1.740) |
| LSMean difference (95% CI) | | −1.115 (−1.761, −0.470) | −1.184 (−1.789, −0.579) |
| p-value^{d} | | <0.001 | <0.001 |
| Week 24 | | | |
| Subjects evaluable^{b} | | | |
| N | 92 | 65 | 82 |
| Mean (SD) | −1.369 (2.3488) | −2.589 (2.4080) | −2.560 (2.0137) |
| Median | −0.770 | −2.180 | −2.535 |
| Range | (−9.12; 3.19) | (−8.19; 1.07) | (−7.30; 1.09) |
| IQ range | (−2.885; 0.020) | (−4.150; −0.610) | (−4.190; −1.060) |
| Model Based Estimates of the Mean | | | |
| Change^{a,c} | | | |
| LSMean (95% CI)^{d} | −1.224 (−1.681, −0.767) | −2.431 (−2.989, −1.873) | −2.500 (−2.981, −2.019) |
| LSMean difference (95% CI) | | −1.207 (−1.877, −0.538) | −1.276(−1.902, −0.651) |
| p-value^{d} | | <0.001 | <0.001 |

^{a}Defined as the change from baseline using observed data or 0 (no improvement) if a subject met Treatment Failure (TF) criteria.
^{b}Subjects either have an observed change from baseline at this visit or met TF criteria prior to this visit.
^{c}The missing data is assumed to be MAR.
^{d}The LS means and p-values are based on the MMRM analysis.
^{h}The BASDAI is based on 6 questions relating to 5 major symptoms of ankylosing spondylitis through a patient's self assessment. A higher score indicates greater disease severity.

Subjects Achieving 5-Point Improvement from Baseline in SF 36 MCS Scores Through Week 24
The proportions of subjects who achieved clinically meaningful ≥5-point improvement from baseline in SF-36 MCS scores were numerically greater in both guselkumab groups compared with the placebo group from Week 8 through Week 24. The proportions increased over time through Week 24 in the guselkumab 100 mg q4w group. The proportion of subjects achieving ≥5-point improvement from baseline was highest at Week 16 for the guselkumab 100 mg q8w group (42.3%). The response rate was numerically higher in the guselkumab 100 mg q8w group compared with the guselkumab 100 mg q4w group from Week 8 through Week 24.
At Week 24, the proportion of subjects who achieved≥5-point improvement from baseline in SF-36 MCS score was 34.3% and 37.5% in the guselkumab 100 mg q4w and guselkumab 100 mg q8w groups, respectively, compared with 30.9% in the placebo group (nominal p=0.424 and p=0.124, respectively) based on the composite estimand.
For each SF-36 scale evaluated, a numerically greater increase from baseline in norm-based scores was observed in both guselkumab groups compared with the placebo group from Week 8 through Week 24. The increase from baseline in norm-based scores were generally higher in the guselkumab 100 mg q8w group compared with the guselkumab 100 mg q4w group.
At Week 24, the estimated LSmean of change from baseline in norm-based SF-36 subscales in the guselkumab 100 mg q4w and 100 mg q8w groups compared with the placebo group were as follows:
physical functioning: 6.624 and 6.703 compared with 3.254, respectively
role-physical: 6.241 and 6.549 compared with 3.365, respectively
bodily pain: 7.739 and 7.811 compared with 3.482, respectively
general health: 5.269 and 5.794 compared with 2.290, respectively
vitality: 7.009 and 7.373 compared with 3.835, respectively
social functioning: 5.922 and 5.806 compared with 2.978, respectively
role-emotional: 4.255 and 4.382 compared with 1.813, respectively
mental health: 4.767 and 4.490 compared with 2.335, respectively

### FACIT-Fatigue Score

Change from Baseline in FACIT-Fatigue Score Through Week 24
A numerically greater increase from baseline (improvement) in FACIT-Fatigue scores was observed in both guselkumab groups compared with the placebo group at each visit the FACIT Fatigue was evaluated (Weeks 8, 16, and 24; all nominal p<0.001; Table 22). The scores continued to increase in the guselkumab groups over time through Week 24 and were numerically higher in the guselkumab 100 mg q8w compared with the guselkumab 100 mg q4w group at each visit.

TABLE 22
Summary of the Change from Baseline in FACIT-Fatigue Score by Visit Through Week 24,
Based on the Composite Estimand Using an MMRM Model; Full Analysis Set 1
(Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Change from baseline in FACIT-Fatigue | | | |
| score^{a,h} | | | |
| Week 8 | | | |
| Subjects evaluable^{b} | | | |
| N | 245 | 247 | 245 |
| Mean (SD) | 2.657 (7.8676) | 5.194 (8.3307) | 4.441 (7.8590) |
| Median | 3.000 | 5.000 | 4.000 |
| Range | (−23.00; 35.00) | (−19.00; 36.00) | (−32.00; 31.00) |
| IQ range | (−3.000; 7.000) | (0.000; 10.000) | (0.000; 8.000) |
| Model Based Estimates of the Mean | | | |
| Change^{a,c} | | | |
| LSMean (95% CI)^{d} | 2.451 (1.508, 3.395) | 5.031 (4.092, 5.970) | 4.850 (3.905, 5.795) |
| LSMean difference (95% CI) | | 2.580 (1.283, 3.876) | 2.398 (1.096, 3.701) |
| p-value^{d} | | <0.001 | <0.001 |
| Week 16 | | | |
| Subjects evaluable^{b} | | | |
| N | 244 | 248 | 243 |
| Mean (SD) | 3.943 (8.4140) | 7.101 (9.3559) | 6.169 (8.7188) |
| Median | 4.000 | 7.000 | 5.000 |
| Range | (−25.00; 38.00) | (−17.00; 37.00) | (−26.00; 35.00) |
| IQ range | (−1.000; 9.000) | (0.000; 13.000) | (0.000; 11.000) |
| Model Based Estimates of the Mean | | | |
| Change^{a,c} | | | |
| LSMean (95% CI)^{d} | 3.696 (2.675, 4.717) | 6.977 (5.963, 7.992) | 6.598 (5.574, 7.622) |
| LSMean difference (95% CI) | | 3.281 (1.874, 4.689) | 2.902 (1.486, 4.318) |
| p-value^{d} | | <0.001 | <0.001 |
| Week 24 | | | |
| Subjects evaluable^{b} | | | |
| N | 244 | 246 | 245 |
| Mean (SD) | 3.734 (8.6950) | 7.691 (9.8682) | 6.702 (8.6340) |
| Median | 2.000 | 6.000 | 5.000 |
| Range | (−16.00; 37.00) | (−19.00; 41.00) | (−26.00; 35.00) |
| IQ range | (−1.000; 9.000) | (1.000; 14.000) | (1.000; 11.000) |
| Model Based Estimates of the Mean | | | |
| Change^{a,c} | | | |
| LSMean (95% CI)^{d} | 3.559 (2.500, 4.619) | 7.550 (6.496, 8.603) | 7.111 (6.051, 8.171) |
| LSMean difference (95% CI) | | 3.990 (2.526, 5.454) | 3.551 (2.082, 5.021) |
| p-value^{d} | | <0.001 | <0.001 |

^{a}Defined as the change from baseline using observed data or 0 (no improvement) if a subject met Treatment Failure (TF) criteria.
^{b}Subjects either have an observed change from baseline at this visit or met TF criteria prior to this visit.
^{c}The missing data is assumed to be MAR.
^{d}The LS means and p-values are based on the MMRM analysis.
^{h}The FACIT-fatigue score is calculated based on the FACIT-fatigue questionnaire that comprises of 13 questions, with each question graded on a 5-point scale (0-4). The FACIT-fatigue scores can range from 0 to 52 with higher scores indicating less fatigue.

### EQ-5D-5L Questionnaire

At Week 24, a numerically greater increase from baseline in EQ-5D index scores was observed in both the guselkumab 100 mg q4w group (LSmean: 0.116) and the guselkumab 100 mg q8w group (LSmean: 0.115) compared with the placebo group (LSmean: 0.053; both nominal p<0.001) based on the composite estimand.

At Week 24, a numerically greater increase from baseline in EQ-5D health state VAS score was observed in both the guselkumab 100 mg q4w group (LSmean: 18.089) and the guselkumab 100 mg q8w group (LSmean: 18.371) compared with the placebo group (LSmean: 6.796; both nominal p<0.001) based on the composite estimand.

Change from Baseline in PASDAS Through Week 24

A numerically greater reduction from baseline (improvement) in PASDAS score was observed in both guselkumab groups compared with the placebo group at each visit PASDAS was evaluated (Weeks 8, 16, and 24; all nominal p<0.001).

At Week 24, a numerically greater reduction from baseline in PASDAS score was observed in both the guselkumab 100 mg q4w group (LSmean: −2.399) and the guselkumab 100 mg q8w group (LSmean: −2.403) compared with the placebo group (LSmean: −1.336; both nominal p<0.001) based on the composite estimand.

Change from Baseline in GRACE Index Through Week 24

A numerically greater reduction from baseline (improvement) in GRACE index was observed in both guselkumab groups compared with the placebo group at each visit the GRACE index was evaluated (Week 16 and Week 24; all nominal p<0.001. The reduction in GRACE index was similar between the guselkumab groups at each visit.

At Week 24, a numerically greater reduction from baseline in GRACE index was observed in both the guselkumab 100 mg q4w group (LSmean: −2.589) and the guselkumab 100 mg q8w group (LSmean: −2.592) compared with the placebo group (LSmean: −1.197; both nominal p<0.001) based on the composite estimand.

Change from Baseline in mCPDAI Through Week 24

A numerically greater reduction from baseline (improvement) in mCPDAI scores were observed in both guselkumab groups compared with the placebo group at each visit the mCPDAI score was evaluated (Week 16 and Week 24; all nominal p<0.001). The reduction in mCPDAI score was slightly higher in the guselkumab 100 mg q4w group compared with the guselkumab 100 mg q8w group at both visits.

At Week 24, a numerically greater reduction from baseline in mCPDAI score was observed in both the guselkumab 100 mg q4w group (LSmean: −3.09) and the guselkumab 100 mg q8w group (LSmean: −2.94) compared with the placebo group (LSmean: −1.30; both nominal p<0.001) based on the composite estimand.

Low Disease Activity Based on mCPDAI Through Week 24

At baseline, the proportion of subjects with low disease activity based on the mCPDAI index was 1.6%, 6.5%, and 1.6% in the guselkumab 100 mg q4w, guselkumab 100 mg q8w, and placebo groups, respectively.

Consistent with the change from baseline in mCPDAI score over time, the proportion of subjects achieving low disease activity based on the mCPDAI score was higher in the guselkumab 100 mg q4w and guselkumab 100 mg q8w groups (34.4% and 34.7%, respectively) compared with the placebo group (12.6%; both nominal p<0.001) at Week 16. The proportions increased in the guselkumab groups from Week 16 to Week 24 and were numerically higher in the guselkumab 100 mg q8w group compared with the guselkumab 100 mg q4w group.

At Week 24, the proportion of subjects achieving low disease activity based on the mCPDAI score was 41.2% and 46.4% in the guselkumab 100 mg q4w and guselkumab 100 mg q8w groups, respectively, compared with 14.2% in the placebo group (both nominal p<0.001) based on the composite estimand.

### MDA Criteria Through Week 24

At baseline, 1 (0.4%) subject in the guselkumab 100 mg q4w group met MDA criteria (Table 23).

The proportions of subjects who met MDA criteria at Week 16 and Week 24 were numerically greater in both guselkumab groups compared with the placebo group (all nominal p<0.001). The proportions who met MDA criteria were numerically higher in the guselkumab 100 mg q8w group compared with the guselkumab 100 mg q4w group at both visits.

TABLE 23
Number of Subjects Who Achieved the Minimal Disease Activity (MDA) Criteria by Visit
Through Week 24, Based on the Composite Estimand; Full Analysis Set 1
(Study CNTO1959PSA3002)

| | | Guselkumab | |
|---|---|---|---|
| | Placebo | 100 mg q8w | 100 mg q4w |
| Analysis set: Full Analysis Set 1 | 246 | 248 | 245 |
| Baseline | | | |
| Subjects evaluable for MDA response^{a} | 246 | 248 | 245 |
| Subjects with MDA response^{b,h} | 0 | 0 | 1 (0.4%) |
| Week 16 | | | |
| Subjects evaluable for MDA response^{a} | 245 | 248 | 243 |
| Subjects with MDA response^{b,h} | 8 (3.3%) | 42 (16.9%) | 32 (13.2%) |
| All subjects (including those with imputed | 246 | 248 | 245 |
| data) | | | |
| Subjects with MDA response^{b,c,h} | 8 (3.3%) | 42 (16.9%) | 32 (13.1%) |
| % Difference (95% CI)^{d} | | 13.7 (8.5, 18.8) | 9.8 (5.1, 14.5) |
| p-value^{e} | | <0.001 | <0.001 |
| Week 24 | | | |
| Subjects evaluable for MDA response^{a} | 245 | 246 | 245 |
| Subjects with MDA response^{b,h} | 15 (6.1%) | 62 (25.2%) | 46 (18.8%) |
| All subjects (including those with imputed | | | |
| data) | 246 | 248 | 245 |
| Subjects with MDA response^{b,c,h} | 15 (6.1%) | 62 (25.0%) | 46 (18.8%) |
| % Difference (95% CI)^{d} | | 18.9 (12.8, 25.0) | 12.7 (7.0, 18.4) |
| p-value^{e} | | <0.001 | <0.001 |

^{a}Subjects either have an observed MDA response status or met a Treatment Failure (TF) criterion.
^{b}Defined as observed responders who had not met any TF criteria prior to the specific visit at which the endpoint was assessed.
^{c}Subjects with missing data at a visit are assumed to be non-responders at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and CRP prior to randomization (<2.0 mg/dL vs ≥2.0 mg/dL) is used to calculate the p-values. The symbol “†” will be attached as a superscript to those p-values that are calculated using the Fisher's exact test.
^{h}MDA is achieved if at least 5 of the 7 criteria are met (tender joint count ≤1, swollen joint count ≤1, psoriasis activity and severity index ≤1, patient's assessment of pain ≤15, patient's global assessment of disease activity ≤20, HAQ-DI score ≤0.5, Tender entheseal points ≤1).

### VLDA Criteria Through Week 24

At baseline, no subjects in the guselkumab groups or the placebo group met VLDA criteria. The proportions of subjects who met VLDA criteria at Week 16 and Week 24 were low but numerically greater in both guselkumab groups compared with the placebo group. The proportions were slightly higher in the guselkumab 100 mg q4w group compared with the guselkumab 100 mg q8w group at both visits.

At Week 24, the proportion of subjects who met VLDA criteria were 4.9% and 4.4% in the guselkumab 100 mg q4w and guselkumab 100 mg q8w groups, respectively, compared with 1.2% in the placebo group (nominal p=0.018 and p=0.032, respectively) based on the composite

### Efficacy and Pharmacokinetics

The relationships between selected efficacy endpoints and trough serum guselkumab concentrations were assessed based on the PK analysis set. Clinical efficacy data (composite estimand) with no missing data imputation and respective trough serum guselkumab concentrations were used in the following analyses:
ACR 20 or ACR 50 responses or change from baseline in DAS28 (CRP) at Week 12 by trough serum guselkumab concentration at Week 12.
ACR 20 or ACR 50 responses or change from baseline in DAS28 (CRP) at Week 20 or Week 24 by steady-state trough serum guselkumab concentration at Week 20.
IGA response at Weeks 24 by steady-state trough serum guselkumab concentration at Week 20 (in subjects with ≥3% BSA psoriatic involvement and an IGA score of ≥2 at baseline).

### ACR 20 and ACR 50 Responses and Trough Serum Guselkumab Concentrations

There were no apparent exposure-response relationships for ACR 20 or ACR 50 response rates at Week 12 by trough guselkumab concentration quartiles at Week.

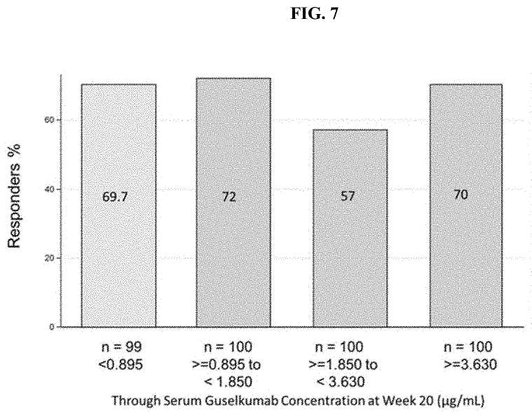

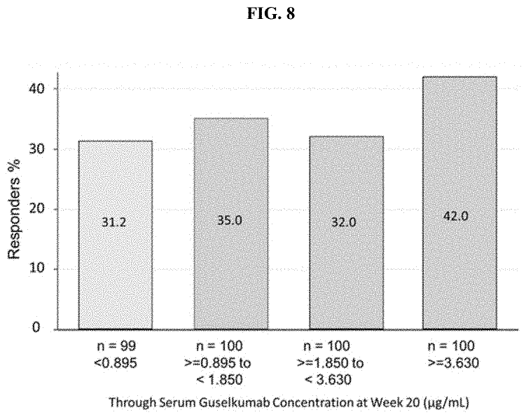

No consistent exposure-response relationships were observed for ACR 20 response rates at Week 20 or Week 24 by trough guselkumab concentration quartiles at Week 20 (FIG. 7). There appeared to be weak exposure-response relationships for ACR 50 response rates at Week 20 or Week 24 by trough guselkumab concentration quartiles at Week 20 (FIG. 8).

Change from Baseline in DAS28 (CRP) by Trough Serum Guselkumab Concentrations

There was no apparent exposure-response relationship for mean change from baseline in DAS28 (CRP) at Week 12 by trough guselkumab concentration quartiles at Week 12 (There were also no apparent exposure-response relationships for mean changes from baseline in DAS28 (CRP) at Week 20 or Week 24 by trough guselkumab concentration quartiles at Week 20

### IGA Response and Trough Serum Guselkumab Concentrations

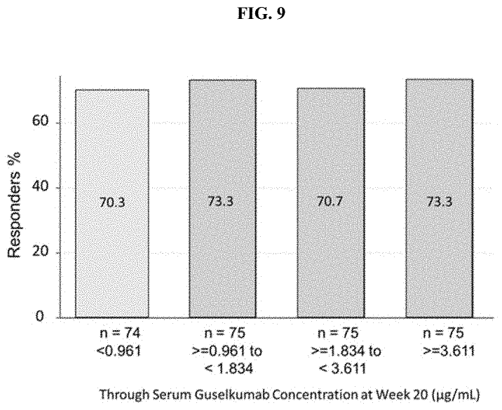

There was no apparent exposure-response relationship in IGA response at Week 24 by trough guselkumab concentration quartiles at Week 20 in subjects with ≥3% BSA psoriatic involvement and an IGA score of ≥2 at baseline (FIG. 9).

### Efficacy Summary

### Primary Endpoint

A significantly greater proportion of subjects in both the guselkumab 100 mg q4w and guselkumab 100 mg q8w groups (63.7% and 64.1%, respectively) achieved an ACR 20 response at Week 24 compared with subjects in the placebo group (32.9%) based on the global (ex-US) and US-specific multiplicity testing procedures (both adjusted p<0.001).

### Major Secondary Endpoints

Major Secondary Endpoints Controlled for Multiplicity in Both the Global (ex-US) and US-specific Testing Procedures
A significantly greater reduction from baseline in HAQ-DI score at Week 24 was observed in both the guselkumab 100 mg q4w (LSmean: −0.4004) and the guselkumab 100 mg q8w groups (LSmean: −0.3672) compared with the placebo group (LSmean: −0.1300; both global and US-specific adjusted p<0.001).
Among the 543 (73.5%) subjects with ≥3% BSA of psoriatic involvement and an IGA score of ≥2 (mild) at baseline, a significantly greater proportion of subjects in both the guselkumab 100 mg q4w and the guselkumab 100 mg q8w groups (68.5% and 70.5%, respectively) achieved a psoriasis IGA response of 0 (cleared) or 1 (minimal) and ≥2-grade reduction from baseline in the IGA psoriasis score at Week 24 compared with the placebo group (19.1%; both global and US-specific adjusted p<0.001).
A numerically smaller (less progression) change from baseline in modified vdH-S score at Week 24 was observed in both the guselkumab 100 mg q4w (LSmean: 0.29) and the guselkumab 100 mg q8w groups (LSmean: 0.52) compared with the placebo group (LSmean: 0.95). Based on the global (ex-US)-specific and US-specific multiplicity testing procedures, the difference in LSmean change was statistically significant in the guselkumab 100 mg q4w group compared with the placebo group (adjusted global p=0.006 and adjusted US-specific p=0.011, respectively), but was not significant in the guselkumab 100 mg q8w group (adjusted global p=0.068 and adjusted US-specific p=0.072, respectively). Statistical significance was not formally tested in the global (ex-US)-specific testing procedure for the guselkumab 100 mg q8w group for the remaining major secondary endpoints as the change from baseline in modified vdH-S score at Week 24 was not significant for this group (adjusted p=0.068).
A numerically greater improvement from baseline in SF-36 PCS score at Week 24 was observed in both the guselkumab 100 mg q4w (LSmean: 7.04) and guselkumab 100 mg q8w groups (LSmean: 7.39) compared with the placebo group (LSmean: 3.42). Based on the global (ex-US)-specific multiplicity testing procedure, the mean change was statistically significant in the guselkumab 100 mg q4w group compared with the placebo group (adjusted p=0.006) and was not formally tested in the guselkumab 100 mg q8w group. Based on the US-specific testing procedure, the mean change was statistically significant in both the guselkumab 100 mg q4w and guselkumab 100 mg q8w groups compared with the placebo group (both adjusted p=0.011).
A numerically greater improvement from baseline in SF-36 MCS score at Week 24 was observed in both the guselkumab 100 mg q4w (LSmean: 4.22) and guselkumab 100 mg q8w groups (LSmean: 4.17) compared with the placebo group (LSmean: 2.14). Based on the global (ex-US)-specific multiplicity testing procedure, the mean change was statistically significant in the guselkumab 100 mg q4w group compared with the placebo group (adjusted p=0.006) and was not formally tested in the guselkumab 100 mg q8w group. Based on the US-specific multiplicity testing procedure, the mean change was not statistically significant in the guselkumab 100 mg q4w or guselkumab 100 mg q8w groups compared with the placebo group (both adjusted p=0.072).
Among the 728 (65.0%) subjects with enthesitis at baseline based on pooled data from CNTO1959PSA3001 and CNTO1959PSA3002, a numerically greater proportion of subjects in both the guselkumab 100 mg q4w and the guselkumab 100 mg q8w groups (44.9% and 49.6%, respectively) achieved enthesitis resolution at Week 24 compared with the placebo group (29.4%). Based on the global (ex-US)-specific multiplicity testing procedure, the proportion of subjects with enthesitis resolution was significantly greater in the guselkumab 100 mg q4w group compared with the placebo group (adjusted p=0.006) and was not formally tested in the guselkumab 100 mg q8w group. Based on the US-specific multiplicity testing procedure, the proportion of subjects with enthesitis resolution was significantly greater in both the guselkumab 100 mg q4w and the guselkumab 100 mg q8w groups compared with the placebo group (both adjusted p=0.030).
Among the 473 (42.2%) subjects with dactylitis at baseline based on pooled data from CNTO1959PSA3001 and CNTO1959PSA3002, a numerically greater proportion of subjects in both the guselkumab 100 mg q4w and the guselkumab 100 mg q8w groups (63.5% and 59.4%, respectively) achieved dactylitis resolution at Week 24 compared with the placebo group (42.2%). Based on the global (ex-US)-specific multiplicity testing procedure, the proportion of subjects with dactylitis resolution was significantly higher in the guselkumab 100 mg q4w group compared with the placebo group (adjusted p=0.006) and was not formally tested in the guselkumab 100 mg q8w group. Based on the US-specific multiplicity testing procedure, the proportion of subjects with dactylitis resolution was significantly greater in both the guselkumab 100 mg q4w and the guselkumab 100 mg q8w groups compared with the placebo group (adjusted p=0.011 and p=0.030, respectively).
Major Secondary Endpoints Controlled for Multiplicity in the Global (ex-US) Testing Procedure and Conditionally Controlled in the US-specific Testing Procedure
The following major secondary endpoints were controlled for multiplicity in the global (ex-US) testing procedure. In addition, these endpoints were also tested for both guselkumab doses based on the US-specific testing procedure (all nominal p<0.001) since these endpoints were highly correlated with the primary endpoint and statistical significance was achieved for ACR 20 response at Week 24 in both the guselkumab 100 mg q4w and guselkumab 100 mg q8w groups compared with the placebo group.
A significantly greater reduction from baseline in DAS28 (CRP) score at Week 24 was observed in both the guselkumab 100 mg q4w (LSmean: −1.62) and guselkumab 100 mg q8w groups (LSmean: −1.59) compared with the placebo group (LSmean: −0.97; both global adjusted p<0.001).
For the following major secondary endpoints, the guselkumab 100 mg q4w group demonstrated statistical significance compared with the placebo group (adjusted p=0.006) based on the global (ex-US) multiplicity testing procedure. Statistical significance could not be assessed for the guselkumab 100 mg q8w group compared with the placebo group as the endpoint for change from baseline in modified vdH-S score at Week 24 was not significant in the guselkumab 100 mg q8w group
The proportion of subjects who achieved an ACR 20 response at Week 16 was numerically higher in both the guselkumab 100 mg q4w and guselkumab 100 mg q8w groups (55.9% and 55.2%, respectively) compared with the placebo group (33.7%; nominal p<0.001).
The proportion of subjects who achieved an ACR 50 response at Week 24 was numerically higher in both the guselkumab 100 mg q4w and the guselkumab 100 mg q8w groups (33.1% and 31.5%, respectively) compared with the placebo group (14.2%; nominal p<0.001).
The proportion of subjects who achieved an ACR 50 response at Week 16 was numerically higher in both the guselkumab 100 mg q4w and the guselkumab 100 mg q8w groups (20.8% and 28.6%, respectively) compared with the placebo group (9.3%; nominal p<0.001).
The proportion of subjects who achieved an ACR 70 response at Week 24 was numerically higher in the guselkumab 100 mg q4w and the guselkumab 100 mg q8w groups (13.1% and 18.5%, respectively) compared with the placebo group (4.1%; nominal p<0.001).
Major Secondary Endpoints Conditionally Controlled Only in the US-specific Testing Procedure
Change from baseline in enthesitis score at Week 24 and change from baseline in dactylitis score at Week 24 were formally tested in the US-specific testing procedure for both guselkumab doses based on pooled data from CNTO1959PSA3001 and CNTO1959PSA3002 since resolution of enthesitis at Week 24 and resolution of dactylitis at Week 24, respectively, achieved statistical significance in both the guselkumab 100 mg q4w and guselkumab 100 mg q8w groups compared with the placebo group.
Among the 728 (65.0%) subjects with enthesitis at baseline based on pooled data from CNTO1959PSA3001 and CNTO1959PSA3002, a numerically greater reduction from baseline in LEI score at Week 24 was observed in both the guselkumab 100 mg q4w (LSmean: −1.59) and guselkumab 100 mg q8w groups (LSmean: −1.52) compared with the placebo group (LSmean: −1.02; both nominal p<0.001).
Among the 473 (42.2%) subjects with dactylitis at baseline based on pooled data from CNTO1959PSA3001 and CNTO1959PSA3002, a numerically greater reduction from baseline in dactylitis score at Week 24 was observed in both the guselkumab 100 mg q4w (LSmean: −5.97) and guselkumab 100 mg q8w groups (LSmean: −6.10) compared with the placebo group (LSmean: −4.21; nominal p=0.002 and p<0.001, respectively).
Other Secondary Efficacy Analyses
Other Efficacy Endpoints Related to Reduction of Joint Signs and Symptoms
The median percent improvement from baseline was numerically greater for both guselkumab groups compared with the placebo group for each ACR component from Week 2 through Week 24, with the exception of swollen joint counts at Week 2.
At Week 24, the proportion of subjects achieving a modified PsARC response was 68.6% and 72.6% in the guselkumab 100 mg q4w and guselkumab 100 mg q8w groups, respectively, compared with 44.7% in the placebo group (both nominal p<0.001).
At Week 24, the proportion of subjects achieving low disease activity or remission based on the DAPSA index was 35.5% and 38.7% in the guselkumab 100 mg q4w and guselkumab 100 mg q8w groups, respectively, compared with 18.3% in the placebo group (both nominal p<0.001).

### Other Efficacy Endpoints Related to Physical Function

At Week 24, the HAQ-DI response rate (defined as ≥0.35 improvement from baseline among the subjects with a HAQ-DI score ≥0.35 at baseline) was 56.1% and 50.0% in the guselkumab 100 mg q4w and the guselkumab 100 mg q8w groups, respectively, compared with 31.4% in the placebo group (both nominal p<0.001).
Other Efficacy Endpoints Related to Skin Disease
Among the 543 (73.5%) subjects with ≥3% BSA of psoriatic involvement and an IGA score≥2 (mild) at baseline:
Numerically greater proportions of subjects with PASI 50, PASI 75, PASI 90, and PASI 100 responses were observed in both guselkumab groups compared with the placebo group at Week 16 and Week 24 (all nominal p<0.001).
At Week 24, the proportions of subjects who achieved both a PASI 75 and an ACR 20 response were 57.1% and 56.8% in the guselkumab 100 mg q4w and guselkumab 100 mg q8w groups, respectively, compared with 11.5% in the placebo group (both nominal p<0.001).
At Week 24, the proportions of subjects who achieved both a PASI 75 and a modified PsARC response were 60.9% and 65.3% in the guselkumab 100 mg q4w and guselkumab 100 mg q8w groups, respectively, compared with 15.3% in the placebo group (both nominal p<0.001).
At Week 24, the proportions of subjects who achieved an IGA score of 0 (clear) were 50.5% and 50.0% in the guselkumab 100 mg q4w and guselkumab 100 mg q8w groups, respectively, compared with 7.7% in the placebo group (both nominal p<0.001).
At Week 24, a numerically greater proportion of subjects achieved clinically meaningful ≥5 point improvement from baseline in DLQI score in the guselkumab 100 mg q4w group (86.8%) and the guselkumab 100 mg q8w group (83.3%) compared with the placebo group (37.8%; both nominal p<0.001).
Other Efficacy Endpoints Related to Enthesitis and Dactylitis
Among the 506 (68.5%) subjects with enthesitis at baseline based on CNTO1959PSA3002 data only, the number of subjects achieving enthesitis resolution was numerically higher in both guselkumab groups compared with the placebo group at each visit through from Week 2 to Week 24.
Among the 331 (44.8%) subjects with dactylitis at baseline based on CNTO1959PSA3002 data only, the number of subjects achieving dactylitis resolution was numerically higher in both guselkumab groups compared with the placebo group at each visit from Week 2 through Week 24.
Other Efficacy Endpoints Related to BASDAI
Among the 258 (34.9%) subjects with spondylitis and peripheral arthritis at baseline, a numerically greater reduction from baseline in BASDAI was observed in both guselkumab groups compared with the placebo group at each visit BASDAI was evaluated from Week 8 through Week 24
The proportions of subjects achieving ≥20%, ≥50%, and ≥70% improvement in BASDAI scores were numerically greater in both guselkumab groups compared with the placebo group from Week 8 through Week 24.

### Other Efficacy Endpoints Related to Joint Structural Damage

The proportions of subjects with a change of ≤0 from baseline in modified vdH-S scores were 67.3% in the guselkumab 100 mg q4w group and 63.4% in the guselkumab 100 mg q8w group compared with 64.7% in the placebo group (nominal p=0.555 and p=0.751, respectively).
The proportions of subjects with a change of ≤0 from baseline in modified vdH-S erosion scores were 71.4% in the guselkumab 100 mg q4w group and 66.3% in the guselkumab 100 mg q8w group compared with 66.8% in the placebo group (nominal p=0.268 and p=0.86′7, respectively).
The proportions of subjects with a change of ≤0 from baseline in modified vdH-S JSN scores at Week 24 were 80.2% in the guselkumab 100 mg q4w group and 78.8% in the guselkumab 100 mg q8w group compared with 78.6% in the placebo group (nominal p=0.669 and p=0.903, respectively).

### Other Efficacy Endpoints Related to Health-Related Quality of Life and Other Patient Reported Outcomes

At Week 24, the proportion of subjects who achieved clinically meaningful ≥5-point improvement from baseline in SF-36 PCS score was 55.9% and 60.1% in the guselkumab 100 mg q4w and guselkumab 100 mg q8w groups, respectively, compared with 40.2% in the placebo group (both nominal p<0.001).
At Week 24, the proportion of subjects who achieved clinically meaningful ≥5-point improvement from baseline in SF-36 MCS score was 34.3% and 37.5% in the guselkumab 100 mg q4w and guselkumab 100 mg q8w groups, respectively, compared with 30.9% in the placebo group (nominal p=0.424 and p=0.124, respectively).
At Week 24, the proportion of subjects who achieved≥4-point improvement from baseline in FACIT-Fatigue score was 59.6% and 60.5% in the guselkumab 100 mg q4w and guselkumab 100 mg q8w groups, respectively, compared with 45.5% in the placebo group (nominal p=0.002 and p<0.001, respectively).
At Week 24, a numerically greater increase from baseline in EQ-5D index scores was observed in both the guselkumab 100 mg q4w group (LSmean: 0.116) and the guselkumab 100 mg q8w group (LSmean: 0.115) compared with the placebo group (LSmean: 0.053; both nominal p<0.001).
At Week 24, a numerically greater increase from baseline in EQ-5D health state VAS score was observed in both the guselkumab 100 mg q4w group (LSmean: 18.089) and the guselkumab 100 mg q8w group (LSmean: 18.371) compared with the placebo group (LSmean: 6.796; both nominal p<0.001).

### Improvements in Composite Disease Activity Scores

At Week 24, the proportion of subjects who met MDA criteria was 18.8% and 25.0% in the guselkumab 100 mg q4w and guselkumab 100 mg q8w groups, respectively, compared with 6.1% in the placebo group (both nominal p<0.001). Greater improvements in other PsA composite disease activity scores including PASDAS, GRACE index, and mCPDAI score were also observed in both guselkumab groups compared with the placebo group at Week 24 (all nominal p<0.001).

### Efficacy and Pharmacokinetics

There appeared to be a weak exposure-response relationship for ACR 50 response rate at Week 24 by steady-state trough guselkumab concentration quartiles at Week 20, while no consistent exposure-response relationship was observed for ACR 20 response rate at Week 24.
There was no apparent exposure-response relationship for mean changes from baseline in DAS28 (CRP) at Week 20 or Week 24 by steady-state trough guselkumab concentration quartiles at Week 20.
There was no apparent exposure-response relationship in IGA response at Week 24 by steady state trough guselkumab concentration quartiles at Week 20 in subjects with ≥3% BSA psoriatic involvement and an IGA score of ≥2 at baseline.

### Efficacy and Antibodies to Guselkumab

The presence of antibodies to guselkumab did not preclude ACR responses for subjects who were positive for antibodies to guselkumab through Week 24. However, the small number of subjects who were positive for antibodies to guselkumab (n=10) limits a definitive conclusion on the impact of antibodies to guselkumab on clinical efficacy.

### Safety Results

### Adverse Events

An overall summary of AEs reported through Week 24 is provided in Table 24. The average number of study agent administrations was consistent across treatment groups.

TABLE 24
Overall Summary of Treatment-emergent Adverse Events through Week 24; Safety
Analysis Set (Study CNTO1959PSA3002)

| | | Guselkumab | | |
|---|---|---|---|---|
| | Placebo | 100 mg q8w | 100 mg q4w | Combined |
| Analysis set: Safety Analysis Set | 246 | 248 | 245 | 493 |
| Average duration of follow up (weeks) | 24.0 | 23.9 | 23.8 | 23.9 |
| Average number of study agent administrations | 5.9 | 5.9 | 5.9 | 5.9 |
| Average number of placebo administrations | 5.9 | 2.0 | 0.0 | 1.0 |
| Average number of guselkumab | 0.0 | 3.9 | 5.9 | 4.9 |
| administrations | | | | |
| Subjects with 1 or more adverse events | 100 (40.7%) | 114 (46.0%) | 113 (46.1%) | 227 (46.0%) |
| Subjects with 1 or more serious adverse events | 7 (2.8%) | 3 (1.2%) | 8 (3.3%) | 11 (2.2%) |
| Subjects with 1 or more adverse events leading | 4 (1.6%) | 2 (0.8%) | 6 (2.4%) | 8 (1.6%) |
| to discontinuation of study agent | | | | |
| Subjects with 1 or more adverse events with | 2 (0.8%) | 1(0.4%) | 2 (0.8%) | 3 (0.6%) |
| severe intensity | | | | |
| Subjects with 1 or more infections | 45 (18.3%) | 40 (16.1%) | 49 (20.0%) | 89 (18.1%) |
| Subjects with 1 or more serious infections | 1 (0.4%) | 1 (0.4%) | 3 (1.2%) | 4 (0.8%) |
| Subjects with 1 or more injection site reactions | 1 (0.4%) | 3 (1.2%) | 3 (1.2%) | 6 (1.2%) |
| Subjects with 1 or more events of malignancy | 1 (0.4%) | 1 (0.4%) | 0 | 1 (0.2%) |
| Subjects with 1 or more opportunistic | 0 | 0 | 0 | 0 |
| infections | | | | |
| Subjects with 1 or more events leading to death | 0 | 0 | 0 | 0 |

Note:
Subjects are counted only once for any given event, regardless of the number of times they actually experienced the event. Adverse events are coded using MedDRA Version 21.1

The proportions of subjects experiencing 1 or more AEs through Week 24 were slightly higher in the guselkumab treatment groups compared with the placebo group: 46.1% in the guselkumab 100 mg q4w group, 46.0% in the guselkumab 100 mg q8w group, and 40.7% in the placebo group.

The most frequent SOC of reported AEs was Infections and infestations and the overall frequency of events in this SOC was comparable across treatment groups (17.6% in the guselkumab 100 mg q4w group, 15.7% in the guselkumab 100 mg q8w group, and 17.1% in the placebo group). The second most frequent SOC was Investigations among which AEs occurred more frequently in the guselkumab treatment groups than in the placebo group (14.3% in the guselkumab 100 mg q4w group, 14.5% in the guselkumab 100 mg q8w group, and 7.7% in the placebo group).

The most common PTs with a frequency ≥5% in any treatment group excluding serious AEs through Week 24 are presented in Table 25. The most common PTs reported were ALT increased (10.2% in the guselkumab 100 mg q4w group, 6.0% in the guselkumab 100 mg q8w group, and 4.5% in the placebo group) followed by AST increased (4.5% in the guselkumab 100 mg q4w group, 5.6% in the guselkumab 100 mg q8w group, and 2.4% in the placebo group). The AEs of ALT increased were more frequently reported in the guselkumab treatment groups compared with the placebo group and higher in the guselkumab 100 mg q4w group compared with the guselkumab 100 mg q8w group.

TABLE 25
Number of Subjects with Treatment-Emergent Adverse Events (Excluding Serious Adverse
Events) with Frequency of at least 5% in Any Treatment Group through Week 24 by
MedDRA System-organ Class and Preferred Term; Safety Analysis Set
(Study CNTO1959PSA3002)

| | Guselkumab | | | |
|---|---|---|---|---|
| | Placebo | 100 mg q8w | 100 mg q4w | Combined |
| Analysis set: Safety Analysis Set | 246 | 248 | 245 | 493 |
| Average duration of follow up (weeks) | 24.0 | 23.9 | 23.8 | 23.9 |
| Average number of study agent administrations | 5.9 | 5.9 | 5.9 | 5.9 |
| Subjects with 1 or more adverse events (excluding | 99 (40.2%) | 113 (45.6%) | 109 (44.5%) | 222 (45.0%) |
| serious events) | | | | |
| MedDRA system - organ class/preferred term | | | | |
| Investigations | 19 (7.7%) | 36 (14.5%) | 35 (14.3%) | 71(14.4%) |
| Alanine aminotransferase increased | 11 (4.5%) | 15 (6.0%) | 25 (10.2%) | 40 (8.1%) |
| Aspartate aminotransferase increased | 6 (2.4%) | 14 (5.6%) | 11 (4.5%) | 25 (5.1%) |

Note:
Subjects are counted only once for any given event, regardless of the number of times they actually experienced the event. Adverse events are coded using MedDRA Version 21.1

### Adverse Events Through Week 24 by Baseline Age Group

Age was separated into the following groups: <45 years (n=340), ≥45 to <65 years (n=366), ≥65 years (n=33), and ≥75 years (n=1). The proportions of subjects reporting AEs in the guselkumab treatment groups were higher in the <45 years age group and similar in the ≥45 to <65 years age group compared with the placebo group. In the ≥65 years age group, the proportion of subjects reporting AEs was higher in the guselkumab 100 mg q4w group than in the guselkumab 100 mg q8w and placebo groups; however, the number of subjects in this age group was small:
<45 years (n=340): 47.2%, 47.7%, and 33.7% in the guselkumab 100 mg q4w, guselkumab 100 mg q8w, and the placebo groups, respectively.
≥45 to <65 years (n=366): 44.4%, 45.9%, and 46.6% in the guselkumab 100 mg q4w, guselkumab 100 mg q8w, and the placebo groups, respectively.
≥65 years (n=33): 54.5%, 27.3%, and 36.4% in the guselkumab 100 mg q4w, guselkumab 100 mg q8w, and the placebo groups, respectively.

### Adverse Events Through Week 24 by Baseline Use of Non-Biologic DMARDs

Subjects were separated into the following groups: none (n=227), MTX (n=443), any non-MTX DMARDs (n=69), SSZ (n=31), HCQ (n=3), LEF (n=35), and any DMARDs (n=512).

The proportions of subjects with AEs reported through Week 24 were slightly higher in the guselkumab treatment groups compared with the placebo group for each subgroup. Overall, the proportions of subjects reporting AEs were generally higher in the MTX and any DMARDs subgroups compared with the none at baseline subgroup:
None (n=227): 46.7%, 34.6%, and 29.7% in the guselkumab 100 mg q4w, guselkumab 100 mg q8w, and the placebo groups, respectively.
Methotrexate (n=443): 46.6%, 52.5%, and 45.5% in the guselkumab 100 mg q4w, guselkumab 100 mg q8w, and the placebo groups, respectively.
any DMARDs (n=512): 45.9%, 51.2%, 45.3% in the guselkumab 100 mg q4w, guselkumab 100 mg q8w, and the placebo groups, respectively.
   The number of subjects in remaining subgroups was very small. The AE profiles in these subjects were generally consistent with the overall population and there was no specific pattern identified in these subjects.

Consistent with the overall population, the most frequent SOC of reported AEs was Infections and infestations in all the subgroups except in the no use of non-biologic DMARDs subgroup in which Investigations was most frequent.

### Adverse Events of Severe Intensity

The proportion of subjects reporting 1 or more AEs of severe intensity was low, 0.8% in the guselkumab 100 mg q4w group, 0.4% in the guselkumab 100 mg q8w group, and 0.8% in the placebo group. All events were singular in occurrence.

### Reasonably-Related Adverse Events

Through Week 24, the proportions of subjects who experienced at least 1 reasonably-related AE were similar across the treatment groups (16.3% in the guselkumab 100 mg q4w group, 16.9% in the guselkumab 100 mg q8w group, and 14.2% in the placebo group).

### Deaths

There were no deaths reported in this study through Week 24.

### Serious Adverse Events

The proportions of subjects who experienced 1 or more SAEs through Week 24 were 3.3% in the guselkumab 100 mg q4w group, 1.2% in the guselkumab 100 mg q8w group, and 2.8% in the placebo group (Table 26). All events were singular in occurrence and no specific pattern of SAEs was identified.

TABLE 26
Number of Subjects with 1 or More Treatment-emergent Serious Adverse Events through
Week 24 by MedDRA System-organ Class and Preferred Term; Safety Analysis Set
(Study CNTO1959PSA3002)

| | | Guselkumab | | |
|---|---|---|---|---|
| | Placebo | 100 mg q8w | 100 mg q4w | Combined |
| Analysis set: Safety Analysis Set | 246 | 248 | 245 | 493 |
| Average duration of follow up (weeks) | 24.0 | 23.9 | 23.8 | 23.9 |
| Average number of study agent administrations | 5.9 | 5.9 | 5.9 | 5.9 |
| Subjects with 1 or more serious adverse events | 7 (2.8%) | 3 (1.2%) | 8 (3.3%) | 11(2.2%) |
| MedDRA system - organ class/preferred term | | | | |
| Infections and infestations | 0 | 0 | 3 (1.2%) | 3 (0.6%) |
| Acute hepatitis B | 0 | 0 | 1 (0.4%) | 1 (0.2%) |
| Oophoritis | 0 | 0 | 1 (0.4%) | 1 (0.2%) |
| Pneumonia influenzal | 0 | 0 | 1 (0.4%) | 1 (0.2%) |
| Injury, poisoning and procedural complications | 1 (0.4%) | 1 (0.4%) | 2 (0.8%) | 3 (0.6%) |
| Ankle fracture | 0 | 1 (0.4%) | 0 | 1 (0.2%) |
| Femur fracture | 0 | 0 | 1 (0.4%) | 1 (0.2%) |
| Lower limb fracture | 0 | 0 | 1 (0.4%) | 1 (0.2%) |
| Metal poisoning | 0 | 0 | 1 (0.4%) | 1 (0.2%) |
| Post procedural fistula | 1 (0.4%) | 0 | 0 | 0 |
| Cardiac disorders | 1 (0.4%) | 1 (0.4%) | 0 | 1 (0.2%) |
| Coronary artery disease | 0 | 1 (0.4%) | 0 | 1 (0.2%) |
| Angina unstable | 1 (0.4%) | 0 | 0 | 0 |
| General disorders and administration site conditions | 0 | 1 (0.4%) | 0 | 1 (0.2%) |
| Pyrexia | 0 | 1 (0.4%) | 0 | 1 (0.2%) |
| Musculoskeletal and connective tissue disorders | 0 | 0 | 1 (0.4%) | 1 (0.2%) |
| Osteoarthritis | 0 | 0 | 1 (0.4%) | 1 (0.2%) |
| Nervous system disorders | 0 | 0 | 1 (0.4%) | 1 (0.2%) |
| Ischaemic stroke | 0 | 0 | 1 (0.4%) | 1 (0.2%) |
| Vascular disorders | 0 | 0 | 1 (0.4%) | 1 (0.2%) |
| Blue toe syndrome | 0 | 0 | 1 (0.4%) | 1 (0.2%) |
| Gastrointestinal disorders | 1 (0.4%) | 0 | 0 | 0 |
| Inflammatory bowel disease | 1 (0.4%) | 0 | 0 | 0 |
| Hepatobiliary disorders | 1 (0.4%) | 0 | 0 | 0 |
| Drug-induced liver injury | 1 (0.4%) | 0 | 0 | 0 |
| Metabolism and nutrition disorders | 1 (0.4%) | 0 | 0 | 0 |
| Obesity | 1 (0.4%) | 0 | 0 | 0 |
| Neoplasms benign, malignant and unspecified | 1 (0.4%) | 0 | 0 | 0 |
| (inclcysts and polyps) | | | | |
| Clear cell renal cell carcinoma | 1 (0.4%) | 0 | 0 | 0 |
| Renal and urinary disorders | 1 (0.4%) | 0 | 0 | 0 |
| Tubulointerstitial nephritis | 1 (0.4%) | 0 | 0 | 0 |

Note:
Subjects are counted only once for any given event, regardless of the number of times they actually experienced the event. Adverse events are coded using MedDRA Version 21.1

### Serious Adverse Events Through Week 24 by Baseline Age Group

There was no specific pattern of association between SAEs and age at baseline.
<45 years (n=340): 4.6%, 0, and 1.0% in the guselkumab 100 mg q4w, guselkumab 100 mg q8w, and the placebo groups, respectively.
≥45 to <65 years (n=366): 2.4%, 2.8%, and 4.6% in the guselkumab 100 mg q4w, guselkumab 100 mg q8w, and the placebo groups, respectively.
≥65 years (n=33): No events were reported.

### Serious Adverse Events Through Week 24 by Baseline Use of Non-Biologic DMARDs

The proportions of subjects with SAEs were generally comparable across the treatment groups for each subgroup in which SAEs were reported.
None (n=227): 4.0%, 0, and 2.7% in the guselkumab 100 mg q4w, guselkumab 100 mg q8w, and the placebo groups, respectively.
Methotrexate (n=443): 3.4%, 2.1%, and 3.2% in the guselkumab 100 mg q4w, guselkumab 100 mg q8w, and the placebo groups, respectively.
any DMARDs (n=512): 2.9%, 1.8%, and 2.9% in the guselkumab 100 mg q4w, guselkumab 100 mg q8w, and the placebo groups, respectively.
   No SAEs were reported in the remaining subgroups.

### Reasonably-Related Serious Adverse Events

Through Week 24, the proportions of subjects who experienced at least 1 reasonably-related SAE were low (0.4% in the guselkumab 100 mg q4w group, 0.4% in the guselkumab 100 mg q8w group, and 1.2% in the placebo group).

### Example 2: A Phase 3, Multicenter, Randomized, Double-Blind, Placebo-Controlled Study Evaluating the Efficacy and Safety of Guselkumab Administered Subcutaneously in Subjects with Active Psoriatic Arthritis Including Those Previously Treated with Biologic Anti-TNFα Agent(s) (CNTO1959PSA3001)

Study (CNTO1959PSA3001) is a Phase 3, multicenter, randomized, double-blind, placebo-controlled, 3-arm study of guselkumab in subjects with active PsA who had an inadequate response to standard therapies (eg, non-biologic DMARDs, apremilast, or NSAIDs). In addition, subjects (approximately 30%) may have been previously treated with up to 2 anti TNFα agents. The study consisted of a screening phase of up to 6 weeks, a blinded treatment phase of approximately 1 year (ie, 52 weeks), including a placebo-controlled period from Week 0 to Week 24 and an active treatment phase from Week 24 to Week 52, and a safety follow-up phase of 8 weeks after Week 52. The study was to enroll approximately 360 subjects. The study was conducted to evaluate the clinical efficacy, safety, and pharmacokinetics (PK) of guselkumab in subjects with active psoriatic arthritis (PsA). The secondary objectives were to assess the following for guselkumab treatment:
Efficacy in improving psoriatic skin lesions
Improvement in physical function

### Methods

### Overview of Study Design

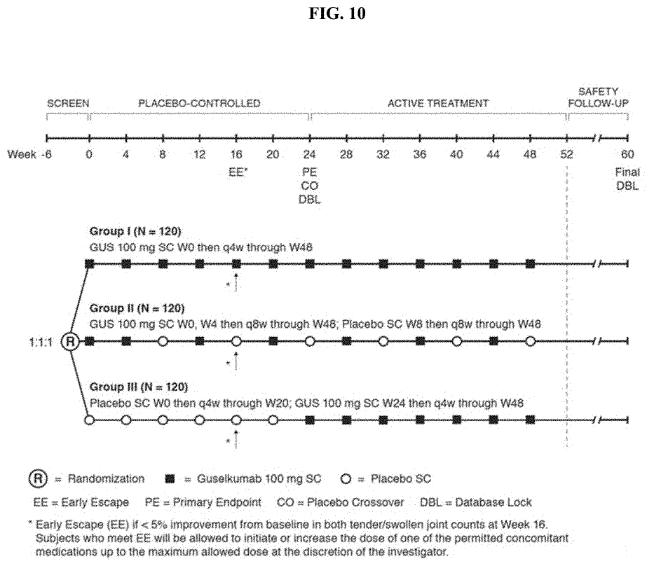

A diagrammatic representation of the study design is presented in FIG. 10.

At Week 0, approximately 360 subjects who satisfied all inclusion and exclusion criteria were to be randomly assigned to 1 of the following 3 treatment groups in a 1:1:1 ratio using permuted block randomization stratified by baseline non-biologic DMARD use (yes, no) and by prior exposure to anti-TNFα agents (yes, no):
Group I (n=120): Guselkumab SC 100 mg every 4 weeks (q4w) from Week 0 through Week 48.
Group II (n=120): Guselkumab SC 100 mg at Weeks 0 and 4, then q8w (Weeks 12, 20, 28, 36, and 44) and placebo injections at other visits (Weeks 8, 16, 24, 32, 40, and 48) to maintain the blind.
Group III (n=120): Placebo SC q4w from Week 0 to Week 20 and crossed over at Week 24 to receive guselkumab 100 mg q4w through Week 48.

At Week 16, all subjects in Groups I, II, and III with <5% improvement from baseline in both tender and swollen joint counts were considered as meeting early escape (EE) criteria. These subjects remained on the dose regimen they were randomized to at Week 0, but were allowed to initiate or increase the dose of one of the permitted concomitant medications up to the maximum allowed dose as specified in the protocol, with titration to a stable dose to be completed by the Week 24 visit.

Efficacy evaluations included joint assessments (swollen and tender joint counts), patient's assessment of pain, patient's global assessment of disease activity (arthritis and psoriasis), patient's global assessment of disease activity (arthritis), physician's global assessment of disease activity, Health Assessment Questionnaire-Disability Index (HAQ-DI), C-reactive protein (CRP), patient's assessment of skin disease activity, body surface area (BSA) of psoriasis, Psoriasis Area and Severity Index (PAST), Investigator's Global Assessment of Psoriasis (IGA), dactylitis assessment, enthesitis assessments based on Leeds Enthesitis Index (LEI) and Spondyloarthritis Research Consortium of Canada (SPARCC) criteria, Bath Ankylosing Spondylitis Disease Activity Index (BASDAI; for subjects with primary PsA subtype of spondylitis with peripheral arthritis), American College of Rheumatology (ACR) response, Minimal Disease Activity (MDA) and Very Low Disease Activity (VLDA), Psoriatic Arthritis Disease Activity Score (PASDAS), Group Research and Assessment of Psoriasis and Psoriatic Arthritis (GRAPPA) Composite Score (GRACE) index, Disease Activity Score 28 (DAS28) using CRP, Disease Activity Index for Psoriatic Arthritis (DAPSA), and Psoriatic Arthritis Response Criteria (PsARC), 36-Item Short-form Health Survey (SF-36), Functional Assessment of Chronic Illness Therapy (FACIT)-Fatigue, Patient Reported Outcomes Measurement Information System (PROMIS)-29.

Safety assessments included adverse events (AEs), serious adverse events (SAEs), injection site and allergic reactions, clinical laboratory parameters (hematology and chemistry; urine pregnancy test), electronic Columbia-Suicide Severity Rating Scale (eC-SSRS), physical examinations, vital signs, electrocardiogram (ECG; Week 0 only), and early detection of tuberculosis (TB).

Samples for the analysis of pharmacodynamic biomarkers were collected from all subjects.

### Study Population

The target population consisted of adult men or women with active PsA who have had inadequate response to standard therapies (eg, non-biologic DMARDs, apremilast or NSAIDs). In addition, approximately 30% of the study population may have been previously exposed to up to 2 anti TNFα agents.

To be eligible for this study, subjects had to be at least 18 years of age at the time of informed consent, diagnosed with PsA for at least 6 months prior to the first administration of study agent, and meet ClASsification criteria for Psoriatic ARthritis (CASPAR)42 at screening. Subjects must have had active PsA as defined by ≥3 tender and ≥3 swollen joints at both screening and baseline, and CRP≥0.3 mg/dL at screening. Subjects must have documented evidence of inadequate response or evidence of intolerance to standard PsA therapies including non-biologic DMARD (≥3 months), apremilast (≥4 months), and/or NSAID therapy (≥4 weeks) prior to the first administration of study agent. Subjects with prior exposure to up to 2 anti-TNFα agents were allowed but limited to approximately 30% of the study population.

Subjects had to have at least 1 of the PsA subsets: distal interphalangeal (DIP) joint involvement, polyarticular arthritis with absence of rheumatoid nodules, arthritis mutilans, asymmetric peripheral arthritis, or spondylitis with peripheral arthritis. In addition, subjects must have had active plaque psoriasis with at least 1 psoriatic plaque of ≥2 cm in diameter or nail changes consistent with psoriasis or documented history of plaque psoriasis.

Subjects were permitted to continue stable doses of non-biologic DMARDs (limited to MTX [≤25 mg/week], SSZ [≤3 g/day], HCQ [≤400 mg/day], or LEF [≤20 mg/day]), low-dose oral corticosteroid (≤10 mg of prednisone per day or equivalent), or NSAIDs and other analgesics treatment during the study. If subjects were not using these medications at baseline, these medications must have been stopped ≥4 weeks (for MTX, SSZ, or HCQ), ≥12 weeks (LEF), or ≥2 weeks (for NSAIDs and other analgesics or oral corticosteroid) prior to the first administration of study agent. In addition, subjects had to meet criteria for screening laboratory test results and TB history and testing results, agree to use adequate birth control measures, avoid prolonged sun exposure, and avoid the use of tanning booths or other ultraviolet light sources during the study.

### Dosage and Administration

All study agents (guselkumab and placebo) were administered through SC injection. Based upon guselkumab clinical efficacy, safety, PK data, and exposure response modeling analysis using data from the Phase 2 study (CNTO1959PSA2001) in subjects with PsA, 2 dose regimens were chosen for evaluation in the guselkumab Phase 3 PsA program, and eligible subjects were randomly assigned to receive 1 of the following 3 treatments at Week 0:
Guselkumab 100 mg q4w: Subjects received SC guselkumab 100 mg q4w from Week 0 through Week 48.
Guselkumab 100 mg at Weeks 0 and 4 then q8w (hereafter referred to as the guselkumab 100 mg q8w group): Subjects received SC guselkumab 100 mg at Weeks 0 and 4, then q8w (at Weeks 12, 20, 28, 36, 44) and placebo injections at other visits (Weeks 8, 16, 24, 32, 40, 48) to maintain the blind.
Placebo: Subjects received SC placebo q4w from Week 0 to Week 20, and crossed over at Week 24 to receive SC guselkumab 100 mg q4w from Week 24 through Week 48.
   Rationale for Guselkumab 100 mg at Weeks 0 and 4 then Every 8 Weeks Dose Regimen
This dose regimen was evaluated in the Phase 2 PsA study (CNTO1959PSA2001) and in the 3 global Phase 3 studies in psoriasis. In the CNTO1959PSA2001 study, robust efficacy and clinically meaningful improvement was observed with this dose regimen in all important domains of PsA including joint signs and symptoms, physical function, psoriasis, enthesitis, dactylitis, and quality of life in patients with active PsA and ≥3% BSA of psoriasis. Additionally, significant benefit was also observed with this dose regimen on plaque psoriasis in patients with moderate-to-severe psoriasis in the Phase 3 psoriasis studies.
An additional dose was included at Week 4 to ensure that trough guselkumab levels do not fall below those obtained at steady state levels. This additional Week 4 dose results in a slightly higher Cmax and Ctrough in the first 12 weeks than those at steady state (˜21% and ˜18%, respectively) and may result in a more rapid onset of response. However, this dose regimen is not expected to result in substantially higher levels of efficacy at Week 24 than would be achieved by q8w dosing during maintenance, ie, from Week 24 and onwards.
The safety of this dose regimen has been established in a large psoriasis development program. Furthermore, the safety profile in the Phase 2 studies in patients with PsA and RA is consistent with that seen in the psoriasis program.

### Rationale for Guselkumab 100 mg Every 4 Weeks Dose Regimen

A dose regimen of 100 mg q4w was included to determine if more frequent dosing may achieve higher efficacy in PsA.
Modeling analyses based on data from CNTO1959PSA2001 suggested that a higher or more frequent dose regimen may achieve better efficacy in PsA.
Patients who have had inadequate response to anti-TNFα or other biologic treatments are more difficult to treat and may benefit from a higher dose.25
Treatment with the 100 mg q4w dose regimen was expected to result in acceptable safety based on the exposure-safety analysis in the Phase 3 psoriasis program.
Guselkumab has been shown to have an acceptable safety profile in multiple patient populations, including with a higher dose regimen that was studied in a Phase 2 RA study (200 mg q8w).

Overall, the 2 dose regimens of guselkumab (100 mg q4w and 100 mg q8w) selected for this study were expected to provide an adequate assessment of the optimal benefit/risk profile of guselkumab in PsA.

Study agent was administered at the site by a health care professional (HCP) at Week 0 and Week 4. Beginning at Week 8, at the discretion of the investigator and subject, and after appropriate and documented training, subjects had the option to self administer study agent at the investigative site under the supervision of an HCP or continue to have study agent injections performed by an HCP.

Through Week 24, study agent administration at the site was to occur ±4 days from the scheduled day of study agent administration. Study agent administrations were to be at least 14 days apart.

### Efficacy Evaluation—End Points

### Primary Endpoint

The primary endpoint was the proportion of subjects who achieved an ACR 20 response at Week 24.

### Major Secondary Endpoints

1. Proportion of subjects with a psoriasis response of an IGA (ie, an IGA psoriasis score of 0 [cleared] or 1 [minimal] AND ≥2 grade reduction from baseline) at Week 24 among subjects with ≥3% BSA psoriatic involvement and an IGA score of ≥2 (mild) at baseline.
2. Change from baseline in HAQ DI score at Week 24.
3. Change from baseline in SF-36 PCS at Week 24.
4. Change from baseline in DAS28 (CRP) at Week 24.
5. Proportion of subjects who achieve an ACR 20 response at Week 16.
6. Proportion of subjects who achieve an ACR 50 response at Week 24.
7 Proportion of subjects who achieve an ACR 70 response at Week 24.
8. Proportion of subjects who achieve an ACR 50 response at Week 16.
9. Proportion of subjects with resolution of enthesitis at Week 24 among the subjects with enthesitis at baseline.
10. Change from baseline in enthesitis score (based on LEI) at Week 24 among the subjects with enthesitis at baseline.
11. Proportion of subjects with resolution of dactylitis at Week 24 among the subjects with dactylitis at baseline.
12. Change from baseline in dactylitis scores at Week 24 among the subjects with dactylitis at baseline.
13. Change from baseline in SF-36 MCS at Week 24.

### Other Secondary Endpoints

### Endpoints Related to Reduction of Signs and Symptoms and Physical Function

1. Proportion of subjects who achieve ACR 20, ACR 50, and ACR 70 responses by visit over time through Week 24.
2. ACR components by visit through Week 24.
3. Percent change from baseline in ACR components by visit over time through Week 24.
4. Change from baseline in HAQ-DI score by visit over time through Week 24.
5. Proportion of subjects who achieve a clinically meaningful improvement (a ≥0.35 improvement from baseline) in HAQ-DI score by visit over time through Week 24 among those subjects with HAQ-DI score≥0.35 at baseline.
6. Proportion of subjects who achieve a DAS28 (CRP) response by visit over time through Week 24.
7. Proportion of subjects who achieve a DAS28 (CRP) remission by visit over time through Week 24.
8. Change from baseline in DAS28 (CRP) by visit over time through Week 24.
9. Proportion of subjects who achieve a response based on modified PsARC by visit over time through Week 24.
10. Proportion of subjects with resolution of enthesitis by visit over time through Week 24 among the subjects with enthesitis at baseline.
11. Change from baseline in enthesitis score by visit over time through Week 24 among the subjects with enthesitis at baseline.
12. Proportion of subjects with resolution of dactylitis by visit over time through Week 24 among subjects with dactylitis at baseline.
13. Change from baseline in dactylitis score by visit over time through Week 24 among the subjects with dactylitis at baseline.
14. Change from baseline in PASDAS by visit score over time through Week 24.
15. Change from baseline in GRACE index by visit over time through Week 24.
16. Change from baseline in DAPSA score by visit over time through Week 24.
17. Proportion of subjects who achieve MDA by visit over time through Week 24.
18. Proportions of subjects who achieve a ≥20%, ≥50%, ≥70%, and ≥90% improvement from baseline in BASDAI score by visit over time through Week 24 among subjects with spondylitis and peripheral joint involvement as their primary arthritic presentation of PsA and BASDAI score>0 at baseline.
19. Change from baseline in BASDAI score by visit over time through Week 24 among subjects with spondylitis and peripheral arthritic presentation of PsA and BASDAI>0 at baseline.
20. Proportion of subjects with low or very low disease activity based on PASDAS by visit over time through Week 24.
21. Proportion of subjects with low or very low disease activity based on GRACE score by visit over time through Week 24.
22. Proportion of subjects with low disease activity or remission based on DAPSA by visit over time through Week 24.
23. Proportion of subjects with very low disease activity by visit over time through Week 24.

### Endpoints Related to Skin Disease

1. Proportions of subjects who achieve≥75%, ≥90%, and 100% improvement in PASI score from baseline by visit over time through Week 24 among subjects with ≥3% BSA psoriatic involvement and an IGA score of ≥2 (mild) at baseline.
2. Proportion of subjects who achieve both PASI 75 and ACR 20 responses by visit over time through Week 24 among subjects with ≥3% BSA psoriatic involvement and an IGA score of ≥2 (mild) at baseline.
3. Proportion of subjects who achieve both PASI 75 and modified PsARC response by visit over time through Week 24 among subjects with ≥3% BSA psoriatic involvement and an IGA score of ≥2 (mild) at baseline.
4. Proportion of subjects with an IGA score of 0 (cleared) by visit over time through Week 24 among subjects with ≥3% BSA psoriatic involvement and an IGA score of ≥2 (mild) at baseline.
5. Change from baseline in PASI score by visit over time through Week 24 among subjects with ≥3% BSA psoriatic involvement and an IGA score of ≥2 (mild) at baseline.

### Endpoints Related to Health-Related Quality of Life

Change from baseline in SF-36 PCS score by visit over time through Week 24.
2. Change from baseline in SF-36 MCS score by visit over time through Week 24.
3. Change from baseline in domain scales scores of SF-36 by visit over time through Week 24.
4. Proportion of subjects who achieve≥5-point improvement from baseline in SF-36 MCS score by visit over time through Week 24.
5. Proportion of subjects who achieve≥5-point improvement from baseline in SF 36 PCS score by visit over time through Week 24.
6. Change from baseline in FACIT Fatigue by visit over time through Week 24.
7. Proportion of subjects who achieve≥4-point improvement from baseline in FACIT Fatigue score improvement by visit over time through Week 24.
8. Change from baseline in PROMIS 29 scores by visit over time through Week 24.
9. Change from baseline in FACIT-Fatigue score at Week 24 by ACR 20 response (primary endpoint) at Week 24.
10. Proportion of subjects who achieve≥4-point improvement from baseline in FACIT-Fatigue score at Week 24 by ACR 20 response (primary endpoint) at Week 24.
11. Proportion of subjects who achieve an improvement of ≥3 points in PROMIS-29 domain scores by visit through Week 24.
12. Proportion of subjects who achieve an improvement of ≥5 points in PROMIS-29 domain scores by visit through Week 24.

### Results

### Pharmacokinetic, Immunogenicity, Pharmacodynamic, and Pharmacogenomic Results

A total of 254 subjects who received at least 1 dose of guselkumab and had at least 1 valid sample collected after guselkumab administration were included in the PK evaluation. Subjects who received placebo only were excluded from the PK evaluation.

### Serum Guselkumab Concentrations Over Time

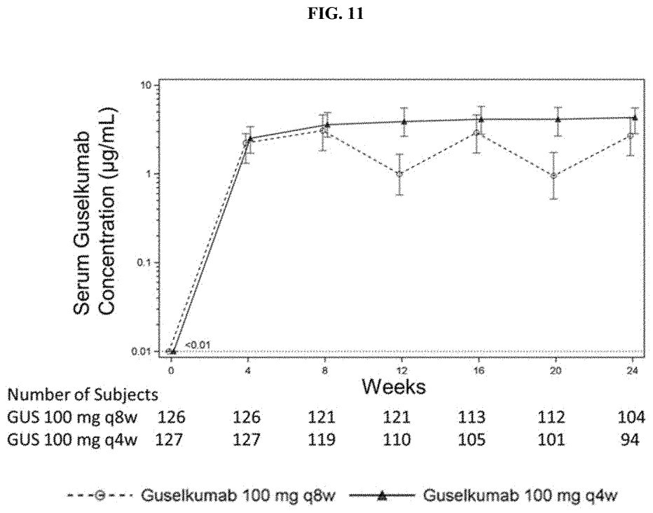

The median and IQ range of trough serum guselkumab concentrations by guselkumab treatment group and visit through Week 24 are graphically displayed in FIG. 11.

Following SC administration of guselkumab, trough serum guselkumab concentrations generally reached steady state by Week 12 for the guselkumab 100 mg q4w group and by Week 20 for the 100 mg q8w group (FIG. 11). In the guselkumab 100 mg q4w group, the median steady-state trough serum guselkumab concentration was 3.90 μg/mL at Week 12 and was maintained through Week 24 (4.34 μg/mL). In the guselkumab 100 mg q8w group, the median steady-state trough serum guselkumab concentrations was 0.95 μg/mL at Week 20. The median steady-state trough serum guselkumab concentrations in the guselkumab 100 mg q4w group were approximately 4- to 5-fold higher compared with those in the guselkumab 100 mg q8w group (FIG. 11).

In the guselkumab 100 mg q4w group, the median steady-state trough guselkumab concentrations at Week 12 in subjects who met or did not meet EE criteria were 1.41 and 3.99 μg/mL, respectively. In the guselkumab 100 mg q8w group, the median steady-state trough guselkumab concentrations at Week 20 in subjects who met or did not meet EE criteria were 0.89 and 0.96 μg/mL, respectively. Median steady-state trough guselkumab concentrations appeared to be lower in subjects who met EE criteria. However, it should be noted that the number of subjects who met EE criteria was low for each treatment group (n≤4).

### Incidence of Antibodies to Guselkumab

A total of 254 subjects who received at least 1 dose of guselkumab and had appropriate samples for the detection of antibodies to guselkumab were included in the antibodies to guselkumab evaluation.

The overall incidence of antibodies to guselkumab through Week 24 was low (2.0%, 5/254) in subjects with PsA (Table 27). In the guselkumab 100 mg q4w group, the incidence of antibodies to guselkumab through Week 24 was 3.1% (4/128). In the guselkumab 100 mg q8w group, the incidence of antibodies to guselkumab through Week 24 was 0.8% (1/126). The highest titer of antibodies to guselkumab observed was 1:5120 in the 100 mg q4w group.

Of the 5 subjects with positive antibodies to guselkumab status, 1 (20%) subject in the guselkumab 100 mg q4w group was positive for NAbs to guselkumab.

The incidence of antibodies to guselkumab with or without MTX at baseline was 1.4% (2/139) and 2.6% (3/115), respectively. The incidence of antibodies to guselkumab with or without DMARD use at baseline was 1.2% (2/164) and 3.3% (3/90), respectively. Overall, the incidence of antibodies to guselkumab through Week 24 appeared to be lower in subjects with concomitant use of MTX or DMARDs compared with subjects without concomitant use of MTX of DMARDs. However, it should be noted that the number of subjects with positive antibodies to guselkumab status was small and the incidence of antibodies to guselkumab was low, regardless of concomitant MTX or DMARD use.

In addition, prior anti-TNFα use did not have an apparent impact on the incidence of antibodies to guselkumab. The incidence of antibodies to guselkumab with or without prior anti-TNFα use was 2.5% (2/79) and 1.7% (3/175), respectively.

TABLE 27
Summary of Anti-Guselkumab Antibodies Status Through Week 24; Immunogenicity
Analysis Set (Study CNTO1959PSA3001)

| | Guselkumab | | |
|---|---|---|---|
| | 100 mg q8w | 100 mg q4w | Combined |
| Analysis set: Immunogenicity Analysis Set | 126 | 128 | 254 |
| Subjects with appropriate samples^{a} | 126 | 128 | 254 |
| Subjects positive for anti-Guselkumab antibodies^{b,c} | 1 (0.8%) | 4 (3.1%) | 5 (2.0%) |
| Peak titers | | | |
| 1:40 | 0 | 1 | 1 |
| 1:80 | 1 | 0 | 1 |
| 1:160 | 0 | 2 | 2 |
| 1:5120 | 0 | 1 | 1 |
| Subjects negative for anti-Guselkumab antibodies^{b,d} | 125 (99.2%) | 124 (96.9%) | 249 (98.0%) |

^{a}Subjects with appropriate samples had 1 or more evaluable samples obtained after their first Guselkumab administration
^{b}Denominator is subjects with appropriate samples.
^{c}Includes all subjects who had at least 1 positive sample at any time post-baseline through Week 24.
^{d}Includes all subjects with negative samples at all times through Week 24 and excludes subjects who were positive at any time through Week 24.

### Antibodies to Guselkumab and Pharmacokinetics

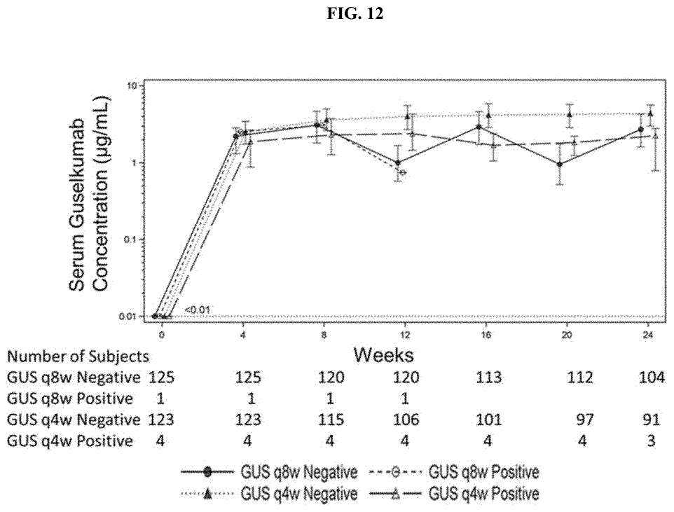

Serum guselkumab concentrations in subjects treated with guselkumab are summarized by treatment group and antibody to guselkumab status through Week 24. The median and IQ range of serum guselkumab concentrations through Week 24 by antibody to guselkumab status through Week 24 are graphed in FIG. 12. Individual serum guselkumab concentrations through Week 24 are also listed for subjects who were positive for antibodies to guselkumab.

In the guselkumab 100 mg q4w group, median serum guselkumab concentrations appeared to be lower in the 4 subjects with positive antibodies to guselkumab status compared to subjects with negative antibodies to guselkumab. In the guselkumab 100 mg q8w group, only 1 subject had positive antibodies to guselkumab, and this subject only had serum concentrations through Week 12. It should be noted that the number of subjects who were positive for antibodies to guselkumab was very small (n=5) which limits a definitive conclusion on the effect of immunogenicity on guselkumab PK (FIG. 12).

### Efficacy Results

### Primary Efficacy Endpoint Analysis

### ACR 20 Response at Week 24

At Week 24, a significantly greater proportion of subjects in both the guselkumab 100 mg q4w group (59.4%) and guselkumab 100 mg q8w group (52.0%) achieved an ACR 20 response compared with subjects in the placebo group (22.2%) based on both the global (ex-US) and US specific multiplicity testing procedures (both adjusted p<0.001; Table 28)). The ACR 20 response rate was slightly higher for the guselkumab 100 mg q4w group compared with the guselkumab 100 mg q8w group.

TABLE 28
Number of Subjects Achieving ACR 20 Response at Week 24 (Primary Analysis) Based on
the Composite Estimand; Full Analysis Set 1 (Study CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Subjects evaluable for ACR 20 Response at | 126 | 127 | 128 |
| Week 24^{a} | | | |
| Subjects with ACR 20 Response^{b,h} | 28 (22.2%) | 66 (52.0%) | 76 (59.4%) |
| All subjects (including those with imputed | 126 | 127 | 128 |
| data) | | | |
| Subjects with ACR 20 Response^{b,c,h} | 28 (22.2%) | 66 (52.0%) | 76 (59.4%) |
| % Difference (95% CI)^{d} | | 29.8 (18.6, 41.1) | 37.1 (26.1, 48.2) |
| p-value^{e} | | <0.001 | <0.001 |

^{a}Subjects either have an observed ACR 20 response status or met a Treatment Failure (TF) criterion.
^{b}Defined as observed responders who had not met any TF criteria prior to Week 24.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and prior exposure to anti-TNFα agents (yes/no).
^{h}ACR 20 response is defined as ≥20% improvement from baseline in both tender joint count (68 joints) and swollen joint count (66 joints), and ≥20% improvement from baseline in at least 3 of the 5 assessments: patient's assessment of pain, patient's global assessment of disease activity, physician's global assessment of disease activity, HAQ-DI, and CRP.

Improvements over placebo were consistently observed for ACR 20 response at Week 24 across all demographic subgroups for both guselkumab dose groups. In the majority of the subgroups defined by gender, race, age, weight or BMI, and participating countries, the lower bound of the 95% CI of the odds ratio was above 1 and the lower bound of the 95% CI of the difference in proportion of ACR 20 responders was above 0 for each guselkumab treatment compared with placebo, in favor of guselkumab.

Improvement over placebo was consistently observed for ACR 20 response at Week 24 in each of the 2 guselkumab dose groups in the majority of the subgroups defined by prior non-biologic DMARDs or anti-TNFα agent exposure, or baseline use of NSAID, oral corticosteroid, or non biologic DMARD. In the majority of these subgroups, the lower bound of the 95% CI of the odds ratio was above 1 and the lower bound of the 95% CI of the difference in proportion of ACR 20 responders was above 0 for each guselkumab treatment compared with placebo, in favor of guselkumab. Improvement over placebo was also observed in subjects who had prior inadequate response to non-biologic DMARDs or anti TNFα agents.

### Major Secondary Efficacy Endpoint Analyses

### Major Secondary Endpoints Controlled for Multiplicity in Both the Global (Ex-US) and US-Specific Testing Procedures

### Psoriasis IGA Response at Week 24

At baseline, 89 subjects in the guselkumab 100 mg q4w group, 82 subjects in the guselkumab 100 mg q8w group, and 78 subjects in placebo group had ≥3% BSA of psoriatic involvement and an IGA score≥2 at baseline. Among these subjects, a significantly greater proportion of subjects in both guselkumab groups achieved an IGA score of 0 (cleared) or 1 (minimal) and a ≥2-grade reduction from baseline in the IGA score at Week 24 compared with placebo, (both global and US-specific adjusted p<0.001; Table 29).

TABLE 29
Number of Subjects Achieving an Investigator Global Assessment (IGA) Score of 0
(Cleared) or 1 (Minimal), and ≥2 Grade Reduction from Baseline at Week 24, Based on the
Composite Estimand; Full Analysis Set 1 Among the Subjects with ≥3% Body Surface Area
(BSA) of Psoriatic Involvement and an IGA Score ≥2 (mild) at Baseline (Study
CNTO1959PSA3001)

| | | Guselkumab | |
|---|---|---|---|
| | Placebo | 100 mg q8w | 100 mg q4w |
| Analysis set: Full Analysis Set 1 Among | 78 | 82 | 89 |
| the Subjects with ≥3% Body Surface Area | | | |
| (BSA) Psoriatic Involvement and an IGA | | | |
| score of ≥2 (mild) at Baseline | | | |
| Subjects evaluable for IGA response at | 78 | 81 | 89 |
| Week 24^{a} | | | |
| Subjects with IGA response^{b,h} | 12 (15.4%) | 47 (58.0%) | 67 (75.3%) |
| All subjects (including those with imputed | 78 | 82 | 89 |
| data) | | | |
| Subjects with IGA response^{b,c,h} | 12 (15.4%) | 47 (57.3%) | 67 (75.3%) |
| % Difference (95% CI)^{d} | | 42.0 (28.9, 55.1) | 60.0 (48.3, 71.8) |
| p-value^{e} | | <0.001 | <0.001 |

^{a}Subjects either have an observed IGA response status or met a Treatment Failure (TF) criterion.
^{b}Defined as observed responders who had not met any TF criteria prior to Week 24.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and prior exposure to anti-TNFα agents (yes/no).
hThe IGA documents the investigator's assessment of the patient's psoriasis and lesions are graded for induration, erythema and scaling, each using a 5 point scale: 0 (no evidence), 1 (minimal), 2 (mild), 3 (moderate), and 4 (severe). The IGA score of psoriasis is based upon the average of induration, erythema and scaling scores. An IGA response is defined as an IGA score of 0 (cleared) or 1 (minimal) and ≥2 grade reduction from baseline.

Change from Baseline in HAQ-DI Score at Week 24

Physical function was assessed via HAQ-DI. At Week 24, a significantly greater reduction from baseline in HAQ-DI score was observed in both guselkumab groups compared with placebo, based on the composite estimand (both global and US-specific adjusted p<0.001; Table 30)

TABLE 30
Summary of the Change from Baseline in HAQ-DI Score at Week 24 Based on the
Composite Estimand Using MI and an ANCOVA Model; Full Analysis Set 1 (Study
CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Change from baseline in HAQ- | | | |
| DI^{a,h} | | | |
| Subjects evaluable^{b} | | | |
| N | 126 | 127 | 128 |
| Mean (SD) | −0.0873 (0.48638) | −0.3248 (0.56371) | −0.3652 (0.45723) |
| Median | 0.0000 | −0.2500 | −0.2500 |
| Range | (−1.625; 2.000) | (−1.875; 1.750) | (−1.750; 0.750) |
| IQ range | (−0.3750; 0.1250) | (−0.7500; 0.0000) | (−0.6250; 0.0000) |
| All subjects (including those with | | | |
| imputed data)^{a,c,h} | | | |
| N | 126 | 127 | 128 |
| Mean (SE)^{d} | −0.0873 (0.04333) | −0.3248 (0.05002) | −0.3652 (0.04041) |
| Model Based Estimates of the | | | |
| Mean Change^{a,c,h} | | | |
| LSMean (95% CI)e | −0.0743 (-0.1605, 0.0119) | −0.3225 (−0.4082, −0.2369) | −0.3968 (−0.4825, −0.3112) |
| LSMean difference (95% CI) | | −0.2483 (−0.3640, −0.1325) | −0.3226 (−0.4385, −0.2066) |
| p-value^{f} | | <0.001 | <0.001 |

^{a}Defined as the change from baseline using observed data or 0 (no improvement) if a subject met Treatment Failure (TF) criteria prior to Week 24.
^{b}Subjects either have an observed change from baseline at this visit or met TF criteria prior to this visit.
^{c}Missing data is assumed to be Missing at Random (MAR) and is imputed using Multiple Imputation (MI).
^{d}The average of the mean, taken over all the MI data sets, is presented. The variance of the mean is the weighted sum of the average within-imputation variance and the between-imputation variance.
^{e}The LSmean for each MI data set is calculated based on an Analysis of Covariance (ANCOVA) model for the change from baseline at Week 24. The combined LSmean which is the average of the LSmean, taken over all the MI data sets, is presented.
^{f}The p-values (nominal) are based on the approximately normal distribution of the combined LSmean.
^{h}The HAQ score is the average of the computed categories scores (dressing, arising, eating, walking, hygiene, gripping and daily living). Lower scores are indicative of better functioning.

Change from Baseline in SF-36 PCS at Week 24
The health-related quality of life was assessed using the SF-36. At Week 24, a significantly greater improvement from baseline in SF-36 PCS score was observed in both guselkumab groups compared with placebo, based on the composite estimand (both global and US-specific adjusted p<0.001; Table 31).

TABLE 31
Summary of the Change from Baseline in SF-36 PCS Score at Week 24 Based on the
Composite Estimand Using MI and an ANCOVA Model; Full Analysis Set 1 (Study
CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Change from baseline in SF-36 PCS | | | |
| score^{a,h} | | | |
| Subjects evaluable^{b} | | | |
| N | 126 | 127 | 127 |
| Mean (SD) | 2.175 (6.6929) | 6.213 (7.6629) | 6.405 (7.7287) |
| Median | 0.710 | 5.200 | 5.530 |
| Range | (−18.09; 25.49) | (−10.07; 30.21) | (−15.02; 32.83) |
| IQ range | (−1.780; 5.610) | (0.830; 10.280) | (1.040; 11.520) |
| All subjects (including those with imputed | | | |
| data)^{a,c,h} | | | |
| N | 126 | 127 | 128 |
| Mean (SE)^{d} | 2.175 (0.5962) | 6.213 (0.6800) | 6.419 (0.6826) |
| Model Based Estimates of the Mean | | | |
| Change^{a,c,h} | | | |
| LSMean (95% CI)^{e} | 1.96 (0.69, 3.24) | 6.10 (4.83, 7.37) | 6.87 (5.60, 8.14) |
| LSMean difference (95% CI) | | 4.14 (2.42, 5.85) | 4.91 (3.19, 6.63) |
| p-value^{f} | | <0.001 | <0.001 |

^{a}Defined as the change from baseline using observed data or 0 (no improvement) if a subject met Treatment Failure (TF) criteria prior to Week 24.
^{b}Subjects either have an observed change from baseline at this visit or met TF criteria prior to this visit.
^{c}Missing data is assumed to be Missing at Random (MAR) and is imputed using Multiple Imputation (MI).
^{d}The average of the mean, taken over all the MI data sets, is presented. The variance of the mean is the weighted sum of the average within-imputation variance and the between-imputation variance.
^{e}The LSmean for each MI data set is calculated based on an Analysis of Covariance (ANCOVA) model for the change from baseline at Week 24. The combined LSmean which is the average of the LSmean, taken over all the MI data sets, is presented.
^{f}The p-values (nominal) are based on the approximately normal distribution of the combined LSmean.
^{h}The physical component summary (PCS) and mental component summary (MCS) scores are calculated based on the 8 scales of the SF-36 Health Related Quality of Life instrument with 36 questions. Higher scores indicate better health.

Change from Baseline in DAS28 (CRP) at Week 24

At Week 24, a significantly greater reduction from baseline in DAS28 (CRP) score was observed in both guselkumab groups, compared with placebo (both global adjusted p<0.001; Table 32).

TABLE 32
Summary of the Change from Baseline in DAS 28 (CRP) Score at Week 24 Based on the
Composite Estimand Using MI and an ANCOVA Model; Full Analysis Set 1 (Study
CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Change from baseline in DAS28 (CRP)^{a,h} | | | |
| Subjects evaluable^{b} | | | |
| N | 126 | 126 | 128 |
| Mean (SD) | −0.72 (1.015) | −1.44 (1.144) | −1.53 (1.060) |
| Median | −0.46 | −1.36 | −1.50 |
| Range | (−4.0; 1.8) | (−4.5; 1.2) | (−4.4; 0.5) |
| IQ range | (−1.26; 0.00) | (−2.06; -0.61) | (−2.30; −0.76) |
| All subjects (including those with imputed | | | |
| data)^{a,c,h} | | | |
| N | 126 | 127 | 128 |
| Mean (SE)^{d} | −0.72 (0.090) | −1.44 (0.101) | −1.53 (0.094) |
| Model Based Estimates of the Mean | | | |
| Change^{a,c,h} | | | |
| LSMean (95% CI)^{e} | −0.70 (−0.89, −0.51) | −1.43 (−1.61, −1.24) | −1.61 (−1.80, −1.42) |
| LSMean difference (95% CI) | | −0.73 (−0.98, −0.48) | −0.91 (−1.16, −0.66) |
| p-value^{f} | | <0.001 | <0.001 |

^{a}Defined as the change from baseline using observed data or 0 (no improvement) if a subject met Treatment Failure (TF) criteria prior to Week 24.
^{b}Subjects either have an observed change from baseline at this visit or met TF criteria prior to this visit.
^{c}Missing data is assumed to be Missing at Random (MAR) and is imputed using Multiple Imputation (MI).
^{d}The average of the mean, taken over all the MI data sets, is presented. The variance of the mean is the weighted sum of the average within-imputation variance and the between-imputation variance.
^{e}The LSmean for each MI data set is calculated based on an Analysis of Covariance (ANCOVA) model for the change from baseline at Week 24. The combined LSmean which is the average of the LSmean, taken over all the MI data sets, is presented.
^{f}The p-values (nominal) are based on the approximately normal distribution of the combined LSmean.
^{h}The DAS 28 (CRP) score is calculated based on the tender joints (28), swollen joints (28), patient's global assessment of disease activity, and CRP.

### ACR 20 Response at Week 16

At Week 16, significantly greater proportions of subjects in both guselkumab groups achieved an ACR 20 response compared with subjects in the placebo group (both global adjusted p<0.001; Table 33).

TABLE 33
Number of Subjects Achieving ACR 20 Response at Week 16 Based on the Composite
Estimand; Full Analysis Set 1 (Study CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Subjects evaluable for ACR 20 Response at | 125 | 127 | 128 |
| Week 16^{a} | | | |
| Subjects with ACR 20 Response^{b,h} | 32 (25.6%) | 66 (52.0%) | 77 (60.2%) |
| All subjects (including those with imputed | 126 | 127 | 128 |
| data) | | | |
| Subjects with ACR 20 Response^{b,c,h} | 32 (25.4%) | 66 (52.0%) | 77 (60.2%) |
| % Difference (95% CI)^{d} | | 26.7 (15.3, 38.1) | 34.8 (23.5, 46.0) |
| p-value^{e} | | <0.001 | <0.001 |

^{a}Subjects either have an observed ACR 20 response status or met a Treatment Failure (TF) criterion.
^{b}Defined as observed responders who had not met any TF criteria prior to Week 16.
cSubjects with missing data are assumed to be non-responders.
^{d}The confidence intervals are based on the Wald statistic.
^{e}The p-values (nominal) are based on the CMH test, stratified by baseline use of non-biologic DMARD (yes, no) and prior exposure to anti-TNFα agents (yes/no).
^{h}ACR 20 response is defined as ≥20% improvement from baseline in both tender joint count (68 joints) and swollen joint count (66 joints), and ≥20% improvement from baseline in at least 3 of the 5 assessments: patient's assessment of pain, patient's global assessment of disease activity, physician's global assessment of disease activity, HAQ-DI, and CRP.

### ACR 50 Response at Week 24

At Week 24, significantly greater proportions of subjects in both guselkumab groups achieved an ACR 50 response compared with subjects in the placebo group (both global adjusted p<0.001; Table 34).

TABLE 34
Number of Subjects Achieving ACR 50 Response at Week 24 Based on the Composite
Estimand; Full Analysis Set 1 (Study CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Subjects evaluable for ACR 50 Response at | 126 | 127 | 128 |
| Week 24^{a} | | | |
| Subjects with ACR 50 Response^{b,c,h} | 11 (8.7%) | 38 (29.9%) | 46 (35.9%) |
| All subjects (including those with imputed | 126 | 127 | 128 |
| data) | | | |
| Subjects with ACR 50 Response^{b,c,h} | 11 (8.7%) | 38 (29.9%) | 46 (35.9%) |
| % Difference (95% CI)^{d} | | 21.4 (12.1, 30.7) | 27.2 (17.6, 36.8) |
| p-value^{e} | | <0.001 | <0.001 |

^{a}Subjects either have an observed ACR 50 response status or met a Treatment Failure (TF) criterion.
^{b}Defined as observed responders who had not met any TF criteria prior to Week 24.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and prior exposure to anti-TNFα agents (yes/no).
^{h}ACR 50 response is defined as ≥50% improvement from baseline in both tender joint count (68 joints) and swollen joint count (66 joints), and ≥50% improvement from baseline in at least 3 of the 5 assessments: patient's assessment of pain, patient's global assessment of disease activity, physician's global assessment of disease activity, HAQ-DI, and CRP.

### ACR 70 Response at Week 24

Guselkumab 100 mg q4w dose regimen. At Week 24, a significantly greater proportion of subjects in the guselkumab 100 mg q4w group achieved an ACR 70 response compared with subjects in the placebo group (global adjusted p<0.001; Table 35).

Guselkumab 100 mg q8w dose regimen. A numerically greater proportion of subjects in the guselkumab 100 mg q8w group achieved an ACR 70 response at Week 24 compared with subjects in the placebo group; however, a statistical significance was not achieved (global adjusted p=0.086; Table 35).

TABLE 35
Number of Subjects Achieving ACR 70 Response at Week 24 Based on the Composite
Estimand; Full Analysis Set 1 (Study CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Subjects evaluable for ACR 70 Response at | 126 | 127 | 128 |
| Week 24^{a} | | | |
| Subjects with ACR 70 Response^{b,h} | 7 (5.6%) | 15 (11.8%) | 26 (20.3%) |
| All subjects (including those with imputed | 126 | 127 | 128 |
| data) | | | |
| Subjects with ACR 70 Response^{b,c,h} | 7 (5.6%) | 15 (11.8%) | 26 (20.3%) |
| % Difference (95% CI)^{d} | | 6.4 (−0.3, 13.1) | 14.8 (6.9, 22.7) |
| p-value^{e} | | 0.069 | <0.001 |

^{a}Subjects either have an observed ACR 70 response status or met a Treatment Failure (TF) criterion.
^{b}Defined as observed responders who had not met any TF criteria prior to Week 24.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and prior exposure to anti-TNFα agents (yes/no).
^{h}ACR 70 response is defined as ≥70% improvement from baseline in both tender joint count (68 joints) and swollen joint count (66 joints), and ≥70% improvement from baseline in at least 3 of the 5 assessments: patient's assessment of pain, patient's global assessment of disease activity, physician's global assessment of disease activity, HAQ-DI, and CRP.

### ACR 50 Response at Week 16

Guselkumab 100 mg q4w dose regimen. At Week 16, a significantly greater proportion of subjects in the guselkumab 100 mg q4w group achieved an ACR 50 response compared with subjects in the placebo group (global adjusted p=0.006; Table 36).

Guselkumab 100 mg q8w dose regimen. A numerically greater proportion of subjects in the guselkumab 100 mg q8w group achieved an ACR 50 response at Week 16 compared with subjects in the placebo group; however, a statistical significance was not achieved after multiplicity adjustment (global adjusted p=0.086; Table 36).

TABLE 36
Number of Subjects Achieving ACR 50 Response at Week 16 Based
on the Composite Estimand; Full Analysis Set 1 (Study CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Subjects evaluable for ACR 50 Response at | 125 | 127 | 128 |
| Week 16^{a} | | | |
| Subjects with ACR 50 Response^{b, h} | 16 (12.8%) | 29 (22.8%) | 34 (26.6%) |
| All subjects (including those with imputed | 126 | 127 | 128 |
| data) | | | |
| Subjects with ACR 50 Response^{b, c, h} | 16 (12.7%) | 29 (22.8%) | 34 (26.6%) |
| % Difference (95% CI)^{d} | | 10.2 (1.0, 19.3) | 13.9 (4.4, 23.4) |
| p-value^{e} | | 0.036 | 0.006 |

^{a}Subjects either have an observed ACR 50 response status or met a Treatment Failure (TF) criterion.
^{b}Defined as observed responders who had not met any TF criteria prior to Week 16.
^{c}Subjects with missing data are assumed to be non-responders.
^{d}The confidence intervals are based on the Wald statistic.
^{e}The p-values (nominal) are based on the CMH test, stratified by baseline use of non-biologic DMARD (yes, no) and prior exposure to anti-TNFα agents (yes/no).
^{h}ACR 50 response is defined as ≥50% improvement from baseline in both tender joint count (68 joints) and swollen joint count (66 joints), and ≥50% improvement from baseline in at least 3 of the 5 assessments: patient's assessment of pain, patient's global assessment of disease activity, physician's global assessment of disease activity, HAQ-DI, and CRP.

### Major Secondary Endpoints not Controlled for Multiplicity

### Enthesitis Assessed Using LEI

Endpoints related to enthesitis were evaluated in subjects with enthesitis assessed by LEI at baseline: 73 subjects in the guselkumab 100 mg q4w group, 72 subjects in the guselkumab 100 mg q8w group, and 77 subjects in the placebo group.

The impact of guselkumab on enthesitis was assessed using 2 approaches: the number of subjects who achieved resolution of enthesitis (LEI) at Week 24 and the change from baseline in the enthesitis score (LEI) at Week 24 based on the composite estimand. Non-responder imputation was used for missing resolution of enthesitis and MI was used for missing change from baseline in LEI.

### Resolution of Enthesitis at Week 24

At Week 24, among the 222 (58.3%) subjects with enthesitis at baseline, 47.9% of subjects in the guselkumab 100 mg q4w group and 40.3% of subjects in the guselkumab 100 mg q8w group achieved enthesitis resolution compared to 27.3% of subjects in the placebo group (nominal p=0.013 and p=0.094, respectively;).

Change from Baseline in Enthesitis Score at Week 24

At Week 24, among the 222 (58.3%) subjects with enthesitis at baseline, LSmean change from baseline in LEI scores were −1.75 in the guselkumab 100 mg q4w group and −1.35 in the guselkumab 100 mg q8w group compared to −1.01 in the placebo group (nominal p=0.004 and nominal p=0.185, respectively).

### Dactylitis

Endpoints related to dactylitis were evaluated in subjects with dactylitis at baseline: 38 subjects in the guselkumab 100 mg q4w group, 49 subjects in the guselkumab 100 mg q8w group, and 55 subjects in the placebo group.

The impact of guselkumab on dactylitis was assessed using 2 approaches: the number of subjects who achieved resolution of dactylitis at Week 24 and the change from baseline in the dactylitis score at Week 24 based on the composite estimand. Non-responder imputation was used for missing resolution of dactylitis and MI was used for missing change from baseline in dactylitis score.

### Resolution of Dactylitis at Week 24

At Week 24, among the 142 (37.3%) subjects with dactylitis at baseline, numerically greater proportions of subjects in the guselkumab 100 mg q4w group (63.2%, nominal p=0.212) and the guselkumab 100 mg q8w group (65.3%, nominal p=0.088) achieved dactylitis resolution compared to the placebo group (49.1%).

Change from Baseline in Dactylitis Score at Week 24

At Week 24, among the 142 (37.3%) subjects with dactylitis at baseline, a numerically greater reduction from baseline in dactylitis score was observed in the guselkumab 100 mg q4w group (LSmean change from baseline: −5.82, nominal p=0.225) and the guselkumab 100 mg q8w group (LSmean change from baseline: −6.11, nominal p=0.121) compared to the placebo group (LSmean change from baseline: −4.30).

Change from Baseline in SF-36 MCS at Week 24

At Week 24, a numerically greater improvement from baseline in SF-36 MCS score was observed in the guselkumab 100 mg q4w group (LSmean: 3.60, nominal p=0.214) and the guselkumab 100 mg q8w group (LSmean: 3.20, nominal p=0.398) compared to the placebo group (LSmean: 2.37).

### Other Efficacy Endpoints Related to Reduction of Joint Signs and Symptoms

### ACR 20, ACR 50, and ACR 70 Responses Through Week 24

Through Week 24, ACR 20, ACR 50, and ACR 70 response rates were consistently higher in the 2 guselkumab groups than those in the placebo group over time.

For the guselkumab 100 mg q4w group, separations from placebo (defined as nominal p<0.05, hereafter) for ACR 20, ACR 50, and ACR 70 response rates were first observed at Week 4, Week 12, and Week 20, respectively. For the guselkumab 100 mg q8w group, separations from placebo on ACR 20 and ACR 50 response rates were first observed at Week 8 and Week 12, respectively. The greatest ACR 20 response was observed at Week 20 for guselkumab 100 mg q4w and at Week 16 for guselkumab 100 mg q8w.

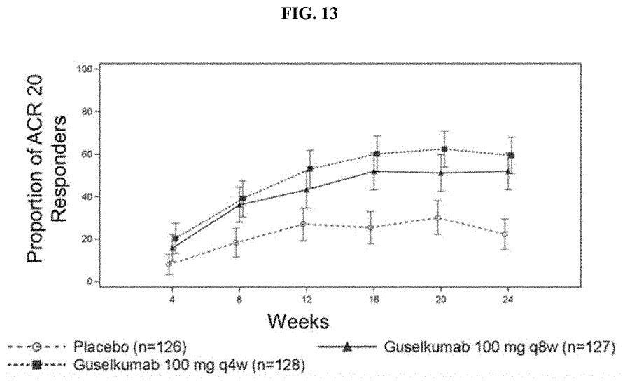

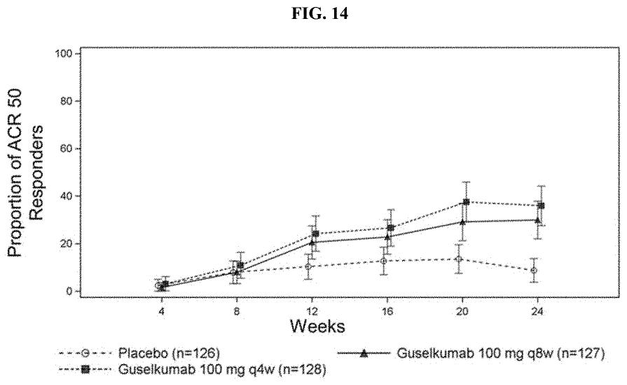

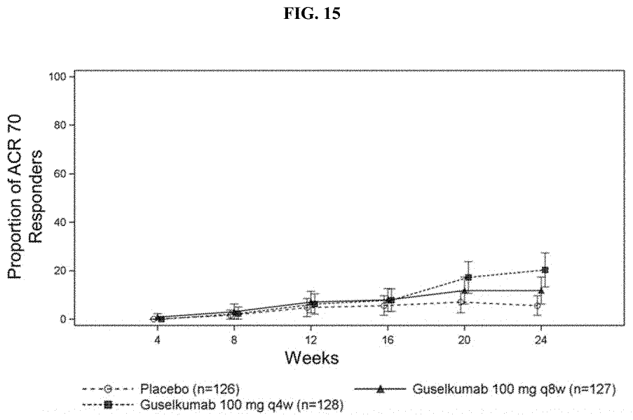

The ACR 20, ACR 50, and ACR 70 response rates were numerically higher in the guselkumab 100 mg q4w group than those in the guselkumab 100 mg q8w group over time through Week 24, with the greatest difference observed for ACR 70 response rate at Week 24 (FIG. 13, FIG. 14, FIG. 15).

### ACR Components

The 7 components of the ACR response are: swollen and tender joint count, patient's assessment of pain (by VAS), patient's and physician's global assessment of disease activity (by VAS), HAQ-DI, and CRP.

The median percent reduction from baseline for each ACR component generally increased over time for both guselkumab treatment groups through Week 24. A numerically greater percent reduction from baseline compared with placebo was observed from Week 4 for most of the ACR components except HAQ-DI in both guselkumab treatment groups. For HAQ-DI, numerical difference from placebo was observed from Week 4 for the guselkumab 100 mg q4w group and from Week 8 for the guselkumab 100 mg q8w group.

At Week 24, the median percent change from baseline in ACR components in the guselkumab 100 mg q4w and 100 mg q8w groups compared with the placebo group were as follows:
Number of swollen joints: −87.5% and −83.3% compared with −60.0%, respectively
Number of tender joints: −66.7% and −66.7% compared with −37.8%, respectively
Patient's assessment of pain: −39.33% and −37.50% compared with −8.20%, respectively
Patient's global assessment of disease activity: −44.00% and −42.86% compared with −10.23%, respectively
Physician's global assessment of disease activity: −70.21% and −58.31% compared with −32.43%, respectively
HAQ-DI score: −33.3333% and −25.0000% compared with −6.9048%, respectively
CRP: −37.423% and −24.423% compared with −21.185%, respectively

There was no consistent difference between the 2 guselkumab treatment groups observed among the ACR components over time through Week 24.

### DAS28 (CRP)

As early as the first evaluation at Week 4, separations from placebo in change from baseline in DAS28 (CRP) score were observed in both guselkumab treatment groups. The treatment effect increased over time through Week 24 for both guselkumab 100 mg q4w and q8w groups compared with placebo (both nominal p<0.001; Table 32). The treatment effect was numerically greater in the guselkumab 100 mg q4w group than in the guselkumab 100 mg q8w group, most notably from Week 16 through Week 24.

A tipping point analysis based on the treatment policy estimand was performed for the change in baseline in DAS28 (CRP) score at Week 16 using MI for missing data.

### DAS28 (CRP) Responses Through Week 24

The proportion of subjects achieving a DAS28 (CRP) good or moderate response in both guselkumab treatment groups increased over time reaching peak at Week 12 (Separation from placebo was observed from Week 4 for the guselkumab 100 mg q4w group and from Week 8 for the guselkumab 100 mg q8w group.

At Week 24, the proportion of subjects achieving a DAS28 (CRP) good or moderate response was 76.6% and 70.9% in the guselkumab 100 mg q4w and guselkumab 100 mg q8w groups, respectively, compared with 44.4% (both nominal p<0.001) in the placebo group.

The effect size was numerically greater in the guselkumab 100 mg q4w group than in the guselkumab 100 mg q8w group at Week 4 and from Week 12 through Week 24.

Through Week 24, the proportion of subjects who achieved DAS28 (CRP) remission (<2.6) was consistently higher in the 2 guselkumab groups compared with placebo over time. Separation from placebo was observed from Weeks 12 through Week 24 for the guselkumab 100 mg q4w group and at Weeks 12, 16, and 24, but not Week 20 (due to high placebo response) for the guselkumab 100 mg q8w group. Peak response was observed at Week 20 for both guselkumab treatment groups and the treatment effect was numerically greater in the guselkumab 100 mg q4w group than that in the guselkumab 100 mg q8w group from Week 16 through Week 24.

At Week 24, DAS28 (CRP) remission was achieved by a greater proportion of subjects in the guselkumab 100 mg q4w and guselkumab 100 mg q8w groups (35.9% and 23.6%, respectively) compared with the placebo group (12.7%; nominal p<0.001 and nominal p=0.025, respectively).

### Responses Based on Modified PsARC Through Week 24

The proportion of subjects achieving a modified PsARC response in both guselkumab treatment groups increased over time from Week 4 through Week 24. Separation from placebo was observed from Week 4 for the guselkumab 100 mg q4w group and from Week 8 for the guselkumab 100 mg q8w group. Peak response was observed at Week 20 for both guselkumab treatment groups and the treatment effect was numerically greater in the guselkumab 100 mg q4w group than that in the guselkumab 100 mg q8w group at Week 4 and from Week 12 through Week 24.

At Week 24, the proportion of subjects achieving a modified PsARC response was 72.7% in the guselkumab 100 mg q4w group and 59.8% in the guselkumab 100 mg q8w group compared with 31.0% in the placebo group (both nominal p<0.001).

### DAPSA Index

Change from Baseline in DAPSA Through Week 24. Greater improvements in change from baseline in DAPSA index were observed in the guselkumab 100 mg q4w and 100 mg q8w groups compared with the placebo group over time from Week 4 through Week 24 (all nominal p<0.05). Peak effect was observed from Week 16 through Week 24 for both guselkumab treatment groups and the effect size was comparable between the 2 guselkumab treatment groups from Week 4 through Week 24.

At Week 24, the reduction from baseline in DAPSA index was numerically greater in the guselkumab 100 mg q4w group (LSmean change from baseline: −20.621) and the guselkumab 100 mg q8w group (LSmean change from baseline: −21.332) compared with the placebo group (LSmean change from baseline: −10.749; both nominal p<0.001).

### Low Disease Activity or Remission Based on DAPSA

Low disease activity: Through Week 24, the proportions of subjects achieving low disease activity based on the DAPSA index were consistently higher in the 2 guselkumab groups compared with the placebo group. Separation from placebo was observed from Week 8 through Week 24 for the guselkumab 100 mg q4w group and from Week 16 through Week 24 for the guselkumab 100 mg q8w group. At Week 24, the proportion of subjects achieving low disease activity based on the DAPSA index was 49.2% in the guselkumab 100 mg q4w group and 40.9% in the guselkumab 100 mg q8w group compared with 16.7% in the placebo group (both nominal p<0.001).

Remission: Through Week 24, the proportions of subjects achieving remission based on the DAPSA index were numerically higher in the 2 guselkumab groups compared with the placebo group. Separation from placebo was observed at Week 20 and Week 24 for the guselkumab 100 mg q4w group and not observed for the guselkumab 100 mg q8w group through Week 24. At Week 24, the proportion of subjects achieving remission based on the DAPSA index was 14.1% in the guselkumab 100 mg q4w group (nominal p=0.017) and 6.3% in the guselkumab 100 mg q8w group (nominal p=0.785) compared with 4.8% in the placebo group.

### Other Efficacy Endpoints Related to Physical Function

Change from Baseline in HAQ-DI Score Through Week 24

Through Week 24, numerically greater reduction from baseline in HAQ-DI were consistently observed in the 2 guselkumab groups compared with placebo over time. Separation from placebo was observed from Week 4 through Week 24 for the guselkumab 100 mg q4w group and from Week 12 through Week 24 for the guselkumab 100 mg q8w group, with the greatest effect observed at Week 24 for the guselkumab 100 mg q4w group and at Week 20 for the guselkumab 100 mg q8w group. The effect size was numerically greater in the guselkumab 100 mg q4w group than that in the guselkumab 100 mg q8w group from Week 4 through Week 24.

A tipping point analysis based on the treatment policy estimand using MI and ANCOVA was performed for the change in baseline in HAQ-DI score at Week 16. The results based on the treatment policy estimand were consistent with those of the main analysis. There were 1, 3, and 4 subjects with missing data in the guselkumab 100 mg q4w, guselkumab 100 mg q8w, and placebo groups, respectively; the tipping point analysis indicated that the result only tipped under unrealistic assumptions penalizing guselkumab and/or favoring placebo, demonstrating the robustness of the results.

### HAQ DI Response Through Week 24

At baseline, 110 subjects in the guselkumab 100 mg q4w group, 112 subjects in the guselkumab 100 mg q8w, and 110 subjects in the placebo group had a HAQ-DI score≥0.35. Through Week 24, higher HAQ-DI response rates (defined as ≥0.35 improvement from baseline) were consistently observed in the 2 guselkumab groups compared with placebo over time. Separation from placebo was observed from Week 8 through Week 24 for both guselkumab treatment groups. Peak effect was observed at Week 16 for the guselkumab 100 mg q4w group and at Week 20 for the guselkumab 100 mg q8w group. The effect size was numerically greater in the guselkumab q4w group than that in the guselkumab 100 mg q8w group from Week 12 through Week 24. At Week 24, among subjects with HAQ≥0.35 at baseline, the proportion of subjects achieving HAQ-DI response was 57.3% in the guselkumab 100 mg q4w group (nominal p<0.001) and 50.9% in the guselkumab 100 mg q8w group (nominal p=0.001) compared with 29.1% in the placebo group.

### Other Efficacy Endpoints Related to Skin Disease

Endpoints related to skin disease were evaluated in subjects with ≥3% BSA psoriasis skin involvement and an IGA score of ≥2 (mild) at baseline: 89 subjects in the guselkumab 100 mg q4w group, 82 subjects in the guselkumab 100 mg q8w group, and 78 subjects in the placebo group. Assessments of IGA and PASI were collected at Weeks 0, 16, and 24.

### IGA

### Psoriasis IGA Response Through Week 24

Among the 249 (65.4%) subjects with ≥3% BSA psoriasis skin involvement and an IGA score of ≥2 at baseline, greater proportions of subjects in the guselkumab 100 mg q4w (64.0%) and 100 mg q8w (62.2%) groups achieved a psoriasis response (IGA of 0 [cleared] or 1 [minimal] and a ≥2-grade reduction from baseline) at Week 16 compared with the placebo group (16.7%; nominal p<0.001). At Week 24, the proportion of subjects achieving an IGA response further increased in the guselkumab 100 mg q4w group and remained higher in the guselkumab 100 mg q8w group compared with the placebo group (both nominal p<0.001; Table 29). The effect size was comparable between the 2 guselkumab treatment groups at Week 16 and numerically higher in the guselkumab 100 mg q4w group compared with the q8w group at Week 24.

A tipping point analysis based on the treatment policy estimand using MI was performed for the number of subjects achieving an IGA score of 0 (clear) or 1 (minimal) and ≥2 grade reduction from baseline at Week 16.

### IGA Score of 0 (Clear) Through Week 24

Among the 249 (65.4%) subjects with ≥3% BSA psoriasis skin involvement and an IGA score of ≥2 at baseline, greater proportions of subjects in the guselkumab 100 mg q4w and 100 mg q8w groups achieved an IGA score of 0 (clear) compared to the placebo group at Week 16 (both nominal p<0.001; Table 37). At Week 24, the proportions of subjects who achieved an IGA score of 0 (clear) were further increased to 53.9% and 38.3% in the guselkumab 100 mg q4w and guselkumab 100 mg q8w groups, respectively, compared with 7.7% in the placebo group (both nominal p<0.001). The effect size was numerically greater in the guselkumab 100 mg q4w group compared to the guselkumab 100 mg q8w group at Week 16 and the difference between the 2 guselkumab treatment groups was further increased at Week 24.

The number of subjects achieving an IGA score of 0 (clear) in evaluable subjects through Week 24 based on the treatment policy estimand among subjects with ≥3% BSA psoriatic involvement

TABLE 37
Number of Subjects with an IGA Score of 0 by Visit Through
Week 24, Based on the Composite Estimand; Full Analysis Set 1
Among the Subjects with ≥3% Body Surface Area (BSA) of Psoriatic
Involvement and an IGA Score ≥ 2 (mild) at Baseline (Study
CNTO1959PSA3001)

| | | Guselkumab | |
|---|---|---|---|
| | Placebo | 100 mg q8w | 100 mg q4w |
| Analysis set: Full Analysis | 78 | 82 | 89 |
| Set 1 Among the Subjects | | | |
| Who had ≥3% Body | | | |
| Surface Area (BSA) of | | | |
| Psoriatic Involvement | | | |
| and an IGA Score ≥ 2 | | | |
| (mild) at Baseline | | | |
| Week 16 | | | |
| Subjects evaluable for an | 76 | 81 | 86 |
| IGA score of 0^{a} | | | |
| Subjects with an IGA | 7 (9.2%) | 27 (33.3%) | 36 (41.9%) |
| score of 0^{b, h} | | | |
| All subjects (including | 78 | 82 | 89 |
| those with imputed data) | | | |
| Subjects with an IGA | 7 (9.0%) | 27 (32.9%) | 36 (40.4%) |
| score of 0^{b, c, h} | | | |
| % Difference (95% CI)^{d} | | 24.3 (12.4, 36.1) | 31.6 (19.8, |
| | | | 43.3) |
| p-value^{e} | | <0.001 | <0.001 |
| Week 24 | | | |
| Subjects evaluable for an | 78 | 81 | 89 |
| IGA score of 0^{a} | | | |
| Subjects with an IGA | 6 (7.7%) | 31 (38.3%) | 48 (53.9%) |
| score of 0^{b, h} | | | |
| All subjects (including | 78 | 82 | 89 |
| those with imputed data) | | | |
| Subjects with an IGA | 6 (7.7%) | 31 (37.8%) | 48 (53.9%) |
| score of 0^{b, c, h} | | | |
| % Difference (95% CI)^{d} | | 30.5 (18.8, 42.2) | 46.4 (34.6, |
| | | | 58.1) |
| p-value^{e} | | <0.001 | <0.001 |

^{a}Subjects either have an observed IGA response status or met a Treatment Failure (TF) criterion.
^{b}Defined as observed responders who had not met any TF criteria prior to the specific visit at which the endpoint was assessed.
^{c}Subjects with missing data at a visit are assumed to be non-responders at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and prior exposure to anti-TNFα agents (yes/no) is used to calculate the p-values. The symbol “†” will be attached as a superscript to those p-values that are calculated using the Fisher's exact test.
^{h}The IGA documents the investigator's assessment of the patient's psoriasis and lesions are graded for induration, erythema and scaling, each using a 5 point scale: 0 (no evidence), 1 (minimal), 2 (mild), 3 (moderate), and 4 (severe). The IGA score of psoriasis is based upon the average of induration, erythema and scaling scores.

### PASI

### PASI Responses Through Week 24

The number of subjects who achieved PASI 50, PASI 75, PASI 90, and PASI 100 responses through Week 24 among the 249 (65.4%) subjects with ≥3% BSA psoriatic involvement and an IGA score of ≥2 at baseline are provided in Table 38 and Table 39.

Among these subjects, greater proportions of subjects with PASI 50, PASI 75, PASI 90, and PASI 100 responses at Week 16 were observed in both guselkumab treatment groups compared with the placebo group (all nominal p<0.006). Response rates increased at Week 24 for both guselkumab treatment groups.

At Week 24, the proportions of subjects who achieved PASI 100 response was 44.9% in the guselkumab 100 mg q4w group and 25.6% in the guselkumab 100 mg q8w group compared with 6.4% in the placebo group (both nominal p<0.001).

The effect size was numerically greater in the guselkumab 100 mg q4w group compared to the guselkumab 100 mg q8w group at Week 16 and the difference between the 2 guselkumab treatment groups was further increased at Week 24.

TABLE 38
Number of Subjects Achieving a PASI 75 Response by Visit
Through Week 24, Based on the Composite Estimand; Full Analysis Set
1 Among the Subjects with ≥3% Body Surface Area (BSA) of Psoriatic
Involvement and an IGA Score ≥ 2 (mild) at Baseline (Study
CNTO1959PSA3001)

| | | Guselkumab | |
|---|---|---|---|
| | Placebo | 100 mg q8w | 100 mg q4w |
| Analysis set: Full Analysis Set 1 | 78 | 82 | 89 |
| Among the Subjects Who had | | | |
| >3% Body Surface Area (BSA) | | | |
| of Psoriatic Involvement and | | | |
| an IGA Score ≥ 2 (mild) at | | | |
| Baseline | | | |
| Week 16 | | | |
| Subjects evaluable for PASI 75 | 76 | 81 | 87 |
| response^{a} | | | |
| Subjects with PASI 75 | 16 (21.1%) | 52 (64.2%) | 65 (74.7%) |
| response^{b, h} | | | |
| All subjects (including those | 78 | 82 | 89 |
| with imputed data) | | | |
| Subjects with PASI 75 | 16 (20.5%) | 52 (63.4%) | 65 (73.0%) |
| response^{b, c, h} | | | |
| % Difference (95% CI)^{d} | | 43.0 (29.4, | 52.5 (39.9, |
| | | 56.6) | 65.1) |
| p-value^{e} | | <0.001 | <0.001 |
| Week 24 | | | |
| Subjects evaluable for PASI 75 | 78 | 81 | 89 |
| response^{a} | | | |
| Subjects with PASI 75 | 11 (14.1%) | 62 (76.5%) | 77 (86.5%) |
| response^{b, h} | | | |
| All subjects (including those | 78 | 82 | 89 |
| with imputed data) | | | |
| Subjects with PASI 75 | 11 (14.1%) | 62 (75.6%) | 77 (86.5%) |
| response^{b, c, h} | | | |
| % Difference (95% CI)^{d} | | 61.7 (49.8, | 72.6 (62.3, |
| | | 73.7) | 82.8) |
| p-value^{e} | | <0.001 | <0.001 |

^{a}Subjects either have an observed PASI 75 response status or met a Treatment Failure (TF) criterion.
^{b}Defined as observed responders who had not met any TF criteria prior to the specific visit at which the endpoint was assessed.
^{c}Subjects with missing data at a visit are assumed to be non-responders at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and prior exposure to anti-TNFα agents (yes/no) is used to calculate the p-values. The symbol “†” will be attached as a superscript to those p-values that are calculated using the Fisher's exact test.
^{h}The PASI score is a composite of the state of erythema, induration and scaling over the body along with the area of the involvement of psoriatic lesions. The PASI score ranges from 0 to 72, with a higher score indicating more severe disease. PASI 75 response is defined as ≥75% improvement from baseline in PASI score.

TABLE 39
Number of Subjects Achieving a PASI 90 Response by Visit
Through Week 24, Based on the Composite Estimand; Full Analysis
Set 1 Among the Subjects with ≥3% Body Surface Area (BSA) of
Psoriatic Involvement and an IGA Score ≥ 2 (mild) at Baseline (Study
CNTO1959PSA3001)

| | | Guselkumab | |
|---|---|---|---|
| | Placebo | 100 mg q8w | 100 mg q4w |
| Analysis set: Full Analysis Set 1 | 78 | 82 | 89 |
| Among the Subjects Who had | | | |
| ≥3% Body Surface Area (BSA) | | | |
| of Psoriatic Involvement and an | | | |
| IGA Score ≥ 2 (mild) at Baseline | | | |
| Week 16 | | | |
| Subjects evaluable for PASI 90 | 76 | 81 | 87 |
| response^{a} | | | |
| Subjects with PASI 90 | 8 (10.5%) | 37 (45.7%) | 47 (54.0%) |
| response^{b, h} | | | |
| All subjects (including those | 78 | 82 | 89 |
| with imputed data) | | | |
| Subjects with PASI 90 | 8 (10.3%) | 37 (45.1%) | 47 (52.8%) |
| response^{b, c, h} | | | |
| % Difference (95% CI)^{d} | | 34.9 (22.2, | 42.6 (30.5, |
| | | 47.6) | 54.8) |
| p-value^{e} | | <0.001 | <0.001 |
| Week 24 | | | |
| Subjects evaluable for PASI 90 | 78 | 81 | 89 |
| response^{a} | | | |
| Subjects with PASI 90 | 9 (11.5%) | 41 (50.6%) | 56 (62.9%) |
| response^{b, h} | | | |
| All subjects (including those | 78 | 82 | 89 |
| with imputed data) | | | |
| Subjects with PASI 90 | 9 (11.5%) | 41 (50.0%) | 56 (62.9%) |
| response^{b, c, h} | | | |
| % Difference (95% CI)^{d} | | 38.6 (25.8, | 51.7 (39.7, |
| | | 51.4) | 63.7) |
| p-value^{e} | | <0.001 | <0.001 |

^{a}Subjects either have an observed PASI 90 response status or met a Treatment Failure (TF) criterion.
^{b}Defined as observed responders who had not met any TF criteria prior to the specific visit at which the endpoint was assessed.
^{c}Subjects with missing data at a visit are assumed to be non-responders at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and prior exposure to anti-TNFα agents (yes/no) is used to calculate the p-values. The symbol “†” will be attached as a superscript to those p-values that are calculated using the Fisher's exact test.
^{h}The PASI score is a composite of the state of erythema, induration and scaling over the body along with the area of the involvement of psoriatic lesions. The PASI score ranges from 0 to 72, with a higher score indicating more severe disease. PASI 90 response is defined as ≥90% improvement from baseline in PASI score.

Change from Baseline in PASI Through Week 24

Consistent with data on the proportion of subjects achieving a PASI response over time, greater reductions in PASI score from baseline was observed in both guselkumab treatment groups compared with the placebo group at Week 16 and Week 24 (all nominal p<0.001).

At Week 24, the reduction in PASI score from baseline was greater in the guselkumab 100 mg q4w group (LSmean change from baseline: −10.915) and the guselkumab 100 mg q8w group (LSmean change from baseline: −9.974) compared with the placebo group (LSmean change from baseline: −2.317; both nominal p<0.001). Of note, the effect size was numerically comparable between the 2 guselkumab doses at Week 16 and slightly greater in the guselkumab 100 mg q4w group compared to the guselkumab 100 mg q8w group at Week 24.

### PASI 75 and ACR 20 Responses Through Week 24

At Week 16, among the 249 (65.4%) subjects with ≥3% BSA psoriatic involvement and an IGA score of ≥2 at baseline, greater proportions of subjects in both guselkumab treatment groups achieved both a PASI 75 and an ACR 20 response compared with the placebo group (both nominal p<0.001; Table 40). The proportion of subjects achieving both PASI 75 and ACR 20 responses increased at Week 24 for both guselkumab groups compared with placebo (both nominal p<0.001). The effect size was numerically greater in the guselkumab 100 mg q4w group compared to the guselkumab 100 mg q8w group at both Week 16 and Week 24.

TABLE 40
Number of Subjects Achieving Both PASI 75 and ACR 20
Responses by Visit Through Week 24, Based on the Composite
Estimand; Full Analysis Set 1 Among the Subjects with ≥3%
Body Surface Area (BSA) of Psoriatic Involvement and an IGA
Score ≥ 2 (mild) at Baseline (Study CNTO1959PSA3001)

| | | Guselkumab | |
|---|---|---|---|
| | Placebo | 100 mg q8w | 100 mg q4w |
| Analysis set: Full Analysis Set 1 | 78 | 82 | 89 |
| Among the Subjects Who had | | | |
| ≥3% Body Surface Area (BSA) | | | |
| of Psoriatic Involvement and an | | | |
| IGA Score ≥ 2 (mild) at Baseline | | | |
| Week 16 | | | |
| Subjects evaluable for PASI 75 | 76 | 81 | 87 |
| and ACR 20 responses^{a} | | | |
| Subjects with PASI 75 and ACR | 5 (6.6%) | 29 (35.8%) | 43 (49.4%) |
| 20 responses^{b, h} | | | |
| All subjects (including those | 78 | 82 | 89 |
| with imputed data) | | | |
| Subjects with PASI 75 and ACR | 5 (6.4%) | 29 (35.4%) | 43 (48.3%) |
| 20 responses^{b, c, h} | | | |
| % Difference (95% CI)^{d} | | 29.1 (17.5, | 41.8 (30.2, |
| | | 40.7) | 53.4) |
| p-value^{e} | | <0.001 | <0.001 |
| Week 24 | | | |
| Subjects evaluable for PASI 75 | 78 | 81 | 89 |
| and ACR 20 responses^{a} | | | |
| Subjects with PASI 75 and ACR | 5 (6.4%) | 33 (40.7%) | 47 (52.8%) |
| 20 responses^{b, h} | | | |
| All subjects (including those | 78 | 82 | 89 |
| with imputed data) | | | |
| Subjects with PASI 75 and ACR | 5 (6.4%) | 33 (40.2%) | 47 (52.8%) |
| 20 responses^{b, c, h} | | | |
| % Difference (95% CI)^{d} | | 33.7 (21.9, | 46.7 (35.1, |
| | | 45.5) | 58.3) |
| p-value^{e} | | <0.001 | <0.001 |

^{a}Subjects either have an observed PASI 75 and ACR 20 responses status or met a Treatment Failure (TF) criterion.
^{b}Defined as observed responders who had not met any TF criteria prior to the specific visit at which the endpoint was assessed.
^{c}Subjects with missing data at a visit are assumed to be non-responders at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and prior exposure to anti-TNFα agents (yes/no) is used to calculate the p-values. The symbol “†” will be attached as a superscript to those p-values that are calculated using the Fisher's exact test.
^{h}The PASI score is a composite of the state of erythema, induration and scaling over the body along with the area of the involvement of psoriatic lesions. The PASI score ranges from 0 to 72, with a higher score indicating more severe disease. PASI 75 response is defined as ≥75% improvement from baseline in PASI score. ACR 20 response is defined as ≥20% improvement from baseline in both tender joint count (68 joints) and swollen joint count (66 joints), and ≥20% improvement from baseline in at least 3 of the 5 assessments: patient's assessment of pain, patient's global assessment of disease activity, physician's global assessment of disease activity, HAQ-DI, and CRP.

### PASI 75 and Modified PsARC Responses Through Week 24

Among the 249 (65.4%) subjects with ≥3% BSA psoriatic involvement and an IGA score of ≥2 at baseline, greater proportions of subjects in both guselkumab 100 mg q4w (55.1%) and 100 mg q8w (48.8%) groups achieved both a PASI 75 response and a modified PsARC response compared with the placebo group at Week 16 (9.0%; both nominal p<0.001). The proportion of subjects achieving both PASI 75 and PsARC responses increased at Week 24 for the guselkumab 100 mg q4w group (62.9%) and remained higher in the guselkumab 100 mg q8w group (50.0%) compared with the placebo group (5.1%; both nominal p<0.001). The effect size was numerically greater in the guselkumab 100 mg q4w group compared with the guselkumab 100 mg q8w group at both Week 16 and Week 24.

### Other Efficacy Endpoints Related to Enthesitis

### Leeds Enthesitis Index

The LEI (0-6) assesses the tenderness of the following entheses: left and right lateral epicondyle humerus, left and right medial femoral condyle, and left and right achilles tendon insertion. LEI was collected at Weeks 0, 4, 8, 16 and 24. At baseline, 73 subjects in the guselkumab 100 mg q4w group, 72 subjects in the guselkumab 100 mg q8w group, and 77 subjects in the placebo group had LEI>0 (Table 41).

Among the 222 (58.3%) subjects with enthesitis at baseline:
The number of subjects achieving enthesitis resolution was numerically greater in the guselkumab 100 mg q4w group compared with the placebo group from Week 4 through Week 24, but separation from placebo was only observed at Week 24.
The number of subjects achieving enthesitis resolution was numerically greater in the guselkumab 100 mg q8w group compared with the placebo group at Week 8 and at Week 24.

TABLE 41
Number of Subjects Achieving Resolution of Enthesitis (LEI) by Visit Through
Week 24, Based on the Composite Estimand; Full Analysis Set 1 Among
the Subjects with Enthesitis (LEI) at Baseline (Study CNTO1959PSA3001)

| | | Guselkumab | |
|---|---|---|---|
| | Placebo | 100 mg q8w | 100 mg q4w |
| Analysis set: Full Analysis Set 1 Among | 77 | 72 | 73 |
| the Subjects with Enthesitis (LEI) at | | | |
| Baseline | | | |
| Week 4 | | | |
| Subjects evaluable for enthesitis (LEI) | 76 | 71 | 73 |
| resolution^{a} | | | |
| Subjects with enthesitis (LEI) | 17 (22.4%) | 13 (18.3%) | 20 (27.4%) |
| resolution^{b, h} | | | |
| All subjects (including those with | 77 | 72 | 73 |
| imputed data) | | | |
| Subjects with enthesitis (LEI) | 17 (22.1%) | 13 (18.1%) | 20 (27.4%) |
| resolution^{b, c, h} | | | |
| % Difference (95% CI)^{d} | | −4.2 (−16.9, 8.4) | 4.7 (−8.9, 18.2) |
| p-value^{e} | | 0.525 | 0.511 |
| Week 8 | | | |
| Subjects evaluable for enthesitis (LEI) | 76 | 72 | 73 |
| resolution^{a} | | | |
| Subjects with enthesitis (LEI) | 18 (23.7%) | 22 (30.6%) | 22 (30.1%) |
| resolution^{b, h} | | | |
| All subjects (including those with | 77 | 72 | 73 |
| imputed data) | | | |
| Subjects with enthesitis (LEI) | 18 (23.4%) | 22 (30.6%) | 22 (30.1%) |
| resolution^{b, c, h} | | | |
| % Difference (95% CI)^{d} | | 6.9 (−7.1, 20.9) | 5.3 (−8.4, 19.1) |
| p-value^{e} | | 0.346 | 0.457 |
| Week 16 | | | |
| Subjects evaluable for enthesitis (LEI) | 75 | 72 | 72 |
| resolution^{a} | | | |
| Subjects with enthesitis (LEI) | 29 (38.7%) | 25 (34.7%) | 33 (45.8%) |
| resolution^{b, h} | | | |
| All subjects (including those with | 77 | 72 | 73 |
| imputed data) | | | |
| Subjects with enthesitis (LEI) | 29 (37.7%) | 25 (34.7%) | 33 (45.2%) |
| resolution^{b, c, h} | | | |
| % Difference (95% CI)^{d} | | −2.8 (−17.8, 12.1) | 7.0 (−8.4, 22.4) |
| p-value^{e} | | 0.721 | 0.389 |
| Week 24 | | | |
| Subjects evaluable for enthesitis (LEI) | 77 | 72 | 73 |
| resolution^{a} | | | |
| Subjects with enthesitis (LEI) | 21 (27.3%) | 29 (40.3%) | 35 (47.9%) |
| resolution^{b, h} | | | |
| All subjects (including those with | 77 | 72 | 73 |
| imputed data) | | | |
| Subjects with enthesitis (LEI) | 21 (27.3%) | 29 (40.3%) | 35 (47.9%) |
| resolution^{b, c, h} | | | |
| % Difference (95% CI)^{d} | | 13.0 (−1.6, 27.5) | 19.8 (4.9, 34.6) |
| p-value^{e} | | 0.094 | 0.013 |

^{a}Subjects either have an observed Enthesitis resolution status or met a Treatment Failure (TF) criterion.
^{b}Defined as subjects who achieved resolution based on observed data and who had not met any TF criteria prior to the specific visit at which the endpoint was assessed.
^{c}Subjects with missing data at a visit are assumed to not have achieved resolution at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and prior exposure to anti-TNFα agents (yes/no) is used to calculate the p-values. The symbol “†” will be attached as a superscript to those p-values that are calculated using the Fisher's exact test.
^{b}Enthesitis score is a total score of 6 evaluated sites (left and right: lateral epicondyle humerus, medial femoral condyle, achilles tendon insertion) with a range from 0 to 6. A negative change from baseline indicates improvement. Enthesitis resolution is established when a subject with at least one tender entheses at baseline has no tender entheses among the 6 sites included in the LEI.

Change from Baseline in Enthesitis LEI Score Over Time

Among the 222 (58.3%) subjects with enthesitis (LEI>0) at baseline, except guselkumab 100 mg q8w at Week 16, a numerically greater reduction from baseline in LEI score was observed in both guselkumab treatment groups from Week 4 through Week 24, with the greatest effect observed at Week 24. Separations from placebo was observed at Week 4 and Week 24 for the guselkumab 100 mg q4w group, but not for the guselkumab 100 mg q8w group.

### SPARCC Enthesitis Index

The SPARCC enthesitis index was collected at Weeks 0, 4, 8, 16 and 24. At baseline, 84 subjects in the guselkumab 100 mg q4w group, 86 subjects in the guselkumab 100 mg q8w group, and 84 subjects in the placebo group had SPARCC enthesitis index score>0. Resolution of enthesitis and change from baseline based on SPARCC enthesitis index were evaluated in this subpopulation.

Resolution of Enthesitis Based on SPARCC Enthesitis Index Through Week 24. Among the 254 (66.7%) subjects with SPARCC enthesitis index score>0 at baseline, the number of subjects achieving enthesitis resolution was numerically greater in both guselkumab treatment groups compared with the placebo group from Week 8 through Week 24. At Week 24, the proportions of subjects achieving enthesitis resolution were 42.9% in the guselkumab 100 mg q4w group and 37.2% in the guselkumab 100 mg q8w group compared with 25.0% in the placebo group (nominal p=0.019 and p=0.106, respectively).

Change from Baseline in Enthesitis Based on the SPARCC Enthesitis Index Through Week 24. Among the 254 (66.7%) subjects with SPARCC enthesitis index score>0 at baseline, a numerically greater reduction from baseline in SPARCC enthesitis index was observed in both guselkumab treatment groups from Week 4 through Week 24, with the greatest reduction observed at Week 24. Separation from placebo was observed at Week 8 and Week 24 for the guselkumab 100 mg q4w group and at Week 24 for the guselkumab 100 mg q8w group). At Week 24, the estimated LSmean of change from baseline in SPARCC enthesitis index in the guselkumab 100 mg q4w group was −2.94 and −2.61 in the guselkumab 100 mg q8w group compared with −1.66 in the placebo group (nominal p=0.008 and p=0.048, respectively).

### Other Efficacy Endpoints Related to Dactylitis

Dactylitis was assessed at Weeks 0, 4, 8, 16 and 24. At baseline, 38 subjects in the guselkumab 100 mg q4w group, 49 subjects in the guselkumab 100 mg q8w group, and 55 subjects in the placebo group had dactylitis.

Tenderness was also assessed if dactylitis was present. At baseline, 36 subjects in the guselkumab 100 mg q4w group, 49 subjects in the guselkumab 100 mg q8w group, and 49 subjects in the placebo group had tender dactylitis.

### Dactylitis Resolution Through Week 24

Among the 142 (37.3%) subjects with dactylitis at baseline, the proportions of subjects who achieved dactylitis resolution were numerically greater in both guselkumab treatment groups compared to placebo at Week 16 and Week 24 and the effect size was comparable between the 2 guselkumab dose groups.

Results based on the treatment policy estimand were generally consistent with those based on the composite estimand, except the high placebo response observed at Week 24.

Change from Baseline in the Dactylitis Score Through Week 24

Among the 142 (37.3%) subjects with dactylitis at baseline, a numerically greater reduction from baseline in dactylitis score was observed in both guselkumab treatment groups compared with the placebo group from Week 8 through Week 24, and the effect size was comparable between the 2 guselkumab dose groups.

Results based on the treatment policy estimand were consistent with those based on the composite estimand.

### Tender Dactylitis

Among the 134 (35.2%) subjects with tender dactylitis at baseline, the proportions of subjects who did not have tender dactylitis were numerically greater in both the guselkumab 100 mg q4w and 100 mg q8w treatment groups compared to placebo at Week 16 (65.7% and 70.8% compared with 52.2%, respectively) and Week 24 (74.3% and 75.5% compared with 69.8%, respectively;).

Change from Baseline in Tender Dactylitis Through Week 24

Among the 134 (35.2%) subjects with tender dactylitis at baseline, a numerically greater reduction from baseline in tender dactylitis score was observed from Week 16 in the guselkumab 100 mg q4w group and from Week 8 in the guselkumab 100 mg q8w group through Week 24 compared with the placebo group.

At Week 24, the estimated LSmean of change from baseline in tender dactylitis score in the guselkumab 100 mg q4w group was −3.2 and −3.1 in the guselkumab 100 mg q8w group compared with −2.1 in the placebo group (nominal p=0.078 and p=0.080, respectively).

### Other Efficacy Endpoints Related to BASDAI

The BASDAI score was collected in subjects with spondylitis with peripheral arthritis as their primary arthritic presentation of PsA at Week 0, 8, 16, and 24. At baseline, there were 20 subjects in the guselkumab 100 mg q4w, 24 subjects in the guselkumab 100 mg q8w, and 23 subjects in the placebo group with spondylitis with peripheral arthritis who had a BASDAI score at baseline (Table 42). All baseline BASDAI scores among these subjects were >0.

Among these subjects, 16 subjects in the guselkumab 100 mg q4w, 22 subjects in the guselkumab 100 mg q8w, and 21 subjects in the placebo group also had imaging confirmation of spondylitis in the past.

Change from Baseline in BASDAI Through Week 24

Among the 67 (17.6%) subjects with spondylitis and peripheral arthritis and a BASDAI score>0 at baseline, the LSmean change from baseline in BASDAI at Week 24 was −2.074 the guselkumab 100 mg q4w group and −2.665 in the guselkumab 100 mg q8w group compared with −0.919 in the placebo group (nominal p=0.06′7 and p=0.004, in the 100 mg q4w and 100 mg q8w, respectively; Table 42).

TABLE 42
Summary of the Change from Baseline in the Bath Ankylosing Spondylitis
Disease Activity Index (BASDAI) by Visit Through Week 24, Based on the
Composite Estimand Using an MMRNI Model; Full Analysis Set 1 Among
the Subjects with Spondylitis and Peripheral Arthritis at Baseline (Study CNTO1959PSA3001)

| | | Guselkumab | |
|---|---|---|---|
| | Placebo | 100 mg q8w | 100 mg q4w |
| Analysis set: Full Analysis Set 1 Among | 24 | 26 | 25 |
| the Subjects with Spondylitis and | | | |
| Peripheral Arthritis at Baseline | | | |
| Subjects with a baseline BASDAI = 0^{a, h} | 0 | 0 | 0 |
| Subjects with a baseline BASDAI > 0^{a, h} | 23 | 24 | 20 |
| Week 8 | | | |
| Subjects evaluable^{b} | | | |
| N | 23 | 24 | 20 |
| Mean (SD) | −0.557 (1.2190) | −1.542 (1.4921) | −1.740 (2.3517) |
| Median | −0.540 | −1.855 | −1.140 |
| Range | (−3.59; 1.75) | (−4.48; 1.54) | (−7.55; 2.47) |
| IQ range | (−1.070; 0.180) | (−2.245; −0.330) | (−3.120; −0.300) |
| Model Based Estimates of the Mean | | | |
| Change^{a, c} | | | |
| LSMean (95% CI)^{d} | −0.595 (−1.351, 0.162) | −1.577 (−2.296, −0.859) | −1.976 (−2.779, −1.174) |
| LSMean difference (95% CI) | | −0.982 (−1.988, 0.023) | −1.382 (−2.435, −0.329) |
| p-value^{d} | | 0.055 | 0.011 |
| Week 16 | | | |
| Subjects evaluable^{b} | | | |
| N | 23 | 24 | 20 |
| Mean (SD) | −1.566 (1.9359) | −2.384 (2.3112) | −2.232 (2.2327) |
| Median | −1.310 | −2.290 | −2.260 |
| Range | (−5.11; 1.97) | (−8.94; 1.65) | (−6.80; 0.83) |
| IQ range | (−3.150; −0.140) | (−3.670; −1.050) | (−3.960; 0.120) |
| Model Based Estimates of the Mean | | | |
| Change^{a, c} | | | |
| LSMean (95% CI)^{d} | −1.604 (−2.483, −0.725) | −2.419 (−3.261, −1.577) | −2.469 (−3.405, −1.533) |
| LSMean difference (95% CI) | | −0.815 (−2.000, 0.370) | −0.865 (−2.107, 0.377) |
| p-value^{d} | | 0.174 | 0.169 |
| Week 24 | | | |
| Subjects evaluable^{b} | | | |
| N | 23 | 24 | 20 |
| Mean (SD) | −0.881 (1.5480) | −2.630 (2.4939) | −1.837 (2.0792) |
| Median | −0.450 | −2.225 | −1.900 |
| Range | (−5.09; 1.60) | (−9.23; 1.94) | (−7.65; 1.67) |
| IQ range | (−1.080; 0.000) | (−4.285; −1.170) | (−2.990; −0.360) |
| Model Based Estimates of the Mean | | | |
| Change^{a, c} | | | |
| LSMean (95% CI)^{d} | −0.919 (−1.795, −0.043) | −2.665 (−3.503, −1.826) | −2.074 (−3.006, −1.142) |
| LSMean difference (95% CI) | | −1.746 (−2.926, −0.565) | −1.155 (−2.391, 0.082) |
| p-value^{d} | | 0.004 | 0.067 |

^{a}Defined as the change from baseline using observed data or 0 (no improvement) if a subject met Treatment Failure (TF) criteria.
^{b}Subjects either have an observed change from baseline at this visit or met TF criteria prior to this visit.
^{c}The missing data is assumed to be MAR.
^{d}The LS means and p-values are based on the MMRM analysis.
^{h}The BASDAI is based on 6 questions relating to 5 major symptoms of ankylosing spondylitis through a patient's self assessment. A higher score indicates greater disease severity.

Among Subjects with Imaging Confirmation of Spondylitis in the Past

Subjects Achieving≥20%, ≥50%, ≥70%, and ≥90% Improvement from Baseline in BASDAI Through Week 24

Among the 67 (17.6%) subjects with spondylitis with peripheral arthritis and a BASDAI score>0 at baseline, the proportion of subjects achieving≥20% or ≥50% BASDAI improvement was numerically greater in both guselkumab treatment groups compared with the placebo group from Week 8 through Week 24. At Week 24, the proportions of subjects achieving BASDAI≥20% or ≥50% in the guselkumab 100 mg q4w and guselkumab 100 mg q8w groups compared with the placebo group were as follows:
≥20% improvement: 65.0% and 70.8% compared with 26.1% (nominal p=0.044 and p=0.007, respectively)
>50% improvement: 35.0% and 41.7% compared with 13.0% (nominal p=0.148 and p=0.082, respectively)

Few subjects achieved≥70% improvement in BASDAI through Week 24, of which, the majority were in the guselkumab 100 mg q8w group (7 [29.2%] subjects) compared with 1 [5.0] subject in the guselkumab 100 mg q4w group and 2 (8.7%) subjects in the placebo group. All 4 subjects who achieved≥90% improvement in BASDAI through Week 24 were in the guselkumab 100 mg q8w group (16.7%).

Change from Baseline in BASDAI Components Through Week 24

Through Week 24, numerically greater improvements over time above placebo were only consistently observed for fatigue and spinal pain in both guselkumab treatment groups.

At Week 24, the median of change from baseline in BASDAI components in the guselkumab 100 mg q4w and 100 mg q8w groups compared with the placebo group were as follows:
enthesitis: −1.700 and −2.250 compared with −1.350, respectively
fatigue: −1.250 and −3.250 compared with −0.650, respectively
joint pain: −1.250 and −2.000 compared with −1.300, respectively
qualitative morning stiffness: −1.450 and −1.700 compared with −1.200, respectively
quantitative morning stiffness: −0.700 and −1.800 compared with −0.100, respectively
spinal pain: −1.750 and −2.550 compared with −0.750, respectively

### Other Efficacy Endpoints Related to Health-Related Quality of Life and Other Patient Reported Outcomes

### SF-36 Scores

SF-36 version 2 was used to assess health-related quality of life. SF-36 was collected at Weeks 0, 8, 16, and 24. The results for SF-36 PCS, MCS, and 8 norm-based subscale scores are described below.

### SF-36 PCS Scores

Change from Baseline in SF-36 PCS Scores Through Week 24

A numerically greater improvement in SF-36 PCS score from baseline was observed in both guselkumab treatment groups compared with the placebo group from Week 8 through Week 24, with separation from placebo at nominal p<0.05 observed from Week 8 in the guselkumab 100 mg q4w group and from Week 16 in the guselkumab 100 mg q8w group. The greatest effect was observed at Week 24 for both the guselkumab 100 mg q4w and 100 mg q8w groups and the effect size was numerically greater in the guselkumab 100 mg q4w group than that in the guselkumab 100 mg q8w group. A tipping point analysis was performed for the change in baseline in SF-36 PCS score at Week 16 based on the treatment policy estimand and MI.

5-Point Improvement from Baseline in SF-36 PCS Through Week 24

A numerically greater proportion of subjects achieved a ≥5 point improvement from baseline in SF-36 PCS score from Week 8 (nominal p=0.013) through Week 24 in the guselkumab 100 mg q4w group and from Week 16 (nominal p=0.002) in the guselkumab 100 mg q8w group compared with the placebo group. The greatest effect was observed at Week 24 for both the guselkumab 100 mg q4w (53.9%) and q8w (51.2%) groups compared with placebo (28.6%, both nominal p<0.001) and the effect size was comparable between the 2 guselkumab doses at Week 16 and Week 24.

### SF-36 MCS Scores

Change from Baseline in SF-36 MCS Scores Through Week 24

In comparison to the placebo group, a numerically greater improvement in SF-36 MCS score from baseline was observed in both guselkumab treatment groups from Week 8 through Week 24. The greatest effect was observed at Week 24 for both the guselkumab 100 mg q4w and 100 mg q8w groups and the effect size was comparable between the guselkumab doses.

5-Point Improvement from Baseline in SF-36 MCS Through Week 24

A numerically greater proportion of subjects achieved a ≥5 point improvement from baseline in SF-36 MCS score from Week 8 through Week 24 in the guselkumab 100 mg q4w group and at Weeks 8 and 24 in the guselkumab 100 mg q8w group compared with the placebo group. The greatest effect was observed at Week 24 for both the guselkumab 100 mg q4w (43.0%) and 100 mg q8w (37.8%) groups compared with placebo (25.4%; nominal p=0.003 and p=0.036, respectively) and the effect size was numerically greater in the guselkumab 100 mg q4w group than that in the guselkumab 100 mg q8w group at Week 16 and Week 24.

Change from Baseline in Norm-Based Scores of SF-36 Scales

With few exceptions, the improvements in norm-based SF-36 subscale scores were in general numerically greater in both guselkumab treatment groups compared with the placebo group, from Week 8 through Week 24, with the greatest effect for each subscale at Week 24.

In the guselkumab 100 mg q4w group, separation from placebo was observed from Week 8 for physical function, role-physical, bodily pain, and vitality; from Week 16 for general health and social function; and at Week 24 for mental health; numerically greater improvement for role emotional was observed at Week 16 and Week 24 compared with placebo (nominal p=0.147 and p=0.187, respectively).

In the guselkumab 100 mg q8w group, separation from placebo was observed from Week 16 for physical function, role-physical, bodily pain, and general health; and at Week 24 for vitality and social function; numerically greater improvement was observed at Week 16 for role-emotional and mental health (nominal p=0.487 and p=0.212, respectively) and at Week 24 for mental health (nominal p=0.074) compared with placebo.

At Week 24, the estimated LSmean of change from baseline in norm-based SF-36 subscales in the guselkumab 100 mg q4w and 100 mg q8w groups compared with the placebo group were as follows:
physical functioning: 6.952 and 5.776 compared with 1.636, respectively, both nominal p<0.001
role-physical: 5.442 and 4.878 compared with 2.319, nominal p<0.001 and p=0.004, respectively
bodily pain: 7.490 and 6.840 compared with 2.854, respectively, both nominal p<0.001
general health: 5.174 and 4.349 compared with 1.690, nominal p<0.001 and p=0.001, respectively
vitality: 6.426 and 5.596 compared with 2.311, nominal p<0.001 and p=0.001, respectively respectively
social functioning: 5.227 and 5.426 compared with 2.582, nominal p=0.005 and p=0.002, respectively
role-emotional: 3.531 and 2.415 compared with 2.201, nominal p=0.18′7 and p=0.832, respectively
mental health: 4.356 and 3.818 compared with 2.062, nominal p=0.020 and p=0.074, respectively

### FACIT-Fatigue Score

Fatigue was assessed using the FACIT-Fatigue scale at Weeks 0, 8, 16, and 24.
Change from Baseline in FACIT-Fatigue Score Through Week 24

A numerically greater improvement from baseline in FACIT-Fatigue scores was observed in both guselkumab groups compared with placebo from Week 8 through Week 24 (Table 43). For both guselkumab treatment groups, separation from placebo was observed from Week 16 and the greatest effect was seen at Week 24 (both nominal p<0.001), with the effect size comparable between the 2 guselkumab doses.

TABLE 43
Summary of the Change from Baseline in FACIT-Fatigue
Score by Visit Through Week 24, Based on the Composite Estimand
Using an MMRM Model; Full Analysis Set 1 (Study CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Change from baseline in FACIT-Fatigue | | | |
| score^{a, h} | | | |
| Week 8 | | | |
| Subjects evaluable^{b} | | | |
| N | 126 | 126 | 128 |
| Mean (SD) | 2.302 (7.5834) | 3.730 (7.9442) | 3.180 (6.5706) |
| Median | 2.000 | 2.000 | 3.000 |
| Range | (−27.00; 37.00) | (−12.00; 40.00) | (−14.00; 20.00) |
| IQ range | (−1.000; 5.000) | (−1.000; 8.000) | (−1.000; 7.000) |
| Model Based Estimates of the Mean | | | |
| Change^{a, c} | | | |
| LSMean (95% CI)^{d} | 2.356 (1.081, 3.632) | 3.643 (2.369, 4.917) | 3.576 (2.306, 4.845) |
| LSMean difference (95% CI) | | 1.287 (−0.447, 3.020) | 1.219 (−0.510, 2.948) |
| p-value^{d} | | 0.145 | 0.166 |
| Week 16 | | | |
| Subjects evaluable^{b} | | | |
| N | 125 | 127 | 128 |
| Mean (SD) | 2.080 (8.1375) | 5.000 (8.4815) | 4.148 (8.0247) |
| Median | 1.000 | 4.000 | 4.000 |
| Range | (−20.00; 29.00) | (−14.00; 33.00) | (−24.00; 23.00) |
| IQ range | (−2.000; 6.000) | (0.000; 9.000) | (0.000; 9.000) |
| Model Based Estimates of the Mean | | | |
| Change^{a, c} | | | |
| LSMean (95% CI)^{d} | 2.164 (0.782, 3.547) | 4.853 (3.478, 6.228) | 4.544 (3.171, 5.918) |
| LSMean difference (95% CI) | | 2.688 (0.802, 4.574) | 2.380 (0.497, 4.263) |
| p-value^{d} | | 0.005 | 0.013 |
| Week 24 | | | |
| Subjects evaluable^{b} | | | |
| N | 126 | 127 | 128 |
| Mean (SD) | 2.151 (7.8374) | 5.756 (10.1776) | 5.445 (7.7213) |
| Median | 0.000 | 5.000 | 5.000 |
| Range | (−22.00; 28.00) | (−20.00; 40.00) | (−20.00; 24.00) |
| IQ range | (−1.000; 5.000) | (−1.000; 12.000) | (0.000; 11.000) |
| Model Based Estimates of the Mean | | | |
| Change^{a, c} | | | |
| LSMean (95% CI)^{d} | 2.206 (0.773, 3.638) | 5.609 (4.181, 7.036) | 5.841 (4.416, 7.267) |
| LSMean difference (95% CI) | | 3.403 (1.442, 5.364) | 3.636 (1.677, 5.594) |
| p-value^{d} | | <0.001 | <0.001 |

^{a}Defined as the change from baseline using observed data or 0 (no improvement) if a subject met Treatment Failure (TF) criteria.
^{b}Subjects either have an observed change from baseline at this visit or met TF criteria prior to this visit.
^{c}The missing data is assumed to be MAR.
^{d}The LS means and p-values are based on the MMRM analysis.
^{h}The FACIT-Fatigue score is calculated based on the FACIT-Fatigue questionnaire that comprises of 13 questions, with each question graded on a 5-point scale (0-4). The FACIT-Fatigue scores can range from 0 to 52 with higher scores indicating less fatigue.

Among ACR 20 responders, the median improvement from baseline was 7.0, 8.0, and 5.5 in the guselkumab 100 mg q4w, q8w and placebo groups respectively. Among ACR 20 non-responders, and the median improvement from baseline was 2.0, 1.0, and 0 in the guselkumab 100 mg q4w, q8w and placebo groups respectively.

FACIT-Fatigue Improvement ≥4 from Baseline Through Week 24

The proportions of subjects who achieved≥4-point improvement from baseline in FACIT Fatigue scores were numerically greater in both the guselkumab 100 mg q4w and 100 mg q8w groups compared with the placebo group from Week 8 through Week 24, with separation from placebo observed from Week 16 and the greatest effect seen at Week 24 (63.3% and 53.5% compared with 34.9%, nominal p<0.001 and p=0.003 respectively). The effect size was comparable between the 2 guselkumab doses at Week 8 and Week 16 but at Week 24, the proportion of subjects who achieved≥4-point improvement from baseline in FACIT Fatigue scores was numerically higher in the guselkumab 100 mg q4w group than that in the guselkumab 100 mg q8w group.

Additional analysis by cumulative distribution function curve at Week 24 showed that separations of both guselkumab 100 mg q4w and 100 mg q8w groups from placebo were observed from a range of cut-offs from ≥2-point through 10-point improvement. The distribution of change in FACIT-Fatigue from probability density plot at Week 24 demonstrated separations from placebo for both guselkumab 100 mg q4w and 100 mg q8w groups. Item level analysis at Week 24 showed that the improvements were consistent and similar across 13 individual items of the FACIT-Fatigue instrument.

In all treatment groups, the proportions of subjects who achieved a ≥4-point improvement in FACIT-Fatigue score at Week 24 were much higher in ACR 20 responders than non-responders.

Among ACR 20 responders, the proportion of subjects achieving a ≥4-point improvement in FACIT-Fatigue score at Week 24 was 73.7%, 68.2%, and 67.9% in the guselkumab 100 mg q4w group, the guselkumab 100 mg q8w group, and the placebo group respectively.

Among ACR 20 non-responders, the proportion of subjects achieving a ≥4-point improvement in FACIT-Fatigue score at Week 24 was 48.1%, 37.7%, and 25.5% in the guselkumab 100 mg q4w group, the guselkumab 100 mg q8w group, and the placebo group respectively.

### Mediation and Propensity Score Analysis on FACIT-Fatigue

Mediation analysis was conducted to investigate the mediation role of ACR20 response for the effect of guselkumab on the change from baseline in fatigue score at Week 24. The results demonstrated that 28.9% and 83.4% of the treatment effect on FACIT-Fatigue was mediated through ACR 20 response (natural indirect effect) in the guselkumab 100 mg q4w and q8w groups (nominal p=0.032 and p<0.001 respectively). The proportion of natural direct effect was 71.1% (2.70/3.80, norminal p=0.005) and 16.8% (0.52/3.10, normimal p=0.619) in the guselkumab 100 mg q4w and q8w groups respectively.

In the subgroup analysis by ACR 20 responders and non-responders using propensity score weighted analysis, demographic and baseline clinical characteristics including age, sex, BMI, baseline fatigue score, CRP (mg/dL), PsA duration (years), physician global assessment, patient global assessment, HAQ-DI score, pain assessment, and number of swollen and tender joints were adjusted as covariates in the statistical model for propensity score. The weighted standardized differences between the treatment groups of these baseline parameters indicated that imbalances with these baseline parameters were largely adjusted (majority≤0.02 or approaching 0.02,). The results demonstrated an independent treatment effect of guselkumab 100 mg q4w on FACIT-Fatigue among ACR 20 non-responders (nominal p=0.002,) but not among ACR20 responders. An independent treatment effect of guselkumab 100 mg q8w on FACIT-Fatigue was not observed regardless ACR 20 response at Week 24.

### PROMIS-29 Score

Change from Baseline in PROMIS-29 Scores Through Week 24

Numerically greater improvement from baseline in each PROMIS-29 domain was observed in both guselkumab treatment groups compared with the placebo group over time through Week 24. Separation from placebo was observed in both guselkumab treatment groups from Week 8 for satisfaction with participation in social roles and activities and pain intensity, from Week 16 for depression, fatigue, and physical function. For anxiety, separation from placebo was observed at Week 24 in guselkumab 100 mg q8w group, but not in guselkumab 100 mg q4w group. For pain interference, separation from placebo was observed from Week 16 in the guselkumab 100 mg q4w group and at Week 24 in the guselkumab 100 mg q8w group. For sleep disturbance, separation from placebo was observed at Week 16 but not at Week 24 in guselkumab 100 mg q4w group and at Week 16 and Week 24 in guselkumab 100 mg q8w group.

### PROMIS-29 Domain Scores Improvement ≥3 and ≥5 Through Week 24

Over time through Week 24, numerically greater proportion of subjects achieved a ≥3 point improvement from baseline on each of 8 domains assessed by PROMIS-29 (anxiety, depression, fatigue, pain interference, physical function, sleep disturbance, satisfaction with participation in social roles and activities, and pain intensity) in both guselkumab treatment groups compared with the placebo group. At Week 24, a greater proportion of subjects in guselkumab 100 mg q4w and 100 mg q8w groups achieved improvements of ≥3 and ≥5 points in domain scores related to symptoms and impact of PsA, including pain interference, pain intensity, fatigue, physical function, and ability to participate in social roles and activities, compared with placebo. Additionally, greater proportions of subjects in the guselkumab 100 mg q4w and 100 mg q8w groups achieved≥3- or ≥5-point improvements in PROMIS-29 domains of anxiety, depression or sleep disturbance at Week 24 compared with the placebo group.

### Improvements in Composite Disease Activity Scores

The effect of guselkumab on multiple PsA composite disease activity scores including

PASDAS, GRACE index, and MDA/VLDA were evaluated.

### PASDAS

The PASDAS, evaluated at Weeks 0, 8, 16, and 24, is composed of assessments for arthritis/psoriasis, enthesitis, dactylitis, and the physical component of quality of life. The cut-off values for disease activities are: very low (≤1.9), low (≤3.2), moderate (>3.2 and <5.4), and high (≥5.4).
Change from Baseline in PASDAS Through Week 24

A greater reduction from baseline in PASDAS score was observed in both guselkumab groups compared with the placebo group from Week 8 through Week 24 (all nominal p<0.001), with the greatest effect seen at Week 24 and the effect size numerically greater in the guselkumab 100 mg q4w group than that in the guselkumab 100 mg q8w group.

At Week 24, the estimated LSmean of change from baseline in PASDAS score was −2.407 in the guselkumab 100 mg q4w group and −2.124 in the guselkumab 100 mg q8w group compared with −0.959 in the placebo group (both nominal p<0.001).

### Low or Very Low Disease Activity Based on PASDAS Through Week 24

Low Disease Activity: The proportion of subjects achieving low disease activity based on the PASDAS was numerically higher in both guselkumab treatment groups from Week 8 through Week 24. Separation from placebo was observed from Week 8 in the guselkumab 100 mg q4w group and from Week 16 in the guselkumab 100 mg q8w group. At Week 24, the proportion of subjects achieving low disease activity based on PASDAS was 36.7% in the guselkumab 100 mg q4w group and 30.7% in the guselkumab 100 mg q8w group compared with 11.1% in the placebo group (both nominal p<0.001).

Very Low Disease Activity: Compared with the placebo group, more subjects in both guselkumab treatment groups achieved VLDA based on PASDAS over time through Week 24. At Week 24, the proportion of subjects achieving VLDA based on PASDAS was 10.2% in the guselkumab 100 mg q4w group (nominal p=0.006) and 5.5% in the guselkumab 100 mg q8w group (nominal p=0.172) compared with 1.6% in the placebo group.

### GRACE Index

The GRACE index, evaluated at Week 0, 16 and 24, is composed of assessments for arthritis, psoriasis, physical function, and PsA quality of life. The cut-off values for disease activities are: low (≤2.3), moderate (>2.3 and <4.7) and high (≥4.7).

Change from Baseline in GRACE Index Through Week 24

A greater reduction from baseline in GRACE index was observed in both guselkumab groups compared with the placebo group at both Week 16 and Week 24 (all nominal p<0.001), with the greatest effect seen at Week 24 and the effect size numerically greater in the guselkumab 100 mg q4w group than that in the guselkumab 100 mg q8w group. At Week 24, the estimated LSmean of change from baseline in GRACE index was −2.735 in the guselkumab 100 mg q4w group and −2.368 in the guselkumab 100 mg q8w group compared with −0.854 in the placebo group (both nominal p<0.001).

### Low Disease Activity Based on GRACE Index

The proportion of subjects achieving low disease activity based on the GRACE index was higher at Week 16 and Week 24 in the guselkumab 100 mg q4w (28.9% and 42.2%, respectively; both nominal p<0.001) and the guselkumab 100 mg q8w (22.0% and 30.7%, respectively; nominal p=0.016 and p<0.001, respectively) groups compared with the placebo group (10.3% and 11.9%, respectively;).

### MDA and VLDA

Minimal disease activity (MDA) was considered achieved if 5 of the following 7 criteria were met: tender joint count≤1; swollen joint count≤1; PASI≤1; patient pain VAS score of ≤15; patient global disease activity VAS (arthritis and psoriasis) score of ≤20; HAQ≤0.5; and LEI≤1.

Very Low Disease Activity (VLDA) was considered achieved if all 7 criteria were met.
Both MDA and VLDA were evaluated at Weeks 0, 16, and 24.

### MDA Criteria Through Week 24

The proportion of subjects achieving MDA was higher at both Week 16 and Week 24 in the guselkumab 100 mg q4w (18.0% and 30.5%; nominal p=0.010 and p<0.001, respectively) and guselkumab 100 mg q8w (15.7% and 22.8%, nominal p=0.034 and p=0.012, respectively) groups compared with the placebo group (7.1% and 11.1%, respectively; Table 44).

TABLE 3: 44
Number of Subjects Who Achieved the Minimal Disease
Activity (MDA) Criteria by Visit Through Week 24, Based on the
Composite Estimand; Full Analysis Set 1 (Study CNTO1959PSA3001)

| | | Guselkumab | |
|---|---|---|---|
| | Placebo | 100 mg q8w | 100 mg q4w |
| Analysis set: Full Analysis Set 1 | 126 | 127 | 128 |
| Baseline | | | |
| Subjects evaluable for MDA response^{a} | 126 | 127 | 128 |
| Subjects with MDA response^{b, h} | 1 (0.8%) | 1 (0.8%) | 0 |
| Week 16 | | | |
| Subjects evaluable for MDA response^{a} | 125 | 126 | 127 |
| Subjects with MDA response^{b, h} | 9 (7.2%) | 20 (15.9%) | 23 (18.1%) |
| All subjects (including those with imputed | 126 | 127 | 128 |
| data) | | | |
| Subjects with MDA response^{b, c, h} | 9 (7.1%) | 20 (15.7%) | 23 (18.0%) |
| % Difference (95% CI)^{d} | | 8.6 (0.9, 16.2) | 10.8 (2.8, 18.7) |
| p-value^{e} | | 0.034 | 0.010 |
| Week 24 | | | |
| Subjects evaluable for MDA response^{a} | 126 | 127 | 128 |
| Subjects with MDA response^{b, h} | 14 (11.1%) | 29 (22.8%) | 39 (30.5%) |
| All subjects (including those with imputed | 126 | 127 | 128 |
| data) | | | |
| Subjects with MDA response^{b, c, h} | 14 (11.1%) | 29 (22.8%) | 39 (30.5%) |
| % Difference (95% CI)^{d} | | 11.9 (2.9, 20.9) | 19.3 (9.7, 28.9) |
| p-value^{e} | | 0.012 | <0.001 |

^{a}Subjects either have an observed MDA response status or met a Treatment Failure (TF) criterion.
^{b}Defined as observed responders who had not met any TF criteria prior to the specific visit at which the endpoint was assessed.
^{c}Subjects with missing data at a visit are assumed to be non-responders at that visit.
^{d}The confidence intervals are based on the Wald statistic.
^{e}If the Mantel Fleiss criterion is not satisfied the Fisher's exact test is used. Otherwise, the CMH test stratified by baseline use of non-biologic DMARD (yes, no) and prior exposure to anti-TNFα agents (yes/no) is used to calculate the p-values. The symbol “†” will be attached as a superscript to those p-values that are calculated using the Fisher's exact test.
^{h}MDA is achieved if at least 5 of the 7 criteria are met (tender joint count ≤ 1, swollen joint count ≤ 1, psoriasis activity and severity index ≤ 1, patient's assessment of pain ≤ 15, patient's global assessment of disease activity ≤ 20, HAQ-DI score ≤ 0.5, Tender entheseal points ≤ 1).

### VLDA Criteria Through Week 24

The proportions of subjects who met VLDA criteria at Week 16 were low and comparable among all treatment groups. At Week 24, 12 (9.4%) subjects in the guselkumab 100 mg q4w group and 5 (3.9%) subjects in the guselkumab 100 mg q8w group achieved VLDA compared with 2 (1.6%) subjects in the placebo group (nominal p=0.007 and p=0.447, respectively).

### Efficacy and Pharmacokinetics

The relationships between selected efficacy endpoints and trough serum guselkumab concentrations were assessed based on the PK analysis set. Clinical efficacy data (composite estimand) with no missing data imputation and respective trough serum guselkumab concentrations were used in the following analyses:
ACR 20/50 responses or change from baseline in DAS28 (CRP) at Week 12 by trough serum guselkumab concentration at Week 12
ACR 20/50 responses or change from baseline in DAS28 (CRP) at Weeks 20/24 by steady state trough serum guselkumab concentration at Week 20
IGA response at Weeks 24 by steady-state trough serum guselkumab concentration at Week 20 (in subjects with ≥3% BSA psoriatic involvement and an IGA score of ≥2 at baseline) ACR 20/50 Responses and Trough Serum Guselkumab Concentrations

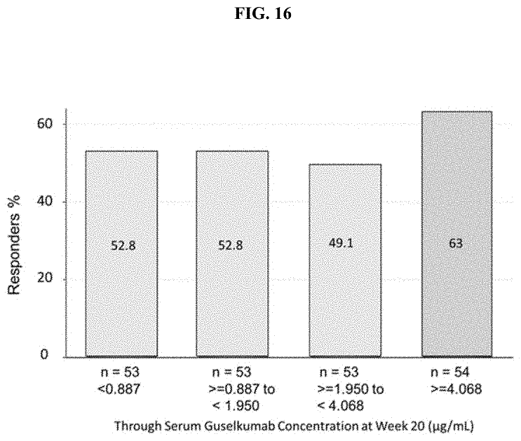

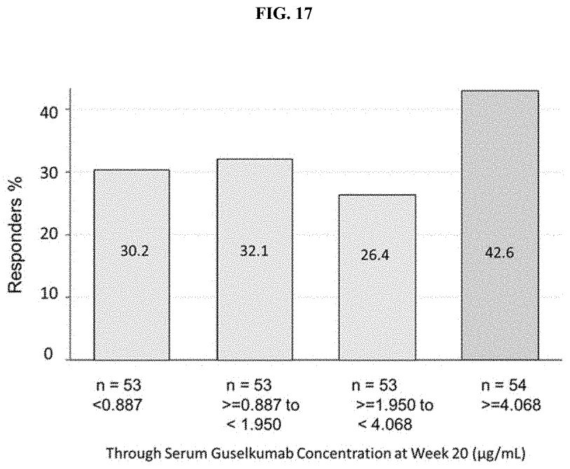

There appeared to be a weak exposure-response relationship for the ACR 20 response rate at Weeks 12 or 20 by trough guselkumab concentration quartiles at Weeks 12 or 20, respectively. No exposure-response relationships were observed for ACR 20 response rate at Week 24 by trough guselkumab concentration quartiles at Week 20 (FIG. 16). In addition, there appeared to be a weak exposure-response relationship for the ACR 50 response rate at Week 24 by trough guselkumab concentration quartiles at Week 20 (FIG. 17). However, no consistent trend of exposure-response relationship was observed for ACR 50 response rates at Weeks 12 or 20 by trough guselkumab concentration quartiles at Weeks 12 or 20.

Change from Baseline in DAS28 (CRP) by Trough Serum Guselkumab Concentrations

There was no apparent exposure-response relationship for mean change from baseline in DAS28 (CRP) at Week 12 by trough guselkumab concentration quartiles at Week 12. There were also no apparent exposure-response relationships for mean changes from baseline in DAS28 (CRP) at Weeks 20 or 24 by trough guselkumab concentration quartiles at Week 20.

### IGA Response and Trough Serum Guselkumab Concentrations

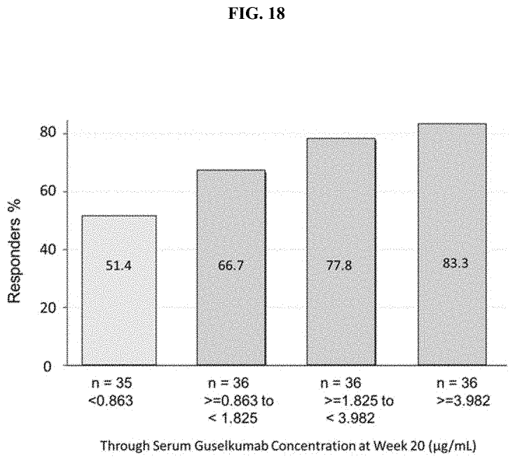

There was an apparent exposure-response relationship in IGA response rate at Week 24 by trough guselkumab concentration quartiles at Week 20 in subjects with ≥3% BSA psoriatic involvement and an IGA score of ≥2 at baseline (FIG. 18).

### Efficacy Summary

In this Phase 3 study, both guselkumab 100 mg q4w and 100 mg q8w dose regimens demonstrated statistically significant superiority compared with placebo for the following endpoints based on both the global (ex-US) and the US-specific multiplicity adjustment procedures: proportion of subjects achieving ACR 20 response at Week 24, proportion of subjects who achieved psoriasis IGA response at Week 24 among subjects with ≥3% BSA of psoriatic involvement and an IGA score≥2 (mild) at baseline, change from baseline in HAQ-DI score at Week 24; and change from baseline in the SF-36 PCS score at Week 24.

In addition, based on the global (ex-US) multiplicity adjustment procedure, both guselkumab 100 mg q4w and 100 mg q8w dose regimens also demonstrated statistically significant improvement compared with placebo for the following endpoints: change from baseline in DAS 28 (CRP) score at Week 24, proportion of subjects with ACR 20 response at Week 16, and proportion of subjects with ACR 50 response at Week 24.

Guselkumab 100 mg q4w also demonstrated statistically significant improvement compared to placebo for ACR 50 at Week 16 and ACR 70 at Week 24 based on global (ex-US) testing procedure. Improvements on these endpoints were numerically higher in the guselkumab 100 mg q8w group compared to placebo, but the differences were not statistically significant.

### Primary Endpoint

A significantly greater proportion of subjects in both the guselkumab 100 mg q4w and guselkumab 100 mg q8w groups (59.4% and 52.0%, respectively) achieved an ACR 20 response at Week 24 compared with subjects in the placebo group (22.2%) based on the global (ex-US) and US-specific multiplicity testing procedures (both adjusted p<0.001).

### Major Secondary Endpoints

Major Secondary Endpoints Controlled for Multiplicity in Both the Global (ex-US) and US specific Testing Procedures
Among the 249 (65.4%) subjects with ≥3% BSA of psoriatic involvement and an IGA score≥2 (mild) at baseline, a significantly greater proportion of subjects in both the guselkumab 100 mg q4w and the guselkumab 100 mg q8w groups (75.3% and 57.3%, respectively) achieved a psoriasis IGA response of 0 (cleared) or 1 (minimal) and ≥2-grade reduction from baseline in the IGA psoriasis score at Week 24 compared with the placebo group (15.4%; both global and US specific adjusted p<0.001).
A significantly greater reduction from baseline in HAQ-DI score at Week 24 was observed in both the guselkumab 100 mg q4w (LSmean change from baseline: −0.3968) and the guselkumab 100 mg q8w groups (LSmean change from baseline: −0.3225) compared with the placebo group (LSmean change from baseline: −0.0743; both global and US-specific adjusted p<0.001).
A significantly greater improvement from baseline in SF-36 PCS score was observed in both the guselkumab 100 mg q4w (LSmean: 6.87) and the guselkumab 100 mg q8w groups (LSmean: 6.10) at Week 24 compared with the placebo group (LSmean: 1.96; both global and US specific adjusted p<0.001).

### Major Secondary Endpoints Controlled for Multiplicity in the Global (Ex-US) Testing Procedure

A significantly greater reduction from baseline in DAS28 (CRP) score at Week 24 was observed in both the guselkumab 100 mg q4w (LSmean change from baseline: −1.61) and guselkumab 100 mg q8w groups (LSmean change from baseline: −1.43) compared with the placebo group (LSmean change from baseline: −0.70; both global adjusted p<0.001).
A significantly greater proportion of subjects in both the guselkumab 100 mg q4w and the guselkumab 100 mg q8w groups (60.2% and 52.0%, respectively) achieved an ACR 20 response at Week 16 compared with the placebo group (25.4%; both global adjusted p<0.001).
A significantly greater proportion of subjects in both the guselkumab 100 mg q4w and the guselkumab 100 mg q8w groups (35.9% and 29.9%, respectively) achieved an ACR 50 response at Week 24 compared with the placebo group (8.7%; both global adjusted p<0.001).
A significantly greater proportion of subjects in the guselkumab 100 mg q4w group (26.6%) achieved ACR50 response at Week 16 than in the placebo group (12.7%, global adjusted p=0.006); The proportion of subjects who achieved ACR50 response at Week 16 was numerically greater in the guselkumab 100 mg q8w group (22.8%) than that in the placebo group (12.7%), but did not reach statistical significance after multiplicity adjustment (global adjusted p=0.086).
A significantly greater proportion of subjects in the guselkumab 100 mg q4w group (20.3%) achieved ACR70 response at Week 24 than in the placebo group (5.6%, global adjusted p<0.001); The proportion of subjects who achieved ACR70 response at Week 24 was numerically greater in the guselkumab 100 mg q8w group (11.8%) than that in the placebo group (5.6%), but did not reach statistical significance (global adjusted p=0.069).

### Major Secondary Endpoints not Controlled for Multiplicity

Among the 222 (58.3%) subjects with enthesitis at baseline:
At Week 24, 47.9% of subjects in the guselkumab 100 mg q4w group and 40.3% of subjects in the guselkumab 100 mg q8w group achieved enthesitis resolution compared with 27.3% of subjects in the placebo group (nominal p=0.013 and p=0.094, respectively).
At Week 24, the LSmean change from baseline in LEI score was −1.75 in the guselkumab 100 mg q4w group and −1.35 in the guselkumab 100 mg q8w group compared with −1.01 in the placebo group (nominal p=0.004 and p=0.185, respectively).
Among the 142 (37.3%) subjects with dactylitis at baseline:
A numerically greater proportion of subjects in the guselkumab 100 mg q4w and the guselkumab 100 mg q8w groups (63.2% and 65.3%, respectively) achieved dactylitis resolution at Week 24 compared with the placebo group (49.1%; nominal p=0.212 and p=0.088, respectively).
A numerically greater reduction from baseline in dactylitis score at Week 24 was observed in both the guselkumab 100 mg q4w group (LSmean change from baseline: −5.82) and the guselkumab 100 mg q8w group (LSmean change from baseline: −6.11) compared with the placebo group (LSmean change from baseline: −4.30; nominal p=0.225 and p=0.121, respectively).
A numerically greater improvement from baseline in SF-36 MCS score at Week 24 was observed in both the guselkumab 100 mg q4w group (LSmean: 3.60) and the guselkumab 100 mg q8w group (LSmean: 3.20) compared with the placebo group (LSmean: 2.37; nominal p=0.214 and p=0.398, respectively).

### Other Secondary Efficacy Analyses

### Other Efficacy Endpoints Related to Reduction of Joint Signs and Symptoms

Over time through Week 24, ACR 20, ACR 50, and ACR 70 response rates were consistently higher in the 2 guselkumab groups than those in the placebo group.
Numerically greater improvement was consistently observed for both guselkumab treatment groups compared with the placebo group for each ACR component through Week 24.
Improvement in DAS28 (CRP) from baseline, DAS28 (CRP) response rate and DAS28 (CRP) remission rate were consistently higher in the 2 guselkumab groups than those in the placebo group over time. At Week 24, 35.9% of subjects in the guselkumab 100 mg q4w group and 23.6% of subjects in the guselkumab 100 mg q8w group achieved DAS28 (CRP) remission compared with the placebo group (12.7%; nominal p<0.001 and nominal p=0.025, respectively).
Through Week 24, the proportion of subjects achieving a modified PsARC response were consistently higher in both guselkumab treatment groups compared with placebo. At Week 24, the proportion of subjects achieving a modified PsARC response was 72.7% and 59.8% in the guselkumab 100 mg q4w and guselkumab 100 mg q8w groups, respectively, compared with 31.0% in the placebo group (both nominal p<0.001).
Improvement in DAPSA change from baseline and the proportions of subjects achieving low disease activity or remission based on the DAPSA index were consistently higher in the 2 guselkumab groups than those in the placebo group over time. At Week 24, the proportion of subjects achieving low disease activity based on the DAPSA index was 49.2% and 40.9% in the guselkumab 100 mg q4w and guselkumab 100 mg q8w groups, respectively, compared with 16.7% in the placebo group (both nominal p<0.001, respectively).

### Other Efficacy Endpoints Related to Physical Function

Greater reduction from baseline in HAQ-DI and higher HAQ-DI response (defined as ≥0.35 improvement from baseline) rates were consistently observed in the 2 guselkumab groups compared with placebo over time through Week 24. At Week 24, the HAQ-DI response rate among the subjects with a HAQ-DI score≥0.35 at baseline was 57.3% and 50.9% in the guselkumab 100 mg q4w and the guselkumab 100 mg q8w groups, respectively, compared with 29.1% in the placebo group (nominal p<0.001 and p=0.001, respectively).

### Other Efficacy Endpoints Related to Skin Disease

Among the 249 (65.4%) subjects with ≥3% BSA of psoriatic involvement and an IGA score≥2 (mild) at baseline:
Consistently more subjects in the 2 guselkumab treatment groups achieved an IGA score of 0 (clear) or 1 (minimal) and ≥2 grade reduction from baseline or an IGA score of 0 (clear) than placebo through Week 24. At Week 24, the proportions of subjects who achieved an IGA score of 0 (clear) were 53.9% and 38.3% in the guselkumab 100 mg q4w and guselkumab 100 mg q8w groups, respectively, compared with 7.7% in the placebo group (both nominal p<0.001).
Through Week 24, PASI 50, PASI 75, PASI 90, and PASI 100 response rates were consistently higher in both guselkumab treatment groups compared with the placebo group. At Week 24, PASI 75, PASI 90, and PASI 100 response rates were 87.6%, 64.0% and 44.9% in the guselkumab 100 mg q4w group, 76.5%, 50.6%, and 25.9% in the guselkumab 100 mg q8w group compared with 20.0%, 12.9%, and 7.1% in the placebo group (all nominal p<0.001).

### Other Efficacy Endpoints Related to Enthesitis and Dactylitis

Among the 222 (58.3%) subjects with enthesitis at baseline, the proportion of subjects achieving enthesitis resolution was higher in both guselkumab treatment groups compared with the placebo group through Week 24, and a numerically greater reduction from baseline in LEI score was also consistently observed in both guselkumab treatment groups through Week 24. Similar results were observed using SPARCC enthesitis index.
Among the 142 (37.3%) subjects with dactylitis at baseline, the proportion of subjects achieving dactylitis resolution was higher in both guselkumab treatment groups compared with the placebo group over time through Week 24, and a numerically greater reduction from baseline in dactylitis score was also consistently observed in both guselkumab treatment groups through Week 24. Consistent results were observed for tender dactylitis.

### Other Efficacy Endpoints Related to BASDAI

Among the 67 (17.6%) subjects with spondylitis and peripheral arthritis and a BASDAI score>0 at baseline:
At Week 24, LSmean change from baseline in BASDAI was −2.074 the guselkumab 100 mg q4w group and −2.665 in the guselkumab 100 mg q8w group compared with −0.919 in the placebo group (nominal p=0.067 and p=0.004, respectively).
At Week 24, 35.0% of subjects in the guselkumab 100 mg q4w group and 41.7% of subjects in the guselkumab 100 mg q8w group achieved≥50% BASDAI improvement compared with 13.0% in the placebo group (nominal p=0.148 and p=0.082, respectively).
Through Week 24, numerically greater improvements over time above placebo among BASDAI components were only consistently observed for fatigue and spinal pain in both guselkumab treatment groups.

### Other Efficacy Endpoints Related to Health-Related Quality of Life and Other Patient Reported Outcomes

Through Week 24, a numerically greater improvement in SF-36 PCS score and a greater proportion of subjects achieving≥5-point improvement in SF-36 PCS were observed in both guselkumab treatment groups compared with the placebo group. At Week 24, the proportion of subjects who achieved≥5-point improvement from baseline in SF-36 PCS score was 53.9% and 51.2% in the guselkumab 100 mg q4w and guselkumab 100 mg q8w groups, respectively, compared with 28.6% in the placebo group (both nominal p<0.001).
Through Week 24, a numerically greater improvement in SF-36 MCS score and a greater proportion of subjects achieving≥5-point improvement in SF-36 MCS were observed in both guselkumab treatment groups compared with the placebo group. At Week 24, the proportion of subjects who achieved≥5-point improvement from baseline in SF-36 MCS score was 43.0% and 37.8% in the guselkumab 100 mg q4w and guselkumab 100 mg q8w groups, respectively, compared with 25.4% in the placebo group (nominal p=0.003 and p=0.036, respectively).
A numerically greater improvement from baseline in FACIT-Fatigue scores was observed in both guselkumab groups compared with placebo through Week 24. At Week 24, the estimated LSmean of change from baseline in FACIT-Fatigue score was 5.841 for the guselkumab 100 mg q4w and 5.609 for the guselkumab 100 mg q8w groups compared with 2.206 in the placebo group (both nominal p<0.001), and 63.3% and 53.5% in the guselkumab 100 mg q4w and guselkumab 100 mg q8w groups achieved≥4-point improvement from baseline in FACIT-Fatigue score, respectively, compared with 34.9% in the placebo group (nominal p<0.001 and p=0.003, respectively).
Through Week 24, numerically greater improvements from baseline in each of 7 PROMIS 29 domain T scores were observed in both guselkumab treatment groups compared with the placebo group. At Week 24, the proportions of subjects who achieved≥3-point or ≥5-point improvement from baseline in scores of PROMIS-29 domains that are directly related to symptoms and impact of PsA, including pain interference, pain intensity, fatigue, physical function, and ability to participate in social roles and activities, were numerically greater in both guselkumab treatment groups compared with the placebo group.

### Improvements in Composite Disease Activity Scores

Through Week 24, more subjects in the 2 guselkumab treatment groups achieved MDA compared with placebo. At Week 24, the proportion of subjects achieving MDA was 30.5% and 22.8% in the guselkumab 100 mg q4w and guselkumab 100 mg q8w groups, respectively, compared with 11.1% in the placebo group (nominal p<0.001 and p=0.012, respectively). Greater improvements in PASDAS and GRACE index were also observed in both guselkumab treatment groups compared with the placebo group at Week 24 (all nominal p<0.001).

### Efficacy and Pharmacokinetics

There appeared to be a weak exposure-response relationship for ACR 50 response rate at Week 24 by steady-state trough guselkumab concentration quartiles at Week 20 while no apparent exposure-response relationship was observed for ACR 20 response rate at Week 24.
There were no apparent exposure-response relationships for mean changes from baseline in DAS28 (CRP) at Weeks 20 or 24 by steady-state trough guselkumab concentration quartiles at Week 20.
There was an apparent exposure-response relationship in IGA response rate at Week 24 by steady-state trough guselkumab concentration quartiles at Week 20 in subjects with ≥3% BSA psoriatic involvement and an IGA score of ≥2 at baseline.

### Efficacy and Antibodies to Guselkumab

The presence of antibodies to guselkumab did not seem to preclude ACR 20 response for subjects who were positive for antibodies to guselkumab through Week 24 (3 of 5 subjects were ACR 20 responders at Week 24). However, the small number of subjects who were positive for antibodies to guselkumab (n=5) limits a definitive conclusion on the impact of antibodies to guselkumab on clinical efficacy.

### Safety Results

An overall summary of key safety findings from AEs reported through Week 24 is provided in Table 45. The average duration of follow-up and number of study agent administrations were comparable across the treatment groups.

TABLE 45
Overall Summary of Treatment-Emergent Adverse Events
Through Week 24; Safety Analysis Set (Study CNTO1959PSA3001)

| | | Guselkumab | | |
|---|---|---|---|---|
| | | | 100 mg | |
| | Placebo | 100 mg q8w | q4w | Combined |
| Analysis set: Safety Analysis Set | 126 | 127 | 128 | 255 |
| Average duration of follow up (weeks) | 23.7 | 23.9 | 23.9 | 23.9 |
| Average number of study agent administrations | 5.8 | 5.9 | 5.9 | 5.9 |
| Average number of placebo administrations | 5.8 | 2.0 | 0.0 | 1.0 |
| Average number of guselkumab administrations | 0.0 | 4.0 | 5.9 | 4.9 |
| Subjects with 1 or more adverse events | 75 (59.5%) | 68 (53.5%) | 71 | 139 (54.5%) |
| | | | (55.5%) | |
| Subjects with 1 or more serious adverse events | 5 (4.0%) | 4 (3.1%) | 0 | 4 (1.6%) |
| Subjects with 1 or more adverse events leading | 3 (2.4%) | 3 (2.4%) | 1 (0.8%) | 4 (1.6%) |
| to discontinuation of study agent | | | | |
| Subjects with 1 or more adverse events with | 3 (2.4%) | 2 (1.6%) | 0 | 2 (0.8%) |
| severe intensity | | | | |
| Subjects with 1 or more infections | 32 (25.4%) | 33 (26.0%) | 31 | 64 (25.1%) |
| | | | (24.2%) | |
| Subjects with 1 or more serious infections | 2 (1.6%) | 0 | 0 | 0 |
| Subjects with 1 or more injection site reactions | 0 | 2 (1.6%) | 1 (0.8%) | 3 (1.2%) |
| Subjects with 1 or more events of malignancy | 0 | 1 (0.8%) | 0 | 1 (0.4%) |
| Subjects with 1 or more opportunistic infections | 0 | 0 | 0 | 0 |
| Subjects with 1 or more events leading to death | 1 (0.8%) | 0 | 0 | 0 |

Note:
Subjects are counted only once for any given event, regardless of the number of times they actually experienced the event. Adverse events are coded using MedDRA Version 21.1

The proportion of subjects experiencing AEs through Week 24 was generally comparable across the treatment groups: 55.5% in the guselkumab 100 mg q4w group, 53.5% in the guselkumab 100 mg q8w group, and 59.5% in the placebo group.

The most frequent SOC of reported AEs was Infections and infestations (22.7% in the guselkumab 100 mg q4w group, 26.8% in the guselkumab 100 mg q8w group, and 25.4% in the placebo group), followed by Musculoskeletal and connective tissue disorders (17.2% in the guselkumab 100 mg q4w group, 14.2% in the guselkumab 100 mg q8w group, and 19.0% in the placebo group).

The most common PTs with a frequency ≥5% in any treatment group through Week 24 are presented in Table 46. The most common AEs reported were nasopharyngitis (5.5% in the guselkumab 100 mg q4w group, 12.6% in the guselkumab 100 mg q8w group, and 6.3% in the placebo group) followed by upper respiratory tract infection (8.6% in the guselkumab 100 mg q4w group, 5.5% in the guselkumab 100 mg q8w group, and 6.3% in the placebo group). Overall, transaminase increases were reported as AEs more frequently in guselkumab-treated subjects than in placebo-treated subjects, but no dose-related trend was observed in these AEs.

TABLE 46
Number of Subjects with Treatment-Emergent Adverse
Events (Excluding Serious Adverse Events) with Frequency of at
Least 5% in Any Treatment Group Through Week 24 by MedDRA
System-organ Class and Preferred Term; Safety Analysis Set (Study
CNTO1959PSA3001)

| | | Guselkumab | | |
|---|---|---|---|---|
| | Placebo | 100 mg q8w | 100 mg q4w | Combined |
| Analysis set: Safety Analysis Set | 126 | 127 | 128 | 255 |
| Average duration of follow up (weeks) | 23.7 | 23.9 | 23.9 | 23.9 |
| Average number of study agent administrations | 5.8 | 5.9 | 5.9 | 5.9 |
| Subjects with 1 or more adverse events (excluding | 75 (59.5%) | 67 (52.8%) | 71 (55.5%) | 138 (54.1%) |
| serious events) | | | | |
| MedDRA system-organ class/preferred term | | | | |
| Infections and infestations | 32 (25.4%) | 34 (26.8%) | 29 (22.7%) | 63 (24.7%) |
| Nasopharyngitis | 8 (6.3%) | 16 (12.6%) | 7 (5.5%) | 23 (9.0%) |
| Upper respiratory tract infection | 8 (6.3%) | 7 (5.5%) | 11 (8.6%) | 18 (7.1%) |
| Investigations | 7 (5.6%) | 15 (11.8%) | 9 (7.0%) | 24 (9.4%) |
| Alanine aminotransferase increased | 3 (2.4%) | 8 (6.3%) | 5 (3.9%) | 13 (5.1%) |
| Aspartate aminotransferase increased | 3 (2.4%) | 9 (7.1%) | 3 (2.3%) | 12 (4.7%) |

Note:
Subjects are counted only once for any given event, regardless of the number of times they actually experienced the event. Adverse events are coded using MedDRA Version 21.1

### Example 3. Guselkumab Demonstrated an Improvement in PROMIS-29 and Independent Treatment Effect on Fatigue after Adjustment for Clinical Response (ACR20) in Patients with Psoriatic Arthritis Who are Biologicaly Naïve and Patients Previously Treated with Biologic Anti-TNFα Agent(s)

### Patient-Reported Outcomes Measurement Information System-29-PROMIS-29 (PROMIS-29)

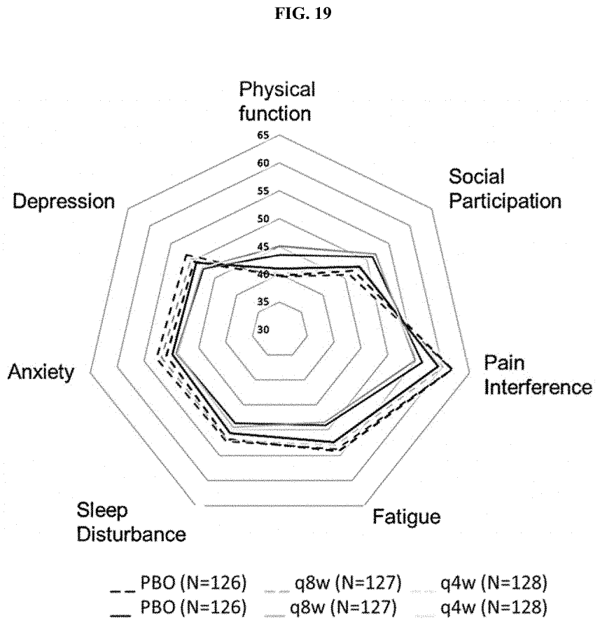

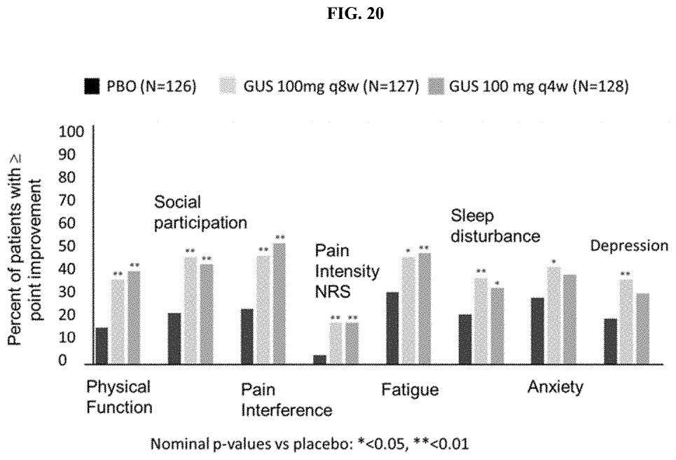

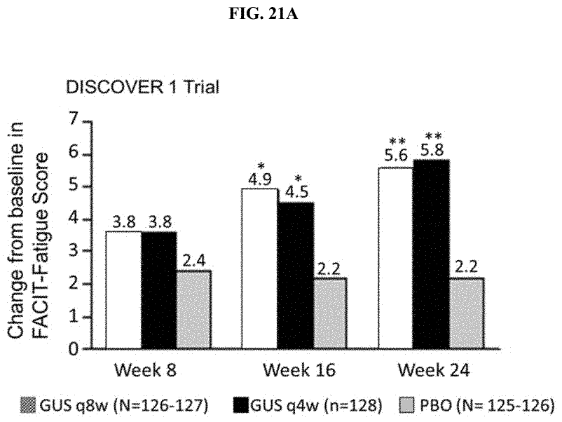

PROMIS-29 at Week 24: Patients with psoriatic arthritis (PsA) experience broad systemic symptoms including pain, fatigue, depression, sleep disturbance, poor physical function, and diminished social participation. PROMIS-29 (Patient-Reported Outcomes Measurement Information System-29), is a validated generic health instrument, used to asses the treatment effect of GUS on symptoms in patients with PsA. PROMIS-29 consists of 7 domains (Depression, Anxiety, Physical Function, Pain Interference, Fatigue, Sleep Disturbance, and Social Participation) and a pain intensity 0-10 numeric rating scale (NRS). The raw score of each domain is converted into a standardized T-score with a mean of 50 (general population mean) and a standard deviation (SD) of 10. Higher PROMIS scores represent more of the concept being measured. A >5-point improvement (1/2 SD of T-score) is defined as clinically meaningful. At baseline, mean PROMIS-29 T-scores for physical function, social participation, sleep disturbance, pain, and fatigue were worse than the general US population. At W24, GUS q8W-treated pts achieved greater improvements from baseline in all PROMIS-29 domains vs PBO (p<0.05) (Table 47 and FIG. 19). Results were consistent in the GUS q4W group except for anxiety and sleep disturbance. More pts receiving GUS achieved clinically meaningful improvement vs PBO except for depression and anxiety in the GUS q4W group, which were numerically improved (FIG. 6). The p-values are based on the Cochran-Mantel-Hanszel test stratified by baseline use of csDMARDs (yes, no) and prior exposure to anti-TNFα agents (yes/no). Active PsA pts treated with GUS achieved clinically meaningful reduction in symptoms and improvement in physical function and social participation vs PBO at W24 (FIG. 20).

TABLE 47
PROMIS-29 Domain T-Scores Least Square (LS) Mean
Change from Baseline

| | LS Mean Change from Baseline | | |
|---|---|---|---|
| | | GUS | |
| | PBO | q8W | GUS q4W |
| Anxiety | −1.37 | −3.23* | −2.92 |
| Depression | −0.85 | −3.4** | −2.67* |
| Fatigue | −1.86 | −4.79** | −5.08** |
| Pain | −2.30 | −5.49** | −5.69** |
| interference | | | |
| Physical | 1.34 | 3.89** | 5.05** |
| function | | | |
| Sleep | −1.17 | −3.48** | −2.46 |
| disturbance | | | |
| Social | 1.45 | 4.90** | 4.52** |
| participation | | | |
| Pain intensity | −0.56 | −1.98** | −2.32** |

Nominal p-values vs placebo: *<0.05, **<0.01

### FACIT-Fatigue

The patient reported outcome (PRO) FACIT-Fatigue, which has demonstrated content validity and strong psychometric properties in clinical trials, was used to to evaluate the effect of GUS on fatigue in patients used in the studies described above.

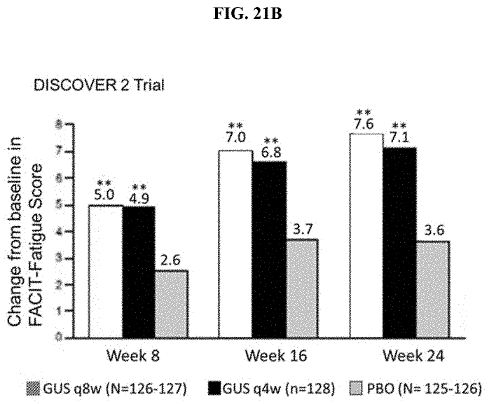

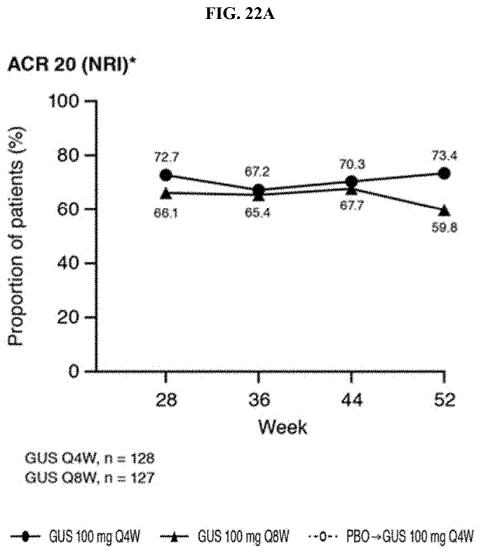

Method. DISC 1 and DISC 2 enrolled patients with active PsA despite nonbiologic DMARDS and/or NSAIDS who were mostly biologic naïve except for ˜30% of patients in DISC 1 who had received 1-2 TNFi. Patients were randomized (1:1:1) in a blinded fashion to subcutaneous GUS 100 mg at WO and W4 then every (q) 8W, to GUS 100 mg q4W, or to matching PBO. Concomitant treatment with select non-biologic DMARDS, oral corticosteroids, and NSAIDs was allowed. The FACIT-Fatigue is a 13-item PRO instrument assessing fatigue and its impact on daily activities and function over the past seven days, with a total score ranging from 0 to 52, higher score denoting less fatigue. A change of ≥4 points is identified as clinically meaningful (Cella et al. Journal of Patient-Reported Outcomes. 2019; 3:30). Change from baseline in FACIT-Fatigue was analyzed using MMRM (FIG. 19). Independence of treatment effect on FACIT-Fatigue from effect on ACR20 was assessed using Mediation Analysis (Valeri et al. Psychologic Meth. 2013; 18:137) (Table 48) to estimate the natural direct effect (NDE) and natural indirect effect (NIE) mediated by ACR20 response.
Results. At baseline in DISC 1 & 2, the mean FACIT-fatigue scores (SD) were 30.4 (10.4) and 29.7 (9.7), respectively, indicating moderate to severe fatigue. In both DISCOVER 1 & 2 trials, treatment with GUS led to significant improvements in FACIT-Fatigue scores compared with PBO as early as W8 (FIGS. 21A-B). 54%-63% of GUS patients compared with 35%-46% of PBO patients achieved clinically meaningful improvement (≥4 points) in FACIT-Fatigue (P≤0.003). Mediation analysis revealed that the independent treatment effects on fatigue after adjustment for ACR20 response (Natural Direct Effect [NDE], Table 26) were 12-36% in the q8W GUS dosing group and 69%-70% in the q4W GUS group (FIGS. 21A-B).

TABLE 48
Mediation Analysis of the Effect of ACR 20 Response on
Change from Baseline in FACIT-Fatigue Score at Week 24

| | | GUS 100 mg | GUS 100 mg |
|---|---|---|---|
| | | q8W vs. PBO | q4W vs. PBO |
| | Effect | Estimate (95% CI) | Estimate (95% CI) |
| DISCOVER | NDE | 0.36 (−1.7, 2.4) | 2.60 (0.6, 4.5)* |
| 1 | | | |
| | NIE | 2.75 (1.4, 4.3)* | 1.20 (0.3, 2.3)* |
| | Total Effect | 3.12 (1.0, 5.2)* | 3.79 (1.9, 5.4)* |
| | Proportion | 11.7% | 68.5% |
| | Independent | | |
| | Proportion | 88.3% | 31.5% |
| | Mediated | | |
| DISCOVER | NDE | 1.44 (−0.1, 3.0) | 2.49 (1.0, 4.1)* |
| 2 | | | |
| | NIE | 2.53 (1.6, 3.6)* | 1.09 (0.4, 1.9)* |
| | Total Effect | 3.97 (2.4, 5.5)* | 3.58 (2.1, 5.0)* |
| | Proportion | 36.3% | 69.7% |
| | Independent | | |
| | Proportion | 63.7% | 30.3% |
| | Mediated | | |

*P vs placebo < 0.02
NDE = Natural Direct Effect (effect on FACIT-F beyond effect on ACR20), NIE = Natural Indirect Effect (effect on FACIT-F mediated by ACR20)
Mediation analysis used linear regression and logistics regression models with Bootstrapping method

Conclusion: In 2 phase-3 trials, treatment with GUS of patients with active PsA led to significant improvements compared to PBO in fatigue, including substantial effects on FACIT-Fatigue that were independent of the effects on ACR 20, especially for the q4W dosing group.

### Example 4. Specific Inhibition of IL-23 with Guselkumab for Active Psoriatic Arthritis: One Year Results of a Phase 3, Randomized, Double-Blind, Placebo-Controlled Study of Patients Who were Biologic-Naïve or TNFα Inhibitor-Experienced

While the objectives of the Week 24 analysis were to compare across treatment groups (i.e. guslekumab to placebo), the focus of the Week 52 study is to present data on maintenance of efficacy from Week 24 through Week 52 (the last scheduled assessment of efficacy data) on improving joint and skin signs and symptoms, physical function and health-related quality of life. The study also summarizes cumulative safety findings from first administration of study agent at Week 0 through Week 60 (End of study). The Week 52 analysis population includes all randomized patients still on study treatment at Week 24.

The Week-52 analysis was not placebo- or active-controlled as all placebo-treated patients at Week 24 crossed over to Q4w treatment. Consequently, no formal statistical testing could be performed for the uncontrolled period (Wk 24-52) and only descriptive statistics are provided. The data are based on an ‘as observed” population and therefore are descriptive only with no formal statical testing performed

### Method

The study involved 381 patients including TNF-experienced patients (31%) over 48 weeks of treatment. Adults with active PsA (≥3 swollen+≥3 tender joints; CRP≥0.3 mg/dL) despite standard therapies were eligible. Approx. 30% of patients could have previously received≤2 TNFi. Patients were randomized 1:1:1, stratified by W0 DMARD [Y/N] & prior TNFi (Y/N) use, to GUS 100 mg Q4W; GUS 100 mg at W0, W4 & Q8W; or PBO. At W24, PBO patients crossed over to GUS 100 mg Q4W (PBO→Q4W). W48 marked the last dose of study agent. ACR response rates at W52, based on nonresponder imputation (NRI) for missing data and as observed in patients still on study agent at W24, are shown. Observed data for additional endpoints are shown. AEs through W60 are reported.

### Results

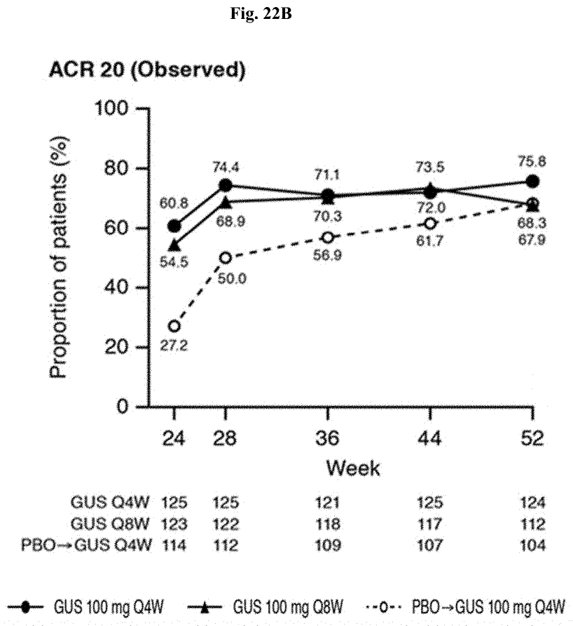

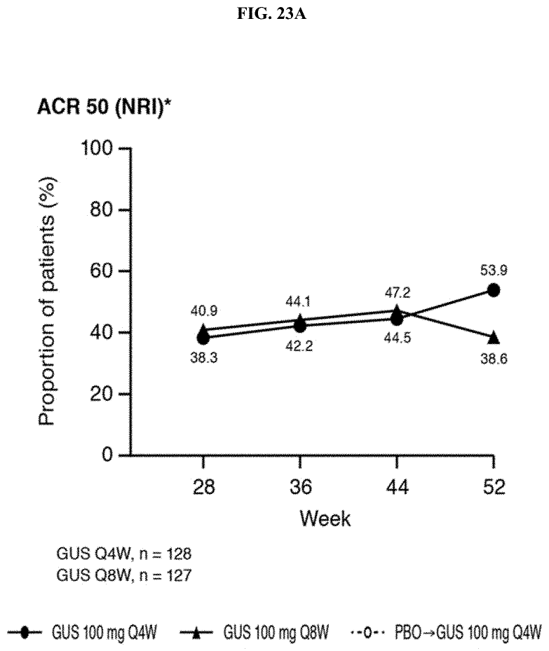

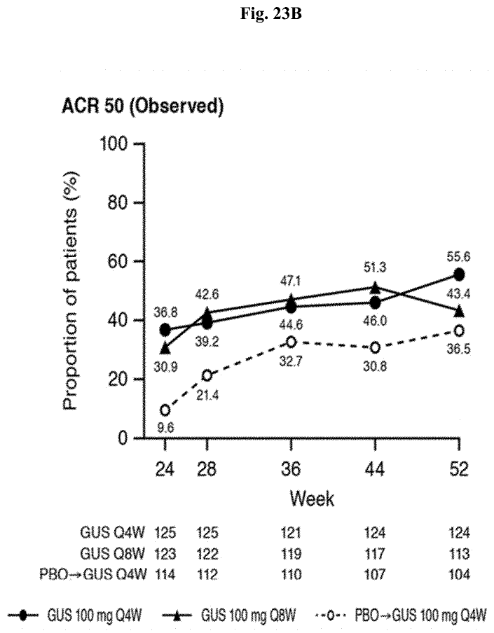

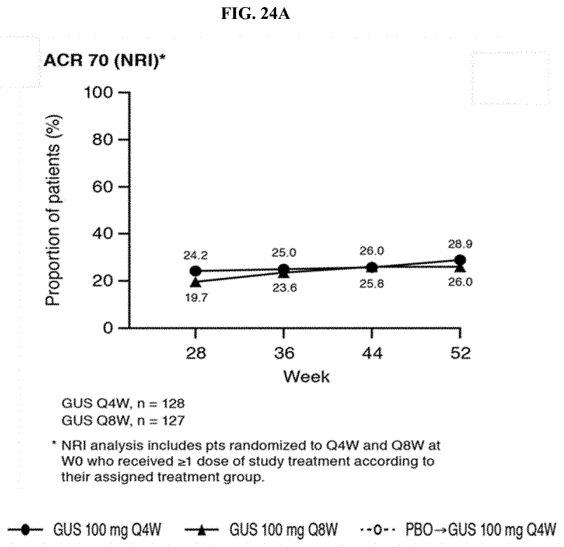

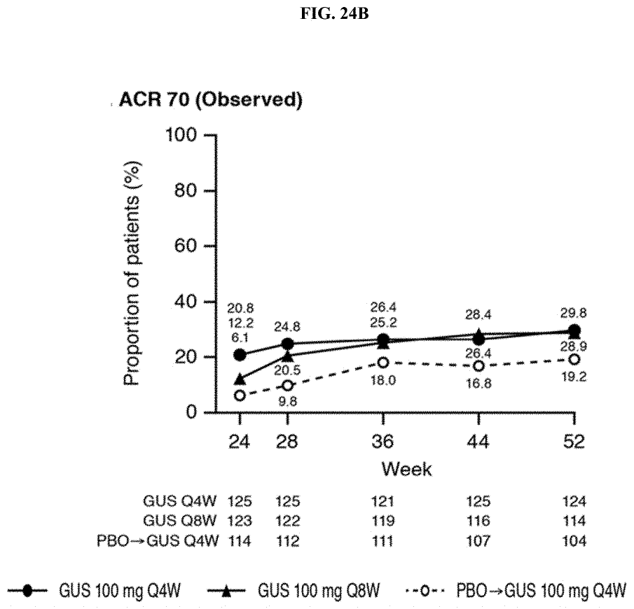

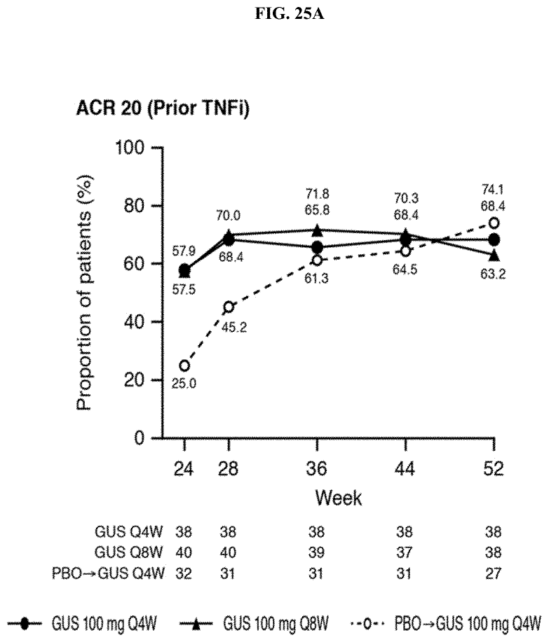

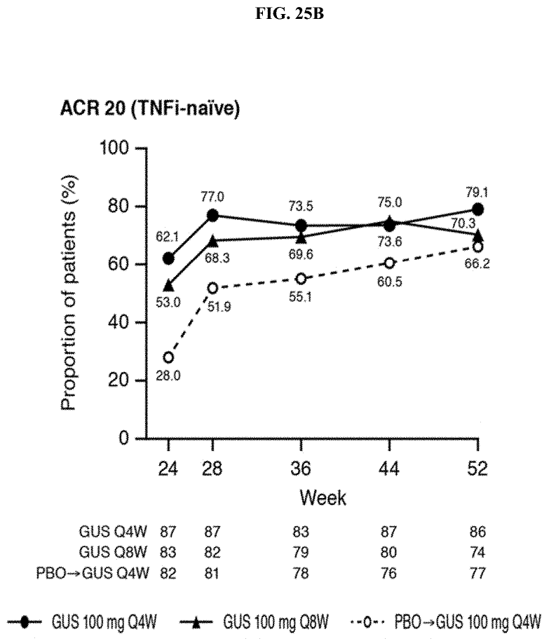

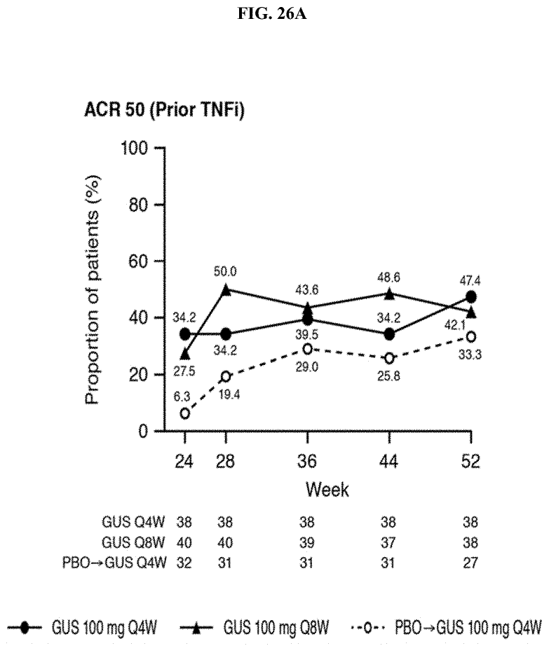

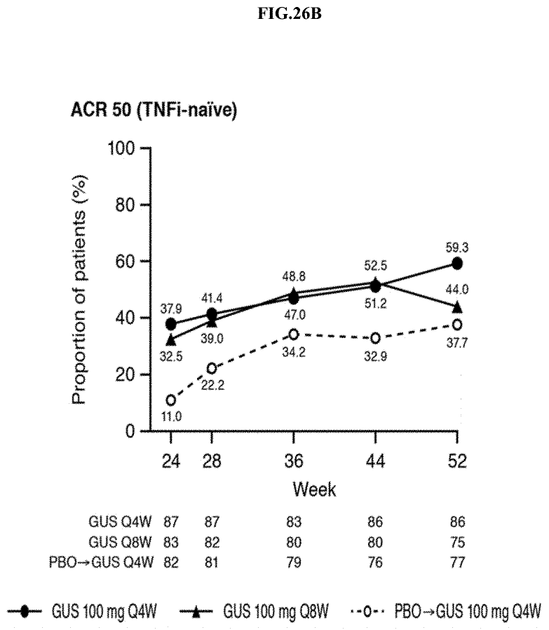

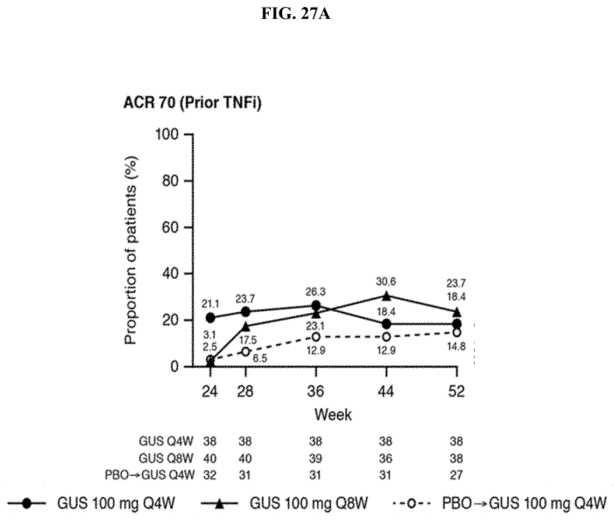

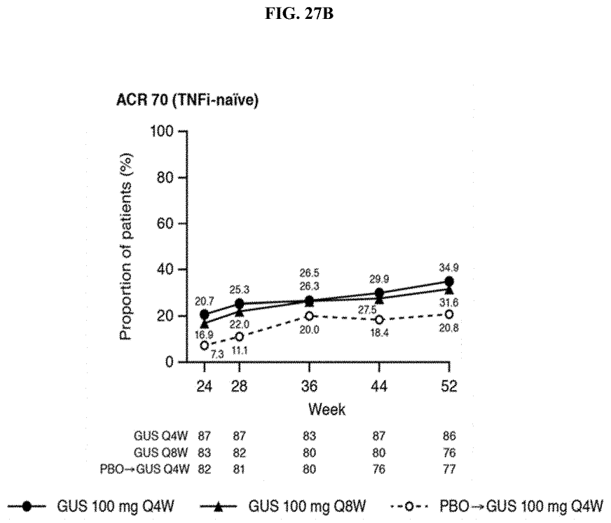

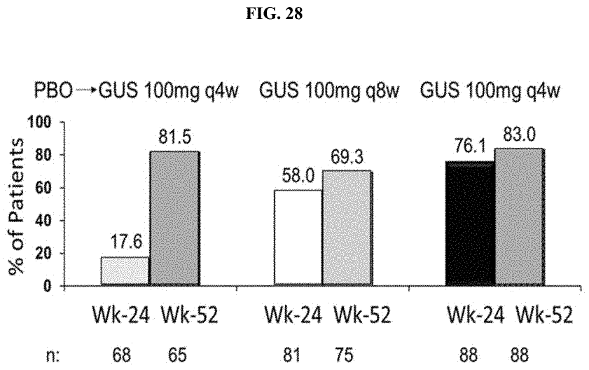

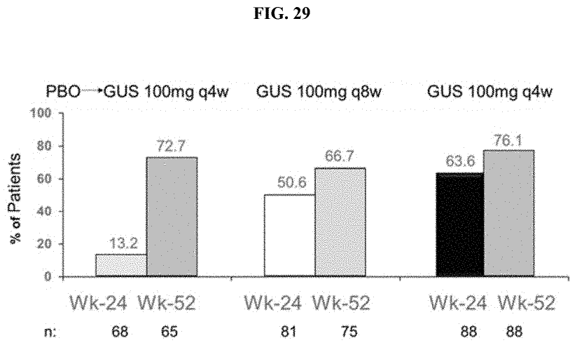

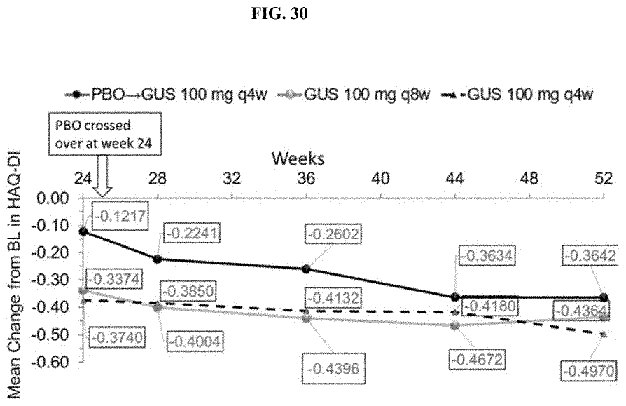

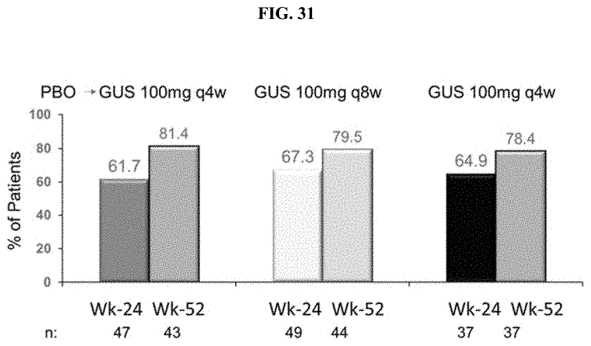

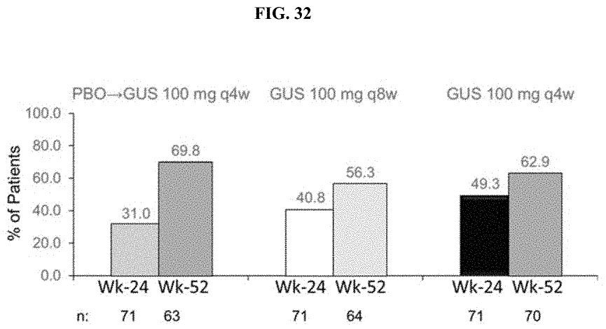

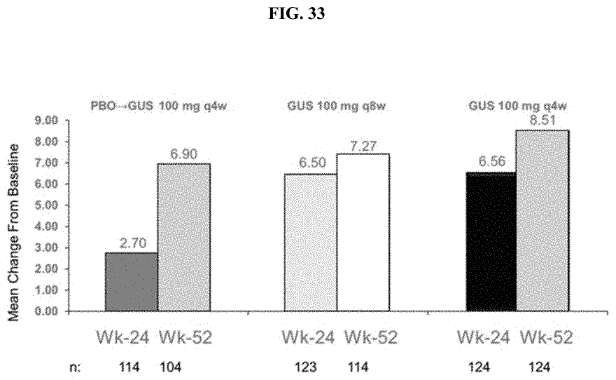

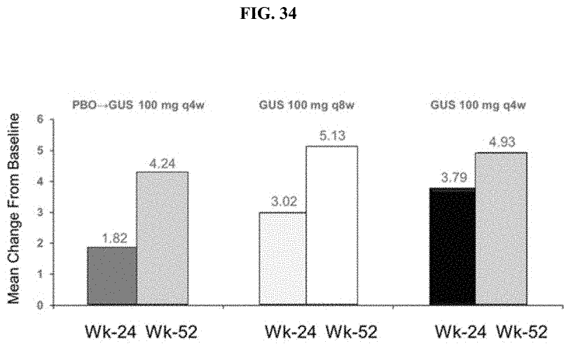

362/381 (95%) randomized patients continued study agent at W24 (125 Q4W, 123 Q8W, 114 PBO→Q4W), 347/381 (91%) patients completed treatment & 343/381 (90%) completed study. NRI ACR20 response rates were maintained at W52 (Q4W 73%, Q8W 60%; FIGS. 22A-B). Similar responses patterns were seen for the more stringent ACR50/70 criteria (FIGS. 23A-B, FIGS. 24A-B)),). Observed ACR responses, overall (FIGS. 25A-B, FIGS. 26A-B. FIGS. 27A-B)) and in patients with (FIG. 25A, FIG. 26A, FIG. 27A) & without (FIG. 25B, FIG. 26B, FIG. 27B) prior TNFi use, were also maintained at W52. Improvements in other clinical outcomes were also maintained at W52 (FIG. 28-FIG. 34), and responses for patients crossing over from PBO→Q4W at W24 were generally consistent with other GUS-treated patients by W52 (Table 49). Through W24, 4 (2%) GUS- and 5 (4%) PBO-treated patients had serious AEs; no GUS-treated and 2 (2%) PBO-treated patients had a serious infection. Through W60, serious AEs and serious infections occurred in 4% & 1%, respectively, of all 369 GUS-treated patients; no GUS-treated pt died or had IBD, opportunistic infections/active TB, or anaphylactic/serum sickness-like reactions.

TABLE 49

| Observed Efficacy¹ | GUS Q4W | | GUS Q8W | | PBO(WO-24)→Q4W(W24-52) | |
|---|---|---|---|---|---|---|
| Data are % unless otherwise stated | W24 | W52 | W24 | W52 | W24 | W52 |
| Dactylitis at W0, n | 37 | 37 | 49 | 44 | 47 | 43 |
| Resolution | 64.9 | 78.4 | 67.3 | 79.5 | 61.7 | 81.4 |
| Enthesitis at W0, n | 71 | 70 | 71 | 64 | 71 | 63 |
| Resolution | 49.3 | 62.9 | 40.8 | 56.3 | 31.0 | 69.8 |
| ≥3% BSA psoriasis, IGA ≥2 at | 88 | 88 | 81 | 75 | 68 | 66 |
| W0, n | | | | | | |
| IGA 0/1 + ≥2-grade decrease | 76.1 | 83.0 | 58.0 | 69.3 | 17.6 | 81.52 |
| PAS175 | 87.5 | 94.3 | 76.5 | 80.0 | 20.6 | 84.8 |
| PASI90 | 63.6 | 76.1 | 50.6 | 66.7 | 13.2 | 72.7 |
| PASI100 | 45.5 | 64.8 | 25.9 | 48.0 | 7.4 | 62.1 |
| HAQ-DI, n | 125 | 124 | 123 | 114 | 114 | 104 |
| Mean change | −0.4 | −0.5 | −0.3 | −0.4 | −0.1 | −0.4 |
| SF-36 scores, n (mean change) | 124 | 124 | 123 | 114 | 114 | 104 |
| Physical Component-PCS | 6.6 | 8.5 | 6.5 | 7.3 | 2.7 | 6.9 |
| Mental Component-MCS | 3.8 | 4.9 | 3.0 | 5.1 | 1.8 | 4.2 |
| MDA, n | 125 | 124 | 123 | 112 | 114 | 103 |
| MDA response | 31.2 | 40.3 | 23.6 | 33.9 | 12.3 | 31.1 |
| VLDA, n | 125 | 124 | 123 | 114 | 113 | 104 |
| VLDA response | 9.6 | 16.9 | 4.1 | 12.3 | 1.8 | 14.4 |

¹Randomized pts still on study agent at W24; ²n = 65

As shown above, both doses of guselkumab (Q4w and Q8w) either maintained or showed numerical improvements in all clinical endpoints beyond Week 24 to Week 52. The data also showed that both doses of guselkumab were safe and well-tolerated through Week 52. The safety profile of guselkumab in this population of psoriatic arthritis patients through Week 52 was generally consistent with that demonstrated in the psoriasis indication. Similar to the primary analyses at Week 24, the 52-week analyses suggest no overall dose response in the domains of efficacy (joint, enthesitis, dactylitis, physical function or QOL) between the Q8w and Q4w dosing regimen. There was a numerical difference in proportion of subjects with skin response between the q4w and q8w dose regimens (i.e., IGA response 83% in q4w and 69% in q8w. This difference is smaller than what was seen in the Week 24 analysis (.ie., IGA response 75.3% in q4w and 57.3% in q8w).

### CONCLUSION

The data shows a marked impact on signs and symptoms that were maintained and further improved in biologic naïve and anti-TNF experienced patients through week 52, confirming the robust and sustained efficacy and safety seen at week 24.

The Week 52 results demonstrated continued improvement from the previously reported Week 24 results, providing additional evidence that durability of response is an important feature of IL-23 inhibition therapy. Both dose regimens showed highly clinically meaningful improvement in efficacy on signs and symptoms of the joints and skin psoriasis, physical function, enthesitis, dactylitis, and health-related quality of life through 1 year of exposure, including on patients who were TNF-experienced patients. Both the guselkumab 100 mg Q4W and Q8W dose regimens were safe and well-tolerated through Week 52.

### Safety Week 24 Through Week 52

Both GUS 100 mg q4w and q8w dose regimens were safe and well-tolerated through end of study (Table 50, Table 51). The safety profile of GUS in this population of psoriatic arthritis patients through end of study was generally consistent with that demonstrated in the psoriasis indication.

TABLE 50
Overall summary of treatement-emergent adverse events
during PBO-controlled period

| | PBO-controlled Period | | | |
|---|---|---|---|---|
| | | GUS | GUS | GUS |
| | PBO | 100 mg q8w | 100 mg q4w | Combined |
| Analysis set: safety analysis set | 126 | 127 | 128 | 255 |
| Avg duration of follow up (weeks) | 24.0 | 24.2 | 24.0 | 24.1 |
| Avg no. of study agent admins | 5.8 | 5.9 | 5.9 | 5.9 |
| AEs | 76 (60.3%) | 68 (53.5%) | 70 (54.7%) | 138 (54.1%) |
| SAEs | 5 (4.0%) | 4 (3.1%) | 0 | 4 (1.6%) |
| AE leading to D/C treatment | 3 (2.4%) | 3 (2.4%) | 1 (0.8%) | 4 (1.6%) |
| AE with severe intensity | 4 (3.2%) | 2 (1.6%) | 0 | 2 (0.8%) |
| Infections | 32 (25.4%) | 33 (26.0%) | 31 (24.2%) | 64 (25.1%) |
| Serious infections | 2 (1.6%) | 0 | 0 | 0 |
| Injection site reactions | 0 | 2 (1.6%) | 1 (0.8%) | 3 (1.2%) |
| Suicidal ideation-Level 1 | 1 (0.8%) | 1 (0.8%) | 0 | 1 (0.4%) |
| MACE | 1 (0.8%) | 0 | 0 | 0 |
| Death | 2 (1.6%) | 0 | 0 | 0 |
| Events of malignancy | 0 | 1 (0.8%) | 0 | 1 (0.4%) |

TABLE 51
Overall summary of treatement-emergent adverse events
reporting period through end of study

| | Reporting Period Through End of Study | | | | |
|---|---|---|---|---|---|
| | | GUS | | GUS | |
| | GUS | 100 mg | PBO→GUS | 100 mg q4w | All GUS |
| | 100 mg q8w | q4w | 100 mg q4w | Combined | Combined |
| Analysis set: safety | 127 | 128 | 114 | 242 | 369 |
| analysis set | | | | | |
| Avg duration of follow up | 58.3 | 59.5 | 35.3 | 48.1 | 51.6 |
| (weeks) | | | | | |
| Avg no. of study agent | 12.4 | 12.7 | 6.8 | 9.9 | 10.8 |
| admins | | | | | |
| AEs | 87 (68.5%) | 89 (69.5%) | 55 (48.2%) | 144 (59.5%) | 231 (62.6%) |
| SAEs | 8 (6.3%) | 4 (3.1%) | 4 (3.5%) | 8 (3.3%) | 16 (4.3%) |
| AE leading to D/C | 5 (3.9%) | 1 (0.8%) | 3 (2.6%) | 4 (1.7%) | 9 (2.4%) |
| treatment | | | | | |
| AE with severe intensity | 5 (3.9%) | 4 (3.1%) | 1 (0.9%) | 5 (2.1%) | 10 (2.7%) |
| Infections | 54 (42.5%) | 49 (38.3%) | 30 (26.3%) | 79 (32.6%) | 133 (36.0%) |
| Serious infections | 2 (1.6%) | 0 | 2 (1.8%) | 2 (0.8%) | 4 (1.1%) |
| Injection site reactions | 2 (1.6%) | 4 (3.1%) | 2 (1.8%) | 6 (2.5%) | 8 (2.2%) |
| Suicidal ideation-Level 1 | 2 (1.6%) | 1 (0.8%) | 1 (0.9%) | 2 (0.8%) | 4 (1.1%) |
| MACE | 0 | 0 | 0 | 0 | 0 |
| Death | 0 | 0 | 0 | 0 | 0 |
| Events of malignancy | 1 (0.8%) | 0 | 1 (0.9%) | 1 (0.4%) | 2 (0.5%) |

### Example 5. Radiographic Read

In DISCOVER-2, radiographic images of hands (posteroanterior) and feet (anteroposterior) were scored in three reading sessions: 1) Completed prior to the Week-24 database lock, including two radiographic images (Week 0 and Week 24 [or discontinuation prior to Week 24]) per patient; 2) Completed prior to the Week-52 database lock, including three radiographic images (Week 0, Week 24 and Week 52 [or discontinuation between Week 24-52]) per patient; and 3) Completed prior to the final database lock, including four radiographic images (Week 0, Week 24, Week 52, and Week 100 [or discontinuation post-Week 52]) per patient.

In each reading session, the designated radiographic images were evaluated independently by the two primary readers. Reader 1 participated in both the first and second reading sessions as a primary reader, Reader 2 in the first session was the adjudicator for the second session, and the adjudicator in the first session was a primary reader in the second session (designated as Reader 2). If the inter-reader difference of the change from baseline in total psoriatic arthritis (PsA)-modified van der Heijde-Sharp (vdH-S) scores at any post-baseline visit exceeded 10 (prespecified), the same set of radiographic images for that patient in a given reading session was read by a third reader (the adjudicator).

For radiographs obtained at Week 0, Week 24, and Week 52, intraclass correlation coefficients indicated good (0.92-0.93 for absolute scores) and moderate (0.58-0.76 for change scores) reader reliability. Smallest detectable changes in PsA-modified vdH-S total, erosion, and JSN scores, respectively, were 1.85, 1.72, and 0.85 during Week 0-24; 1.91, 1.69, and 0.82 during Week 24-52; and 2.39, 2.22, and 1.02 during Week 0-52 (Table 52).

In the guselkumab Q4W group, observed mean changes in total PsA-modified vdH-S scores were 0.46 and 0.62, respectively, during Week 0-24 and Week 24-52. Respective changes in the guselkumab Q8W group were 0.73 and 0.23. In patients who crossed over from placebo to guselkumab Q4W at Week 24, mean changes in total vdH-S scores were 1.00 during Week 0-24 and 0.25 during Week 24-52 (Table 52).

TABLE 52
Observed PsA-modified vdH-S scores from the second reading
session of DISCOVER-2 (images obtained at Week 0, Week 24, and Week 52)

| | | | | | | | Placebo (Week 0-24) → | | |
|---|---|---|---|---|---|---|---|---|---|
| | | | | | | | Guselkumab Q4W | | |
| | Guselkumab Q4W | | | Guselkumab Q8W | | | (Week 24-52) | | |
| Baseline total | 232 | | | 238 | | | 231 | | |
| PsA-modified | | | | | | | | | |
| vdH-S scores, N | | | | | | | | | |
| Mean (SD) | 25.37 (40.24) | | | 22.39 (37.87) | | | 22.96 (39.45) | | |
| Median | 8.00 | | | 10.50 | | | 9.00 | | |
| Range | (0.0-283.0) | | | (0.0-254.5) | | | (0.0-204.4) | | |
| IQR | (3.00-28.75) | | | (2.50-26.50) | | | (3.00-22.00) | | |
| | Study Period | | | Study Period | | | Study Period | | |
| Mean (SD) | Week | Week | Week | Week | Week | Week | Week | Week | Week |
| changes in PsA- | 0-24 | 24-52 | 0-52 | 0-24 | 24-52 | 0-52 | 0-24 | 24-52 | 0-52 |
| modified vdH-S | | | | | | | | | |
| scores | | | | | | | | | |
| N | 232 | 229 | 229 | 238 | 235 | 235 | 231 | 230 | 230 |
| Total^{a} | 0.46 | 0.62 | 1.07 | 0.73 | 0.23 | 0.97 | 1.00 | 0.25 | 1.25 |
| | (2.46) | (2.53) | (3.84) | (2.50) | (1.81) | (3.62) | (3.19) | (1.64) | (3.51) |
| Erosion | 0.31 | 0.39 | 0.70 | 0.57 | 0.10 | 0.67 | 0.75 | 0.17 | 0.92 |
| | (1.88) | (1.72) | (2.63) | (2.04) | (1.42) | (2.71) | (2.31) | (1.28) | (2.50) |
| JSN | 0.15 | 0.23 | 0.38 | 0.16 | 0.13 | 0.29 | 0.25 | 0.07 | 0.33 |
| | (0.97) | (1.09) | (1.63) | (0.78) | (0.70) | (1.27) | (1.14) | (0.64) | (1.36) |

^{a}ICC estimates for the total PsA-modified vdH-S scores at baseline, Week 24, and Week 52 were 0.92, 0.93, and 0.93 respectively; those for changes in the total PsA-modified vdH-S score during Week 0-24, Week 24-52, Week 0-52 were 0.69, 0.58 and 0.76 respectively.
^{b}Total PsA-modified vdH-S score SDC = 1.85 (Week 0-24), 191 (Week 24-52), and 2.39 (Week 0-52).
ICC-intra-class correlation,
IQR-interquartile range,
JSN-joint space narrowing,
PsA-psoriatic arthritis,
Q4/8W-every 4/8 weeks,
SD-standard deviation,
SDC-smallest detectable change, vdH-S-van der Heilde-Sharp

Sequence List:

| SEQ | | |
|---|---|---|
| ID | | |
| NO: | Description | Sequence |
| 1 | HCDR1 | NYWIG |
| 2 | HCDR2 | IIDPSNSYTR YSPSFQG |
| 3 | HCDR3 | WYYKPFDV |
| 4 | LCDR1 | TGSSSNIGSG YDVH |
| 5 | LCDR2 | GNSKRPS |
| 6 | LCDR3 | ASWTDGLSLV V |
| 7 | VH | EVQLVQSGAE VKKPGESLKI SCKGSGYSFS NYWIGWVRQM PGKGLEWMGI |
| | | IDPSNSYTRY SPSFQGQVTI SADKSISTAY LQWSSLKASD TAMYYCARWY |
| | | YKPFDVWGQG TLVTVSS |
| 8 | VL | QSVLTQPPSV SGAPGQRVTI SCTGSSSNIG SGYDVHWYQQ LPGTAPKLLI |
| | | YGNSKRPSGV PDRFSGSKSG TSASLAITGL QSEDEADYYC ASWTDGLSLV |
| | | VFGGGTKLTV L |
| 9 | Heavy Chain | EVQLVQSGAE VKKPGESLKI SCKGSGYSFS NYWIGWVRQM PGKGLEWMGI |
| | | IDPSNSYTRY |
| | | SPSFQGQVTI SADKSISTAY LQWSSLKASD TAMYYCARWY YKPFDVWGQG |
| | | TLVTVSSAST |
| | | KGPSVFPLAP SSKSTSGGTA ALGCLVKDYF PEPVTVSWNS GALTSGVHTF |
| | | PAVLQSSGLY |
| | | SLSSVVTVPS SSLGTQTYIC NVNHKPSNTK VDKKVEPKSC DKTHTCPPCP |
| | | APELLGGPSV |
| | | FLFPPKPKDT LMISRTPEVT CVVVDVSHED PEVKFNWYVD GVEVHNAKTK |
| | | PREEQYNSTY |
| | | RVVSVLTVLH QDWLNGKEYK CKVSNKALPA PIEKTISKAK GQPREPQVYT |
| | | LPPSRDELTK |
| | | NQVSLTCLVK GFYPSDIAVE WESNGQPENN YKTTPPVLDS DGSFFLYSKL |
| | | TVDKSRWQQG |
| | | NVFSCSVMHE ALHNHYTQKS LSLSPGK |
| 10 | Light Chain | QSVLTQPPSV SGAPGQRVTI SCTGSSSNIG SGYDVHWYQQ LPGTAPKLLI |
| | | YGNSKRPSGV |
| | | PDRFSGSKSG TSASLAITGL QSEDEADYYC ASWTDGLSLV VFGGGTKLTV |
| | | LGQPKAAPSV |
| | | TLFPPSSEEL QANKATLVCL ISDFYPGAVT VAWKADSSPV KAGVETTTPS |
| | | KQSNNKYAAS |
| | | SYLSLTPEQW KSHRSYSCQV THEGSTVEKT VAPTECS |

We claim:

1. A method of treating psoriatic arthritis in a subject in need thereof, comprising subcutaneously administering to the subject about 50 mg to about 150 mg of an anti-IL-23 antibody, wherein the antibody comprises a heavy chain variable region and a light chain variable region, the heavy chain variable region comprising a complementarity determining region heavy chain 1 (CDRH1) amino acid sequence of SEQ ID NO: 1, a CDRH2 of SEQ ID NO: 2, and a CDRH3 of SEQ ID NO: 3; and the light chain variable region comprising a complementarity determining region light chain 1 (CDRL1) amino acid sequence of SEQ ID NO: 4, a CDRL2 of SEQ ID NO: 5, and a CDRL3 of SEQ ID NO: 6, and wherein the subject achieves at least a 20% improvement in the American College of Rheumatology core set disease index (ACR20) after the treatment.

2. The method of claim 1, wherein the antibody comprises the heavy chain variable region of the amino acid sequence of SEQ ID NO: 7, and the light chain variable region of the amino acid sequence of SEQ ID NO: 8.

3. The method of claim 1, wherein the antibody comprises the heavy chain amino acid sequence of SEQ ID NO: 9, and the light chain amino acid sequence of SEQ ID NO: 10.

4. The method of claim 1, wherein the antibody is administered at a dose of about 100 mg per administration.

5. The method of claim 4, wherein the antibody is administered once every 4 weeks (q4w)

6. The method of claim 1, wherein the ACR20 is achieved following a treatment period of about 24 weeks.

7. The method of claim 1, wherein the ACR20 is achieved following a treatment period of about 52 weeks.

8. The method of claim 1, wherein, after the treatment, the subject further achieves an improvement in a disease activity determined by at least one criteria selected from the group consisting of a 50% improvement in the American College of Rheumatology core set disease index (ACR50), a 70% improvement in the American College of Rheumatology core set disease index (ACR70), Health Assessment Questionnaire Disability Index (HAQ-DI), Investigator's Global Assessment (IGA), Disease Activity Score 28 (DAS28)C-reactive protein (CRP), resolution of enthesitis, resolution of dactylitis, Leeds enthesitis index (LEI), dactylitis assessment score, Short Form Health survey (SF-36) in the mental and physical component summary (MCS and PCS), achievement of minimal disease activity (MDA), very low disease activity (VLDA), Bath Ankylosing Spondylitis Disease Activity Index (BASDAI), GRAppa Composite score (GRACE), Psoriatic Arthritis Disease Activity Score (PASDAS), modified Composite Psoriatic Disease Activity Index (mCPDAI), Psoriatic Area and Severity Index (PAST), Dermatology Life Quality Index (DLQI), Functional Assessment of Chronic Illness Therapy (FACIT), a Patient-Reported Outcomes Measurement Information System-29 (PROMIS-29), and vdH-S score.

9. The method of claim 8, wherein the subject further achieves at least a 50% improvement in the American College of Rheumatology core set disease index (ACR50) after the treatment.

10. The method of claim 8, wherein the subject further achieves an improvement in the Health Assessment Questionnaire Disability Index (HAQ-DI) following a treatment period of at least about 24 weeks.

11. The method of claim 8, wherein the subject further achieves an improvement in Disease Activity Score 28 (DAS28)C-reactive protein (CRP) following a treatment period of at least about 24 weeks.

12. The method of claim 8, wherein the subject further achieves Investigator's Global Assessment (IGA) of 0 (clear) or 1 (minimal), or 2 or more grade reduction in the IGA, following a treatment period of at least about 24 weeks, wherein the subject has 3% or more body surface area (BSA) psoriatic involvement and an IGA score of 2 or more at the baseline before the treatment.

13. The method of claim 1, wherein the subject has had inadequate response to a standard therapy for the PsA, optionally, the subject is also administered with the standard therapy during the treatment.

14. A method of treating psoriastic arthritis in a subject in need thereof comprising subcutaneously administering to the subject about 50 mg to about 150 mg of an anti-IL-23 antibody once at week 0, once at week 4, and once every 8 weeks (q8w) thereafter, wherein the antibody comprises a heavy chain variable region and a light chain variable region, the heavy chain variable region comprising a complementarity determining region heavy chain 1 (CDRH1) amino acid sequence of SEQ ID NO: 1, a CDRH2 of SEQ ID NO: 2, and a CDRH3 of SEQ ID NO: 3; and the light chain variable region comprising a complementarity determining region light chain 1 (CDRL1) amino acid sequence of SEQ ID NO: 4, a CDRL2 of SEQ ID NO: 5, and a CDRL3 of SEQ ID NO: 6, and wherein the subject has at least one psoriatic plaque of ≥2 cm diameter or nail changes consistent with psoriasis or documented history of plaque psoriasis before the treatment, and the subject achieves at least a 20% improvement in the American College of Rheumatology core set disease index (ACR20).

15. The method of claim 14, wherein the antibody comprises the heavy chain variable region of the amino acid sequence of SEQ ID NO: 7, and the light chain variable region of the amino acid sequence of SEQ ID NO: 8.

16. The method of claim 15, wherein the antibody comprises the heavy chain amino acid sequence of SEQ ID NO: 9, and the light chain amino acid sequence of SEQ ID NO: 10.

17. The method of claim 14, wherein the antibody is administered at a dose of about 100 mg per administration.

18. The method of claim 17, wherein the ACR20 is achieved following a treatment period of about 24 weeks.

19. The method of claim 18, wherein the ACR20 is achieved following a treatment period of about 52 weeks.

20. The method of claim 19, wherein after the treatment the subject further achieves an improvement in a disease activity determined by at least one criteria selected from the group consisting of: a 50% improvement in the American College of Rheumatology core set disease index (ACR50), a 70% improvement in the American College of Rheumatology core set disease index (ACR70), Health Assessment Questionnaire Disability Index (HAQ-DI), Investigator's Global Assessment (IGA), Disease Activity Score 28 (DAS28)C-reactive protein (CRP), resolution of enthesitis, resolution of dactylitis, Leeds enthesitis index (LEI), dactylitis assessment score, Short Form Health survey (SF-36) in the mental and physical component summary (MCS and PCS), achievement of minimal disease activity (MDA), very low disease activity (VLDA), Bath Ankylosing Spondylitis Disease Activity Index (BASDAI), GRAppa Composite score (GRACE), Psoriatic Arthritis Disease Activity Score (PASDAS), modified Composite Psoriatic Disease Activity Index (mCPDAI), Psoriatic Area and Severity Index (PAST), Dermatology Life Quality Index (DLQI), Functional Assessment of Chronic Illness Therapy (FACIT), Patient-Reported Outcomes Measurement Information System-29 (PROMIS-29), and vdH-S score.

21. The method of claim 20, wherein the subject further achieves at least a 50% improvement in the American College of Rheumatology core set disease index (ACR50) after the treatment.

22. The method of claim 20, wherein the subject further achieves an improvement in the Health Assessment Questionnaire Disability Index (HAQ-DI) following a treatment period of at least about 24 weeks.

23. The method of claim 20, wherein the subject further achieves an improvement in Disease Activity Score 28 (DAS28)C-reactive protein (CRP) following a treatment period of at least about 24 weeks.

24. The method of claim 20, wherein the subject further achieves Investigator's Global Assessment (IGA) of 0 (clear) or 1 (minimal), or 2 or more grade reduction in the IGA, following a treatment period of at least about 24 weeks, wherein the subject has 3% or more body surface area (BSA) psoriatic involvement and an IGA score of 2 or more at the baseline before the treatment

25. The method of claim 1, wherein the subject has had inadequate response to a standard therapy for the PsA.

26. The method of claim 25, wherein the subject is also administered with the standard therapy during the treatment.
